SEQUENCE LISTING (68 sequences)

SEQ ID NO: 1 - protein, 18 aa, Escherichia coli
NTFYCCELCCNPACAGCY

SEQ ID NO: 2 - DNA, 3222 bp, Homo sapiens
atgaagacgttgctgttggacttggctttgtggtcactgctcttccagcccgggtggctgtcctttagttcccaggtgagtcagaactgccacaatggcagctatgaaatcagcgtcctgatgatgggcaactcagcctttgcagagcccctgaaaaacttggaagatgcggtgaatgaggggctggaaatagtgagaggacgtctgcaaaatgctggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtactttgaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcatcctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaa

SEQ ID NO: 3 - protein, 1073 aa, Homo sapiens
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 4 - protein, 6 aa, Artificial Sequence
  Description of Artificial Sequence Synthetic 6xHis tag
HHHHHH

SEQ ID NO: 5 - protein, 30 aa, Artificial Sequence
  Description of Artificial Sequence Synthetic polypeptide
ILVDLFNDQYFEDNVTAPDYMKNVLVLTLS

SEQ ID NO: 6 - protein, 25 aa, Artificial Sequence
  Description of Artificial Sequence Synthetic peptide
FAHAFRNLTFEGYDGPVTLDDWGDV

SEQ ID NO: 7 - protein, 4 aa, Artificial Sequence
  Description of Artificial Sequence Synthetic peptide
SXKX

SEQ ID NO: 8 - protein, 7 aa, Artificial Sequence
  Description of Artificial Sequence Synthetic peptide
XXSQSXX

SEQ ID NO: 9 - protein, 4 aa, Artificial Sequence
  Description of Artificial Sequence Synthetic peptide
GFLG

SEQ ID NO: 10 - DNA, 15 bp, Artificial Sequence
  Description of Artificial Sequence Synthetic oligonucleotide
ggttactactggagc

SEQ ID NO: 11 - DNA, 45 bp, Artificial Sequence
  Description of Artificial Sequence Synthetic oligonucleotide
gaaatcaatcatcgtggaaacaccaacgacaacccgtccctcaag

SEQ ID NO: 12 - DNA, 33 bp, Artificial Sequence
  Description of Artificial Sequence Synthetic oligonucleotide
gaacgtggatacacctatggtaactttgaccac

SEQ ID NO: 13 - DNA, 33 bp, Artificial Sequence
  Description of Artificial Sequence Synthetic oligonucleotide
agggccagtcagagtgttagcagaaacttagcc

SEQ ID NO: 14 - DNA, 21 bp, Artificial Sequence
  Description of Artificial Sequence Synthetic oligonucleotide
ggtgcatccaccagggccact

SEQ ID NO: 15 - DNA, 27 bp, Artificial Sequence
  Description of Artificial Sequence Synthetic oligonucleotide
cagcagtataaaacctggcctcggacg

SEQ ID NO: 16 - protein, 420 aa, Artificial Sequence
  Description of Artificial Sequence Synthetic polypeptide
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKL

SEQ ID NO: 17 - DNA, 357 bp, Artificial Sequence
  Description of Artificial Sequence Synthetic polynucleotide
caggtgcagctacagcagtggggcgcaggactgttgaagccttcggagaccctgtccctcacctgcgctgtctttggtgggtccttcagtggttactactggagctggatccgccagcccccagggaaggggctggagtggattggggaaatcaatcatcgtggaaacaccaacgacaacccgtccctcaagagtcgagtcaccatatcagtagacacgtccaagaaccagttcgccctgaagctgagttctgtgaccgccgcggacacggctgtttattactgtgcgagagaacgtggatacacctatggtaactttgaccactggggccagggaaccctggtcaccgtctcctca

SEQ ID NO: 18 - protein, 119 aa, Artificial Sequence
  Description of Artificial Sequence Synthetic polypeptide
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 19 - DNA, 321 bp, Artificial Sequence
  Description of Artificial Sequence Synthetic polynucleotide
gaaatagtgatgacgcagtctccagccaccctgtctgtgtctccaggggaaagagccaccctctcctgcagggccagtcagagtgttagcagaaacttagcctggtatcagcagaaacctggccaggctcccaggctcctcatctatggtgcatccaccagggccactggaatcccagccaggttcagtggcagtgggtctgggacagagttcactctcaccatcggcagcctgcagtctgaagattttgcagtttattactgtcagcagtataaaacctggcctcggacgttcggccaagggaccaacgtggaaatcaaa

SEQ ID NO: 20 - protein, 107 aa, Artificial Sequence
  Description of Artificial Sequence Synthetic polypeptide
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 21 - DNA, 112 bp, Artificial Sequence
  Description of Artificial Sequence Synthetic primer
ataagaatgcggccgcctcaccatgggatggagctgtatcatcctcttcttggtagcaacagctacaggtgtccactccgaaatagtgatgacgcagtctccagccaccctg

SEQ ID NO: 22 - DNA, 45 bp, Artificial Sequence
  Description of Artificial Sequence Synthetic primer
gccaccgtacgtttgatttccacgttggtcccttggccgaacgtc

SEQ ID NO: 23 - DNA, 103 bp, Artificial Sequence
  Description of Artificial Sequence Synthetic primer
ccggaattcctcaccatgggatggagctgtatcatcctcttcttggtagcaacagctacaggtgtccactcccaggtgcagctacagcagtggggcgcaggac

SEQ ID NO: 24 - DNA, 45 bp, Artificial Sequence
  Description of Artificial Sequence Synthetic primer
ggaggctgagctgacggtgaccagggttccctggccccagtggtc

SEQ ID NO: 25 - protein, 5 aa, Artificial Sequence
  Description of Artificial Sequence Synthetic peptide
GYYWS

SEQ ID NO: 26 - protein, 16 aa, Artificial Sequence
  Description of Artificial Sequence Synthetic peptide
EINHRGNTNDNPSLKS

SEQ ID NO: 27 - protein, 11 aa, Artificial Sequence
  Description of Artificial Sequence Synthetic peptide
ERGYTYGNFDH

SEQ ID NO: 28 - protein, 11 aa, Artificial Sequence
  Description of Artificial Sequence Synthetic peptide
RASQSVSRNLA

SEQ ID NO: 29 - protein, 7 aa, Artificial Sequence
  Description of Artificial Sequence Synthetic peptide
GASTRAT

SEQ ID NO: 30 - protein, 9 aa, Artificial Sequence
  Description of Artificial Sequence Synthetic peptide
QQYKTWPRT

SEQ ID NO: 31 - DNA, 1444 bp, Artificial Sequence
  Description of Artificial Sequence Synthetic polynucleotide
gaattcctcaccatgggatggagctgtatcatcctcttcttggtagcaacagctacaggtgtccactcccaggtgcagctacagcagtggggcgcaggactgttgaagccttcggagaccctgtccctcacctgcgctgtctttggtgggtctttcagtggttactactggagctggatccgccagcccccagggaaggggctggagtggattggggaaatcaatcatcgtggaaacaccaacgacaacccgtccctcaagagtcgagtcaccatatcagtagacacgtccaagaaccagttcgccctgaagctgagttctgtgaccgccgcggacacggctgtttattactgtgcgagagaacgtggatacacctatggtaactttgaccactggggccagggaaccctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatagggataacagggtaatactagag

SEQ ID NO: 32 - protein, 468 aa, Artificial Sequence
  Description of Artificial Sequence Synthetic polypeptide
MGWSCIILFLVATATGVHSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 722 bp, Artificial Sequence
  Description of Artificial Sequence Synthetic polynucleotide
gcggccgcctcaccatgggatggagctgtatcatcctcttcttggtagcaacagctacaggtgtccactccgaaatagtgatgacgcagtctccagccaccctgtctgtgtctccaggggaaagagccaccctctcctgcagggccagtcagagtgttagcagaaacttagcctggtatcagcagaaacctggccaggctcccaggctcctcatctatggtgcatccaccagggccactggaatcccagccaggttcagtggcagtgggtctgggacagagttcactctcaccatcggcagcctgcagtctgaagattttgcagtttattactgtcagcagtataaaacctggcctcggacgttcggccaagggaccaacgtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgttagtctaga

SEQ ID NO: 34 - protein, 233 aa, Artificial Sequence
  Description of Artificial Sequence Synthetic polypeptide
MGWSCIILFLVATATGVHSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 702 bp, Artificial Sequence
  Description of Artificial Sequence Synthetic polynucleotide
atgggatggagctgtatcatcctcttcttggtagcaacagctacaggtgtccactccgaaatagtgatgacgcagtctccagccaccctgtctgtgtctccaggggaaagagccaccctctcctgcagggccagtcagagtgttagcagaaacttagcctggtatcagcagaaacctggccaggctcccaggctcctcatctatggtgcatccaccagggccactggaatcccagccaggttcagtggcagtgggtctgggacagagttcactctcaccatcggcagcctgcagtctgaagattttgcagtttattactgtcagcagtataaaacctggcctcggacgttcggccaagggaccaacgtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgttag

SEQ ID NO: 36 - DNA, 1407 bp, Artificial Sequence
  Description of Artificial Sequence Synthetic polynucleotide
atgggatggagctgtatcatcctcttcttggtagcaacagctacaggtgtccactcccaggtgcagctacagcagtggggcgcaggactgttgaagccttcggagaccctgtccctcacctgcgctgtctttggtgggtctttcagtggttactactggagctggatccgccagcccccagggaaggggctggagtggattggggaaatcaatcatcgtggaaacaccaacgacaacccgtccctcaagagtcgagtcaccatatcagtagacacgtccaagaaccagttcgccctgaagctgagttctgtgaccgccgcggacacggctgtttattactgtgcgagagaacgtggatacacctatggtaactttgaccactggggccagggaaccctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 37 - DNA, 1410 bp, Artificial Sequence
  Description of Artificial Sequence Synthetic polynucleotide
atggagtttgggctgagctggctttttcttgtggctattttaaaaggtgtccagtgtgaggtgcagctgttggagtctgggggaggcttggtacagcctggggggtccctgagactctcctgtgcagcctctggattcacctttagccgctatgccatgaactgggtccgccaggctccagggaaggggctggagtgggtctcaggtattagtgggagtggtggtaggacatactacgcagactccgtgaagggccggttcaccatctccagagacaattccaagaacacactatatctgcaaatgaacagcctgagagccgaggacacggccgtatattactgtgcgaaagatcgcgatttttggagtggtccatttgactactggggccagggaaccctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 38 - DNA, 705 bp, Artificial Sequence
  Description of Artificial Sequence Synthetic polynucleotide
atgaggctccctgctcagcttctcttcctcctgctactctggctcccagataccactggagaaatagtgatgacgccgtcttcagccaccctgtctgtgtctccaggggagagagccaccctctcctgcagggccagtcagagtgttagtagaaacttagcctggtaccagcagaaacctggccaggctcccaggctcctcatctatggtgcatccaccagggccactggtatcccagccaggttcagtggcagtgggtctgggacagaattcactctcaccatcagcagcctgcagtctgaagattttgcagtttattactgtcaccagtatagtaactggatgtgcagttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgttag

SEQ ID NO: 39 - DNA, 1380 bp, Artificial Sequence
  Description of Artificial Sequence Synthetic polynucleotide
atggagactgggctgcgctggcttctcctggtcgctgtgctcaaaggtgtccagtgtcagtcagtgaaggagtccgggggaggcctcttcaagccaacggataccctgacactcacctgcaccgtctctggattctccctcagtagtcatagaatgaactgggtccgccagactccagggaaggggctggaatggatcgcaatcattactcataatagtatcacatactacgcgagctgggcgaaaagccgatccaccatcaccagaaacaccagcgagaacacggtgactctgaaaatgaccagtctgacagccgcggacacggccacttatttctgtgccagagaggatagtatggggtattattttgacttgtggggcccaggcaccctggtcaccatctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 40 - protein, 459 aa, Artificial Sequence
  Description of Artificial Sequence Synthetic polypeptide
METGLRWLLLVAVLKGVQCQSVKESGGGLFKPTDTLTLTCTVSGFSLSSHRMNWVRQTPGKGLEWIAIITHNSITYYASWAKSRSTITRNTSENTVTLKMTSLTAADTATYFCAREDSMGYYFDLWGPGTLVTISSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 41 - DNA, 711 bp, Artificial Sequence
  Description of Artificial Sequence Synthetic polynucleotide
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtcagagcattagtaactggttagcctggtatcagcagaaaccagggcagtctcccaagcccctgatctacagggcatccactctggcatctggggtctcatcgcggttcagaggcagtggatctgggacacagttcactctcaccatcagtggcgtggagtgtgccgatgctgccacttactactgtcagcagacttatactaataatcatcttgataatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcagggtgacccagggcacgacctcagtcgtccagagcttcaataggggtgactgttag

SEQ ID NO: 42 - protein, 236 aa, Artificial Sequence
  Description of Artificial Sequence Synthetic polypeptide
MDTRAPTQLLGLLLLWLPGARCAYDMTQTPASVEVAVGGTVTIKCQASQSISNWLAWYQQKPGQSPKPLIYRASTLASGVSSRFRGSGSGTQFTLTISGVECADAATYYCQQTYTNNHLDNGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCRVTQGTTSVVQSFNRGDC

SEQ ID NO: 43 - protein, 117 aa, Artificial Sequence
  Description of Artificial Sequence Synthetic polypeptide
QSVKESGGGLFKPTDTLTLTCTVSGFSLSSHRMNWVRQTPGKGLEWIAIITHNSITYYASWAKSRSTITRNTSENTVTLKMTSLTAADTATYFCAREDSMGYYFDLWGPGTLVTISS

SEQ ID NO: 44 - protein, 110 aa, Artificial Sequence
  Description of Artificial Sequence Synthetic polypeptide
AYDMTQTPASVEVAVGGTVTIKCQASQSISNWLAWYQQKPGQSPKPLIYRASTLASGVSSRFRGSGSGTQFTLTISGVECADAATYYCQQTYTNNHLDNGFGGGTEVVVK

SEQ ID NO: 45 - DNA, 351 bp, Artificial Sequence
  Description of Artificial Sequence Synthetic polynucleotide
cagtcagtgaaggagtccgggggaggcctcttcaagccaacggataccctgacactcacctgcaccgtctctggattctccctcagtagtcatagaatgaactgggtccgccagactccagggaaggggctggaatggatcgcaatcattactcataatagtatcacatactacgcgagctgggcgaaaagccgatccaccatcaccagaaacaccagcgagaacacggtgactctgaaaatgaccagtctgacagccgcggacacggccacttatttctgtgccagagaggatagtatggggtattattttgacttgtggggcccaggcaccctggtcaccatctcctca

SEQ ID NO: 46 - protein, 120 aa, Artificial Sequence
  Description of Artificial Sequence Synthetic polypeptide
EVQLLESGGGLVQPGGSLRLSCAASGFTFSRYAMNWVRQAPGKGLEWVSGISGSGGRTYYADSVKGRFTISRDNSKNTLYLQMNSLRAEDTAVYYCAKDRDFWSGPFDYWGQGTLVTVSS

SEQ ID NO: 47 - DNA, 330 bp, Artificial Sequence
  Description of Artificial Sequence Synthetic polynucleotide
gcctatgatatgacccagactccagcctctgtggaggtagctgtgggaggcacagtcaccatcaagtgccaggccagtcagagcattagtaactggttagcctggtatcagcagaaaccagggcagtctcccaagcccctgatctacagggcatccactctggcatctggggtctcatcgcggttcagaggcagtggatctgggacacagttcactctcaccatcagtggcgtggagtgtgccgatgctgccacttactactgtcagcagacttatactaataatcatcttgataatggtttcggcggagggaccgaggtggtggtcaaa

SEQ ID NO: 48 - protein, 107 aa, Artificial Sequence
  Description of Artificial Sequence Synthetic polypeptide
EIVMTPSSATLSVSPGERATLSCRASQSVSRNLAWYQQKPGQAPRLLIYGASTRATGIPARFSGSGSGTEFTLTISSLQSEDFAVYYCHQYSNWMCSFGQGTKLEIK

SEQ ID NO: 49 - protein, 5 aa, Artificial Sequence
  Description of Artificial Sequence Synthetic peptide
SHRMN

SEQ ID NO: 50 - protein, 16 aa, Artificial Sequence
  Description of Artificial Sequence Synthetic peptide
IITHNSITYYASWAKS

SEQ ID NO: 51 - protein, 10 aa, Artificial Sequence
  Description of Artificial Sequence Synthetic peptide
EDSMGYYFDL

SEQ ID NO: 52 - protein, 11 aa, Artificial Sequence
  Description of Artificial Sequence Synthetic peptide
QASQSISNWLA

SEQ ID NO: 53 - protein, 7 aa, Artificial Sequence
  Description of Artificial Sequence Synthetic peptide
RASTLAS

SEQ ID NO: 54 - protein, 12 aa, Artificial Sequence
  Description of Artificial Sequence Synthetic peptide
QQTYTNNHLDNG

SEQ ID NO: 55 - DNA, 15 bp, Artificial Sequence
  Description of Artificial Sequence Synthetic oligonucleotide
agtcatagaatgaac

SEQ ID NO: 56 - DNA, 48 bp, Artificial Sequence
  Description of Artificial Sequence Synthetic oligonucleotide
atcattactcataatagtatcacatactacgcgagctgggcgaaaagc

SEQ ID NO: 57 - DNA, 30 bp, Artificial Sequence
  Description of Artificial Sequence Synthetic oligonucleotide
gaggatagtatggggtattattttgacttg

SEQ ID NO: 58 - DNA, 33 bp, Artificial Sequence
  Description of Artificial Sequence Synthetic oligonucleotide
caggccagtcagagcattagtaactggttagcc

SEQ ID NO: 59 - DNA, 21 bp, Artificial Sequence
  Description of Artificial Sequence Synthetic oligonucleotide
agggcatccactctggcatct

SEQ ID NO: 60 - DNA, 36 bp, Artificial Sequence
  Description of Artificial Sequence Synthetic oligonucleotide
cagcagacttatactaataatcatcttgataatggt

SEQ ID NO: 61 - protein, 707 aa, Artificial Sequence [sequence omitted]

SEQ ID NO: 62 - protein, 638 aa, Artificial Sequence
  Description of Artificial Sequence Synthetic polypeptide
SQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYLEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQPKSSDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 5 aa, Artificial Sequence
  Description of Artificial Sequence Synthetic consensus sequence
XXXXX

SEQ ID NO: 64 - protein, 15 aa, Artificial Sequence
  Description of Artificial Sequence Synthetic consensus sequence
XIXXSGXXXXXXXKX

SEQ ID NO: 65 - protein, 12 aa, Artificial Sequence
  Description of Artificial Sequence Synthetic consensus sequence
XXXXXXGXXXDY

SEQ ID NO: 66 - protein, 16 aa, Artificial Sequence
  Description of Artificial Sequence Synthetic consensus sequence
XXSQSXXXXXXXXXXX

SEQ ID NO: 67 - protein, 7 aa, Artificial Sequence
  Description of Artificial Sequence Synthetic consensus sequence
XXSXRXX

SEQ ID NO: 68 - protein, 10 aa, Artificial Sequence
  Description of Artificial Sequence Synthetic consensus sequence
XQXXXXXXXX

(19) United States
(12) Patent Application Publication
(10) Pub. No.: US 2014/0356383 A1
(43) Pub. Date: Dec. 4, 2014

(54) ADMINISTRATION OF AN ANTI-GCC ANTIBODY-DRUG CONJUGATE AND A DNA DAMAGING AGENT IN THE TREATMENT OF CANCER

(71) Applicant: Ole Petter Veiby, Westborough, MA (US)

(72) Inventor: Ole Petter Veiby, Westborough, MA (US)

(21) Appl. No.: 14/192,632

(22) Filed: Feb. 27, 2014

Related U.S. Application Data
(60) Provisional application No. 61/892,854, filed on Oct. 18, 2013.
(60) Provisional application No. 61/770,802, filed on Feb. 28, 2013.

(51) Int. Cl. A61K 38/05; A61K 31/7068; A61K 33/24; A61K 31/513; A61K 47/48; A61K 31/4745

(52) U.S. Cl. CPC A61K 38/05; A61K 47/48646; A61K 31/4745; A61K 33/24; A61K 31/513; A61K 31/7068
USPC 424/179.1

(57) ABSTRACT

The present invention relates to methods for the treatment of gastrointestinal cancers. In particular, the invention provides methods for treatment of a gastrointestinal cancer by administering an immunoconjugate comprising an anti-GCC antibody molecule in combination with a DNA damaging agent.

### RELATED APPLICATIONS

The present application claims the benefit of U.S. Provisional Application Ser. No. 61/770,802, filed Feb. 28, 2013, and U.S. Provisional Application Ser. No. 61/892,854, filed Oct. 18, 2013; the entire content of these U.S. Provisional Applications is incorporated herein by this reference.

### SEQUENCE LISTING

The instant application contains a Sequence Listing which has been submitted electronically in ASCII format and is hereby incorporated by reference in its entirety. Said ASCII copy, created on Feb. 20, 2014, is named M2051-703410_SL.txt and is 75,817 bytes in size.

### FIELD OF THE INVENTION

The present invention relates to the field of oncology and provides methods for treating cancer, e.g., gastrointestinal cancer. More particularly, the invention relates to methods for treating a cancer, e.g., a gastrointestinal cancer, by administering an anti-GCC antibody molecule conjugated to monomethyl auristatin E via a cleavable linker in combination with a DNA damaging agent. The invention also provides pharmaceutical compositions and kits comprising an anti-GCC antibody molecule conjugated to monomethyl auristatin E via a cleavable linker in combination with a DNA damaging agent.

### BACKGROUND

Gastrointestinal cancers include tumors of the colon, rectum, gastric, pancreas, esophagus, anus, gallbladder, liver, and bile duct. Colorectal, gastric, and pancreatic cancers are the most common gastrointestinal cancers in the United States. Each year more than 275,000 people are diagnosed with gastrointestinal cancers and nearly 136,000 die of these diseases. The American Cancer Society estimates that gastrointestinal cancers accounted for 19% of all new cancer diagnoses and more than 24% of all cancer deaths in 2009.

Many chemotherapeutic agents have been used to treat patients with gastrointestinal cancer. However, the emergence of drug resistance has prevented successful treatment in many cases of colorectal, gastric, and pancreatic cancer. Most gastrointestinal cancer deaths results from the metastatic spread of chemotherapy-resistant (“chemoresistant”) cells to the liver and other organs and thus, metastasis remains a poor prognostic indicator. The two major forms of drug resistance are intrinsic resistance, in which previously untreated tumor cells are inherently insensitive to the chemotherapeutic agent, and acquired resistance, in which treated tumor cells become insensitive after drug exposure. To date, many research groups have studied the various mechanisms of drug resistance, hoping to overcome this major obstacle in chemotherapy. Researchers have determined that acquired drug resistance is multifactoral, in that it involves host factors and genetic and epigenetic changes, as well as numerous molecular events. The resistance itself may be due to decreased drug accumulation, alteration of intracellular drug distribution, reduced drug-target interaction, increased detoxification response, cell-cycle deregulation, increased damaged-DNA repair, and reduced apoptotic response.

Overcoming chemoresistance remains a therapeutic challenge.

Guanylyl cyclase C (GCC) is a transmembrane cell surface receptor that functions in the maintenance of intestinal fluid, electrolyte homeostasis and cell proliferation, see, e.g., Carrithers et al., Proc. Natl. Acad. Sci. USA 100:3018-3020 (2003). GCC is expressed at the mucosal cells lining the small intestine, large intestine and rectum (Carrithers et al., Dis Colon Rectum 39: 171-181 (1996)). GCC expression is maintained upon neoplastic transformation of intestinal epithelial cells, with expression in all primary and metastatic colorectal tumors (Carrithers et al., Dis Colon Rectum 39: 171-181 (1996); Buc et al. Eur J Cancer 41: 1618-1627 (2005); Carrithers et al., Gastroenterology 107: 1653-1661 (1994)), a majority of primary and metastatic gastric and esophageal tumors, and subsets of primary and metastatic pancreatic cancer.

GCC is an attractive target for the discovery of new targeted therapeutics due to its anatomically compartmentalized surface expression in a majority of gastrointestinal malignancies. US Published Patent Application No. US 2011/0110936 describes anti-GCC antibody-drug conjugates (“ADCs”; sometimes referred to herein as “immunoconjugates”) which have been shown to have single agent activity in mouse xenograft models of primary human tumor explants derived from metastatic colorectal cancer patients.

### SUMMARY

It has now been discovered that immunoconjugates having an anti-GCC antibody molecule conjugated to the potent microtubule inhibitor monomethyl auristatin E (MMAE) via a cleavable linker, e.g., a protease cleavable linker, administered in combination with a DNA damaging agent provides synergistic activity against gastrointestinal malignancies. Surprisingly, such anti-GCC immunoconjugates sensitize gastrointestinal tumors to DNA damaging agent activity, regardless of the tumor's sensitivity to such immunoconjugates when administered alone. The combined therapy synergistically reduces tumor volume and also prevents tumor regrowth over an unexpectedly prolonged period of time as compared to the activity of either agent alone, and provides an attractive treatment option for tumors that are resistance to the immunoconjugate activity as a single agent, whether such resistance is inherent or acquired.

In one aspect, the invention relates to a method for treating a cancer, e.g., a gastrointestinal cancer, by administering an immunoconjugate comprising an anti-GCC antibody molecule conjugated to the potent microtubule inhibitor monomethyl auristatin E (MMAE) via a protease cleavable linker in combination with a DNA damaging agent to a subject in need of such treatment. Each of the immunoconjugate and the DNA damaging agent are administered in an amount that is therapeutically effective when the two agents are used in combination. In certain embodiments, the cancer, e.g., the gastrointestinal cancer, to be treated by the methods provided herein is one that is resistant or refractory to the activity of an immunoconjugate comprising an anti-GCC antibody molecule conjugated to MMAE via a protease cleavable linker.

In one embodiment of the invention, the immunoconjugate administered in combination with the DNA damaging agent has the following formula I-5:

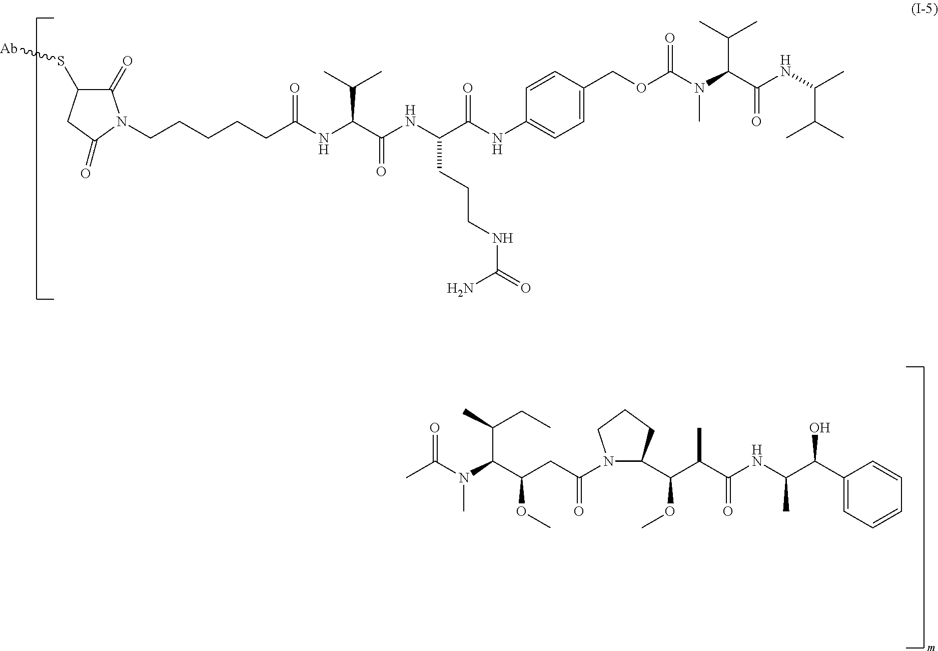

or a pharmaceutically acceptable salt thereof, wherein:
Ab is an anti-GCC antibody molecule, and

m is an integer from 1-8. In embodiments of the invention, m is an integer from 3-5. In a particular embodiment, m is about 4.

In certain embodiments, the immunoconjugate of Formula (I-5) comprises an anti-GCC antibody molecule, which includes:
a) three heavy chain complementarity determining regions (CDRs) comprising the following amino acid sequences:

VH CDR1
(SEQ ID NO: 25)
GYYWS;
VH CDR2
(SEQ ID NO: 26)
EINHRGNTNDNPSLKS;
and
VH CDR3
(SEQ ID NO: 27)
ERGYTYGNFDH;

and
b) three light chain CDRs comprising the following amino acid sequences:

VL CDR1
(SEQ ID NO: 28)
RASQSVSRNLA;
VL CDR2
(SEQ ID NO: 29)
GASTRAT;
and
VL CDR3
(SEQ ID NO: 30)
QQYKTWPRT.

In some embodiments, the anti-GCC antibody molecule is an antibody molecule that comprises a light chain variable region comprising the amino acid sequence of SEQ ID NO:20, and a heavy chain variable region comprising the amino acid sequence of SEQ ID NO:18. In some embodiments, the anti-GCC antibody molecule is an antibody molecule that comprises a light chain variable region comprising the amino acid sequence of SEQ ID NO:20, a light chain k constant region, or fragment thereof, a heavy chain variable region comprising an amino acid sequence of SEQ ID NO:18, and a heavy chain IgG1 or IgG2 constant region or fragment thereof.

In one embodiment, the anti-GCC antibody molecule is a 5F9 antibody molecule described herein.

The DNA damaging agent that is administered in combination with the immunoconjugate of Formula (I-5) can be a topoisomerase I inhibitor, a topoisomerase II inhibitor, an alkylating agent, an alkylating-like agent, an anthracycline, a DNA intercalator, a DNA minor groove alkylating agent, or an antimetabolite agent. Combination therapy including an immunoconjugate of the present invention and a DNA damaging agent may have therapeutic synergistic effects on cancer and reduce sides affects associated with these chemotherapeutic agents.

Examples of topoisomerase I inhibitors that are suitable for use in the methods of the invention include but are not limited to irinotecan, topotecan, camptothecin, SN-38, lamellarin D, and any analogs, derivatives, or metabolites thereof.

Examples of topoisomerase II inhibitors that are suitable for use in the methods of the invention include but are not limited to etoposide, teniposide, amsacrine and mitoxantrone, and any analogs, derivatives or metabolites thereof.

Alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin, semustine, uramustine), nonclassic alkylating agents (altretamine, dacarbazine, and procarbazine), and platinum compounds (carboplatin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. Other examples of alkylating agents that are suitable for use in the methods of the invention include but are not limited to mitomycin C, dibromomannitol, tetranitrate, mitozolomide, temozolomide, and any analogs, derivatives or metabolites thereof.

Examples of alkylating-like agents that are suitable for use in the methods of the invention include but are not limited to platinum compounds such as cisplatin, carboplatin, nedaplatin, oxaliplatin, satraplatin, triplatin, and any analogs, derivatives or metabolites thereof.

Examples of anthracyclines that are suitable for use in the methods of the invention include but are not limited to daunorubicin, doxorubicin, epirubicin, idarubicin, valrubicin, and any analogs, derivatives or metabolites thereof.

Examples of DNA intercalators and free radical generators suitable for use in the methods of the invention include but are not limited to bleomycin.

Examples of DNA minor groove alkylating agents suitable for use in the methods of the invention include but are not limited to duocarmycins, and any analogs, or derivatives thereof, such as those described in U.S. Pat. Nos. 5,101,038; 5,641,780; 5,187,186; 5,070,092; 5,703,080; 5,070,092; 5,641,780; 5,101,038; 5,084,468; 5,739,350; 4,978,757; 5,332,837; 4,912,227; 5,985,908; 6,060,608; 6,262,271; 6,281,354; 6,310,209; 6,486,326; and 6,548,530; in PCT Publication Nos. WO 96/10405; WO 97/32850; WO 97/45411; WO 98/52925; WO 99/19298; WO 99/29642; WO 01/83482; WO 97/12862; WO 03/022806; and WO 04/101767; and in published European application 0 537 575 A1, the contents of each of which are hereby incorporated by reference in their entireties; and pyrrolobenzodiazepene compounds (“PBDs”) such as those described in WO2000/012508, WO2011/130598, WO2011/130616, WO2005/085251, WO2010/043880, WO2012/003266, WO2000/012506, WO2005/023814, the contents of each of which are hereby incorporated by reference in their entireties.

Antimetabolite agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolite agents suitable for use in the methods of the invention include but are not limited to fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, pemetrexed and any analogs, derivatives or metabolites thereof.

In some embodiments, the gastrointestinal cancer is a GCC-expressing gastrointestinal cancer. Examples of GCC-expressing gastrointestinal cancers include but are not limited to primary or metastatic colorectal cancer, primary or metastatic gastric cancer, primary or metastatic pancreatic cancer, and primary or metastatic esophageal cancer. In certain embodiments, the gastrointestinal cancer is resistant to the immunoconjugate of Formula (I-5) when administered as a single agent.

In one particular embodiment, the invention relates to a method for treating a gastrointestinal cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with a DNA damaging agent to a subject in need of such treatment, wherein the DNA damaging agent is a topoisomerase I inhibitor, and wherein each of the immunoconjugate and the topoisomerase I inhibitor are administered in an amount that is therapeutically effective when the two agents are used in combination.

In another particular embodiment, the invention relates to a method for treating a gastrointestinal cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with a topoisomerase I inhibitor to a subject in need of such treatment, wherein the toposiomerase I inhibitor is irinotecan, and wherein each of the immunoconjugate and irinotecan are administered in an amount that is therapeutically effective when the two agents are used in combination.

In still another particular embodiment, the invention relates to a method for treating primary or metastatic colorectal cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with irinotecan to a subject in need of such treatment, wherein each of the immunoconjugate and irinotecan are administered in an amount that is therapeutically effective when the two agents are used in combination. In a particular embodiment, the immunoconjugate and irinotecan are comprised in separate formulations that are concomitantly or sequentially administered.

In still another particular embodiment, the invention relates to a method for treating a gastrointestinal cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with an alkylating-like agent to a subject in need of such treatment, wherein each of the immunoconjugate and alkylating-like agent are administered in an amount that is therapeutically effective when the two agents are used in combination. For example, the alkylating-like agent is cisplatin, carboplatin, nedaplatin, oxaliplatin, satraplatin, or triplatin.

In a particular embodiment, the invention relates to a method for treating primary or metastatic colorectal cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with cisplatin or oxaliplatin to a subject in need of such treatment, wherein each of the immunoconjugate and either cisplatin or oxaliplatin are administered in an amount that is therapeutically effective when the two agents are used in combination. In a particular embodiment, the immunoconjugate and cisplatin or oxaliplatin are comprised in separate formulations that are concomitantly or sequentially administered.

In yet another embodiment, the invention relates to a method for treating a gastrointestinal cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with an antimetabolite to a subject in need of such treatment, wherein each of the immunoconjugate and antimetabolite are administered in an amount that is therapeutically effective when the two agents are used in combination. For example, the antimetabolite is fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, or pemetrexed.

In one particular embodiment, the invention relates to a method for treating primary or metastatic colorectal cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with 5-fluorouracil to a subject in need of such treatment, wherein each of the immunoconjugate and 5-fluorouracil are administered in an amount that is therapeutically effective when the two agents are used in combination. In a particular embodiment, the immunoconjugate and 5-fluorouracil are comprised in separate formulations that are concomitantly or sequentially administered.

In another particular embodiment, the invention relates to a method for treating primary or metastatic pancreatic cancer by administering an immunoconjugate according to Formula (I-5), wherein the anti-GCC antibody molecule is an anti-GCC antibody molecule described herein, e.g., 5F9, and m is an integer from 3-5 (e.g., 4), in combination with gemcitabine to a subject in need of such treatment, wherein each of the immunoconjugate and gemcitabine are administered in an amount that is therapeutically effective when the two agents are used in combination. In a particular embodiment, the immunoconjugate and gemcitabine are comprised in separate formulations that are concomitantly or sequentially administered.

In an embodiment of the invention, the anti-GCC antibody molecule of the immunoconjugate is a monoclonal antibody, or an antigen binding fragment thereof. In another embodiment, the anti-GCC antibody molecule of the immunoconjugate is an IgG1 or IgG2 antibody. In yet another embodiment, the anti-GCC antibody molecule of the immunoconjugate comprises human or human-derived light and heavy variable region frameworks. In a particular embodiment, the anti-GCC antibody molecule of the invention is an isolated monoclonal IgG1 antibody, or antigen binding fragment thereof, that comprises human or human-derived light and heavy variable region frameworks.

In certain embodiments, the immunoconjugate and the DNA damaging agent are concomitantly administered. In other certain embodiments, the immunoconjugate and the DNA damaging agent are sequentially administered. The immunoconjugate and the DNA damaging agent can be administered as separate formulations. Alternatively, the immunoconjugate and the DNA damaging agent are co-formulated as a single dosage form of a therapeutically effective total amount of each agent.

In one embodiment, the immunoconjugate is administered once every three weeks over a certain period of time in combination with a DNA damaging agent administered once a week, twice a week or three times a week, e.g., over the same period of time.

In one embodiment, the immunoconjugate is administered once every three weeks over a certain period of time in combination with a DNA damaging agent administered once every three weeks, e.g., over the same period of time.

In one embodiment, the immunoconjugate is administered once every three weeks over a certain period of time in combination with a DNA damaging agent administered 3 days on/4 days off during each week, e.g., over the same period of time.

In one embodiment, the immunoconjugate is administered once every three weeks over a certain period of time in combination with a DNA damaging agent administered 2 days on/5 days off during each week, e.g., over the same period of time.

In another embodiment, the immunoconjugate is administered once every two weeks over a certain period of time in combination with a DNA damaging agent administered two or three times per week, e.g., over the same period of time.

In yet another embodiment, the immunoconjugate is administered once a week over a certain period of time in combination with a DNA damaging agent administered twice weekly, e.g., over the same period of time.

In still another embodiment, the immunoconjugate is administered once a week over a certain period of time in combination with a DNA damaging agent administered on day 1 and day 3 of each week, e.g., over the same period of time.

In yet another embodiment, the immunoconjugate is administered once a week over a certain period of time in combination with a DNA damaging agent administered three times weekly, e.g., over the same period of time.

In another embodiment, the immunoconjugate is administered once a week over a certain period of time in combination with a DNA damaging agent administered once during the first week, e.g., of the same period of time.

In another embodiment, the immunoconjugate is administered once a week over a certain period of time in combination with a DNA damaging agent administered once a week during the first and second weeks, e.g., of the same period of time.

In one embodiment, the immunoconjugate is administered once a week over a certain period of time in combination with a DNA damaging agent administered 3 days on/4 days off during each week, e.g., over the same period of time.

In one embodiment, the immunoconjugate is administered once a week over a certain period of time in combination with a DNA damaging agent administered 2 days on/5 days off during each week, e.g., over the same period of time.

In one embodiment, the immunoconjugate is administered once a week over a certain period of time in combination with a DNA damaging agent administered once every three weeks, e.g., over the same period of time.

In yet another embodiment, the dosing schedule of the DNA damaging agent overlaps with the dosing schedule of the immunoconjugate on at least one day per week. In other embodiments, the dosing schedule of the DNA damaging agent does not overlap with the dosing schedule of the immunoconjugate such that each agent is administered on different days of the week.

In one aspect, the invention relates to a kit comprising at least one medicament for use in treating a cancer, e.g., a gastrointestinal cancer, in a subject in need of such treatment. In one embodiment, the kit comprises a medicament comprising an immunoconjugate according to Formula (I-5), and instructions for administering the immunoconjugate in combination with a DNA damaging agent selected from a topoisomerase I inhibitor, a topoisomerase II inhibitor, an alkylating agent, an alkylating-like agent, an anthracycline, a DNA intercalator, a DNA minor groove alkylating agent, or an antimetabolite. In an embodiment, the DNA damaging agent is a DNA damaging agent described herein. In certain embodiments, the kit comprises a medicament comprising an immunoconjugate according to Formula (I-5), and instructions for administering the immunoconjugate in combination with a topoisomerase I inhibitor for treating a gastrointestinal cancer. In a particular embodiment, the topoisomerase I inhibitor for administration in combination with the immunoconjugate is irinotecan. For example, the kit includes a medicament comprising an immunoconjugate of Formula (I-5), wherein the anti-GCC antibody molecule is an anti-GCC antibody molecule described herein, e.g., 5F9, and m is an integer from 3-5 (e.g., 4), and instructions for administering the immunoconjugate in combination with irinotecan to treat a gastrointestinal cancer, e.g., colorectal cancer. In some embodiments, the instructions include a dose or dosing schedule described herein. In some embodiments, the kit further includes irinotecan.

In another certain embodiment, the kit comprises a medicament comprising an immunoconjugate according to Formula (I-5), and instructions for administering the immunoconjugate in combination with an alkylating-like agent for treating a gastrointestinal cancer. In a particular embodiment, the alkylating-like agent for administration in combination with the immunoconjugate is cisplatin or oxaliplatin. For example, the kit includes a medicament comprising an immunoconjugate of Formula (I-5), wherein the anti-GCC antibody molecule is an anti-GCC antibody molecule described herein, e.g., 5F9, and m is an integer from 3-5 (e.g., 4), and instructions for administering the immunoconjugate in combination with cisplatin or oxaliplatin to treat a gastrointestinal cancer, e.g., colorectal cancer. In some embodiments, the instructions include a dose or dosing schedule described herein. In some embodiments, the kit further includes cisplatin or oxaliplatin.

In still another certain embodiment, the kit comprises a medicament comprising an immunoconjugate according to Formula (I-5), and instructions for administering the immunoconjugate in combination with an antimetabolite agent for treating a gastrointestinal cancer. In a particular embodiment, the antimetabolite for administration in combination with the immunoconjugate is 5-fluorouracil. For example, the kit includes a medicament comprising an immunoconjugate of Formula (I-5), wherein the anti-GCC antibody molecule is an anti-GCC antibody molecule described herein, e.g., 5F9, and m is an integer from 3-5 (e.g., 4), and instructions for administering the immunoconjugate in combination with 5-fluorouracil for the treatment a gastrointestinal cancer, e.g., colorectal cancer. In some embodiments, the instructions include a dose or dosing schedule described herein. In some embodiments, the kit further includes 5-fluorouracil.

In yet another certain embodiment, the kit comprises a medicament comprising an immunoconjugate according to Formula (I-5), and instructions for administering the immunoconjugate in combination with an antimetabolite agent for treating a gastrointestinal cancer. In a particular embodiment, the antimetabolite for administration in combination with the immunoconjugate is gemcitabine. For example, the kit includes a medicament comprising an immunoconjugate of Formula (I-5), wherein the anti-GCC antibody molecule is an anti-GCC antibody molecule described herein, e.g., 5F9, and m is an integer from 3-5 (e.g., 4), and instructions for administering the immunoconjugate in combination with gemcitabine for the treatment of pancreatic cancer. In some embodiments, the instructions include a dose or dosing schedule described herein. In some embodiments, the kit further includes gemcitabine.

In some aspects, the present disclosure provides a method of treating a colorectal cancer, comprising administering to a patient in need of such treatment an immunoconjugate of Formula (I-5):

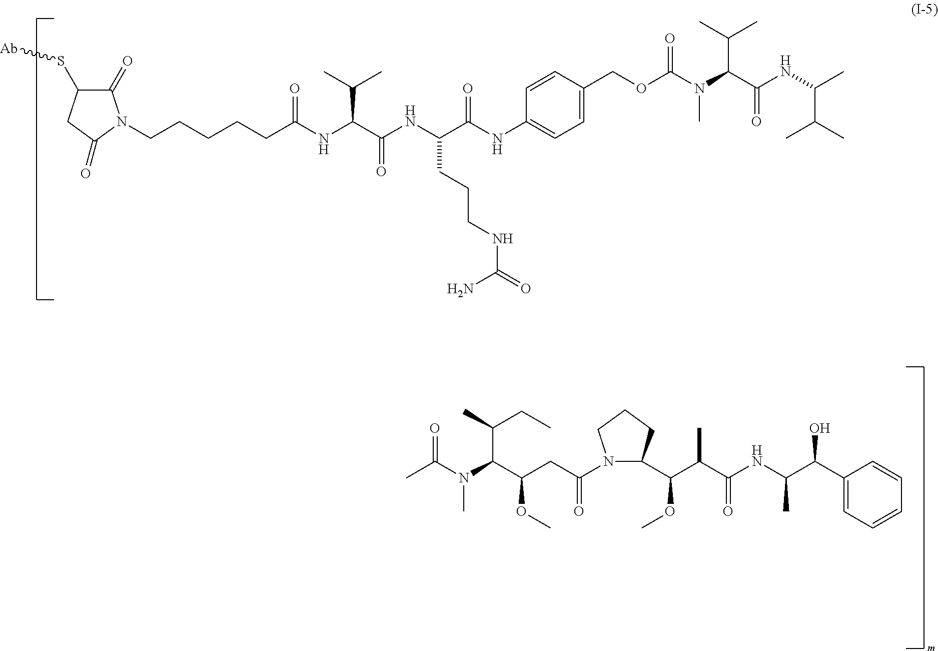

or a pharmaceutically acceptable salt thereof, wherein Ab is an anti-GCC antibody molecule comprising a) three heavy chain complementarity determining regions (CDRs) comprising the following amino acid sequences: VH CDR1 GYYWS (SEQ ID NO: 25); VH CDR2 EINHRGNTNDNPSLKS (SEQ ID NO: 26); and VH CDR3 ERGYTYGNFDH (SEQ ID NO: 27); and b) three light chain CDRs comprising the following amino acid sequences: VL CDR1 RASQSVSRNLA (SEQ ID NO: 28); VL CDR2 GASTRAT (SEQ ID NO: 29); and VL CDR3 QQYKTWPRT (SEQ ID NO: 30), e.g., 5F9, and wherein m is an integer from 1-8, e.g., 3-5; in combination with irinotecan, wherein the amounts of the immunoconjugate and the irinotecan are therapeutically effective (e.g., synergistic) when used in combination. The colorectal cancer may have strong-to-moderate or strong sensitivity to the immunoconjugate alone, or may be resistant to the immunoconjugate alone. The colorectal cancer may have a GCC antigen density that is relatively high, moderate, or low.

In some aspects, the present disclosure provides a method of treating a colorectal cancer, comprising administering to a patient in need of such treatment an immunoconjugate of Formula (I-5):

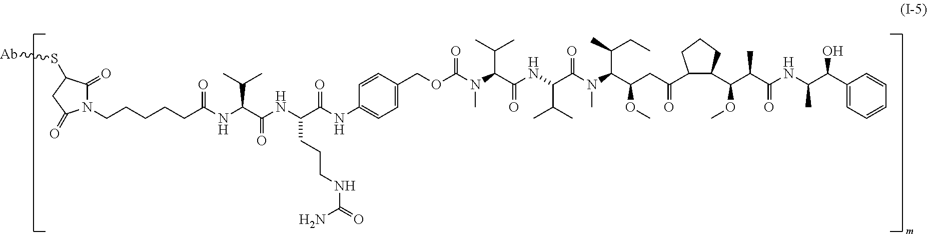

or a pharmaceutically acceptable salt thereof, wherein Ab is an anti-GCC antibody molecule comprising a) three heavy chain complementarity determining regions (CDRs) comprising the following amino acid sequences: VH CDR1 GYYWS (SEQ ID NO: 25); VH CDR2 EINHRGNTNDNPSLKS (SEQ ID NO: 26); and VH CDR3 ERGYTYGNFDH (SEQ ID NO: 27); and b) three light chain CDRs comprising the following amino acid sequences: VL CDR1 RASQSVSRNLA (SEQ ID NO: 28); VL CDR2 GASTRAT (SEQ ID NO: 29); and VL CDR3 QQYKTWPRT (SEQ ID NO: 30), e.g., 5F9, and wherein m is an integer from 1-8, e.g., 3-5; in combination with cisplatin, wherein the amounts of the immunoconjugate and the cisplatin are therapeutically effective (e.g., additive) when used in combination. The colorectal cancer may have strong sensitivity to the immunoconjugate alone. The colorectal cancer may have a relatively high GCC antigen density.

In some aspects, the present disclosure provides a method of treating a colorectal cancer, comprising administering to a patient in need of such treatment an immunoconjugate of Formula (I-5):

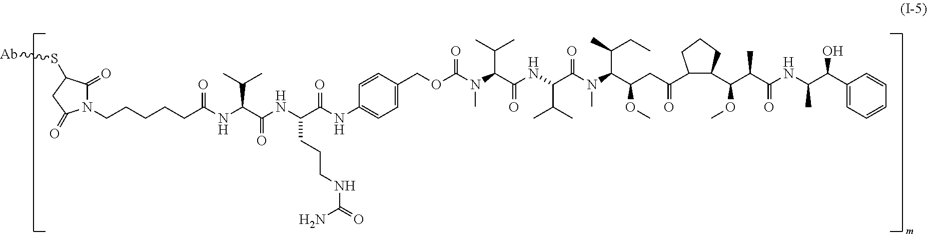

or a pharmaceutically acceptable salt thereof, wherein Ab is an anti-GCC antibody molecule comprising a) three heavy chain complementarity determining regions (CDRs) comprising the following amino acid sequences: VH CDR1 GYYWS (SEQ ID NO: 25); VH CDR2 EINHRGNTNDNPSLKS (SEQ ID NO: 26); and VH CDR3 ERGYTYGNFDH (SEQ ID NO: 27); and b) three light chain CDRs comprising the following amino acid sequences: VL CDR1 RASQSVSRNLA (SEQ ID NO: 28); VL CDR2 GASTRAT (SEQ ID NO: 29); and VL CDR3 QQYKTWPRT (SEQ ID NO: 30), e.g., 5F9, and wherein m is an integer from 1-8, e.g., 3-5; in combination with 5-fluorouracil, wherein the amounts of the immunoconjugate and the 5-fluorouracil are therapeutically effective (e.g., synergistic) when used in combination. The colorectal cancer may have strong-to-moderate sensitivity to the immunoconjugate alone. The colorectal cancer may have a low GCC antigen density.

In some aspects, the present disclosure provides a method of treating a pancreatic cancer, comprising administering to a patient in need of such treatment an immunoconjugate of Formula (I-5):

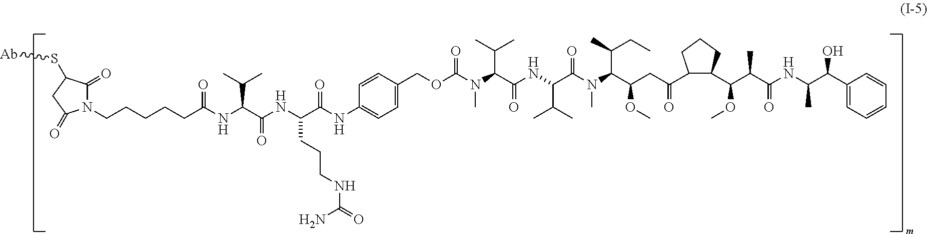

or a pharmaceutically acceptable salt thereof, wherein Ab is an anti-GCC antibody molecule comprising a) three heavy chain complementarity determining regions (CDRs) comprising the following amino acid sequences: VH CDR1 GYYWS (SEQ ID NO: 25); VH CDR2 EINHRGNTNDNPSLKS (SEQ ID NO: 26); and VH CDR3 ERGYTYGNFDH (SEQ ID NO: 27); and b) three light chain CDRs comprising the following amino acid sequences: VL CDR1 RASQSVSRNLA (SEQ ID NO: 28); VL CDR2 GASTRAT (SEQ ID NO: 29); and VL CDR3 QQYKTWPRT (SEQ ID NO: 30), e.g., 5F9, and wherein m is an integer from 1-8, e.g., 3-5; in combination with gemcitabine, wherein the amounts of the immunoconjugate and the gemcitabine are therapeutically effective (e.g., additive) when used in combination. The colorectal cancer may be sensitive to the immunoconjugate alone.

All publications, patent applications, patents and other references mentioned herein are incorporated by references in their entirety.

Advantages and features of the present invention herein disclosed will become apparent from the following description, the accompanying drawings, and the claims. Furthermore, it is to be understood that the features of the various embodiments described herein are not mutually exclusive and can exist in various combinations and permutations.

### BRIEF DESCRIPTION OF THE FIGURES

FIG. 1 depicts tumor growth in 293-GCC#2 bearing SCID mice treated with 5F9vc-MMAF, -DM1, and -DM4 on a q14d schedule.

FIG. 2 depicts lung weight of mice treated with 0.9% NaCl; 209 antibody at 40 mg/kg; or 5F9 antibody at 10 or 40 mg/kg on day 41 p.i.

FIG. 3 depicts the survival curve of CT26-hGCC tumor-bearing mice treated with 5F9 antibody.

FIG. 4 depicts ELISA binding assays to test antibody cross-reactivity of GCC orthologs.

FIGS. 5A-5E are immunohistochemistry slides depicting a wide range of GCC expression in a tumor xenograft derived from HEK293 cells transfected with GCC (FIG. 5A) and primary human tumor xenografts derived from mCRC patient samples (FIGS. 5B-5E).

FIGS. 6A-6E are graphs depicting in vivo anti-tumor activity induced by an immunoconjugate having an anti-GCC mAb conjugated to the potent microtubule inhibitor MMAE via a protease cleavable linker in different tumor xenograft models derived from mCRC patient samples having different levels of GCC antigen density. FIG. 6A depicts in vivo anti-tumor activity in a primary human tumor xenograft having moderate to high GCC expression level (PHTX-09c); FIG. 6B depicts in vivo anti-tumor activity in primary a human tumor xenograft having moderate GCC expression levels (PHTX-17c); FIGS. 6C and 6D depict in vivo anti-tumor activity at varying doses of the immunoconjugate and dosing schedules in a primary human tumor xenograft also having moderate GCC expression levels (PHTX-11c); FIG. 6E depicts in vivo anti-tumor activity in a primary human tumor xenograft having low GCC expression (PHTX-21c).

FIG. 7A depicts the immunohistochemical detection of GCC in the PHTX-11c primary human mCRC tumor xenograft model; FIG. 7B depicts the immunohistochemical detection of an immunoconjugate having an anti-GCC mAb conjugated to MMAE via a protease cleavable linker on day 7 post-administration of the immunoconjugate in the PHTX-11c model.

FIG. 8A is a graph depicting the average percent body weight change in a PHTX-09c primary human mCRC tumor xenograft model treated with an immunoconjugate having an anti-GCC mAb conjugated to MMAE via a protease cleavable linker and a DNA damaging agent (CPT-11), each agent alone and in combination; FIG. 8B is a graph depicting the anti-tumor activity induced by the immunoconjugate and the DNA damaging agent (CPT-11), each alone and in combination, in the PHTX-09c model; FIG. 8C is a graph depicting tumor re-growth in the PHTX-09c model after treatment with the immunoconjugate and the DNA damaging agent, each alone and in combination.

FIG. 9A is a graph depicting the average percent body weight change in a PHTX-21c primary human mCRC tumor xenograft model treated with an immunoconjugate having an anti-GCC mAb conjugated to MMAE via a protease cleavable linker and a DNA damaging agent (CPT-11), each agent alone and in combination; FIG. 9B is a graph depicting the anti-tumor activity induced by the immunoconjugate and the DNA damaging agent (CPT-11), each alone and in combination, in the PHTX-21c model; FIG. 9C is a graph depicting tumor re-growth in the PHTX-21c model after treatment with the immunoconjugate and the DNA damaging agent, each alone and in combination.

FIG. 10A is a graph depicting the average percent body weight change in a PHTX-17c primary human mCRC tumor xenograft model treated with an immunoconjugate having an anti-GCC mAb conjugated to MMAE via a protease cleavable linker and a DNA damaging agent (CPT-11), each agent alone and in combination; FIG. 10B is a graph depicting the anti-tumor activity induced by the immunoconjugate and the DNA damaging agent (CPT-11), each alone and in combination, in the PHTX-17c model; FIG. 10C is a graph depicting tumor re-growth in the PHTX-17c model after treatment with the immunoconjugate and the DNA damaging agent, each alone and in combination.

FIG. 11A is a graph depicting the average percent body weight change in a PHTX-11c primary human mCRC tumor xenograft model treated with an immunoconjugate having an anti-GCC mAb conjugated to MMAE via a protease cleavable linker and a DNA damaging agent (CPT-11), each agent alone and in combination; FIG. 11B is a graph depicting the anti-tumor activity induced by the immunoconjugate and the DNA damaging agent (CPT-11), each alone and in combination, in the PHTX-11c model; FIG. 11C is a graph depicting tumor re-growth in the PHTX-11c model after treatment with the immunoconjugate and the DNA damaging agent, each alone and in combination.

FIG. 12A is a graph depicting the average percent body weight change in a PHTX-09c primary human mCRC tumor xenograft model treated with an immunoconjugate having an anti-GCC mAb conjugated to MMAE via a protease cleavable linker and a DNA damaging agent (cisplatin), each agent alone and in combination; FIG. 12B is a graph depicting the anti-tumor activity induced by the immunoconjugate and the DNA damaging agent (cisplatin), each alone and in combination, in the PHTX-09c model; FIG. 12C is a graph depicting tumor regrowth in the PHTX-09c model after treatment with the immunoconjugate and the DNA damaging agent (cisplatin), each alone and in combination.

FIG. 13A is a graph depicting the average percent body weight change in a PHTX-21c primary human mCRC tumor xenograft model treated with an immunoconjugate having an anti-GCC mAb conjugated to MMAE via a protease cleavable linker and a DNA damaging agent (5-FU), each agent alone and in combination; FIG. 13B is a graph depicting the anti-tumor activity induced by the immunoconjugate and the DNA damaging agent (5-FU), each alone and in combination, in the PHTX-21c model; FIG. 13C is a graph depicting tumor re-growth in the PHTX-21c model after treatment with the immunoconjugate and the DNA damaging agent (5-FU), each alone and in combination.

FIG. 14 is a graph depicting the combined/aggregate H score distribution across samples on various pancreatic tumor microarrays screened for GCC expression.

FIG. 15A is a graph depicting the anti-tumor activity induced by 3.75 mg/kg and 7.5 mg/kg of an anti-GCC mAb conjugated to MMAE via a protease cleavable linker in a PHTX-249a primary human pancreatic tumor xenograft model (control groups treated with free-MMAE and a non-GCC targeting ADC were included in the study). FIG. 15B is a graph depicting the anti-tumor activity induced by 3.75 mg/kg and 7.5 mg/kg of an anti-GCC mAb conjugated to MMAE via a protease cleavable linker in a PHTX-215a primary human pancreatic tumor xenograft model (control groups treated with free-MMAE and a non-GCC targeting ADC were included in the study).

FIG. 16A is a bar graph depicting the comparison of anti-tumor activity induced by an anti-GCC mAb conjugated to MMAE via a protease cleavable linker and gemcitabine, each as a single agent alone and in combination at varying concentrations and dosing schedules; FIG. 16B is a graph depicting the anti-tumor activity induced by an anti-GCC mAb conjugated to MMAE via a protease cleavable linker and gemcitabine, each as a single agent alone and in combination in a PHTX-249a primary human pancreatic tumor xenograft model; FIG. 16C is a graph depicting the anti-tumor activity induced by an anti-GCC mAb conjugated to MMAE via a protease cleavable linker and gemcitabine, each as a single agent alone and in combination in a PHTX-215a primary human pancreatic tumor xenograft model.

### DETAILED DESCRIPTION

The present invention provides new combination therapies for the treatment of cancer, e.g., gastrointestinal cancers. In particular, the present invention provides a method to treat a patient suffering from a gastrointestinal cancer comprising administering to said patient an immunoconjugate that includes an anti-GCC antibody molecule conjugated to MMAE via a protease cleavable linker in combination with a DNA damaging agent, wherein each agent is used in a therapeutically effective total amount. While such immunoconjugates and DNA damaging agents may each prove effective as a single agent in treating certain gastrointestinal cancer types and a certain number of patients, it has been surprisingly discovered that combined therapy with an anti-GCC immunoconjugate of the invention and a DNA damaging agent offers benefits not achieved with either agent individually. The invention also provides pharmaceutical compositions and kits that include an anti-GCC immunoconjugate of the invention and a DNA damaging agent.

As described above, chemoresistance is a well recognized problem in the treatment of gastrointestinal cancer. In particular, microtubule disrupting agents have had limited success in the treatment of colorectal cancer due to inherent or acquired chemoresistance to such agents. MMAE is a potent microtubule inhibitor. The present inventors have discovered that, as with other microtubule disrupting agents, certain colorectal tumor types appear are resistant to the microtubule inhibiting activity of MMAE when used in an immunoconjugate designed to specifically target colorectal cancer cells by targeting GCC. Suprisingly, the present inventors have discovered that the same anti-GCC immunoconjugate having little to no activity when used as a single agent sensitizes MMAE refractory colorectal tumors to DNA damaging agent activity. The combination of an anti-GCC immunoconjugate and DNA damaging agent is shown herein to have improved anti-tumor activity as compared to the activity of either agent alone in different tumor xenograft models that exhibit varying levels of sensitivity to the immunoconjugate as a single agent, despite similar levels of GCC expression in the different models.

### Guanylyl Cyclase C

Guanylyl cyclase C (GCC) (also known as STAR, ST Receptor, GUC2C, and GUCY2C) is a transmembrane cell surface receptor that functions in the maintenance of intestinal fluid, electrolyte homeostasis and cell proliferation (Carrithers et al., Proc Natl Acad Sci USA 100: 3018-3020 (2003); Mann et al., Biochem Biophys Res Commun 239: 463-466 (1997); Pitari et al., Proc Natl Acad Sci USA 100: 2695-2699 (2003)); GenBank Accession No. NM_{—}004963, each of which is incorporated herein by reference). This function is mediated through binding of guanylin (Wiegand et al. FEBS Lett. 311:150-154 (1992)). GCC also is a receptor for heat-stable enterotoxin (ST, e.g., having an amino acid sequence of NTFYCCELCCNPACAGCY, SEQ ID NO:1) which is a peptide produced by E. coli, as well as other infectious organisms (Rao, M. C. Ciba Found. Symp. 112:74-93 (1985); Knoop F. C. and Owens, M. J. Pharmacol. Toxicol. Methods 28:67-72 (1992)). Binding of ST to GCC activates a signal cascade that results in enteric disease, e.g., diarrhea.

Nucleotide sequence for human GCC (GenBank Accession No. NM_{—}004963):

(SEQ ID NO: 2)

| | |
|---|---|
| 1 | atgaagacgt tgctgttgga cttggctttg tggtcactgc |
| | tcttccagcc cgggtggctg |
| 61 | tcctttagtt cccaggtgag tcagaactgc cacaatggca |
| | gctatgaaat cagcgtcctg |
| 121 | atgatgggca actcagcctt tgcagagccc ctgaaaaact |
| | tggaagatgc ggtgaatgag |
| 181 | gggctggaaa tagtgagagg acgtctgcaa aatgctggcc |
| | taaatgtgac tgtgaacgct |
| 241 | actttcatgt attcggatgg tctgattcat aactcaggcg |
| | actgccggag tagcacctgt |
| 301 | gaaggcctcg acctactcag gaaaatttca aatgcacaac |
| | ggatgggctg tgtcctcata |
| 361 | gggccctcat gtacatactc caccttccag atgtaccttg |
| | acacagaatt gagctacccc |
| 421 | atgatctcag ctggaagttt tggattgtca tgtgactata |
| | aagaaacctt aaccaggctg |
| 481 | atgtctccag ctagaaagtt gatgtacttc ttggttaact |
| | tttggaaaac caacgatctg |
| 541 | cccttcaaaa cttattcctg gagcacttcg tatgtttaca |
| | agaatggtac agaaactgag |
| 601 | gactgtttct ggtaccttaa tgctctggag gctagcgttt |
| | cctatttctc ccacgaactc |
| 661 | ggctttaagg tggtgttaag acaagataag gagtttcagg |
| | atatcttaat ggaccacaac |
| 721 | aggaaaagca atgtgattat tatgtgtggt ggtccagagt |
| | tcctctacaa gctgaagggt |
| 781 | gaccgagcag tggctgaaga cattgtcatt attctagtgg |
| | atcttttcaa tgaccagtac |
| 841 | tttgaggaca atgtcacagc ccctgactat atgaaaaatg |
| | tccttgttct gacgctgtct |
| 901 | cctgggaatt cccttctaaa tagctctttc tccaggaatc |
| | tatcaccaac aaaacgagac |
| 961 | tttgctcttg cctatttgaa tggaatcctg ctctttggac |
| | atatgctgaa gatatttctt |
| 1021 | gaaaatggag aaaatattac cacccccaaa tttgctcatg |
| | ctttcaggaa tctcactttt |
| 1081 | gaagggtatg acggtccagt gaccttggat gactgggggg |
| | atgttgacag taccatggtg |
| 1141 | cttctgtata cctctgtgga caccaagaaa tacaaggttc |
| | ttttgaccta tgatacccac |
| 1201 | gtaaataaga cctatcctgt ggatatgagc cccacattca |
| | cttggaagaa ctctaaactt |
| 1261 | cctaatgata ttacaggccg gggccctcag atcctgatga |
| | ttgcagtctt caccctcact |
| 1321 | ggagctgtgg tgctgctcct gctcgtcgct ctcctgatgc |
| | tcagaaaata tagaaaagat |
| 1381 | tatgaacttc gtcagaaaaa atggtcccac attcctcctg |
| | aaaatatctt tcctctggag |
| 1441 | accaatgaga ccaatcatgt tagcctcaag atcgatgatg |
| | acaaaagacg agatacaatc |
| 1501 | cagagactac gacagtgcaa atacgacaaa aagcgagtga |
| | ttctcaaaga tctcaagcac |
| 1561 | aatgatggta atttcactga aaaacagaag atagaattga |
| | acaagttgct tcagattgac |
| 1621 | tattacaacc tgaccaagtt ctacggcaca gtgaaacttg |
| | ataccatgat cttcggggtg |
| 1681 | atagaatact gtgagagagg atccctccgg gaagttttaa |
| | atgacacaat ttcctaccct |
| 1741 | gatggcacat tcatggattg ggagtttaag atctctgtct |
| | tgtatgacat tgctaaggga |
| 1801 | atgtcatatc tgcactccag taagacagaa gtccatggtc |
| | gtctgaaatc taccaactgc |
| 1861 | gtagtggaca gtagaatggt ggtgaagatc actgattttg |
| | gctgcaattc cattttacct |
| 1921 | ccaaaaaagg acctgtggac agctccagag cacctccgcc |
| | aagccaacat ctctcagaaa |
| 1981 | ggagatgtgt acagctatgg gatcatcgca caggagatca |
| | tcctgcggaa agaaaccttc |
| 2041 | tacactttga gctgtcggga ccggaatgag aagattttca |
| | gagtggaaaa ttccaatgga |
| 2101 | atgaaaccct tccgcccaga tttattcttg gaaacagcag |
| | aggaaaaaga gctagaagtg |
| 2161 | tacctacttg taaaaaactg ttgggaggaa gatccagaaa |
| | agagaccaga tttcaaaaaa |
| 2221 | attgagacta cacttgccaa gatatttgga ctttttcatg |
| | accaaaaaaa tgaaagctat |
| 2281 | atggatacct tgatccgacg tctacagcta tattctcgaa |
| | acctggaaca tctggtagag |
| 2341 | gaaaggacac agctgtacaa ggcagagagg gacagggctg |
| | acagacttaa ctttatgttg |
| 2401 | cttccaaggc tagtggtaaa gtctctgaag gagaaaggct |
| | ttgtggagcc ggaactatat |
| 2461 | gaggaagtta caatctactt cagtgacatt gtaggtttca |
| | ctactatctg caaatacagc |
| 2521 | acccccatgg aagtggtgga catgcttaat gacatctata |
| | agagttttga ccacattgtt |
| 2581 | gatcatcatg atgtctacaa ggtggaaacc atcggtgatg |
| | cgtacatggt ggctagtggt |
| 2641 | ttgcctaaga gaaatggcaa tcggcatgca atagacattg |
| | ccaagatggc cttggaaatc |
| 2701 | ctcagcttca tggggacctt tgagctggag catcttcctg |
| | gcctcccaat atggattcgc |
| 2761 | attggagttc actctggtcc ctgtgctgct ggagttgtgg |
| | gaatcaagat gcctcgttat |
| 2821 | tgtctatttg gagatacggt caacacagcc tctaggatgg |
| | aatccactgg cctccctttg |
| 2881 | agaattcacg tgagtggctc caccatagcc atcctgaaga |
| | gaactgagtg ccagttcctt |
| 2941 | tatgaagtga gaggagaaac atacttaaag ggaagaggaa |
| | atgagactac ctactggctg |
| 3001 | actgggatga aggaccagaa attcaacctg ccaacccctc |
| | ctactgtgga gaatcaacag |
| 3061 | cgtttgcaag cagaattttc agacatgatt gccaactctt |
| | tacagaaaag acaggcagca |
| 3121 | gggataagaa gccaaaaacc cagacgggta gccagctata |
| | aaaaaggcac tctggaatac |
| 3181 | ttgcagctga ataccacaga caaggagagc acctattttt |
| | aa |

Amino acid sequence for human GCC (GenPept Accession No. NP_{—}004954):

(SEQ ID NO: 3)

| | |
|---|---|
| 1 | mktllldlal wsllfqpgwl sfssqvsqnc hngsyeisvl |
| | mmgnsafaep lknledavne |
| 61 | gleivrgrlq naglnvtvna tfmysdglih nsgdcrsstc |
| | egldllrkis naqrmgcvli |
| 121 | gpsctystfq myldtelsyp misagsfgls cdyketltrl |
| | msparklmyf lvnfwktndl |
| 181 | pfktyswsts yvykngtete dcfwylnale asvsyfshel |
| | gfkvvlrqdk efqdilmdhn |
| 241 | rksnviimcg gpeflyklkg dravaedivi ilvdlfndqy |
| | fednvtapdy mknvlvltls |
| 301 | pgnsllnssf srnlsptkrd falaylngil lfghmlkifl |
| | engenittpk fahafrnltf |
| 361 | egydgpvtld dwgdvdstmv llytsvdtkk ykvlltydth |
| | vnktypvdms ptftwknskl |
| 421 | pnditgrgpq ilmiavftlt gavvllllva llmlrkyrkd |
| | yelrqkkwsh ippenifple |
| 481 | tnetnhvslk idddkrrdti qrlrqckydk krvilkdlkh |
| | ndgnftekqk ielnkllqid |
| 541 | yynltkfygt vkldtmifgv ieycergslr evlndtisyp |
| | dgtfmdwefk isvlydiakg |
| 601 | msylhsskte vhgrlkstnc vvdsrmvvki tdfgcnsilp |
| | pkkdlwtape hlrqanisqk |
| 661 | gdvysygiia qeiilrketf ytlscrdrne kifrvensng |
| | mkpfrpdlfl etaeekelev |
| 721 | yllvkncwee dpekrpdfkk iettlakifg lfhdqknesy |
| | mdtlirrlql ysrnlehlve |
| 781 | ertqlykaer dradrlnfml lprlvvkslk ekgfvepely |
| | eevtiyfsdi vgfttickys |
| 841 | tpmevvdmln diyksfdhiv dhhdvykvet igdaymvasg |
| | lpkrngnrha idiakmalei |
| 901 | lsfmgtfele hlpglpiwir igvhsgpcaa gvvgikmpry |
| | clfgdtvnta srmestglpl |
| 961 | rihvsgstia ilkrtecqfl yevrgetylk grgnettywl |
| | tgmkdqkfnl ptpptvenqq |
| 1021 | rlqaefsdmi anslqkrqaa girsqkprrv asykkgtley |
| | lqlnttdkes tyf |

The GCC protein has some generally accepted domains each of which contributes a separable function to the GCC molecule. The portions of GCC include a signal sequence (for directing the protein to the cell surface) from amino acid residue 1 to about residue 23, or residue 1 to about residue 21 of SEQ ID NO:3 (excised for maturation to yield functional mature protein from about amino acid residues 22 or 24 to 1073 of SEQ ID NO:3), an extracellular domain for ligand, e.g., guanylin or ST, binding from about amino acid residue 24 to about residue 420, or about residue 54 to about residue 384 of SEQ ID NO:3, a transmembrane domain from about amino acid residue 431 to about residue 454, or about residue 436 to about residue 452 of SEQ ID NO:3, a kinase homology domain, predicted to have tyrosine kinase activity from about amino acid residue 489 to about residue 749, or about residue 508 to about residue 745 of SEQ ID NO:3 and a guanylyl cyclase catalytic domain from about residue 750 to about residue 1007, or about residue 816 to about residue 1002 of SEQ ID NO:3. Preferably, the anti-GCC antibody molecule binds to the extracellular domain of GCC.

In some embodiments, the anti-GCC antibody molecule can bind human GCC. In some embodiments, an anti-GCC antibody molecule of the invention can inhibit the binding of a ligand, e.g., guanylin or heat-stable enterotoxin to GCC. In other embodiments, an anti-GCC antibody molecule of the invention does not inhibit the binding of a ligand, e.g., guanylin or heat-stable enterotoxin to GCC. Definitions and Methods

Unless otherwise defined herein, scientific and technical terms used in connection with the present invention have the meanings that are commonly understood by those of ordinary skill in the art. Generally, nomenclature utilized in connection with, and techniques of, cell and tissue culture, molecular biology, and protein and oligo- or polynucleotide chemistry and hybridization described herein are those known in the art. GenBank or GenPept accession numbers and useful nucleic acid and peptide sequences can be found at the website maintained by the National Center for Biotechnological Information, Bethesda Md. Standard techniques are used for recombinant DNA, oligonucleotide synthesis, and tissue culture and transformation and transfection (e.g., electroporation, lipofection). Enzymatic reactions and purification techniques are performed according to manufacturer's specifications or as commonly accomplished in the art or as described herein. The foregoing techniques and procedures are generally performed according to methods known in the art, e.g., as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2000)) or see generally, Harlow, E. and Lane, D. (1988) Antibodies: A Laboratory Manual, Cold Spring Harbor Laboratory Press, Cold Spring Harbor, N.Y. The nomenclatures utilized in connection with, and the laboratory procedures and techniques of, analytical chemistry, synthetic organic chemistry, and medicinal and pharmaceutical chemistry described herein are known in the art. Standard techniques are used for chemical syntheses, chemical analyses, pharmaceutical preparation, formulation, and delivery, and treatment of patients. Furthermore, unless otherwise required by context, singular terms shall include pluralities and plural terms shall include the singular.

As used herein, the term “antibody molecule” refers to an antibody, antibody peptide(s) or immunoglobulin, or an antigen binding fragment of any of the foregoing, e.g., of an antibody. Antibody molecules include single chain antibody molecules, e.g., scFv, see. e.g., Bird et al. (1988) Science 242:423-426; and Huston et al. (1988) Proc. Natl. Acad. Sci. USA 85:5879-5883), and single domain antibody molecules, see, e.g., WO9404678. Although not within the term “antibody molecules,” the invention also includes “antibody analog(s),” other non-antibody molecule protein-based scaffolds, e.g., fusion proteins and/or immunoconjugates that use CDRs to provide specific antigen binding.

An “anti-GCC antibody molecule” refers to an antibody molecule (i.e., an antibody, antibody peptide, immunoglobulin or antigen-binding fragment of any of the foregoing) which interacts with or recognizes, e.g., binds (e.g., binds specifically) to GCC, e.g., human GCC. An exemplary anti-GCC antibody molecule is summarized in Tables 1 and 2. An exemplary anti-GCC antibody comprises the amino acid sequence of the antibody of Table 1, and may be made in any suitable cell line, e.g., a mammalian cell line, e.g., a human cell line, a NSO cell line or a CHO cell line. An exemplary anti-GCC antibody may comprise the variable regions listed in Table 2. Exemplary anti-GCC antibodies may also comprise the CDRs listed in Table 5.

As used herein, the term “antibody,” “antibody peptide(s)” or “immunoglobulin” refers to single chain, two-chain, and multi-chain proteins and glycoproteins. The term antibody includes polyclonal, monoclonal, chimeric, CDR-grafted and human or humanized antibodies, all of which are discussed in more detail elsewhere herein. Also included within the term are camelid antibodies, see, e.g., US2005/0037421, and nanobodies, e.g., IgNARs (shark antibodies), see, e.g., WO03/014161. The term “antibody” also includes synthetic and genetically engineered variants.

As used herein, the term “antibody fragment” or “antigen binding fragment” of an antibody refers, e.g., to Fab, Fab′, F(ab′)₂, and Fv fragments, single chain antibodies, functional heavy chain antibodies (nanobodies), as well as any portion of an antibody having specificity toward at least one desired epitope, that competes with the intact antibody for specific binding (e.g., a fragment having sufficient CDR sequences and having sufficient framework sequences so as to bind specifically to an epitope). E.g., an antigen binding fragment can compete for binding to an epitope which binds the antibody from which the fragment was derived. Derived, as used in this and similar contexts, does not imply any particular method or process of derivation, but can refer merely to sequence similarity. Antigen binding fragments can be produced by recombinant techniques, or by enzymatic or chemical cleavage of an intact antibody. The term, antigen binding fragment, when used with a single chain, e.g., a heavy chain, of an antibody having a light and heavy chain means that the fragment of the chain is sufficient such that when paired with a complete variable region of the other chain, e.g., the light chain, it will allow binding of at least 25, 50, 75, 85 or 90% of that seen with the whole heavy and light variable region.)

The term, “antigen binding constellation of CDRs” or “a number of CDRs sufficient to allow binding” (and similar language), as used herein, refers to sufficient CDRs of a chain, e.g., the heavy chain, such that when placed in a framework and paired with a complete variable region of the other chain, or with a portion of the other chain's variable region of similar length and having the same number of CDRs, e.g., the light chain, will allow binding, e.g., of at least 25, 50, 75, 85 or 90% of that seen with the whole heavy and light variable region.

As used herein, the term “human antibody” includes an antibody that possesses a sequence that is derived from a human germ-line immunoglobulin sequence, such as an antibody derived from transgenic mice having human immunoglobulin genes (e.g., XENOMOUSE™ genetically engineered mice (Abgenix, Fremont, Calif.)), human phage display libraries, human myeloma cells, or human B cells.

As used herein, the term “humanized antibody” refers to an antibody that is derived from a non-human antibody e.g., rodent (e.g., murine) that retains or substantially retains the antigen-binding properties of the parent antibody but is less immunogenic in humans. Humanized as used herein is intended to include deimmunized antibodies. Typically humanized antibodies include non-human CDRs and human or human derived framework and constant regions.

The term “modified” antibody, as used herein, refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse, sheep or goat) that is transgenic for human immunoglobulin genes or antibodies prepared, expressed, created or isolated by any other means that involves splicing of human immunoglobulin gene sequences to other DNA sequences. Such modified antibodies include humanized, CDR grafted (e.g., an antibody having CDRs from a first antibody and a framework region from a different source, e.g., a second antibody or a consensus framework), chimeric, in vitro generated (e.g., by phage display) antibodies, and may optionally include variable or constant regions derived from human germline immunoglobulin sequences or human immunoglobulin genes or antibodies which have been prepared, expressed, created or isolated by any means that involves splicing of human immunoglobulin gene sequences to alternative immunoglobulin sequences. In embodiments a modified antibody molecule includes an antibody molecule having a sequence change from a reference antibody.

The term “monospecific antibody” refers to an antibody or antibody preparation that displays a single binding specificity and affinity for a particular epitope. This term includes a “monoclonal antibody” or “monoclonal antibody composition.”

The term “bispecific antibody” or “bifunctional antibody” refers to an antibody that displays dual binding specificity for two epitopes, where each binding site differs and recognizes a different epitope.

The terms “non-conjugated antibody” and “naked antibody” are used interchangeably to refer to an antibody molecule that is not conjugated to a non-antibody moiety, e.g., a therapeutic agent or a label.

The terms “immunoconjugate”, “antibody conjugate”, “antibody drug conjugate”, and “ADC” are used interchangeably and refer to an antibody molecule that is conjugated to a non-antibody moiety, e.g., a therapeutic agent or a label.

The term “agent” is used herein to denote a chemical compound, a mixture of chemical compounds, a biological macromolecule, or an extract made from biological materials. The term “therapeutic agent” refers to an agent that has biological activity.

The term “anti-cancer agent” or “chemotherapeutic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis or angiogenesis is frequently a property of anti-cancer or chemotherapeutic agents. A chemotherapeutic agent may be a cytotoxic or cytostatic agent. The term “cytostatic agent” refers to an agent which inhibits or suppresses cell growth and/or multiplication of cells.

“Cytotoxic agents” refer to compounds which cause cell death primarily by interfering directly with the cell's functioning, including, but not limited to, alkylating agents, tumor necrosis factor inhibitors, intercalators, microtubule inhibitors, kinase inhibitors, proteasome inhibitors and topoisomerase inhibitors. A “toxic payload” as used herein refers to a sufficient amount of cytotoxic agent which, when delivered to a cell results in cell death. Delivery of a toxic payload may be accomplished by administration of a sufficient amount of immunoconjugate comprising an antibody or antigen binding fragment of the invention and a cytotoxic agent. Delivery of a toxic payload may also be accomplished by administration of a sufficient amount of an immunoconjugate comprising a cytotoxic agent, wherein the immunoconjugate comprises a secondary antibody or antigen binding fragment thereof which recognizes and binds an antibody or antigen binding fragment of the invention.

As used herein the phrase, a sequence “derived from” or “specific for a designated sequence” refers to a sequence that comprises a contiguous sequence of approximately at least 6 nucleotides or at least 2 amino acids, at least about 9 nucleotides or at least 3 amino acids, at least about 10-12 nucleotides or 4 amino acids, or at least about 15-21 nucleotides or 5-7 amino acids corresponding, i.e., identical or complementary to, e.g., a contiguous region of the designated sequence. In certain embodiments, the sequence comprises all of a designated nucleotide or amino acid sequence. The sequence may be complementary (in the case of a polynucleotide sequence) or identical to a sequence region that is unique to a particular sequence as determined by techniques known in the art. Regions from which sequences may be derived, include but are not limited to, regions encoding specific epitopes, regions encoding CDRs, regions encoding framework sequences, regions encoding constant domain regions, regions encoding variable domain regions, as well as non-translated and/or non-transcribed regions. The derived sequence will not necessarily be derived physically from the sequence of interest under study, but may be generated in any manner, including, but not limited to, chemical synthesis, replication, reverse transcription or transcription, that is based on the information provided by the sequence of bases in the region(s) from which the polynucleotide is derived. As such, it may represent either a sense or an antisense orientation of the original polynucleotide. In addition, combinations of regions corresponding to that of the designated sequence may be modified or combined in ways known in the art to be consistent with the intended use. For example, a sequence may comprise two or more contiguous sequences which each comprise part of a designated sequence, and are interrupted with a region which is not identical to the designated sequence but is intended to represent a sequence derived from the designated sequence. With regard to antibody molecules, “derived therefrom” includes an antibody molecule which is functionally or structurally related to a comparison antibody, e.g., “derived therefrom” includes an antibody molecule having similar or substantially the same sequence or structure, e.g., having the same or similar CDRs, framework or variable regions. “Derived therefrom” for an antibody also includes residues, e.g., one or more, e.g., 2, 3, 4, 5, 6 or more residues, which may or may not be contiguous, but are defined or identified according to a numbering scheme or homology to general antibody structure or three-dimensional proximity, i.e., within a CDR or a framework region, of a comparison sequence. The term “derived therefrom” is not limited to physically derived therefrom but includes generation by any manner, e.g., by use of sequence information from a comparison antibody to design another antibody.

As used herein, the phrase “encoded by” refers to a nucleic acid sequence that codes for a polypeptide sequence, wherein the polypeptide sequence or a portion thereof contains an amino acid sequence of at least 3 to 5 amino acids, at least 8 to 10 amino acids, or at least 15 to 20 amino acids from a polypeptide encoded by the nucleic acid sequence.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 30%, 40%, or 50%, at least 60%, or at least 70%, 80%, 90%, 95%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. The percent homology between two amino acid sequences can be determined using any method known in the art. For example, the Needleman and Wunsch, J. Mol. Biol. 48:444-453 (1970), algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. The percent homology between two nucleotide sequences can also be determined using the GAP program in the GCG software package (Accelerys, Inc. San Diego, Calif.), using an NWSgapdna.CMP matrix and a gap weight of 40, 50, 60, 70, or 80 and a length weight of 1, 2, 3, 4, 5, or 6. An exemplary set of parameters for determination of homology are a Blossum 62 scoring matrix with a gap penalty of 12, a gap extend penalty of 4, and a frameshift gap penalty of 5.

As used herein, the term “hybridizes under stringent conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and 4) very high stringency hybridization conditions are 0.5M sodium phosphate, 7% SDS at 65° C., followed by one or more washes at 0.2×SSC, 1% SDS at 65° C. Very high stringency conditions (4) are often the preferred conditions and the ones that should be used unless otherwise specified.

It is understood that the antibodies and antigen binding fragment thereof of the invention may have additional conservative or non-essential amino acid substitutions, which do not have a substantial effect on the polypeptide functions. Whether or not a particular substitution will be tolerated, i.e., will not adversely affect desired biological properties, such as binding activity, can be determined as described in Bowie, J U et al. Science 247:1306-1310 (1990) or Padlan et al. FASEB J. 9:133-139 (1995). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the binding agent, e.g., the antibody, without abolishing or, without substantially altering a biological activity, whereas an “essential” amino acid residue results in such a change. In an antibody, an essential amino acid residue can be a specificity determining residue (SDR).

As used herein, the term “isolated” refers to material that is removed from its original environment (e.g., the natural environment if it is naturally occurring). For example, a naturally occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, e.g., a mixture, solution or suspension or comprising an isolated cell or a cultured cell which comprises the polynucleotide or polypeptide, and still be isolated in that the vector or composition is not part of its natural environment.

As used herein, the term “purified product” refers to a preparation of the product which has been isolated from the cellular constituents with which the product is normally associated and/or from other types of cells that may be present in the sample of interest.

As used herein, the term “epitope” refers to a protein determinate capable of binding specifically to an antibody. Epitopic determinants usually consist of chemically active surface groupings of molecules such as amino acids or sugar side chains and usually have specific three dimensional structural characteristics, as well as specific charge characteristics. Some epitopes are linear epitopes while others are conformational epitopes. A linear epitope is an epitope wherein a contiguous amino acid primary sequence comprises the epitope recognized. A linear epitope typically includes at least 3, and more usually, at least 5, for example, about 8 to about 10 contiguous amino acids. A conformational epitope can result from at least two situations, such as: a) a linear sequence which is only exposed to antibody binding in certain protein conformations, e.g., dependent on ligand binding, or dependent on modification (e.g., phosphorylation) by signaling molecules; or b) a combination of structural features from more than one part of the protein, or in multisubunit proteins, from more than one subunit, wherein the features are in sufficiently close proximity in 3-dimensional space to participate in binding.

As used herein, “isotype” refers to the antibody class (e.g., IgM or IgG1) that is encoded by heavy chain constant region genes.

As used herein, the terms “detectable agent,” “label” or “labeled” are used to refer to incorporation of a detectable marker on a polypeptide or glycoprotein. Various methods of labeling polypeptides and glycoproteins are known in the art and may be used. Examples of labels for polypeptides include, but are not limited to, the following: radioisotopes or radionuclides (e.g., indium (¹¹¹In), iodine (¹³¹I or ¹²⁵I), yttrium (⁹⁰Y), lutetium (¹⁷⁷Lu), actinium (²²⁵Ac), bismuth (²¹²Bi or ²¹³Bi), sulfur (³⁵S), carbon (¹⁴C), tritium (³H), rhodium (¹⁸⁸Rh), technetium (⁹⁹ mTc), praseodymium, or phosphorous (³²P) or a positron-emitting radionuclide, e.g., carbon 11 (¹¹C), potassium-40 (⁴⁰K), nitrogen-13 (¹³N), oxygen-15 (¹⁵O), fluorine-18 (¹⁸F), and iodine-121 (¹²¹I)), fluorescent labels (e.g., FITC, rhodamine, lanthanide phosphors), enzymatic labels (e.g., horseradish peroxidase, beta-galactosidase, luciferase, alkaline phosphatase), chemiluminescent, biotinyl groups (which can be detected by a marked avidin, e.g., a molecule containing a streptavidin moiety and a fluorescent marker or an enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

As used herein, “specific binding,” “bind(s) specifically” or “binding specificity” means, for an anti-GCC antibody molecule, that the antibody molecule binds to GCC, e.g., human GCC protein, with greater affinity than it does to a non-GCC protein, e.g., BSA. Typically an anti-GCC molecule will have a K_{d} for the non-GCC protein, e.g., BSA, which is greater than 2, greater than 10, greater than 100, greater than 1,000 times, greater than 10⁴, greater than 10⁵, or greater than 10⁶ times its K_{d} for GCC, e.g., human GCC protein. In determination of K_{d}, the K_{d} for GCC and the non-GCC protein, e.g., BSA, should be done under the same conditions.

As used herein, the term “treat” or “treatment” is defined as the administration of an anti-GCC antibody molecule to a subject, e.g., a patient, or administration, e.g., by application, to an isolated tissue or cell from a subject which is returned to the subject. The anti-GCC antibody molecule can be administered alone or in combination with a second agent. The treatment can be to cure, heal, alleviate, relieve, alter, remedy, ameliorate, palliate, improve or affect the disorder, the symptoms of the disorder or the predisposition toward the disorder, e.g., a cancer. While not wishing to be bound by theory, treating is believed to cause the inhibition, ablation, or killing of a cell in vitro or in vivo, or otherwise reducing capacity of a cell, e.g., an aberrant cell, to mediate a disorder, e.g., a disorder as described herein (e.g., a cancer).

As used herein, the term “subject” is intended to include mammals, primates, humans and non-human animals. For example, a subject can be a patient (e.g., a human patient or a veterinary patient), having a cancer, e.g., of gastrointestinal origin (e.g., colon cancer), a symptom of a cancer, e.g., of gastrointestinal origin (e.g., colon cancer), in which at least some of the cells express GCC, or a predisposition toward a cancer, e.g., of gastrointestinal origin (e.g., colon cancer), in which at least some of the cells express GCC. The term “non-human animals” of the invention includes all non-human vertebrates, e.g., non-human mammals and non-mammals, such as non-human primates, sheep, dog, cow, chickens, amphibians, reptiles, etc, unless otherwise noted. In an embodiment subject excludes one or more or all of a mouse, rat, rabbit or goat.

As used herein, an amount of an anti-GCC antibody molecule “effective” or “sufficient” to treat a disorder, or a “therapeutically effective amount” or “therapeutically sufficient amount” refers to an amount of the antibody molecule which is effective, upon single or multiple dose administration to a subject, in treating a cell, e.g., cancer cell (e.g., a GCC-expressing tumor cell), or in prolonging curing, alleviating, relieving or improving a subject with a disorder as described herein beyond that expected in the absence of such treatment. As used herein, “inhibiting the growth” of the tumor or cancer refers to slowing, interrupting, arresting or stopping its growth and/or metastases and does not necessarily indicate a total elimination of the tumor growth.

As used herein, “GCC,” also known as “STAR”, “GUC2C”, “GUCY2C” or “ST receptor” protein refers to mammalian GCC, preferably human GCC protein. Human GCC refers to the protein shown in SEQ ID NO:3 and naturally occurring allelic protein variants thereof. The allele in SEQ ID NO: 3 can be encoded by the nucleic acid sequence of GCC shown in SEQ ID NO:2. Other variants are known in the art. See, e.g., accession number Ensp0000261170, Ensembl Database, European Bioinformatics Institute and Wellcome Trust Sanger Institute, which has a leucine at residue 281; SEQ ID NO: 14 of published US patent application number US 20060035852; or GenBank accession number AAB19934. Typically, a naturally occurring allelic variant has an amino acid sequence at least 95%, 97% or 99% identical to the GCC sequence of SEQ ID NO:3. The transcript encodes a protein product of 1073 amino acids, and is described in GenBank accession no.: NM_{—}004963. GCC protein is characterized as a transmembrane cell surface receptor protein, and is believed to play a critical role in the maintenance of intestinal fluid, electrolyte homeostasis and cell proliferation.

Unless otherwise noted, the term “alkyl” refers to a saturated straight or branched hydrocarbon having from about 1 to about 20 carbon atoms (and all combinations and subcombinations of ranges and specific numbers of carbon atoms therein), with from about 1 to about 8 carbon atoms being preferred. Examples of alkyl groups are methyl, ethyl, n-propyl, iso-propyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl.

Alkyl groups, whether alone or as part of another group, may be referred to as “substituted.” A substituted alkyl group is an alkyl group that is substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, _NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl, and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more groups including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the terms “alkenyl” and “alkynyl” refer to straight and branched carbon chains having from about 2 to about 20 carbon atoms (and all combinations and subcombinations of ranges and specific numbers of carbon atoms therein), with from about 2 to about 8 carbon atoms being preferred. An alkenyl chain has at least one double bond in the chain and an alkynyl chain has at least one triple bond in the chain. Examples of alkenyl groups include, but are not limited to, ethylene or vinyl, allyl, -1-butenyl, -2-butenyl, -isobutylenyl, -1-pentenyl, -2-pentenyl, -3-methyl-1-butenyl, -2-methyl-2-butenyl, and -2,3-dimethyl-2-butenyl. Examples of alkynyl groups include, but are not limited to, acetylenic, propargyl, acetylenyl, propynyl, -1-butynyl, -2-butynyl, -1-pentynyl, -2-pentynyl, and -3-methyl-1 butynyl.

As with alkyl groups, alkenyl and alkynyl groups, can be substituted. A “substituted” alkenyl or alkynyl group is one that is substituted with one or more groups, preferably 1 to 3 groups (and any additional substituents selected from halogen), including but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkyenl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkylene” refers to a saturated branched or straight chain hydrocarbon radical having from about 1 to about 20 carbon atoms (and all combinations and subcombinations of ranges and specific numbers of carbon atoms therein), with from about 1 to about 8 carbon atoms being preferred and having two monovalent radical centers derived by the removal of two hydrogen atoms from the same or two different carbon atoms of a parent alkane. Typical alkylenes include, but are not limited to, methylene, ethylene, propylene, butylene, pentylene, hexylene, heptylene, ocytylene, nonylene, decalene, 1,4-cyclohexylene, and the like. Alkylene groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkenylene” refers to an optionally substituted alkylene group containing at least one carbon-carbon double bond. Exemplary alkenylene groups include, for example, ethenylene (—CH═CH—) and propenylene (—CH═CHCH₂—).

Unless otherwise noted, the term “alkynylene” refers to an optionally substituted alkylene group containing at least one carbon-carbon triple bond. Exemplary alkynylene groups include, for example, acetylene (—C≡C—), propargyl (—CH₂C≡C—), and 4-pentynyl (—CH₂CH₂CH₂C≡CH—).

Unless otherwise noted, the term “aryl” refers to a monovalent aromatic hydrocarbon radical of 6-20 carbon atoms (and all combinations and subcombinations of ranges and specific numbers of carbon atoms therein) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, phenyl, naphthalene, anthracene, biphenyl, and the like.

An aryl group, whether alone or as part of another group, can be optionally substituted with one or more, preferably 1 to 5, or even 1 to 2 groups including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —NO₂, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “arylene” refers to an optionally substituted aryl group which is divalent (i.e., derived by the removal of two hydrogen atoms from the same or two different carbon atoms of a parent aromatic ring system) and can be in the ortho, meta, or para configurations as shown in the following structures with phenyl as the exemplary aryl group:

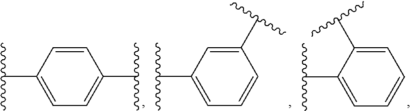

Typical “—(C₁-C₈ alkylene)aryl,” “—(C₂-C₈ alkenylene)aryl”, “and —(C₂-C₈ alkynylene)aryl” groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

Unless otherwise noted, the term “heterocycle,” refers to a monocyclic, bicyclic, or polycyclic ring system having from 3 to 14 ring atoms (also referred to as ring members) wherein at least one ring atom in at least one ring is a heteroatom selected from N, O, P, or S (and all combinations and subcombinations of ranges and specific numbers of carbon atoms and heteroatoms therein). The heterocycle can have from 1 to 4 ring heteroatoms independently selected from N, O, P, or S. One or more N, C, or S atoms in a heterocycle can be oxidized. A monocylic heterocycle preferably has 3 to 7 ring members (e.g., 2 to 6 carbon atoms and 1 to 3 heteroatoms independently selected from N, O, P, or S), and a bicyclic heterocycle preferably has 5 to 10 ring members (e.g., 4 to 9 carbon atoms and 1 to 3 heteroatoms independently selected from N, O, P, or S). The ring that includes the heteroatom can be aromatic or non-aromatic. Unless otherwise noted, the heterocycle is attached to its pendant group at any heteroatom or carbon atom that results in a stable structure.

A heterocycle group, whether alone or as part of another group, can be optionally substituted with one or more groups, preferably 1 to 2 groups, including but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or aryl.

Unless otherwise noted, the term “carbocycle,” refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles preferably have 3 to 6 ring atoms, still more preferably 5 or 6 ring atoms. Bicyclic carbocycles preferably have 7 to 12 ring atoms, e.g., arranged as a bicyclo [4,5], [5,5], [5,6] or [6,6] system, or 9 or 10 ring atoms arranged as a bicyclo [5,6] or [6,6] system. The term “carbocycle” includes, for example, a monocyclic carbocycle ring fused to an aryl ring (e.g., a monocyclic carbocycle ring fused to a benzene ring). Carbocyles preferably have 3 to 8 carbon ring atoms.

Carbocycle groups, whether alone or as part of another group, can be optionally substituted with, for example, one or more groups, preferably 1 or 2 groups (and any additional substituents selected from halogen), including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Examples of monocyclic carbocylic substituents include -cyclopropyl, -cyclobutyl, -cyclopentyl, -1-cyclopent-1-enyl, -1-cyclopent-2-enyl, -1-cyclopent-3-enyl, cyclohexyl, -1-cyclohex-1-enyl, -1-cyclohex-2-enyl, -1-cyclohex-3-enyl, -cycloheptyl, -cyclooctyl. -1,3-cyclohexadienyl, -1,4-cyclohexadienyl, -1,3-cycloheptadienyl, -1,3,5-cycloheptatrienyl, and -cyclooctadienyl.

A “carbocyclo,” whether used alone or as part of another group, refers to an optionally substituted carbocycle group as defined above that is divalent (i.e., derived by the removal of two hydrogen atoms from the same or two different carbon atoms of a parent carbocyclic ring system).

Unless otherwise indicated by context, a hyphen (-) designates the point of attachment to the pendant molecule. Accordingly, the term “—(C₁-C₈ alkylene)aryl” or “—C₁-C₈ alkylene(aryl)” refers to a C₁-C₈ alkylene radical as defined herein wherein the alkylene radical is attached to the pendant molecule at any of the carbon atoms of the alkylene radical and one of the hydrogen atoms bonded to a carbon atom of the alkylene radical is replaced with an aryl radical as defined herein.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The abbreviation “AFP” refers to dimethylvaline-valine-dolaisoleuine-dolaproine-phenylalanine-p-phenylenediamine (see Formula (XVIII) infra).

The abbreviation “MMAE” refers to monomethyl auristatin E (see Formula (XIII) infra).

The abbreviation “AEB” refers to an ester produced by reacting auristatin E with paraacetyl benzoic acid (see Formula (XXII) infra).

The abbreviation “AEVB” refers to an ester produced by reacting auristatin E with benzoylvaleric acid (see Formula (XXIII) infra).

The abbreviation “MMAF” refers to monomethyl auristatin F (see Formula (XXI) infra).

### Antibodies

In certain aspects, the methods, kits and compositions described herein include an anti-GCC antibody molecule described herein, e.g., an anti-GCC antibody molecule having one or more feature summarized in Tables 1-6.

In preferred embodiments, the anti-GCC antibody molecule is antibody 5F9, i.e., an antibody comprising the amino acid sequence of the variable light chain and variable heavy chain provided in Table 3. Antibody 5F9 may be produced by hybridoma 5F9 (PTA-8132) or another suitable cell line, e.g., a mammalian cell line, e.g., a human cell line, a NSO cell line or a CHO cell line. In other preferred embodiments, an anti-GCC antibody molecule is derived from antibody 5F9.

In an embodiment an anti-GCC antibody molecule will have an affinity for GCC, e.g., as measured by direct binding or competition binding assays, in a range described herein. In an embodiment the anti-GCC antibody molecule has a K_{d} of less than 1×10⁻⁶M, less than 1×10⁻⁷ M, less than 1×10⁻⁸ M, less than 1×10⁻⁹ M, less than 1×10⁻¹° M, less than 1×10⁻¹¹ M, less than 1×10⁻¹² M, or less than 1×10⁻¹³ M. In an embodiment the antibody molecule is an IgG, or antigen-binding fragment thereof, and has a K_{d} of less than 1×10⁻⁶M, less than 1×10⁻⁷ M, less than 1×10⁻⁸ M, or less than 1×10⁻⁹ M. In an embodiment, an anti-GCC antibody molecule, e.g., a 5F9 antibody or antibody derived therefrom has a K_{d} of about 80 to about 200 μM, preferably about 100 to about 150 μM or about 120 μM. In an embodiment, an anti-GCC antibody molecule, e.g., a 5F9 antibody or antibody derived therefrom has a kₐ of about 0.9 to about 1.25×10⁵ M⁻¹ s⁻¹, preferably about 1.1×10⁵ M⁻¹ s⁻¹. In an embodiment the antibody molecule is an ScFv and has a K_{d} of less than 1×10⁻⁶M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M.

In some embodiments, the antibody molecule is part of an immunoconjugate and, e.g., the immunoconjugate can both cause a cellular reaction upon binding to GCC and internalize to deliver an agent to the GCC-expressing cell to which it binds.

In some embodiments, an anti-GCC antibody molecule of the invention can block ligand binding to GCC.

In an embodiment, the anti-GCC antibody molecule fails to show substantial cross reaction with one or both of rat GCC and mouse GCC.

The naturally occurring mammalian antibody structural unit is typified by a tetramer. Each tetramer is composed of two pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains can be classified as kappa and lambda light chains. Heavy chains can be classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). The variable regions of each light/heavy chain pair form the antibody binding site. Preferred isotypes for the anti-GCC antibody molecules are IgG immunoglobulins, which can be classified into four subclasses, IgG1, IgG2, IgG3 and IgG4, having different gamma heavy chains. Most therapeutic antibodies are human, chimeric, or humanized antibodies of the IgG1 type. In a particular embodiment, the anti-GCC antibody molecule has the IgG1 isotype.

The variable regions of each heavy and light chain pair form the antigen binding site. Thus, an intact IgG antibody has two binding sites which are the same. However, bifunctional or bispecific antibodies are artificial hybrid constructs which have two different heavy/light chain pairs, resulting in two different binding sites.

The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989). As used herein, CDRs are referred to for each of the heavy (HCDR1, HCDR2, HCDR3) and light (LCDR1, LCDR2, LCDR3) chains.

An anti-GCC antibody molecule can comprise all, or an antigen binding subset of the CDRs, of one or both, the heavy and light chain, of an antibody described herein. For example, an anti-GCC antibody molecule can comprise an amino acid sequence, including variable regions and/or CDRs, described in Table 3 and Table 5. Additional descriptions of anti-GCC antibody molecules are found in International Application WO2011/050242, which is hereby incorporated by references in its entirety.

Thus, in an embodiment the antibody molecule includes one or both of:

(a) one, two, three, or an antigen binding number of, light chain CDRs (LCDR1, LCDR2 and/or LCDR3) of Table 5. In embodiments the CDR(s) may comprise an amino acid sequence of one or more or all of LCDR1-3 as follows: LCDR1, or modified LCDR1 wherein one to seven amino acids are conservatively substituted) LCDR2, or modified LCDR2 wherein one or two amino acids are conservatively substituted); or LCDR3, or modified LCDR3 wherein one or two amino acids are conservatively substituted; and

(b) one, two, three, or an antigen binding number of, heavy chain CDRs (HCDR1, HCDR2 and/or HCDR3) of Table 5. In embodiments the CDR(s) may comprise an amino acid sequence of one or more or all of HCDR1-3 as follows: HCDR1, or modified HCDR1 wherein one or two amino acids are conservatively substituted; HCDR2, or modified HCDR2 wherein one to four amino acids are conservatively substituted; or HCDR3, or modified HCDR3 wherein one or two amino acids are conservatively substituted.

Useful immunogens for production of anti-GCC antibodies include GCC e.g., human GCC-expressing cells (e.g., a tumor cell line, e.g., T84 cells, or fresh or frozen colon tumor cells, recombinant cells expressing GCC); membrane fractions of GCC-expressing cells (e.g., a colon tumor cell line, e.g., T84 cells, or fresh or frozen colonic tumor cells, recombinant cells expressing GCC, e.g., HT-29-GCC#2 cells, which express full-length GCC, or a portion thereof, e.g., CHO GCC #27 cells which express a portion comprising the GCC extracellular domain, e.g., SEQ ID NO:61); isolated or purified GCC, e.g., human GCC protein (e.g., biochemically isolated GCC, e.g., isolated from gastrointestinal tumor cells or recombinant cells expressing GCC or a variant thereof), or a portion thereof (e.g., the extracellular domain of GCC, the kinase homology domain of GCC or the guanylyl cyclase catalytic domain of GCC or peptide corresponding to a portion thereof, e.g., comprising at least about 8, 10, 12, 14, 16, 20, 24, 28 or 32 amino acid residues of SEQ ID NO:3); or an immunogen comprising SEQ ID NO: 16 or comprising a mature portion thereof without the signal sequence (i.e., without amino acid residues 1 to about 21 or 23 of SEQ ID NO:16), e.g., the mature TOK107-hIgG protein, SEQ ID NO:62.

An epitope for an anti-GCC antibody molecule can reside within, or include a residue(s) from, residues 1-50 of SEQ ID NO:3, or a fragment thereof that binds an anti-GCC antibody molecule of the invention, e.g., a 5F9-binding fragment thereof. Such fragments can comprise residues 1-25, 5-30, 10-35, 15-40, 20-45, 25-50, 5-45, 10-40, 15-35, 20-30 or 33-50 of SEQ ID NO:3. In some embodiments, an epitope for an anti-GCC antibody molecule, e.g., a 5F9 antibody, is a conformational epitope further comprising one or more additional amino acid residues in the GCC amino acid sequence beyond residue 50, i.e., selected from about residue 50 to 1073 of SEQ ID NO:3.

In another example, an epitope for an anti-GCC antibody molecule can reside within, or include a residue(s) from, SEQ ID NO:5, or residues 271-300 of SEQ ID NO:3, or a fragment thereof that binds an anti-GCC antibody molecule of the invention. Such fragments can comprise residues 281-290 of SEQ ID NO:3, or residues 281-290 of SEQ ID NO:3 wherein residue 281 is leucine, or residues 281-300 or residues 271-290 of SEQ ID NO:3. In some embodiments, an epitope for an anti-GCC antibody molecule is a conformational epitope further comprising one or more additional amino acid residues, i.e., non-SEQ ID NO:5 residues in the GCC amino acid sequence e.g., selected from about residue 1 to 270 and/or about 301 to 1073 of SEQ ID NO:3.

In an embodiment, the anti-GCC antibody molecule has one or more of the following properties:

a) it competes for binding, e.g., binding to cell surface GCC or purified GCC, with one of the above-referenced anti-GCC antibody molecules summarized in Tables 1 and 2 e.g., 5F9;

b) it binds to the same, or substantially the same, epitope on GCC as one of the above-referenced anti-GCC antibody molecules summarized in Tables 1 and 2, e.g., 5F9. In an embodiment, the antibody binds the same epitope, as determined by one or more of a peptide array assay or by binding to truncation mutants, chimeras or point mutants expressed on the cell surface or membrane preparations, e.g., as those assays are described herein;

c) it binds to an epitope which has at least 1, 2, 3, 4, 5, 8, 10, 15 or 20 contiguous amino acid residues in common with the epitope of one of the above-referenced anti-GCC antibody molecules summarized in Tables 1 and 2, e.g., 5F9;

d) it binds a region of human GCC that is bound by an anti-GCC antibody of the invention, wherein the region e.g., an extracellular or cytoplasmic region, is 10-15, 10-20, 20-30, or 20-40 residues in length, and binding is determined, e.g., by binding to truncation mutants; In an embodiment the anti-GCC antibody molecule binds the extracellular region of human GCC. In an embodiment an anti-GCC antibody molecule can bind the human GCC portion of the extracellular domain defined by amino acid residues 24 to 420 of SEQ ID NO:3. In an embodiment an anti-GCC antibody molecule can bind the guanylate cyclase signature site at amino acid residues 931 to 954 of SEQ ID NO:3; or

e) it binds to a reference epitope described herein.

In an embodiment the anti-GCC antibody molecule binds the GCC sequence ILVDLFNDQYFEDNVTAPDYMKNVLVLTLS (SEQ ID NO:5).

In an embodiment the anti-GCC antibody molecule binds the GCC sequence FAHAFRNLTFEGYDGPVTLDDWGDV (SEQ ID NO: 6).

In an embodiment the antibody molecule binds a conformational epitope. In other embodiments an antibody molecule binds a linear epitope.

The anti-GCC antibody molecules can be polyclonal antibodies, monoclonal antibodies, monospecific antibodies, chimeric antibodies (See U.S. Pat. No. 6,020,153) or human or humanized antibodies or antibody fragments or derivatives thereof. Synthetic and genetically engineered variants (See U.S. Pat. No. 6,331,415) of any of the foregoing are also contemplated by the present invention. Monoclonal antibodies can be produced by a variety of techniques, including conventional murine monoclonal antibody methodology e.g., the standard somatic cell hybridization technique of Kohler and Milstein, Nature 256: 495 (1975). See generally, Harlow, E. and Lane, D. (1988) Antibodies: A Laboratory Manual, Cold Spring Harbor Laboratory Press, Cold Spring Harbor, N.Y. Additional details on producing anti-GCC antibodies are provided in International Application WO2011/050242, which is hereby incorporated by references in its entirety.

In embodiments, for therapeutic applications, the antibodies of the present invention are human or humanized antibodies. The advantage of human or humanized antibodies is that they potentially decrease or eliminate the immunogenicity of the antibody in a host recipient, thereby permitting an increase in the bioavailability and a reduction in the possibility of adverse immune reaction, thus potentially enabling multiple antibody administrations.

Modified antibodies include humanized, chimeric or CDR-grafted antibodies. Details on producing human or humanized anti-GCC antibodies are provided, e.g., in International Application WO2011/050242, which is hereby incorporated by references in its entirety.

Human 5F9 antibody, which in some embodiments includes an IgG2 heavy chain constant region and a kappa light chain constant region, can be produced by hybridoma 5F9, also referred to as hybridoma 46.5F9.8.2, which was deposited on Jan. 10, 2007, on behalf of Millennium Pharmaceuticals Inc., 40 Landsdowne Street, Cambridge, Mass., 02139, USA, at the American Type Culture Collection, 10801 University Boulevard, Manassas, Va. 20110, U.S.A., under Accession No. PTA-8132. (The deposit was made pursuant to, and in satisfaction of, the requirements of the Budapest Treaty on the International Recognition of the Deposit of Microorganisms for the Purposes of Patent Procedure.) Antibody 5F9 can also be produced by other cell lines, e.g., mammalian cell lines, e.g., human cell lines, NSO cell lines or CHO cell lines (see Example 4). As described herein, hybridoma 5F9 produces an IgG2, kappa antibody. However, the IgG2 region may be replaced with, e.g., an IgG1 region to produce a 5F9 IgG1 antibody.

The sequences of human constant region genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Isotypes can be IgG1, IgG2, IgG3 or IgG4. In particular embodiments, antibody molecules of the invention are IgG1 and IgG2. Either of the human light chain constant regions, kappa or lambda, may be used. The chimeric, humanized antibody is then expressed by conventional methods.

In some embodiments, an anti-GCC antibody molecule of the invention can draw antibody-dependent cellular cytotoxicity (ADCC) to a cell expressing GCC, e.g., a tumor cell. Antibodies with the IgG1 and IgG3 isotypes are useful for eliciting effector function in an antibody-dependent cytotoxic capacity, due to their ability to bind the Fc receptor. Antibodies with the IgG2 and IgG4 isotypes are useful to minimize an ADCC response because of their low ability to bind the Fc receptor. In related embodiments substitutions in the Fc region or changes in the glycosylation composition of an antibody, e.g., by growth in a modified eukaryotic cell line, can be made to enhance the ability of Fc receptors to recognize, bind, and/or mediate cytotoxicity of cells to which anti-GCC antibodies bind (see, e.g., U.S. Pat. Nos. 7,317,091, 5,624,821 and publications including WO 00/42072, Shields, et al. J. Biol. Chem. 276:6591-6604 (2001), Lazar et al. Proc. Natl. Acad. Sci. U.S.A. 103:4005-4010 (2006), Satoh et al. Expert Opin Biol. Ther. 6:1161-1173 (2006)). In certain embodiments, the antibody or antigen-binding fragment (e.g., antibody of human origin, human antibody) can include amino acid substitutions or replacements that alter or tailor function (e.g., effector function). For example, a constant region of human origin (e.g., γ1 constant region, γ2 constant region) can be designed to reduce complement activation and/or Fc receptor binding. (See, for example, U.S. Pat. No. 5,648,260 (Winter et al.), U.S. Pat. No. 5,624,821 (Winter et al.) and U.S. Pat. No. 5,834,597 (Tso et al.), the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin.

Humanized antibodies can be made, e.g., using a CDR-grafted approach. Techniques of generation of such humanized antibodies are known in the art. Generally, humanized antibodies are produced by obtaining nucleic acid sequences that encode the variable heavy and variable light sequences of an antibody that binds to GCC, identifying the complementary determining region or “CDR” in the variable heavy and variable light sequences and grafting the CDR nucleic acid sequences on to human framework nucleic acid sequences. (See, for example, U.S. Pat. Nos. 4,816,567 and 5,225,539). The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. J. Mol. Biol. 196:901-917 (1987)). Exemplary anti-GCC antibody molecules described herein have the CDR amino acid sequences and nucleic acid sequences encoding CDRs listed in Tables 5 and 6. In some embodiments sequences from Tables 5 and 6 can be incorporated into molecules which recognize GCC for use in the therapeutic or diagnostic methods described herein. The human framework that is selected is one that is suitable for in vivo administration, meaning that it preferably does not exhibit immunogenicity within a reasonable risk-benefit ratio under the conditions of administration. For example, such a determination can be made by prior experience with in vivo usage of such antibodies and studies of amino acid similarities. A suitable framework region can be selected from an antibody of human origin having at least about 65% amino acid sequence identity, and preferably at least about 70%, 80%, 90% or 95% amino acid sequence identity over the length of the framework region within the amino acid sequence of the equivalent portion (e.g., framework region) of the donor antibody, e.g., an anti-GCC antibody molecule. Amino acid sequence identity can be determined using a suitable amino acid sequence alignment algorithm, such as CLUSTAL W, using the default parameters. (Thompson J. D. et al., Nucleic Acids Res. 22:4673-4680 (1994).)

In other embodiments, reduction of an immunogenic response by a CDR-grafted antibody can be achieved by changes, e.g., deletions, substitutions, of amino acid residues in CDRs (Kashmiri et al. Methods 36:25-34 (2005), U.S. Pat. No. 6,818,749, Tan et al. J. Immunol. 169:1119-1125 (2006)). For example, residues at positions involved in contact with the antigen preferably would not be changed. Typically, such residues, the SDRs, are in positions which display high levels of variability among antibodies. Consensus sequences (e.g., SEQ ID NOs: 63-68,) derived, e.g., by the Clustal method (Higgins D. G. et al., Meth. Enzymol. 266:383-402 (1996)), from anti-GCC antibody molecules, e.g., from antibodies described herein, aid in identifying SDRs. In the human anti-GCC antibody molecules described herein, the SDRs are the following, at least the first residue or in some embodiments, the first four residues of heavy chain CDR1; at least the N-terminal portion, e.g., the first seven, ten or 13 residues of heavy chain CDR2; nearly all of heavy chain CDR3; the C-terminal portion, e.g., after residue six, eight, or nine of light chain CDR1; about the first, middle and/or last residue of light chain CDR2; and most of light chain CDR3, or at least after residue two or three. Accordingly, to maintain binding to GCC protein after humanization or modification of an anti-GCC antibody molecule, such SDR residues in CDRs of the anti-GCC antibody molecules are less amenable to changes, e.g., from murine residues to human consensus residues than are residues in other residues of the CDRs or the framework regions. Conversely, it can be beneficial to change residues in non-human, e.g., murine CDRs to residues identified as consensus in human CDRs, e.g., CDRs of anti-GCC antibody molecules described herein (e.g., the sequences listed in Table 5). For example, a serine can represent a human residue for the C-terminus of heavy chain CDR1, and/or a tyrosine can represent a human residue for the second and/or third residues of heavy chain CDR1; heavy chain CDR2 can end in S-(L/V)-K-(S/G) (SEQ ID NO: 7) to represent a human CDR; to represent a human CDR3, there can be a glycine after four to six residues and/or an aspartate six to nine residues in heavy chain CDR3; light chain CDR1 can begin with (K/R)-(A/S)-SQS-(V/L)-(S/L) (SEQ ID NO: 8) to represent a human CDR; light chain CDR2 can have a serine in the third residue and/or an arginine in the fifth residue represent a human CDR; and/or light chain CDR3 can have a glutamine in the second residue and/or a tyrosine or serine in the third residue represent a human CDR.

Anti-GCC antibodies that are not intact antibodies are also useful in this invention. Such antibodies may be derived from any of the antibodies described above. Useful antibody molecules of this type include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; (vii) a single domain functional heavy chain antibody, which consists of a VHH domain (known as a nanobody) see e.g., Cortez-Retamozo, et al., Cancer Res. 64: 2853-2857 (2004), and references cited therein; and (vii) an isolated CDR, e.g., one or more isolated CDRs together with sufficient framework to provide an antigen binding fragment. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988); and Huston et al. Proc. Natl. Acad. Sci. USA 85:5879-5883 (1988). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antibody fragments, such as Fv, F(ab′)₂ and Fab may be prepared by cleavage of the intact protein, e.g. by protease or chemical cleavage.

Embodiments include an antibody molecule that comprises sufficient CDRs, e.g., all six CDRs from Table 5 to allow binding to cell surface GCC.

In an embodiment the CDRs, e.g., all of the HCDRs, or all of the LCDRs, or all six, are embedded in human or human derived framework region(s). Examples of human framework regions include human germline framework sequences, human germline sequences that have been affinity matured (either in vivo or in vitro), or synthetic human sequences, e.g., consensus sequences. In an embodiment the heavy chain framework is an IgG1 or IgG2 framework. In an embodiment the light chain framework is a kappa framework.

An anti-GCC antibody molecule can comprise all, or an antigen binding fragment of the variable region, of one or both, the heavy and light chain, of one of the above-referenced human hybridoma, selected lymphocyte, or murine antibodies.

In an embodiment the light chain amino acid sequence of (a) can differ from one of the reference amino acid sequence(s) referred to in (a)(i-ii) by as many as 1, 2, 3, 4, 5, 10, or 15 residues. In embodiments the differences are conservative substitutions. In embodiments, the differences are in the framework regions. In an embodiment the heavy chain amino acid sequence of (b) can differ from one of the reference amino acid sequence(s) referred to in (b)(i-ii) by as many as 1, 2, 3, 4, 5, 10, or 15 residues. In embodiments the differences are conservative substitutions. In embodiments the differences are in the framework regions.

In an embodiment the anti-GCC antibody molecule comprises one or both of:

(a) a light chain amino acid sequence of all, or an antigen binding fragment of, either, (i) a light chain variable region amino acid sequence from Table 3, e.g., SEQ ID NO:20, or (ii) a light chain variable region amino acid encoded by a nucleotide sequence from Table 4, e.g., SEQ ID NO:19; and

(b) a heavy chain amino acid sequence of all, or an antigen binding fragment of, either (i) a heavy chain variable region amino acid sequence from Table 3, e.g., SEQ ID NO:18, or (ii) a heavy chain amino acid sequence encoded by a nucleotide sequence from Table 4, e.g., SEQ ID NO:17.

In an embodiment the anti-GCC antibody molecule comprises one or both of:

a) a light chain variable region, or an antigen binding fragment thereof, having at least 85, 90, 95, 97 or 99% homology with the light chain variable region of an anti-GCC antibody molecule of the invention, e.g., one of the above-referenced human hybridoma, selected lymphocyte, or murine antibodies; and

(b) a heavy chain variable region, or an antigen binding fragment thereof, having at least 85, 90, 95, 97 or 99% homology with the heavy chain variable region of an anti-GCC antibody molecule of the invention, e.g., one of the above-referenced human hybridoma, selected lymphocyte, or murine antibodies.

The amino acid sequences of light chain and heavy chain variable regions of an exemplary antibody can be found in Table 3.

In an embodiment, the anti-GCC antibody molecule is a 5F9 antibody molecule and includes one or both of: a) all or a fragment of the heavy chain constant region from SEQ ID NO: 32; and b) all or a fragment of the light chain constant region from SEQ ID NO: 34.

In another embodiment, the anti-GCC antibody molecule is an Abx-229 antibody molecule and includes one or both of: a) all or a GCC-binding fragment of the heavy chain variable region from SEQ ID NO: 46; and b) all or a GCC-binding fragment of the light chain variable region from SEQ ID NO: 48.

In one approach, consensus sequences encoding the heavy and light chain J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, cosmids, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Polyadenylation and transcription termination occur at native chromosomal sites downstream of the coding regions. The resulting chimeric antibody may be joined to any strong promoter. Examples of suitable vectors that can be used include those that are suitable for mammalian hosts and based on viral replication systems, such as simian virus 40 (SV40), Rous sarcoma virus (RSV), adenovirus 2, bovine papilloma virus (BPV), papovavirus BK mutant (BKV), or mouse and human cytomegalovirus (CMV), and moloney murine leukemia virus (MMLV), native Ig promoters, etc. A variety of suitable vectors are known in the art, including vectors which are maintained in single copy or multiple copies, or which become integrated into the host cell chromosome, e.g., via LTRs, or via artificial chromosomes engineered with multiple integration sites (Lindenbaum et al. Nucleic Acids Res. 32:e172 (2004), Kennard et al. Biotechnol. Bioeng. Online May 20, 2009). Additional examples of suitable vectors are listed in a later section.

Thus, the invention provides an expression vector comprising a nucleic acid encoding an antibody, antigen-binding fragment of an antibody (e.g., a human, humanized, chimeric antibody or antigen-binding fragment of any of the foregoing), antibody chain (e.g., heavy chain, light chain) or antigen-binding portion of an antibody chain that binds a GCC protein.

Expression in eukaryotic host cells is useful because such cells are more likely than prokaryotic cells to assemble and secrete a properly folded and immunologically active antibody. However, any antibody produced that is inactive due to improper folding may be renaturable according to known methods (Kim and Baldwin, “Specific Intermediates in the Folding Reactions of Small Proteins and the Mechanism of Protein Folding”, Ann. Rev. Biochem. 51, pp. 459-89 (1982)). It is possible that the host cells will produce portions of intact antibodies, such as light chain dimers or heavy chain dimers, which also are antibody homologs according to the present invention.

It will be appreciated that antibodies that are generated need not initially possess a particular desired isotype but, rather, the antibody as generated can possess any isotype. For example, the antibody produced by the 5F9 hybridoma (ATCC deposit no. PTA-8132) has the IgG2 isotype. The isotype of the antibody can be switched thereafter, e.g., to IgG1 or IgG3 to elicit an ADCC response when the antibody binds GCC on a cell, using conventional techniques that are known in the art. Such techniques include the use of direct recombinant techniques (see e.g., U.S. Pat. No. 4,816,397), cell-cell fusion techniques (see e.g., U.S. Pat. No. 5,916,771), among others. In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

In certain embodiments, the GCC antibody molecule is a human anti-GCC IgG1 antibody. Since such antibodies possess desired binding to the GCC molecule, any one of such antibodies can be readily isotype-switched to generate a human IgG4 isotype, for example, while still possessing the same variable region (which defines the antibody's specificity and affinity, to a certain extent). Accordingly, as antibody candidates are generated that meet desired “structural” attributes as discussed above, they can generally be provided with at least certain additional “functional” attributes that are desired through isotype switching.

In an embodiment the variable region or antigen binding fragment thereof can be coupled to a constant region (or fragment thereof) other than the constant region it was generated with, e.g., a constant region (or fragment thereof) from another antibody or to a synthetic constant region (or fragment thereof). In embodiments the constant region is an IgG1 or IgG2 constant region (or fragment thereof). Sequence changes can be made in the variable or constant regions to modify effector activity of the antibody molecule.

### Design and Generation of Other Therapeutics

The antibodies that are produced and characterized herein with respect to GCC provide for the design of other therapeutic modalities including other antibodies, other antagonists, or chemical moieties other than antibodies is facilitated. Such modalities include, without limitation, antibodies having similar binding activity or functionality, advanced antibody therapeutics, such as bispecific antibodies, immunoconjugates, and radiolabeled therapeutics, generation of peptide therapeutics, particularly intrabodies, and small molecules. Furthermore, as discussed above, the effector function of the antibodies of the invention may be changed by isotype switching to an IgG1, IgG2, IgG3, IgG4, IgD, IgA1, IgA2, IgE, or IgM for various therapeutic uses.

In connection with bispecific antibodies, bispecific antibodies can be generated that comprise (i) two antibodies, one with a specificity to GCC and another to a second molecule that are conjugated together, (ii) a single antibody that has one chain specific to GCC and a second chain specific to a second molecule, or (iii) a single chain antibody that has specificity to GCC and the other molecule. Such bispecific antibodies can be generated using techniques that are known.

In addition, “Kappabodies” (Ill. et al. “Design and construction of a hybrid immunoglobulin domain with properties of both heavy and light chain variable regions” Protein Eng 10:949-57 (1997)), “Minibodies” (Martin et al. EMBO J. 13:5303-9 (1994), U.S. Pat. No. 5,837,821), “Diabodies” (Holliger et al. Proc Natl Acad Sci USA 90:6444-6448 (1993)), or “Janusins” (Traunecker et al. EMBO J. 10:3655-3659 (1991) and Traunecker et al. Int J Cancer Suppl 7:51-52 (1992)) may also be prepared.

### Polypeptides

In another embodiment, the present invention relates to polypeptide sequences that represent the antibody molecules described herein.

The present invention relates to polypeptides that represent the antibodies of the present invention as well as fragments, analogs and derivatives of such polypeptides. The polypeptides may be recombinant polypeptides, naturally produced polypeptides or synthetic polypeptides. The fragment, derivative or analogs of the polypeptides of the present invention may be one in which one or more of the amino acid residues is substituted with a conserved or non-conserved amino acid residue (preferably a conserved amino acid residue) and such substituted amino acid residue may or may not be one encoded by the genetic code; or it may be one in which one or more of the amino acid residues includes a substituent group; or it may be one in which the polypeptide is fused with another compound, such as a compound to increase the half-life of the polypeptide (for example, polyethylene glycol); or it may be one in which the additional amino acids are fused to the polypeptide, such as a leader or secretory sequence or a sequence that is employed for purification of the polypeptide or a proprotein sequence. Such fragments, derivatives and analogs are within the scope of the present invention. In various aspects, the polypeptides of the invention may be partially purified, or purified product.

A polypeptide can have an amino acid sequence that is identical to that of the antibodies described herein, e.g., summarized in Tables 2 or 3, or that is different by minor variations due to one or more amino acid substitutions. The variation may be a “conservative change” typically in the range of about 1 to 5 amino acids, wherein the substituted amino acid has similar structural or chemical properties, e.g., replacement of leucine with isoleucine or threonine with serine; replacement of lysine with arginine or histidine. In contrast, variations may include nonconservative changes, e.g., replacement of a glycine with a tryptophan. Similar minor variations may also include amino acid deletions or insertions or both. Guidance in determining which and how many amino acid residues may be substituted, inserted, or deleted without changing biological or immunological activity may be found using computer programs known in the art, for example DNASTAR software (DNASTAR, Inc., Madison, Wis.).

In additional aspects, the antibody molecule comprises an amino acid sequence of the light chain variable region amino acid sequence of the antibody encoded by the DNA having ATCC Accession Number PTA-8132. In other additional aspects, the antibody molecule comprises an amino acid sequence of the heavy chain variable region sequence of the antibody encoded by the DNA having ATCC Accession Number PTA-8132.

As will be appreciated, antibodies in accordance with the present invention can be expressed in cell lines other than hybridoma cell lines. Sequences encoding the cDNAs or genomic clones for the particular antibodies can be used for a suitable mammalian or nonmammalian host cells. Transformation can be by any known method for introducing polynucleotides into a host cell, including, for example packaging the polynucleotide in a virus (or into a viral vector) and transducing a host cell with the virus (or vector) or by transfection procedures known in the art, for introducing heterologous polynucleotides into mammalian cells, e.g., dextran-mediated transfection, calcium phosphate precipitation, polybrene mediated transfection, protoplast fusion, electroporation, encapsulation of the polynucleotide(s) into liposomes and direct microinjection of the DNA molecule. The transformation procedure used depends upon the host to be transformed. Methods for introduction of heterologous polynucleotides into mammalian cells are known in the art and include, but are not limited to, dextran-mediated transfection, calcium phosphate precipitation, polybrene mediated transfection, protoplast fusion, electroporation, particle bombardment, encapsulation of the polynucleotide(s) in liposomes, peptide conjugates, dendrimers, and direct microinjection of the DNA into nuclei.

### Fusion Proteins and Immunoconjugates

The anti-GCC antibodies described herein can be functionally linked by any suitable method (e.g., chemical coupling, genetic fusion, noncovalent association or otherwise) to one or more non-antibody molecular entities.

Fusion proteins can be produced in which an anti-GCC antibody molecule as described herein and a non-antibody moiety are components of a single continuous polypeptide chain. The non-antibody moiety can be located N-terminally, C-terminally, or internally, with respect to the antibody moiety. For example, some embodiments can be produced by the insertion of a nucleic acid encoding immunoglobulin sequences into a suitable expression vector, such as a pET vector (e.g., pET-15b, Novagen), a phage vector (e.g., pCNATAB 5 E, Pharmacia), or other vector, e.g., pRIT2T Protein A fusion vector, Pharmacia). The resulting construct can be expressed to produce antibody chains that comprise a non-antibody moiety (e.g., Histidine tag, E tag, or Protein A IgG binding domain). Fusion proteins can be isolated or recovered using any suitable technique, such as chromatography using a suitable affinity matrix (see, e.g., Current Protocols in Molecular Biology (Ausubel, F. M et al., eds., Vol. 2, Suppl. 26, pp. 16.4.1-16.7.8 (1991)).

The invention provides anti-GCC antibody molecules which are directed to and, in embodiments, are internalized into cells. They are capable of delivering therapeutic agents or detectable agents to or into cells expressing GCC, but not to or into cells where the target is not expressed. Thus, the invention also provides immunoconjugates comprising an anti-GCC antibody molecule as described herein, which is conjugated to a therapeutic agent or a detectable agent. In embodiments, the affinity for GCC of an immunoconjugate is at least 10, 25, 50, 75, 80, 90, or 95% of that for the unconjugated antibody. This can be determined using cell surface GCC or isolated GCC. In an embodiment the anti-GCC antibody molecule, e.g., an immunoconjugate, has an LD50, as determined by an assay described herein, of less than 1,000, 500, 250, 100, or 50 pM.

The anti-GCC antibody molecule can be modified to act as an immunoconjugate utilizing techniques that are known in the art. See e.g., Vitetta Immunol Today 14:252 (1993). See also U.S. Pat. No. 5,194,594. The preparation of radiolabeled antibodies can also be readily prepared utilizing techniques that are known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (U.S. Re. Pat. No. 35,500), 5,648,471, and 5,697,902.

In some embodiments, the antibody molecule and non-antibody moiety are connected by means of a linker. In such embodiments, the immunoconjugate is represented by Formula (I):

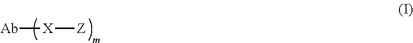

wherein,

Ab is an anti-GCC antibody molecule described herein;

X is a moiety which connects Ab and Z, e.g., the residue of a linker described herein after covalent linkage to one or both of Ab and Z;

Z is a therapeutic agent or label; and

m ranges from about 1 to about 15.

The variable m represents the number of —X—Z moieties per antibody molecule in an immunoconjugate of Formula (I). In various embodiments, m ranges 1 to 8, 1 to 7, 1 to 6, 1 to 5, 1 to 4, 1 to 3, or 1 to 2. In some embodiments, m ranges from 2 to 8, 2 to 7, 2 to 6, 2 to 5, 2 to 4 or 2 to 3. In other embodiments, m is 1, 2, 3, 4, 5 or 6. In compositions comprising a plurality of immunoconjugates of Formula (I), m is the average number of —X—Z moieties per Ab, also referred to as the average drug loading (in examples, where the compositions comprise a plurality of immunoconjugates, m can a number other than an integer (e.g., as averages across the plurality of conjugates). Average drug loading may range from 1 to about 15 —X—Z moieties per Ab. In some embodiments, when m represents the average drug loading, m is about 1, about 2, about 3, about 4, about 5, about 6, about 7, or about 8. In exemplary embodiments, m is from about 2 to about 8. In another embodiment, m is about 4. In another embodiment, m is about 2.

The average number of —X—Z moieties per Ab may be characterized by conventional means such as mass spectroscopy, ELISA assay, and HPLC. The quantitative distribution of immunoconjugates in terms of m may also be determined. In some instances, separation, purification, and characterization of homogeneous immunoconjugates where m is a certain value, as distinguished from immunoconjugates with other drug loadings, may be achieved by means such as reverse phase HPLC or electrophoresis.

A variety of suitable linkers (e.g., heterobifunctional reagents for connecting an antibody molecule to a therapeutic agent or label) and methods for preparing immunoconjugates are known in the art. (See, for example, Chari et al., Cancer Research 52:127-131 (1992).) The linker can be cleavable, e.g., under physiological conditions, e.g., under intracellular conditions, such that cleavage of the linker releases the drug (therapeutic agent or label) in the intracellular environment. In other embodiments, the linker is not cleavable, and the drug is released, for example, by antibody degradation.

The linker can be bonded to a chemically reactive group on the antibody moiety, e.g., to a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues). The site to which the linker is bound can be a natural residue in the amino acid sequence of the antibody moiety or it can be introduced into the antibody moiety, e.g., by DNA recombinant technology (e.g., by introducing a cysteine or protease cleavage site in the amino acid sequence) or by protein biochemistry (e.g., reduction, pH adjustment or proteolysis).

In certain embodiments, an intermediate, which is the precursor of the linker (X), is reacted with the drug (Z) under appropriate conditions. In certain embodiments, reactive groups are used on the drug and/or the intermediate. The product of the reaction between the drug and the intermediate, or the derivatized drug, is subsequently reacted with the antibody molecule under appropriate conditions.

The immunoconjugate can be purified from reactants by employing methodologies well known to those of skill in the art, e.g., column chromatography (e.g., affinity chromatography, ion exchange chromatography, gel filtration, hydrophobic interaction chromatography), dialysis, diafiltration or precipitation. The immunoconjugate can be evaluated by employing methodologies well known to those skilled in the art, e.g., SDS-PAGE, mass spectroscopy, or capillary electrophoresis.

In some embodiments, the linker is cleavable by a cleaving agent that is present in the intracellular environment (e.g., within a lysosome or endosome or caveolea).

In yet other specific embodiments, the linker is a malonate linker (Johnson et al., 1995, Anticancer Res. 15:1387-93), a maleimidobenzoyl linker (Lau et al., 1995, Bioorg Med. Chem. 3(10):1299-1304), or a 3′-N-amide analog (Lau et al., 1995, Bioorg-Med-Chem. 3(10):1305-12).

A variety of exemplary linkers that can be used with the present compositions and methods are described in WO 2004-010957, U.S. Publication No. 20060074008, U.S. Publication No. 20050238649, and U.S. Publication No. 20060024317 (each of which is incorporated by reference herein in its entirety and for all purposes).

Examples of linkers capable of being used to couple an antibody molecule to a therapeutic agent or label include, for example, maleimidocaproyl (mc); maleimidocaproyl-p-aminobenzylcarbamate; maleimidocaproyl-peptide-aminobenzylcarbamate linkers, e.g., maleimidocaproyl-L-phenylalanine-L-lysine-p-aminobenzylcarbamate and maleimidocaproyl-L-valine-L-citrulline-p-aminobenzylcarbamate (vc); N-succinimidyl 3-(2-pyridyldithio)proprionate (also known as N-succinimidyl 4-(2-pyridyldithio)pentanoate or SPP); 4-succinimidyl-oxycarbonyl-2-methyl-2-(2-pyridyldithio)-toluene (SMPT); N-succinimidyl 3-(2-pyridyldithio)propionate (SPDP); N-succinimidyl 4-(2-pyridyldithio)butyrate (SPDB); 2-iminothiolane; S-acetylsuccinic anhydride; disulfide benzyl carbamate; carbonate; hydrazone linkers; N-(α-Maleimidoacetoxy) succinimide ester; N-[4-(p-Azidosalicylamido) butyl]-3′-(2′-pyridyldithio)propionamide (AMAS); N-[β-Maleimidopropyloxy]succinimide ester (BMPS); [N-ε-Maleimidocaproyloxy]succinimide ester (EMCS); N-[γ-Maleimidobutyryloxy]succinimide ester (GMBS); Succinimidyl-4-[N-Maleimidomethyl]cyclohexane-1-carboxy-[6-amidocaproate] (LC-SMCC); Succinimidyl 6-(3-[2-pyridyldithio]-propionamido)hexanoate (LC-SPDP); m-Maleimidobenzoyl-N-hydroxysuccinimide ester (MBS); N-Succinimidyl[4-iodoacetyl]aminobenzoate (SIAB); Succinimidyl 4-[N-maleimidomethyl]cyclohexane-1-carboxylate (SMCC); N-Succinimidyl 3-[2-pyridyldithio]-propionamido (SPDP); [N-ε-Maleimidocaproyloxy]sulfosuccinimide ester (Sulfo-EMCS); N-[γ-Maleimidobutyryloxy]-sulfosuccinimide ester (Sulfo-GMBS); 4-Sulfosuccinimidyl-6-methyl-α-(2-pyridyldithio)toluamido]hexanoate) (Sulfo-LC-SMPT); Sulfosuccinimidyl 6-(3′-[2-pyridyldithio]-propionamido)hexanoate (Sulfo-LC-SPDP); m-Maleimidobenzoyl-N-hydroxysulfosuccinimide ester (Sulfo-MBS); N-Sulfosuccinimidyl[4-iodoacetyl]aminobenzoate (Sulfo-SIAB); Sulfosuccinimidyl 4-[N-maleimidomethyl]cyclohexane-1-carboxylate (Sulfo-SMCC); Sulfosuccinimidyl 4-[p-maleimidophenyl]butyrate (Sulfo-SMPB); ethylene glycol-bis(succinic acid N-hydroxysuccinimide ester) (EGS); disuccinimidyl tartrate (DST); 1,4,7,10-tetraazacyclododecane-1,4,7,10-tetraacetic acid (DOTA); diethylenetriamine-pentaacetic acid (DTPA); and thiourea linkers.

In some embodiments, the linker —X— has the formula -Aₐ-W_{w}—Y_{y}—, and the immunoconjugate of Formula (I) is characterized by Formula (II):

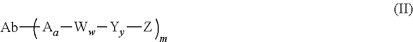

wherein,

Ab is an anti-GCC antibody molecule described herein;

-A- is a Stretcher unit;

a is 0 or 1;

each —W— independently is an Amino Acid unit;

w is an integer ranging from 0 to 12;

—Y— is a self-immolative spacer unit;

y is 0, 1, or 2;

Z is a therapeutic agent or label; and

m ranges from about 1 to about 15.

The Stretcher unit (A), when present, is capable of linking an Ab unit to an Amino Acid unit (—W—), if present, to a Spacer unit (—Y—), if present; or to a therapeutic agent or label (Z). Useful functional groups that can be present on an anti-GCC antibody molecule, either naturally or via chemical manipulation include, but are not limited to, sulfhydryl, amino, hydroxyl, the anomeric hydroxyl group of a carbohydrate, and carboxyl. Suitable functional groups are sulfhydryl and amino. In one example, sulfhydryl groups can be generated by reduction of the intramolecular disulfide bonds of an anti-GCC antibody molecule. In another embodiment, sulfhydryl groups can be generated by reaction of an amino group of a lysine moiety of an anti-GCC antibody molecule with 2-iminothiolane (Traut's reagent) or other sulfhydryl generating reagents. In certain embodiments, the anti-GCC antibody molecule is a recombinant antibody and is engineered to carry one or more lysines. In certain other embodiments, the recombinant anti-GCC antibody molecule is engineered to carry additional sulfhydryl groups, e.g., additional cysteines.

In one embodiment, the Stretcher unit forms a bond with a sulfur atom of the Ab unit. The sulfur atom can be derived from a sulfhydryl group of an Ab. Representative Stretcher units of this embodiment are depicted within the square brackets of Formulas (IIIa) and (IIIb), wherein Ab-, —W—, —Y—, —Z, w and y are as defined above, and R^{a} is selected from —C₁-C₁₀ alkylene-, —C₂-C₁₀ alkenylene-, —C₂-C₁₀ alkynylene-, -carbocyclo-, —O—(C₁-C₈ alkylene)-, O—(C₂-C₈ alkenylene)-, —O—(C₂-C₈ alkynylene)-, -arylene-, —C₁-C₁₀ alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene-(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, heterocyclo-, —C₁-C₁₀ alkylene-(heterocyclo)-, —C₂-C₁₀ alkenylene-(heterocyclo)-, —C₂-C₁₀ alkynylene-(heterocyclo)-, -(heterocyclo)-C₁-C₁₀ alkylene-, -(heterocyclo)-C₂-C₁₀ alkenylene-, -(heterocyclo)-C₂-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynylene, aryl, carbocyle, carbocyclo, heterocyclo, and arylene radicals, whether alone or as part of another group, are optionally substituted. In some embodiments, said alkyl, alkenyl, alkynyl, alkylene, alkenylene, alkynylene, aryl, carbocyle, carbocyclo, heterocyclo, and arylene radicals, whether alone or as part of another group, are unsubstituted. In some embodiments, R^{a} is selected from —C₁-C₁₀ alkylene-, -carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(carbocyclo)-, -(carbocyclo)-C₁-C₁₀ alkylene-, —C₃-C₈ heterocyclo-, —C₁-C₁₀ alkylene-(heterocyclo)-, -(heterocyclo)-C₁-C₁₀ alkylene-, —(CH₂CH₂O)ᵣ—, and —(CH₂CH₂O)ᵣ—CH₂—; and r is an integer ranging from 1-10, wherein said alkylene groups are unsubstituted and the remainder of the groups are optionally substituted.

It is to be understood from all the exemplary embodiments that even where not denoted expressly, from 1 to 15 drug moieties can be linked to an Ab (m=1-15).

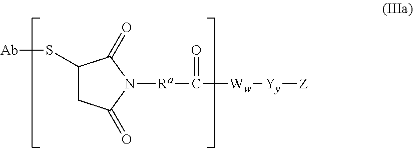

An illustrative Stretcher unit is that of Formula (IIIa) wherein R^{a} is —(CH₂)₅—:

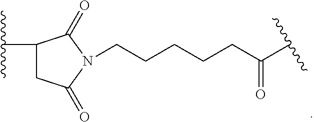 [SMILES: *C(=O)CCCCCN1C(=O)CC(*)C1=O]

It should be noted that throughout this application, the S moiety in the formula below refers to a sulfur atom of the Ab unit, unless otherwise indicated by context.

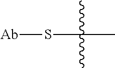

The Amino Acid unit (—W—), when present, links the Stretcher unit to the Spacer unit if the Spacer unit is present, links the Stretcher unit to the Drug moiety if the Spacer unit is absent, and links the Ab unit to the therapeutic agent or label moiety if the Stretcher unit and Spacer unit are absent.

W_{w}— can be, for example, a monopeptide, dipeptide, tripeptide, tetrapeptide, pentapeptide, hexapeptide, heptapeptide, octapeptide, nonapeptide, decapeptide, undecapeptide or dodecapeptide unit.

In certain embodiments, the Amino Acid unit can comprise natural amino acids. In other embodiments, the Amino Acid unit can comprise non-natural amino acids. Illustrative W_{w} units are represented by the formula (VII):

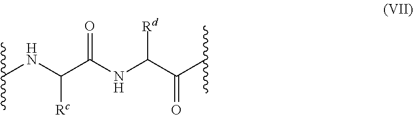

wherein R^{c} and R^{d} are as follows:

| R^{c} | R^{d} |
|---|---|
| Benzyl | (CH₂)₄NH₂; |
| Methyl | (CH₂)₄NH₂; |
| Isopropyl | (CH₂)₄NH₂; |
| Isopropyl | (CH₂)₃NHCONH₂; |
| Benzyl | (CH₂)₃NHCONH₂; |
| Isobutyl | (CH₂)₃NHCONH₂; |
| sec-butyl | (CH₂)₃NHCONH₂; |
| 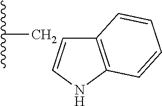 | (CH₂)₃NHCONH₂; |
| Benzyl | methyl; |
| Benzyl | (CH₂)₃NHC(═NH)NH₂; |

In one aspect of the Amino Acid unit, the Amino Acid unit is valine-citrulline (vc or val-cit). In another aspect, the Amino Acid unit is phenylalanine-lysine (i.e., fk). In yet another aspect of the Amino Acid unit, the Amino Acid unit is N-methylvaline-citrulline. In yet another aspect, the Amino Acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

The Spacer unit (—Y—), when present, links an Amino Acid unit to the therapeutic agent or label moiety (—Z—) when an Amino Acid unit is present. Alternatively, the Spacer unit links the Stretcher unit to the therapeutic agent or label moiety when the Amino Acid unit is absent. The Spacer unit also links the therapeutic agent or label moiety to the Ab unit when both the Amino Acid unit and Stretcher unit are absent.

Spacer units are of two general types: non self-immolative or self-immolative. A non self-immolative Spacer unit is one in which part or all of the Spacer unit remains bound to the therapeutic agent or label moiety after cleavage, particularly enzymatic, of an Amino Acid unit from the antibody-drug conjugate. Examples of a non self-immolative Spacer unit include, but are not limited to a (glycine-glycine) Spacer unit and a glycine Spacer unit (both depicted in Scheme 1) (infra). When a conjugate containing a glycine-glycine Spacer unit or a glycine Spacer unit undergoes enzymatic cleavage via an enzyme (e.g., a tumor-cell associated-protease, a cancer-cell-associated protease or a lymphocyte-associated protease), a glycine-glycine-Z moiety or a glycine-Z moiety is cleaved from Ab-Aa-Ww-.

Alternatively, a conjugate containing a self-immolative Spacer unit can release —Z. As used herein, the term “self-immolative Spacer” refers to a bifunctional chemical moiety that is capable of covalently linking together two spaced chemical moieties into a stable tripartite molecule. It will spontaneously separate from the second chemical moiety if its bond to the first moiety is cleaved.

In some embodiments, —Y_{y}— is a p-aminobenzyl alcohol (PAB) unit (see Schemes 2 and 3) whose phenylene portion is substituted with Qₙ wherein Q is —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro or -cyano; and n is an integer ranging from 0-4. The alkyl, alkenyl and alkynyl groups, whether alone or as part of another group, can be optionally substituted.

In some embodiments, —Y— is a PAB group that is linked to —W_{w}— via the amino nitrogen atom of the PAB group, and connected directly to —Z via a carbonate, carbamate or ether group.

Other examples of self-immolative spacers include, but are not limited to, aromatic compounds that are electronically similar to the PAB group such as 2-aminoimidazol-5-methanol derivatives (Hay et al., 1999, Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., 1995, Chemistry Biology 2:223), appropriately substituted bicyclo[2.2.1] and bicyclo[2.2.2] ring systems (Storm et al., 1972, J. Amer. Chem. Soc. 94:5815) and 2-aminophenylpropionic acid amides (Amsberry et al., 1990, J. Org. Chem. 55:5867). Elimination of amine-containing drugs that are substituted at the α-position of glycine (Kingsbury et al., 1984, J. Med. Chem. 27:1447) are also examples of self-immolative spacers.

In some embodiments, the —Z moieties are the same. In yet another embodiment, the —Z moieties are different.

In one aspect, Spacer units (—Y_{y}—) are represented by Formulae (X):

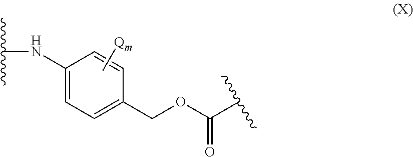

wherein Q is —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro or -cyano; and m is an integer ranging from 0-4. The alkyl, alkenyl and alkynyl groups, whether alone or as part of another group, can be optionally substituted.

In a group of selected embodiments, the conjugates of Formula (I) and II) are:

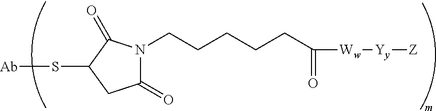

wherein w and y are each 0, 1 or 2:

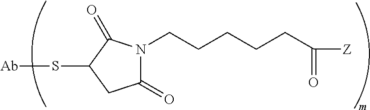

wherein w and y are each 0;

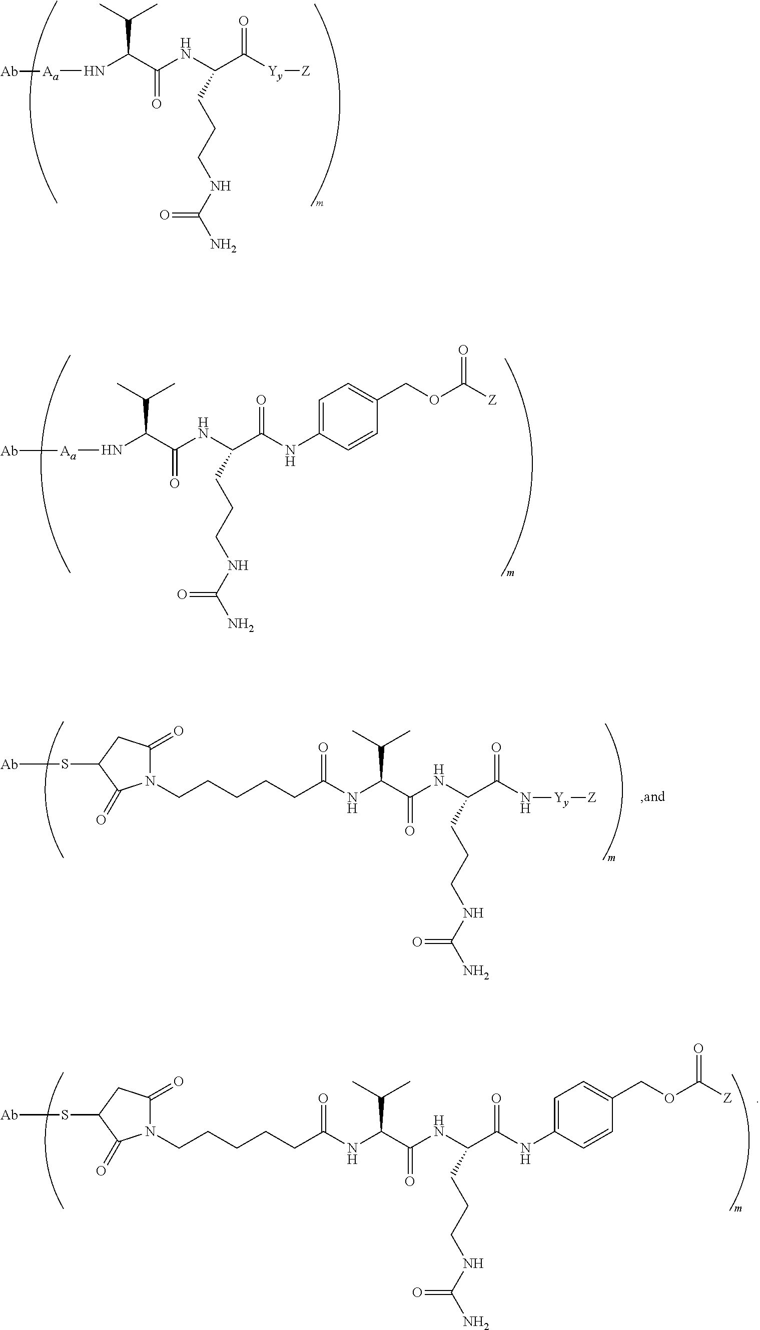

wherein Aₐ, W_{w}, Y_{y}, Z and Ab have the meanings provided above.

The variable Z in Formula (I) is a therapeutic agent or label. The therapeutic agent can be any agent capable of exerting a desired biological effect. In some embodiments, the therapeutic agent sensitizes the cell to a second therapeutic modality, e.g., a chemotherapeutic agent, radiation therapy, immunotherapy.

In some embodiments, the therapeutic agent is a cytostatic or cytotoxic agent. Examples include, without limitation, antimetabolites (e.g., fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, or pemetrexed); alkylating agents (e.g., cmelphalan, chlorambucil, busulfan, thiotepa, ifosfamide, carmustine, lomustine, semustine, streptozocin, dacarbazine, mitomycin C, cyclophosphamide, mechlorethamine, uramustine, dibromomannitol, tetranitrate, procarbazine, altretamine, mitozolomide, or temozolomide); alkylating-like agents (e.g., cisplatin, carboplatin, nedaplatin, oxaliplatin, satraplatin, or triplatin); DNA minor groove alkylating agents (e.g., duocarmycins such as CC-1065, and any analogs or derivatives thereof; pyrrolobenzodiazapenes, or any analogs or derivatives thereof); anthracyclines (e.g., daunorubicin, doxorubicin, epirubicin, idarubicin, or valrubicin); antibiotics (e.g., dactinomycin, bleomycin, mithramycin, anthramycin, streptozotocin, gramicidin D, mitomycins (e.g., mitomycin C); calicheamicins; antimitotic agents (including, e.g., maytansinoids, auristatins, dolastatins, cryptophycins, vinca alkaloids (e.g., vincristine, vinblastine, vindesine, vinorelbine), taxanes (e.g., paclitaxel, docetaxel, or a novel taxane (see, e.g., International Patent Publication No. WO 01/38318, published May 31, 2001)), tubulysins, and colchicines; topoisomerase inhibitors (e.g., irinotecan, topotecan, camptothecin, etoposide, teniposide, amsacrine, or mitoxantrone); and proteasome inhibitors (e.g., peptidyl boronic acids). Additional information on the conjugation of various therapeutic agents to anti-GCC antibodies is found in International Application WO2011/050242, which is hereby incorporated by references in its entirety.

### Dolastatin and Auristatin Immunoconjugates

In some other embodiments the therapeutic agent is a dolastatin. In some embodiments, the therapeutic agent is an auristatin, such as auristatin E (also known in the art as a derivative of dolastatin-10) or a derivative thereof. In some embodiments, the therapeutic agent is a compound selected from compounds of formulae (XIII)-(XXIII), or a pharmaceutically acceptable salt form thereof:

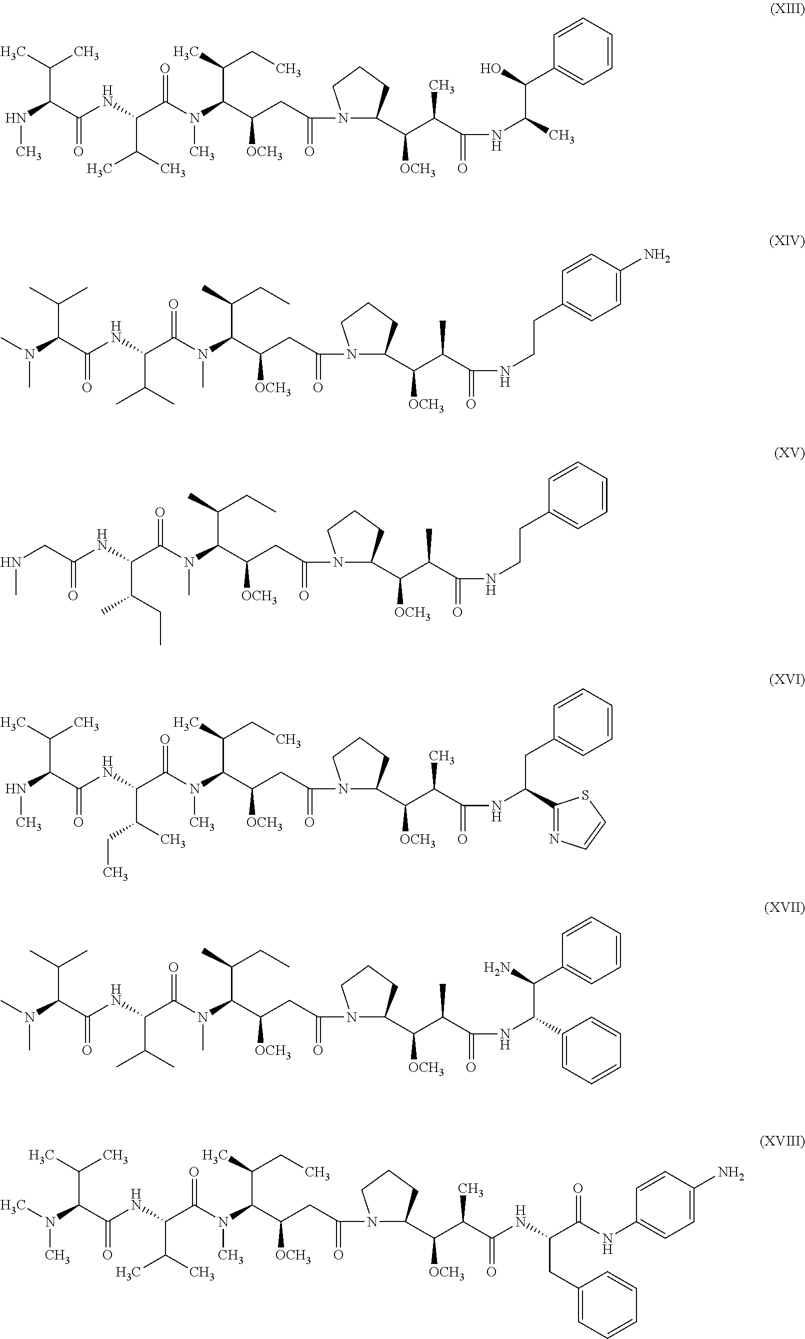

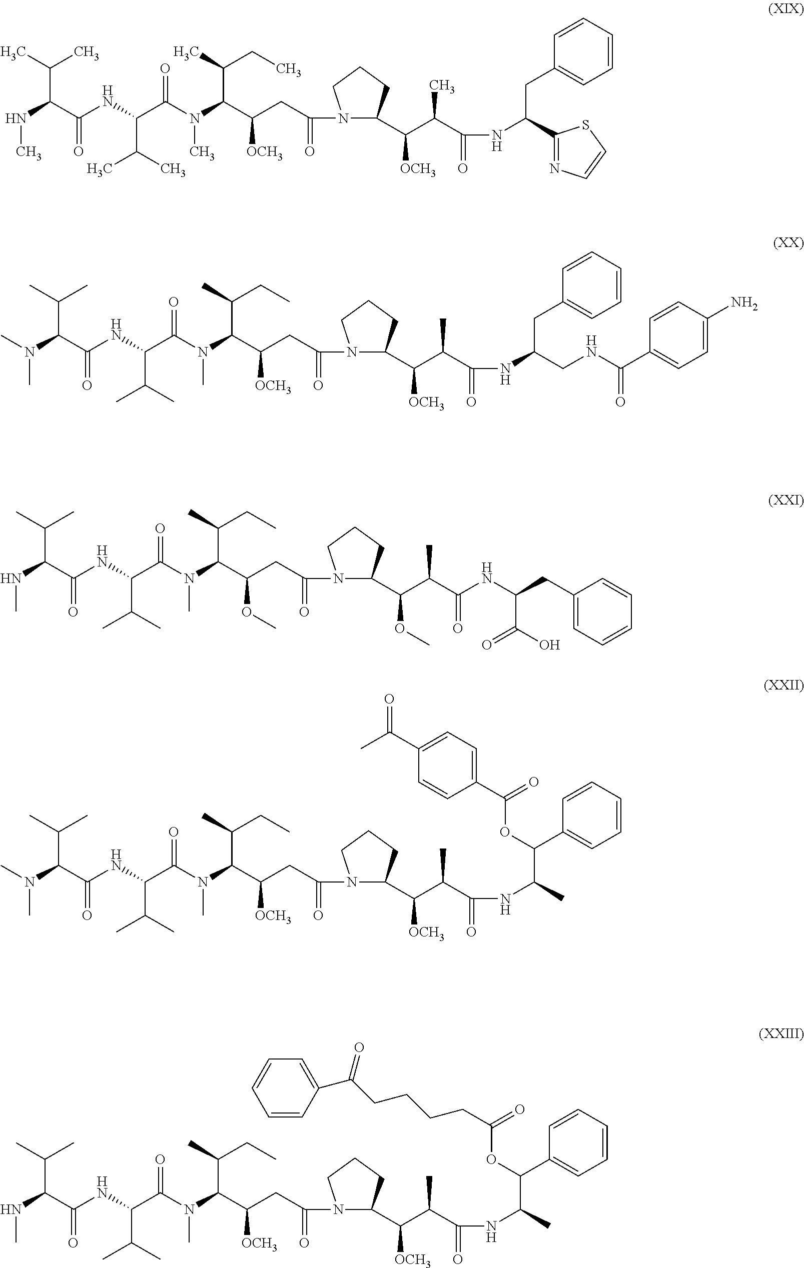

Auristatin compounds and methods for their conjugation to antibodies are described, for example, in Doronina et al., Nature Biotech., 21: 778-784 (2003); Hamblett et al, Clin. Cancer Res., 10: 7063-7070 (2004); Carter and Senter, Cancer J., 14 154-169 (2008); U.S. Pat. Nos. 7,498,298, 7,091,186, 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414; U.S. Patent Publication Nos. 20090010945, 20060074008, 20080300192, 20050009751, 20050238649, and 20030083236; and International Patent Publication Nos. WO 04/010957 and WO 02/088172, each of which is incorporated by reference herein in its entirety and for all purposes.

The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include auristatin phenylalanine phenylenediamine (AFP; (XVIII)), monomethyl auristatin E (MMAE; (XIII)), and monomethyl auristatin F (MMAF; (XXI)).

Auristatins have been shown to interfere with microtubule dynamics and nuclear and cellular division and have anticancer activity. Auristatins for use in the present invention bind tubulin and can exert a cytotoxic or cytostatic effect on a GCC-expressing cell line. Methods for determining whether a compound binds tubulin are known in the art. See, for example, Muller et al., Anal. Chem. 2006, 78, 4390-4397; Hamel et al., Molecular Pharmacology, 1995 47: 965-976; and Hamel et al., The Journal of Biological Chemistry, 1990 265:28, 17141-17149. For purposes of the present invention, the relative affinity of a compound to tubulin can be determined. Some preferred auristatins of the present invention bind tubulin with an affinity ranging from 10-fold lower (weaker affinity) than the binding affinity of MMAE to tubulin to 10-fold, 20-fold or even 100-fold higher (higher affinity) than the binding affinity of MMAE to tubulin.

There are a number of different assays, known in the art, that can be used for determining whether an auristatin or resultant immunoconjugate exerts a cytostatic or cytotoxic effect on a desired cell line. For example, the cytotoxic or cytostatic activity of an immunoconjugate can be measured by: exposing mammalian cells expressing a target protein of the immunoconjugate in a cell culture medium; culturing the cells for a period from about 6 hours to about 5 days; and measuring cell viability. Cell-based in vitro assays can be used to measure viability (proliferation), cytotoxicity, and induction of apoptosis (caspase activation) of the immunoconjugate.

For determining whether an immunoconjugate exerts a cytostatic effect, a thymidine incorporation assay may be used. For example, cancer cells expressing a target antigen at a density of 5,000 cells/well of a 96-well plated can be cultured for a 72-hour period and exposed to 0.5 μCi of ³H-thymidine during the final 8 hours of the 72-hour period. The incorporation of ³H-thymidine into cells of the culture is measured in the presence and absence of the immunoconjugate.

For determining cytotoxicity, necrosis or apoptosis (programmed cell death) can be measured. Necrosis is typically accompanied by increased permeability of the plasma membrane; swelling of the cell, and rupture of the plasma membrane. Apoptosis is typically characterized by membrane blebbing, condensation of cytoplasm, and the activation of endogenous endonucleases. Determination of any of these effects on cancer cells indicates that an immunoconjugate is useful in the treatment of cancers.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytotoxicity (Skehan et al., 1990, J. Natl. Cancer Inst. 82:1107-12).

Alternatively, a tetrazolium salt, such as MTT or WST, is used in a quantitative colorimetric assay for mammalian cell survival and proliferation by detecting living, but not dead, cells (see, e.g., Mosmann, 1983, J. Immunol. Methods 65:55-63).

Apoptosis can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Apoptosis can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

The presence of apoptotic cells can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting apoptosis (e.g., by measuring DNA fragmentation). (See, e.g., Piazza et al., 1995, Cancer Research 55:3110-16).

The effects of immunoconjugates can be tested or validated in animal models. A number of established animal models of cancers are known to the skilled artisan, any of which can be used to assay the efficacy of an immunoconjugate. Non-limiting examples of such models are described infra. Moreover, small animal models to examine the in vivo efficacies of immunoconjugates can be created by implanting human tumor cell lines into appropriate immunodeficient rodent strains, e.g., athymic nude mice or SCID mice.

In some embodiments, the variable —Z in Formula (I) is an auristatin moiety of the Formula (X-A) or Formula (X-B):

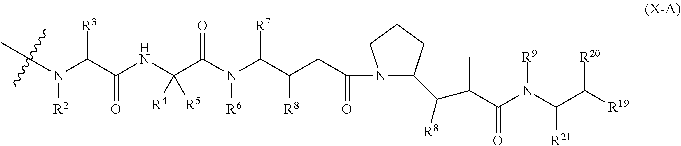

wherein, independently at each location:
the wavy line indicates a bond;
R² is —C₁-C₂₀ alkyl, —C₂-C₂₀ alkenyl, or —C₂-C₂₀ alkynyl;
R³ is —H, —C₁-C₂₀ alkyl, —C₂-C₂₀ alkenyl, —C₂-C₂₀ alkynyl, carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), -heterocycle, —C₁-C₂₀ alkylene(heterocycle), —C₂-C₂₀ alkenylene(heterocycle), or —C₂-C₂₀ alkynylene(heterocycle);
R⁴ is —H, —C₁-C₂₀ alkyl, —C₂-C₂₀ alkenyl, —C₂-C₂₀ alkynyl, carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), -heterocycle, —C₁-C₂₀ alkylene(heterocycle), —C₂-C₂₀ alkenylene(heterocycle), or —C₂-C₂₀ alkynylene(heterocycle);
R⁵ is —H or —C₁-C₈ alkyl;
or R⁴ and R⁵ jointly form a carbocyclic ring and have the formula —(CR^{a}R^{b})ₛ— wherein R^{a} and R^{b} are independently —H, —C₁-C₂₀ alkyl, —C₂-C₂₀ alkenyl, —C₂-C₂₀ alkynyl, or -carbocycle and s is 2, 3, 4, 5 or 6;
R⁶ is —H, —C₁-C₂₀ alkyl, —C₂-C₂₀ alkenyl, or —C₂-C₂₀ alkynyl;
R⁷ is —H, —C₁-C₂₀ alkyl, —C₂-C₂₀ alkenyl, —C₂-C₂₀ alkynyl, -carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), heterocycle, —C₁-C₂₀ alkylene(heterocycle), —C₂-C₂₀ alkenylene(heterocycle), or —C₂-C₂₀ alkynylene(heterocycle);
each R⁸ is independently —H, —OH, —C₁-C₂₀ alkyl, —C₂-C₂₀ alkenyl, —C₂-C₂₀ alkynyl, —O—(C₁-C₂₀ alkyl), —O—(C₂-C₂₀ alkenyl), —O—(C₁-C₂₀ alkynyl), or -carbocycle;
R⁹ is —H, —C₁-C₂₀ alkyl, —C₂-C₂₀ alkenyl, or —C₂-C₂₀ alkynyl;
R¹⁹ is -aryl, -heterocycle, or -carbocycle;
R²⁰ is —H, —C₁-C₂₀ alkyl, —C₂-C₂₀ alkenyl, —C₂-C₂₀ alkynyl, -carbocycle, —O—(C₁-C₂₀ alkyl), —O—(C₂-C₂₀ alkenyl), —O—(C₂-C₂₀ alkynyl), or OR¹⁸ wherein R¹⁸ is —H, a hydroxyl protecting group, or a direct bond where OR¹⁸ represents ═O; and
R²¹ is —H, —C₁-C₂₀ alkyl, —C₂-C₂₀ alkenyl, or —C₂-C₂₀ alkynyl, -aryl, heterocycle, or -carbocycle;

Auristatins of the Formula (X-A) include those wherein said alkyl, alkenyl, alkynyl, alkylene, alkenylene, alkynyklene, aryl, carbocyle, and heterocycle radicals are unsubstituted.

Auristatins of the Formula (X-A) include those wherein the groups of R², R³, R⁴, R⁵, R⁶, R⁷, R⁸, and R⁹ are unsubstituted and the groups of R¹⁹, R²⁰ and R²¹ are optionally substituted as described herein.

Auristatins of the Formula (X-A) include those wherein:

R² is —C₁-C₈ alkyl;
R³, R⁴ and R⁷ are independently selected from —H, —C₁-C₂₀ alkyl, —C₂-C₂₀ alkenyl, —C₂-C₂₀ alkynyl, monocyclic C₃-C₆ carbocycle, —C₁-C₂₀ alkylene(monocyclic C₃-C₆ carbocycle), —C₂-C₂₀ alkenylene(monocyclic C₃-C₆ carbocycle), —C₂-C₂₀ alkynylene(monocyclic C₃-C₆ carbocycle), —C₆-C₁₀ aryl, —C₁-C₂₀ alkylene(C₆-C₁₀ aryl), —C₂-C₂₀ alkenylene(C₆-C₁₀ aryl), —C₂-C₂₀ alkynylene(C₆-C₁₀ aryl), -heterocycle, —C₁-C₂₀ alkylene(heterocycle), —C₂-C₂₀ alkenylene(heterocycle), or —C₂-C₂₀ alkynylene(heterocycle); wherein said alkyl, alkenyl, alkynyl, alkylene, alkenylene, alkynylene, carbocycle, aryl, and heterocycle radicals are optionally substituted;
R⁵ is -hydrogen;
R⁶ is —C₁-C₈ alkyl;
each R⁸ is independently selected from —OH, —O—(C₁-C₂₀ alkyl), —O—(C₂-C₂₀ alkenyl), or —O—(C₂-C₂₀ alkynyl) wherein said alkyl, alkenyl, and alkynyl radicals are optionally substituted;
R⁹ is -hydrogen or —C₁-C₈ alkyl;
R¹⁹ is optionally substituted phenyl;
R²⁰ is OR¹⁸; wherein R¹⁸ is H, a hydroxyl protecting group, or a direct bond where OR¹⁸ represents ═O;
R²¹ is selected from —H, —C₁-C₂₀ alkyl, —C₂-C₂₀ alkenyl, —C₂-C₂₀ alkynyl, or -carbocycle; wherein said alkyl, alkenyl, alkynyl, and carbocycle radicals are optionally substituted; or a pharmaceutically acceptable salt form thereof.

Auristatins of the Formula (X-A) include those wherein:

R² is methyl;
R³ is —H, —C₁-C₈ alkyl, —C₂-C₈ alkenyl, or —C₂-C₈ alkynyl, wherein said alkyl, alkenyl and alkynyl radicals are optionally substituted;
R⁴ is —H, —C₁-C₈ alkyl, —C₂-C₈ alkenyl, —C₂-C₈ alkynyl, monocyclic C₃-C₆ carbocycle, —C₆-C₁₀ aryl, —C₁-C₈ alkylene(C₆-C₁₀ aryl), —C₂-C₈ alkenylene(C₆-C₁₀ aryl), —C₂-C₈ alkynylene(C₆-C₁₀ aryl), —C₁-C₈ alkylene (monocyclic C₃-C₆ carbocycle), —C₂-C₈ alkenylene (monocyclic C₃-C₆ carbocycle), —C₂-C₈ alkynylene(monocyclic C₃-C₆ carbocycle); wherein said alkyl, alkenyl, alkynyl, alkylene, alkenylene, alkynylene, aryl, and carbocycle radicals whether alone or as part of another group are optionally substituted;
R⁵ is H; R⁶ is methyl;
R⁷ is —C₁-C₈ alkyl, —C₂-C₈ alkenyl or —C₂-C₈ alkynyl;
each R⁸ is methoxy;
R⁹ is -hydrogen or —C₁-C₈ alkyl;
R¹⁹ is phenyl;
R²⁰ is OR¹⁸; wherein R¹⁸ is H, a hydroxyl protecting group, or a direct bond where OR¹⁸ represents ═O;
R²¹ is methyl; or a pharmaceutically acceptable salt form thereof.

Auristatins of the Formula (X-A) include those wherein: R² is methyl; R³ is H or C₁-C₃ alkyl; R⁴ is C₁-C₅ alkyl; R⁵ is H; R⁶ is methyl; R⁷ is isopropyl or sec-butyl; R⁸ is methoxy; R⁹ is hydrogen or C₁-C₈ alkyl; R¹⁹ is phenyl; R²⁰ is OR¹⁸; wherein R¹⁸ is H, a hydroxyl protecting group, or a direct bond where OR¹⁸ represents ═O; and R²¹ is methyl; or a pharmaceutically acceptable salt form thereof.

Auristatins of the Formula (X-A) include those wherein: R² is methyl or C₁-C₃ alkyl; R³ is H or C₁-C₃ alkyl; R⁴ is C₁-C₅ alkyl; R⁵ is H; R⁶ is C₁-C₃ alkyl; R⁷ is C₁-C₅ alkyl; R⁸ is C₁-C₃ alkoxy; R⁹ is hydrogen or C₁-C₈ alkyl; R¹⁹ is phenyl; R²⁰ is OR¹⁸; wherein R¹⁸ is H, a hydroxyl protecting group, or a direct bond where OR¹⁸ represents ═O; and R²¹ is C₁-C₃ alkyl; or a pharmaceutically acceptable salt form thereof.

In preferred embodiments of the immunoconjugates of Formula (II), when Z is an auristatin molecule of Formula (X-A), w is an integer ranging from 1 to 12, preferably 2 to 12, y is 1 or 2, and a is preferably 1.

Illustrative therapeutic agents (—Z) include those having the following structures:

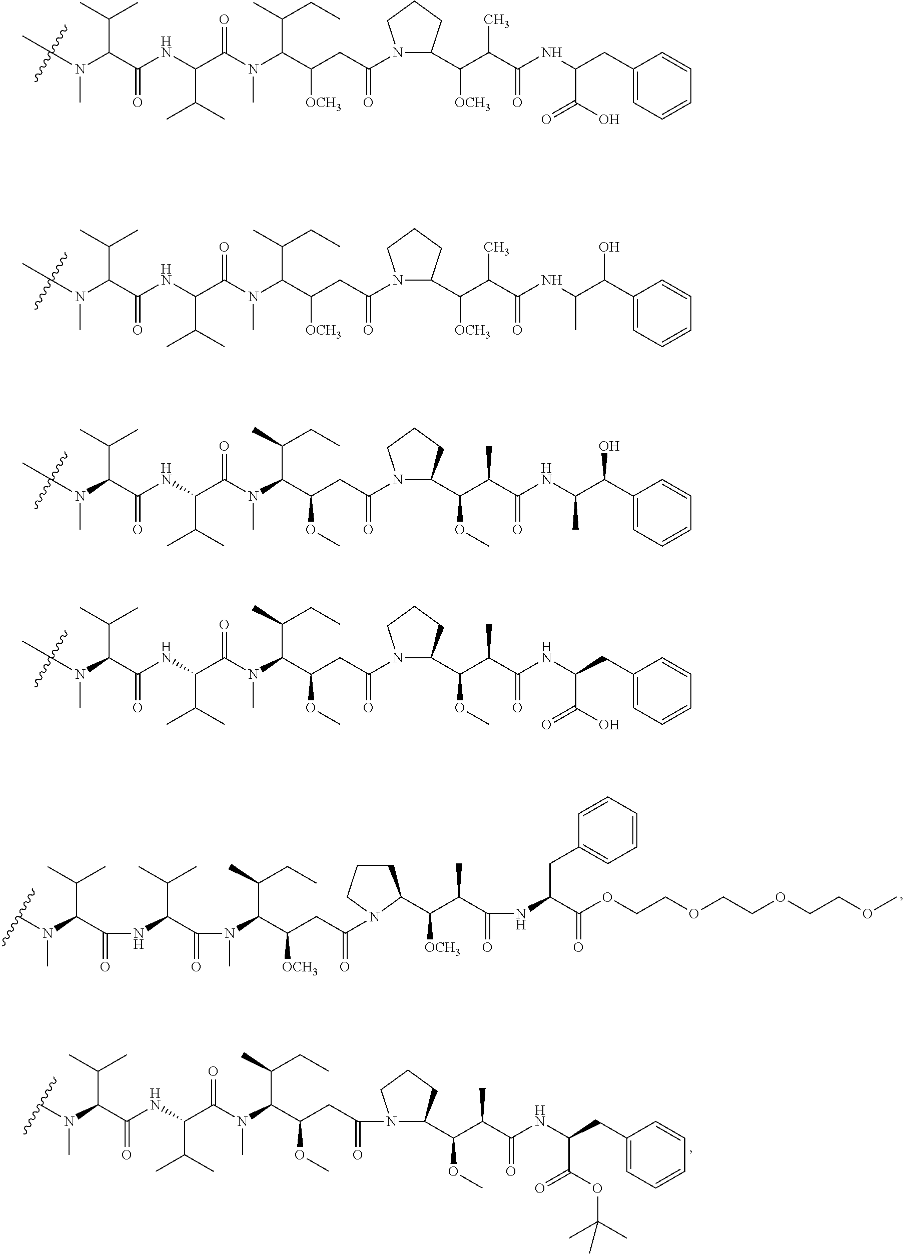

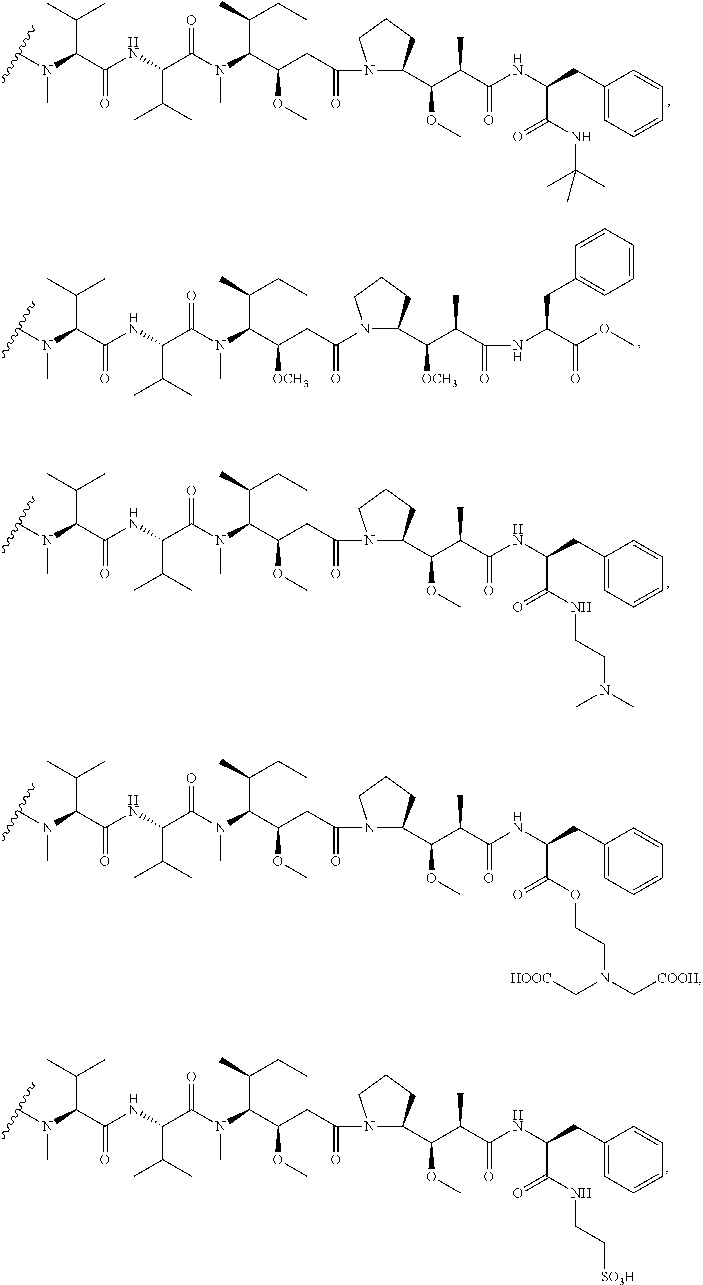

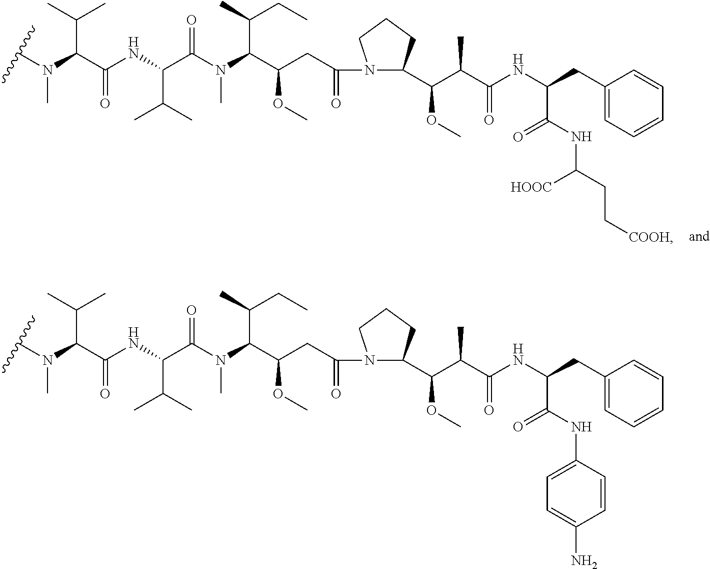 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)NC(CCC(=O)O)C(=O)O)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)Nc1ccc(N)cc1)OC)C(C)C)C(C)C]

In some embodiments, the therapeutic agent is not TZT-1027. In some embodiments, the therapeutic agent is not auristatin E, dolastatin 10, or auristatin PE.

In some embodiments, the auristatin molecule is linked to a cysteine moiety on the antibody molecule by way of a linker containing a maleimide moiety, e.g., a maleimidocaproyl moiety.

In some other embodiments the auristatin molecule is coupled to the antibody using a heterobifunctional linker that is connected to a monomethyl amino group on the auristatin molecule. In some embodiments, the linker comprises a cleavable moiety, e.g., a peptide moiety, and a self-immolative p-aminobenzylcarbamate spacer. Exemplary linkers include maleimidocaproyl (mc), maleimidocaproyl-L-phenylalanine-L-lysine-p-aminobenzyl-carbamate, and maleimidocaproyl-L-valine-L-citrulline-p-aminobenzylcarbamate (vc).

In certain embodiments, the immunoconjugate of Formula (I) is characterized by the formula Ab-(vc-MMAF)ₘ (Formula (I-4)); Ab-(vc-MMAE)ₘ (Formula (I-5)); Ab-(mc-MMAE)ₘ (Formula (I-6)); or Ab-(mc-MMAF)ₘ, (Formula (I-7)), wherein Ab is an anti-GCC antibody molecule as described herein, S is a sulfur atom of the antibody, and m has the values and preferred values described above for Formula (I). In certain embodiments, m is an integer from 1 to about 5.

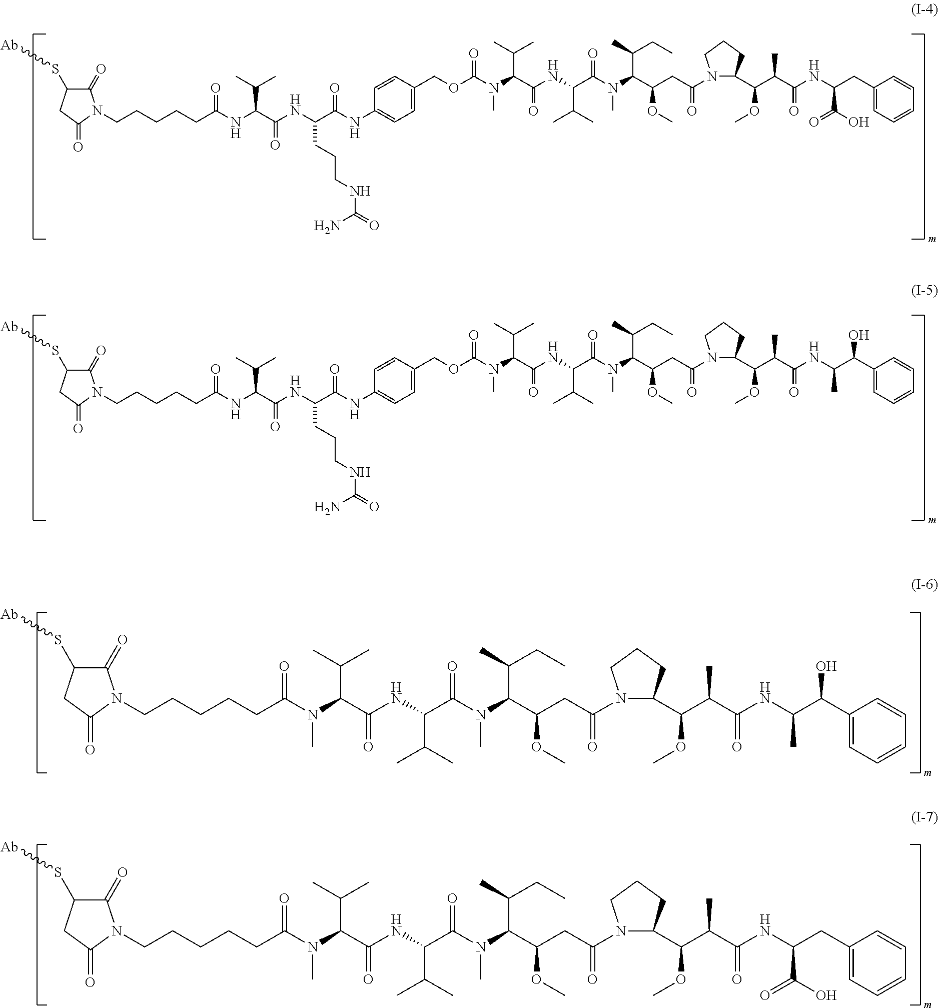

In some embodiments, the variable Ab in Formula (I-4), (I-5), (I-6), or (I-7) is an antibody molecule with one or more features summarized in Tables 1 to 6. In certain embodiments, the variable Ab is a 5F9 antibody molecule or an Abx-229 antibody molecule.

In some embodiments, the variable m in Formula (I-4), (I-5), (I-6), or (I-7) ranges from about 2 to about 10, from about 6 to about 8, or from about 4 to about 6.

In certain particular embodiments, the invention relates to an immunoconjugate of Formula (I-4), (I-5), (I-6), or (I-7), wherein Ab is a 5F9 antibody molecule and m is about 4.

The immunoconjugates disclosed herein can be used for modifying a given biological response. The therapeutic agent is not to be construed as limited to classical chemical therapeutic agents. For example, the therapeutic agent may be a nucleic acid, protein, or polypeptide possessing a desired biological activity. For example, the antibody molecule can be conjugated to an antisense molecule, an siRNA molecule, shRNA molecule or miRNA molecule that can interfere with expression of a gene, thereby producing a desired biological effect.

An anti-GCC antibody molecule described herein can also be conjugated to a prodrug or prodrug activator.

### Pharmaceutical Compositions

In another aspect, the invention features compositions, e.g., pharmaceutically acceptable compositions, kits comprising such compositions and methods of using such compositions. The composition can include an anti-GCC antibody molecule or immunoconjugate thereof, as described herein, formulated together with a pharmaceutically acceptable carrier. In embodiments, the anti-GCC antibody molecule is one with exemplary features summarized in Tables 1-6.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). The pharmaceutical composition can include one or more additional excipients, e.g., salts, buffers, tonicity modifiers, lyoprotectants, nonionic detergents, surfactants, and preservatives.

The compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Some typical compositions are in the form of injectable or infusible solutions, intended for parenteral administration (e.g., intravenous, subcutaneous, intraperitoneal, intramuscular). In some embodiments, the composition is administered by intravenous infusion or injection. In other embodiments, the composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, the pharmaceutical composition is sterile and stable under the conditions of manufacture and storage. The composition can be formulated as a solution, microemulsion, dispersion, liposome, microsphere, or other ordered structure suitable to high antibody concentration. Sterile injectable solutions can be prepared by incorporating the active compound (e.g., antibody, antibody portion, or immunoconjugate) in the required amount in an appropriate solvent with one or a combination of ingredients enumerated above, as required, followed by sterilization, e.g., by filtration. Generally, dispersions are prepared by incorporating the active compound into a sterile vehicle that contains a basic dispersion medium and the required other ingredients from those enumerated above. In the case of sterile powders for the preparation of sterile injectable solutions, the provided methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules and immunoconjugates described herein can be administered by a variety of methods known in the art, although for many therapeutic applications, the route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an anti-GCC antibody molecule or immunoconjugate described herein may be orally administered, for example, with an inert diluent or an assimilable edible carrier. The compound (and other ingredients if desired) may also be enclosed in a hard or soft shell gelatin capsule, compressed into tablets, buccal tablets, troches, capsules, elixirs, suspensions, syrups, wafers, and the like. To administer an antibody molecule or immunoconjugate described herein by other than parenteral administration, it may be necessary to coat the compound with, or co-administer the compound with, a material to prevent its inactivation.

Therapeutic compositions can be administered with medical devices known in the art. For example, pharmaceutical preparations can be disposed within a device, e.g., an air- or liquid-tight container, which contains one or more dosages. Examples of delivery devices include, without limitation, vials, cannulas, needles, drip bags, and lines. The invention also provides methods of placing an antibody molecule or immunoconjugate described herein into such a device.

In some embodiments, the invention provides an anti-GCC antibody molecule or immunoconjugate described herein, which is formulated in a liposome composition. In some embodiments, the liposome is coated with antibody molecule. In some such embodiments, the liposome is filled with a therapeutic agent. Liposomic delivery can allow for the delivery of an agent, e.g., a therapeutic agent that is not linked to the antibody. This approach can be used to deliver an agent, e.g., a therapeutic agent, that is not amenable to cross-linking to the antibody molecule or an agent, e.g., a therapeutic agent, which is to be sequestered, or which contact with non-target cells should be minimized. In particular embodiments, the liposome is filled with a cytostatic or cytotoxic agent. In certain particular embodiments, the therapeutic agent is selected from the group consisting of maytansinoids, auristatins, dolastatins, duocarmycins, cryptophycins, taxanes, DNA alkylating agents, calicheamicins, and derivatives of the foregoing. In other embodiments, the liposome is filled with nucleic acid sequence comprising RNA interference molecules, e.g., antisense molecules, siRNA, hsRNA or miRNA molecules, which are capable of diminishing GCC expression or the expression of another gene, e.g., an oncogene, in cells expressing GCC. In some other embodiments, the liposome is coated or filled with an immunoconjugate comprising an anti-GCC antibody molecule and a therapeutic agent or label.

Dosage regimens are adjusted to provide the optimum desired response (e.g., a therapeutic response). For example, a single bolus of an anti-GCC antibody molecule or immunoconjugate described herein may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form,” as used herein, refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an anti-GCC antibody molecule or immunoconjugate of the invention is 0.1-20 mg/kg, or 1-10 mg/kg. In one particular embodiment, a therapeutically or prophylactically effective amount of an anti-GCC antibody molecule or immunoconjugate of the invention ranges from approximately 1.8 mg/kg-3.5 mg/kg, or any specific value in between such range, such as 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg or 3.5 mg/kg. In some embodiments, the anti-GCC antibody molecule or immunoconjugate thereof is administered at a dose high enough to achieve synergy with a second therapeutic agent, such as a DNA damaging agent. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective” amount of an anti-GCC antibody molecule or immunoconjugate of the invention. A “therapeutically effective” amount refers to an amount effective, at dosages and for periods of time necessary, to achieve the desired therapeutic result. A therapeutically effective amount of the antibody molecule or immunoconjugate may vary according to factors such as the disease state, age, sex, and weight of the individual, and the ability of the antibody molecule or immunoconjugate to elicit a desired response in the individual. A therapeutically effective amount is also one in which any toxic or detrimental effects of the antibody molecule or immunoconjugate is outweighed by the therapeutically beneficial effects. A “therapeutically effective dosage” preferably inhibits a measurable parameter (e.g., tumor burden and/or tumor growth rate) in treated subjects by at least about 20%, at least about 40%, at least about 60%, and in some embodiments at least about 80%, relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

In another aspect the invention features compositions, e.g., pharmaceutically acceptable compositions, which include an anti-GCC antibody molecule or immunoconjugate thereof, as described herein, formulated together with an additional therapeutic agent, and pharmaceutically acceptable carrier. The anti-GCC antibody molecule or immunoconjugate thereof and the additional therapeutic agent are provided in therapeutically effective amounts when used in combination. In certain embodiments, the additional therapeutic agent is a DNA damaging agent, including, for example, topoisomerase I inhibitors, topoisomerase II inhibitors, alkylating agents, alkylating-like agents, anthracyclines, DNA intercalators, DNA minor groove alkylating agents, and antimetabolites. Particular examples of each such DNA damaging agents are described herein. The anti-GCC antibody molecule can be one with exemplary features summarized in Tables 1-6.

In certain embodiments, the invention features a pharmaceutically acceptable composition which includes an immunoconjugate according to Formula (I-4), (I-5), (I-6), or (I-7) as described herein, wherein the variable Ab is an anti-GCC antibody molecule described herein, e.g., with the exemplary features described in one or more of Tables 1-6, and a topoisomerase I inhibitor, wherein each of the immunoconjugate and the topoisomerase I inhibitor are present in a therapeutically effective total amount. For example, without limitation, the pharmaceutically acceptable composition includes the immunoconjugate of formula I-5, wherein the variable Ab is a 5F9 antibody molecule and m is about 4, and irinotecan.

In certain embodiments, an immunoconjugate of the invention may be formulated in combination with a DNA damaging agent in unit dosage for ease of administration and uniformity of dosage. The expression “unit dosage form” as used herein refers to a physically discrete unit of agent appropriate for the patient to be treated. It will be understood however, that the total daily usage of the compositions of the present invention will be decided by the attending physician within the scope of sound medical judgment. In a particular embodiment, a unit dosage form of the immunoconjugate and DNA damaging agent is a liquid solution or a suspension suitable for parenteral administration. In another particular embodiment, the unit dosage form of the immunoconjugate and DNA damaging agent is lyophilized and is suitable for parenteral administration upon resuspension (e.g., in a pharmaceutically acceptable carrier). A unit dose form for parenteral administration may be in ampoules or in multi-dose containers.

Also within the scope of the invention are kits comprising an anti-GCC antibody molecule or immunoconjugate as described herein. Further included are kits comprising liposome compositions comprising an anti-GCC antibody molecule or immunoconjugate. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject, e.g., for therapeutic or diagnostic use. Instructions for use can include instructions for diagnostic applications of an anti-GCC antibody molecule or other molecule, e.g., peptide, to detect GCC, in vitro, e.g., in a sample, e.g., a biopsy or cells from a patient having a cancer, or in vivo. The instructions can include guidance for therapeutic application including suggested dosages and/or modes of administration, e.g., in a patient with a cancer (e.g., a cancer of gastrointestinal origin, such as, for example, colon cancer, gastric cancer, esophageal cancer). Other instructions can include instructions on coupling of the antibody to a therapeutic agent, or for purification of a conjugated antibody, e.g., from unreacted conjugation components. As discussed above, the kit can include a label, e.g., any of the labels described herein. As discussed above, the kit can include a therapeutic agent, e.g., a therapeutic agent described herein. In some applications the antibody will be reacted with other components, e.g., a chelator or a label or therapeutic agent, e.g., a radioisotope, e.g., yttrium or lutetium. In such cases the kit can include one or more of a reaction vessel to carry out the reaction or a separation device, e.g., a chromatographic column, for use in separating the finished product from starting materials or reaction intermediates.

The kit can further contain at least one additional reagent, such as a diagnostic or therapeutic agent, e.g., a diagnostic or therapeutic agent as described herein, and/or one or more additional anti-GCC antibody molecules or immunoconjugates, formulated as appropriate, in one or more separate pharmaceutical preparations, or formulated as a single dosage form.

In certain embodiments, the kit contains an immunoconjugate characterized by Formula (I-4), (I-5), (I-6), or (I-7) as described herein, wherein the variable Ab is an is an anti-GCC antibody molecule with exemplary features summarized in Tables 1-6, and instructions for therapeutic application including suggested dosages and/or modes of administration of the immunoconjugate in combination with a DNA damaging agent, e.g., in a patient with a cancer (e.g., a primary or metastatic cancer of gastrointestinal origin, such as, for example, colon cancer, gastric cancer, pancreatic cancer or esophageal cancer). Optionally, the kit further contains a separate pharmaceutical preparation of the DNA damaging agent for use in combination with the immunoconjugate. Examples of DNA damaging agents suitable for use in combination with a immunoconjugate include, for example, topoisomerase I inhibitors, topoisomerase II inhibitors, alkylating agents, alkylating-like agents, anthracyclines, DNA intercalators, DNA minor groove alkylating agents, and antimetabolites, and particular examples of each such DNA damaging agents are described herein. For example, without limitation, the kit contains a pharmaceutical preparation of the immunoconjugate according to Formula (I-5) as described herein, wherein the variable Ab is a 5F9 antibody molecule and m is about 4, and instructions for therapeutic application including suggested dosages and/or modes of administration of the immunoconjugate in combination with a topoisomerase I inhibitor, such as irinotecan. Optionally, the kit further contains a separate pharmaceutical preparation of the topoisomerase I inhibitor for use in combination with the immunoconjugate.

As yet another example, without limitation, the kit can contain a pharmaceutical preparation of the immunoconjugate according to Formula (I-5), wherein the variable Ab is a 5F9 antibody molecule and m is about 4, and a DNA damaging agent, such as a topoisomerase I inhibitor (e.g., irinotecan) or a DNA minor groove alkylating agent (e.g., a duocarmycin such as CC-1065 or any analog or derivative thereof; a pyrrolobenzodiazepene, or any analog or derivative thereof), where the immunoconjugate and the DNA damaging agent are co-formulated (e.g., a single dosage form), and instructions for therapeutic application including dosing schedule and modes of administration, e.g., in a patient with a cancer (e.g., a primary or metastatic cancer of gastrointestinal origin, such as, for example, colon cancer, gastric cancer, pancreatic cancer or esophageal cancer).

A provided kit can include a chelator—conjugated protein or peptide with a therapeutic radioisotope for administration to a patient. The kit can include (i) a vial containing chelator-conjugated antibody, (ii) a vial containing formulation buffer for stabilizing and administering the radiolabeled antibody to a patient, and (iii) instructions for performing the radiolabeling procedure. The kit provides for exposing a chelator-conjugated antibody to the radioisotope or a salt thereof for a sufficient amount of time under amiable conditions, e.g., as recommended in the instructions. A radiolabeled antibody having sufficient purity, specific activity and binding specificity is produced. The radiolabeled antibody may be diluted to an appropriate concentration, e.g., in formulation buffer, and administered directly to the patient with or without further purification. The chelator-conjugated antibody may be supplied in lyophilized form.

### Uses

The anti-GCC antibody molecules described herein have in vitro and in vivo therapeutic and prophylactic utilities. For example, these antibody molecules can be administered to cells in culture, e.g. in vitro or ex vivo, or administered in a subject, e.g., in vivo, to treat, prevent, and/or diagnose a variety of disorders.

The antibody molecules and immunoconjugates described herein can modulate an activity or function of a GCC protein, such as ligand binding (e.g., binding of ST or guanylin), GCC-mediated signal transduction, maintenance of intestinal fluid, electrolyte homeostasis, intracellular calcium release (calcium flux), cell differentiation, cell proliferation, or cell activation.

In one aspect, the invention features a method of killing, inhibiting or modulating the growth of, or interfering with the metabolism of, a GCC-expressing cell. In one embodiment, the invention provides a method of inhibiting GCC-mediated cell signaling or a method of killing a cell. The method may be used with any cell or tissue which expresses GCC, such as a cancerous cell (e.g., a cell from a cancer of the gastrointestinal system, such as, for example, a cancer of the colon, gastric, pancreas or esophagus), or a metastatic lesion. Nonlimiting examples of GCC-expressing cells include T84 human colonic adenocarcinoma cells, fresh or frozen colonic tumor cells, and cells comprising a recombinant nucleic acid encoding GCC or a portion thereof.

Methods of the invention include the steps of contacting the cell with an anti-GCC antibody molecule or immunoconjugate thereof, as described herein, in an effective amount, i.e., amount sufficient to inhibit GCC-mediated cell signaling or an amount sufficient to kill the cell. The method can be used on GCC-expressing cells in culture, e.g. in vitro, ex vivo, or in situ. For example, cells that express GCC (e.g., cells collected by biopsy of a tumor or metastatic lesion; cells from an established cancer cell line; or recombinant cells), can be cultured in vitro in culture medium and the contacting step can be effected by adding the anti-GCC antibody molecule or immunoconjugate to the culture medium. In methods of killing a cell, the method comprises using a naked anti-GCC antibody molecule, or an immunoconjugate comprising an anti-GCC antibody molecule and a cytotoxic agent, e.g., a DNA damaging agent. The method will result in killing of cells expressing GCC, including in particular tumor cells expressing GCC (e.g., colonic tumor cells).

Anti-GCC antibody molecules of the present invention bind to extracellular domains of GCC or portions thereof in cells expressing the antigen. As a result, when practicing the methods of the present invention to kill and/or suppress cancerous cells, the antibody molecules, bind to all such cells, not only to cells which are fixed or cells whose intracellular antigenic domains are otherwise exposed to the extracellular environment. Consequently, binding of the antibody molecules, is concentrated in areas where there are cells expressing GCC, irrespective of whether these cells are fixed or unfixed, viable or necrotic. Additionally or alternatively, the anti-GCC antibody molecules bind to and are internalized with GCC upon binding cells expressing the antigen.

Table 7 indicates an antibody molecule which was confirmed to internalize after binding GCC. This antibody is useful, e.g., when linked to a cytotoxic moiety, for therapeutic uses.

The method also can be performed on cells present in a subject, as part of an in vivo protocol. In one embodiment, the subject is a human subject. Alternatively, the subject can be a non-human mammal expressing a GCC antigen with which an anti-GCC antibody molecule disclosed herein cross-reacts. In certain embodiments, the subject is a non-human mammal with transplanted human tissue (i.e., a xenograft model). An anti-GCC antibody molecule or immunoconjugate thereof, in combination with a DNA damaging agent, can be administered to a human subject for therapeutic purposes. An anti-GCC antibody molecule or immunoconjugate, in combination with a DNA damaging agent, also can be administered to a non-human mammal expressing the GCC-like antigen with which the antibody cross-reacts (e.g., a primate, pig, rat or mouse) for veterinary purposes or as an animal model of human disease (e.g., xenograft models of primary human tumor explants derived from metastatic colorectal cancer patients). Animal models may be useful for evaluating the therapeutic efficacy of an anti-GCC antibody or immunoconjugate described herein and DNA damaging agent combination described herein (e.g., testing of dosages and time courses of administration). For in vivo embodiments, the contacting step is effected in a subject and includes administering an anti-GCC antibody molecule or immunoconjugate thereof to the subject, in combination with a DNA damaging agent, under conditions effective to permit both binding of the antibody molecule to the extracellular domain of GCC expressed on the cell, and the treating of the cell. In some embodiments, the therapy results in one or more therapeutic effects from List 1 herein, e.g., one or more of synergy, prevention of tumor regrowth after ending administration, or effect on a tumor resistant to the immunoconjugate or DNA damaging agent.

In some embodiments, the immunoconjugate (e.g., an immunoconjugate according to Formula (I-5), optionally with an Ab comprising the CDRs of Table 5) in combination with a DNA damaging agent, produces one or more (e.g., 2, 3, 4, 5, 6, 7, 8, or 9, e.g., all) of the therapeutic effects of List 1:
(a) produces a synergistic response relative to the effect of the immunoconjugate or DNA damaging agent alone, wherein the response is optionally tumor growth inhibition (TGI) or tumor growth delay (TGD) (e.g., prevention of tumor growth or regrowth);
(b) produces an additive response relative to the effect of the immunoconjugate or DNA damaging agent alone, wherein the response is optionally TGI or TGD (e.g., prevention of tumor growth or regrowth);
(c) produces TGI or TGD (e.g., prevention of tumor growth or regrowth) that lasts at least 1, 2, 3, 4, 5, 6, 7, 8, 9, or 10 or more weeks after ending administration of the immunoconjugate, the DNA damaging agent, or both;
(d) results in TGI or TGD (e.g., prevention of tumor growth or regrowth), e.g., synergistic TGI or TGD, in a cancer displaying relatively high, moderate, or low GCC antigen density;
(e) results in synergistic TGI or TGD (e.g., prevention of tumor growth or regrowth) in a cancer displaying strong, moderate-to-strong, or moderate sensitivity to the immunoconjugate when administered alone (i.e., as a single agent therapeutic);
(f) results in TGI or TGD (e.g., prevention of tumor growth or regrowth) in a cancer displaying resistance to the immunoconjugate when administered alone (i.e., as a single agent therapeutic);
(g) results in synergistic TGI or TGD (e.g., prevention of tumor growth or regrowth) in a cancer displaying sensitivity to the DNA damaging agent when administered alone (i.e., as a single agent therapeutic);
(h) results in TGI or TGD (e.g., prevention of tumor growth or regrowth) in a cancer displaying resistance to the DNA damaging agent when administered alone (i.e., as a single agent therapeutic); and
(i) tumor growth inhibition upon administration of the therapy.

In one embodiment, the invention provides a method of treating cancer by administering an anti-GCC antibody molecule or an immunoconjugate comprising an anti-GCC antibody molecule and a cytotoxic agent, in combination with a DNA-damaging agent, to a patient in need of such treatment. The method can be used, e.g., for the treatment of any cancerous disorder which includes at least some cells that express the GCC antigen. As used herein, the term “cancer” is meant to include all types of cancerous growths or oncogenic processes, metastatic tissues or malignantly transformed cells, tissues, or organs, irrespective of histopathologic type or stage of invasiveness. The terms “cancer” and “tumor” may be used interchangeably (e.g., when used in the context of treatment methods, “treatment of a cancer” and “treatment of a tumor” have the same meaning).

In embodiments, the treatment is sufficient to reduce or inhibit the growth of the subject's tumor, reduce or inhibit regrowth of the subject's tumor after administration of such treatment, reduce the number or size of metastatic lesions, reduce tumor load, reduce primary tumor load, reduce invasiveness, prolong survival time, or maintain or improve the quality of life.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting colon. Adenocarcinomas include malignancies such as non-small cell carcinoma of the lung. Metastatic lesions of the aforementioned cancers can also be treated or prevented using the methods and compositions of the invention. In some embodiments, the cancer to be treated is a cancer of the gastrointestinal system (e.g., primary or metastatic colorectal, gastric, pancreatic, or esophageal cancer). In some embodiments, the therapy results in one or more therapeutic effects from List 1 herein against one of the aforementioned types of cancers, e.g., one or more of (a) synergy, (c) prevention of tumor regrowth after ending administration, or effect on a tumor resistant to the immunoconjugate (f) or DNA damaging agent (h), or (i) tumor growth inhibition upon administration of the therapy.

In one embodiment, the cancer is a colorectal cancer, e.g., colorectal adenocarcinoma, colorectal leiomyosarcoma, colorectal lymphoma, colorectal melanoma, or a colorectal neuroendocrine tumor. In a particular embodiment, the cancer is metastatic colon cancer. In another embodiment, the cancer is a gastric cancer (e.g., gastric adenocarcinoma, lymphoma, or sarcoma), or metastasis thereof. In another embodiment, the cancer is an esophageal cancer (e.g., a squamous cell carcinoma or adenocarcinoma of the esophagus). In some embodiments, the therapy results in one or more therapeutic effects from List 1 herein against one of the aforementioned types of cancers, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

The methods of the invention can be useful in treating a relevant disorder at any stage or sub classification. For example, method can be used to treat early or late stage colon cancer, or colon cancer of any of stages 0, I, IIA, IIB, IIIA, IIIB, IIIC, and IV.

In some embodiments, the methods for treating cancer (e.g., primary or metastatic colorectal, gastric, pancreatic, or esophageal cancer) comprises administering to a patient in need of such treatment a naked anti-GCC antibody molecule described herein. In other embodiments, the method comprises administering an immunoconjugate comprising an anti-GCC antibody molecule described herein and a cytotoxic agent, in combination with a DNA damaging agent. In some such embodiments, the immunoconjugate is characterized by Formula (I), as described herein. In certain embodiments, the immunoconjugate is characterized by Formula (I-1), (I-2), (I-3), (I-4), (I-5), (I-6), or (I-7) as described herein. In particular embodiments, the immunoconjugate is characterized by Formula (I), (I-1), (I-2), (I-3), (I-4), (I-5), (I-6), or (I-7), wherein the variable Ab is an antibody molecule with features summarized in Tables 1 to 6. In certain embodiments, the variable Ab is a 5F9 antibody molecule. In certain particular embodiments, the immunoconjugate is characterized by Formula (I-5) or (I-6), wherein the variable Ab is an anti-GCC antibody molecule described herein, e.g., a 5F9 antibody molecule. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

Methods of administering antibody molecules and immunoconjugates are described above. Suitable dosages of the molecules used will depend on the age and weight of the subject and the particular compound used.

In some embodiments, the anti-GCC antibody molecule or immunoconjugate and a DNA damaging agent are administered in treatment cycles. A “treatment cycle” consists of a treatment period, during which the anti-GCC antibody molecule or immunoconjugate and DNA damaging agent is administered as described above, followed by a rest period, during which no anti-GCC antibody molecule or immunoconjugate or DNA damaging agent is administered. The treatment cycle can be repeated as necessary to achieve the desired effect. In some embodiments, the desired effect is one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

The therapies described herein (e.g., anti-GCC immunoconjugates, in combination with a DNA damaging agent) may be used in combination with other therapies. For example, the combination therapy can include a composition of the present invention co-formulated with, and/or co-administered with, one or more additional therapeutic agents, e.g., one or more anti-cancer agents, e.g., cytotoxic or cytostatic agents, hormone treatment, vaccines, and/or other immunotherapies. In other embodiments, the anti-GCC immunoconjugates in combination with a DNA damaging agent are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies. Such combination therapies may also overcome and/or prevent chemoresistance to conventional therapeutic regimens.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concomitant” or “concurrent delivery”. In other embodiments, the delivery of one treatment ends before the delivery of the other treatment begins. This is sometimes referred to herein as “successive” or “sequential delivery” In embodiments of either case, the treatment is more effective because of combined administration. For example, the second treatment is a more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the first treatment delivered is still detectable when the second is delivered.

In some embodiments, the anti-GCC antibody molecule or immunoconjugate thereof (e.g., an immunoconjugate of Formula (I-5), wherein the anti-GCC antibody optionally comprises the CDRs of Table 5) is used in combination with another therapeutic agent, such as a chemotherapeutic agent. Non-limiting examples of DNA damaging chemotherapeutic agents include topoisomerase I inhibitors (e.g., irinotecan, topotecan, SN-38, lamellarin D, or camptothecin); topoisomerase II inhibitors (e.g., etoposide, teniposide, amsacrine, or mitoxantrone); anthracyclines (e.g., daunorubicin, doxorubicin, epirubicin, idarubicin, or valrubicin); alkylating agents (e.g., melphalan, chlorambucil, busulfan, thiotepa, ifosfamide, carmustine, lomustine, semustine, streptozocin, dacarbazine, mitomycin C, cyclophosphamide, mechlorethamine, uramustine, dibromomannitol, tetranitrate, procarbazine, altretamine, mitozolomide, or temozolomide); alkylating-like agents (e.g., cisplatin, oxaliplatin, carboplatin, nedaplatin, satraplatin, or triplatin); DNA intercalators and free radical generators such as bleomycin; DNA minor groove alkylating agents (e.g., duocarmycins such as CC-1065 and analogs or derivatives thereof; pyrrolobenzodiazepenes, or any analogs or derivatives thereof); and antimetabolites (e.g., fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, or pemetrexed). In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In some embodiments, the immunoconjugate is administered in combination with irinotecan, and synergistic efficacy results.

In some embodiments, the immunoconjugate is administered in combination with 5-fluorouracil, and synergistic efficacy results.

In some embodiments, the immunoconjugate is administered in combination with irinotecan to treat a cancer that is resistant to the immunoconjugate alone, and therapeutic efficacy results.

Chemotherapeutic agents that disrupt cell replication (i.e., microtubule depolymerization or stabilizing agents) include, without limitation: taxanes such as paclitaxel, docetaxel, and related analogs; vinca alkaloids such as vinblastine, vincristine, vinorelbine, vindesine, and vinflunine, and related analogs; epothilones such as Epothilone B, ixabepilone and related analogs; halichondrins such as Halichondrin B and related analogs; colchicine site binding agents such as colchicine; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which down regulates cell replication.

The selection of therapeutic agent(s) or treatment modality to be combined with an anti-GCC antibody molecule or immunoconjugate of the invention will depend on the disorder to be treated and the sensitivity of such disorder to particular therapeutic agents. The additional agent(s) or treatment modality may include, for example, standard approved therapies for the indication being treated. For example, when the anti-GCC antibody molecule or immunoconjugate thereof (e.g., an immunoconjugate of Formula (I-5)) is used to treat colon cancer, it may be used in combination with, e.g., surgery; radiation therapy; co-administration with 5-fluorouricil (5-FU), capecitibine, leucovorin, irinotecan (CPT-11), oxaliplatin, cisplatin, bevacizumab, cetuximab, or panitumum, or combinations of any of the agents listed above e.g., oxaliplatin/capecitibine (XELOX), 5-fluorouricil/leucovorin/oxaliplatin (FOLFOX), 5-fluorouricil/leucovorin/irinotecan (FOLFIRI), FOLFOX plus bevacizumab, or FOLFIRI plus bevacizumab). As an additional example, when the anti-GCC antibody molecule or immunoconjugate thereof (e.g., an immunoconjugate of Formula (I-5)) is used to treat, pancreatic cancer, it may be used in combination (e.g., co-administered) with gemcitabine. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

It is been demonstrated that certain gastrointestinal tumors are inherently resistant, or acquire resistance to certain chemotherapeutic agents, such as the microtubule disrupting agents paclitaxel and vincristine. Recently, taxanes have failed to demonstrate significant clinical benefit in phase II trials in colorectal cancer (CRC) (see e.g., Swanton et al. Cell Cycle. 2006 April; 5(8):818-23). Without intending to be bound by theory, the high incidence of chromosomal instability in this disease, coupled with alterations in spindle checkpoint regulators in vivo, may explain the disappointing results associated with taxane based therapies for CRC. Another potent microtubule disrupting agent, MMAE, is utilized in several targeted therapeutic agents that are currently under clinical investigation for the treatment of various cancers, including an anti-GCC antibody molecule conjugated to MMAE that is currently under Phase I investigation for the treatment of colorectal cancer. Example 6 infra demonstrates sensitivity to an immunoconjugate comprising an anti-GCC molecule of the invention conjugated to MMAE (a potent microtubule inhibiting agent) in several tumor xenograft models derived from primary human colorectal tumors. Example 6 also demonstrates chemoresistance to the same anti-GCC mAb-MMAE immunoconjugate in at least one tumor xenograft model derived from a primary human colorectal tumor. Surprisingly, co-administration of the anti-GCC mAb-MMAE immunoconjugate and a DNA damaging agent sensitized the refractory tumor model to the DNA damaging agent's anti-tumor activity.

Accordingly, the invention provides a method of treating a gastrointestinal cancer by administering an immunoconjugate characterized by Formula (I-4), (I-5), (I-6), or (I-7) as described herein, wherein the variable Ab is an antibody molecule described herein, e.g., an antibody molecule with one or more of the features summarized in Tables 1 to 6, in combination with a DNA damaging agent. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In a particular embodiment, the immunoconjugate administered in combination with a DNA damaging agent is characterized by Formula (I-5). For example, without limitation, the invention provides a method of treating primary or metastatic colorectal cancer, gastric, pancreatic or esophageal cancer by co-administering an immunoconjugate according to Formula (I-5), wherein the variable Ab is an anti-GCC antibody molecule described herein e.g., a 5F9 antibody molecule, and m is about 4, and a topoisomerase I inhibitor, wherein each of the immunoconjugate and the topoisomerase I inhibitor are administered in a therapeutically effective total amount. In a particular embodiment, the topoisomerase I inhibitor is irinotecan and an immunoconjugate according to Formula (I-5) is administered in combination with irinotecan for the treatment of primary or metastatic colorectal cancer. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

The invention further provides a method of treating primary or metastatic colorectal cancer, gastric, pancreatic or esophageal cancer by co-administering an immunoconjugate according to Formula (I-5), wherein the variable Ab is an anti-GCC antibody molecule described herein e.g., a 5F9 antibody molecule, and m is about 4, and an alkylating-like agent, wherein each of the immunoconjugate and the alkylating-like agent are administered in a therapeutically effective total amount. In a particular embodiment, the alkylating-like agent is oxaliplatin or cisplatin and an immunoconjugate according to Formula (I-5) administered in combination with oxaliplatin or cisplatin for the treatment of primary or metastatic colorectal cancer. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

The invention even further provides a method of treating primary or metastatic colorectal cancer, gastric, pancreatic, or esophageal cancer by co-administering an immunoconjugate according to Formula (I-5), wherein the variable Ab is an anti-GCC antibody molecule described herein e.g., a 5F9 antibody molecule, and m is about 4, and an antimetabolite, wherein each of the immunoconjugate and the antimetabolite are administered in a therapeutically effective total amount. In a particular embodiment, the antimetabolite is gemcitabine and an immunoconjugate according to Formula (I-5) administered in combination with gemcitabine for the treatment of primary or metastatic pancreatic cancer. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

The immunoconjugate of the invention (e.g., an immunoconjugate according to Formula (I-5), optionally comprising the CDRs of Table 5) may be administered with the DNA damaging agent as separate formulations or as a single dosage form. In one embodiment, when administered as a separate dosage form, the immunoconjugate may be administered prior to, at the same time as, or following administration of the DNA damaging agent. In another embodiment, when administered as a separate dosage form, one or more doses of the immunoconjugate may be administered prior to the DNA damaging agent. In another embodiment, when administered as a separate dosage form, one or more doses of the DNA damaging agent may be administered prior to the immunoconjugate. In some embodiments, the therapy results in one or more therapeutic effects from List 1 herein, e.g., one or more of (a) synergy, (c) prevention of tumor regrowth after ending administration, or effect on a tumor resistant to the immunoconjugate (f) or DNA damaging agent (h), or (i) tumor growth inhibition upon administration of the therapy.

In the methods of the invention, an immunoconjugate as described herein can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a DNA damaging agent to a patient with a gastrointestinal cancer. In some embodiments, the immunoconjugate and a DNA damaging agent are administered within the same patient visit. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In some embodiments, the immunoconjugate (e.g., an immunoconjugate according to Formula (I-5), optionally comprising an Ab with the CDRs of Table 5) and DNA damaging agent are administered to a patient, for example, a mammal, such as a human, in a sequence and within a time interval such that a first agent provided herein can act together with a second agent to provide greater benefit than if they were administered otherwise. For example, the immunoconjugate and DNA damaging agent can be administered at the same time or sequentially in any order at different points in time; however, if not administered at the same time, they should be administered sufficiently close in time so as to provide the desired therapeutic or prophylactic effect. In one embodiment, the immunoconjugate and DNA damaging agent exert their effect at times which overlap. In some embodiments, the immunoconjugate and DNA damaging agent each are administered separately, in any appropriate form and by any suitable route. In other embodiments, the immunoconjugate and DNA damaging agent can be administered simultaneously in a single dosage form. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In some embodiments, courses of treatment are administered concomitantly to a patient, i.e., individual doses of the immunoconjugate and the DNA damaging agent are administered separately yet within a time interval such that the two agents can work together. In other words, the dosing regimens are carried out concomitantly even if the therapeutics are not administered simultaneously or during the same day. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In some embodiments, the immunoconjugate (e.g., an immunoconjugate according to Formula (I-5), optionally comprising an Ab with the CDRs of Table 5) and the DNA damaging agent are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agents) for a period of time and repeating this sequential administration. Cycling therapy can reduce the development of resistance to one or more of the therapies, avoid or reduce the side effects of one of the therapies, and/or improve the efficacy of the treatment. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In some embodiments, the treatment period during which an agent is administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped. Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

It will be appreciated that the frequency with which any of these therapeutic agents (e.g., an immunoconjugate according to Formula (I-5), optionally comprising an Ab with the CDRs of Table 5, and/or a DNA damaging agent) can be administered can be once or more than once over a period of about 2 days, about 3 days, about 4 days, about 5 days, about 6 days, about 7 days, about 8 days, about 9 days, about 10 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

For example, an immunoconjugate (e.g., an immunoconjugate according to Formula (I-5), optionally comprising an Ab with the CDRs of Table 5) of the invention may be administered once a week, twice weekly, every two weeks, every three weeks or monthly for a designated period of time, in combination with a DNA damaging agent. In certain embodiments, the DNA damaging agent has a different dosing schedule than the dosing schedule of the immunoconjugate. For example, the immunoconjugate may be administered once a week or once every three weeks, and the DNA damaging agent may have a dosing schedule whereby a certain amount of the DNA damaging agent may be administered daily for two days, followed by a period of non-treatment for 5 days (2 days on/5 days off) over the designated treatment period. In another embodiment, the immunoconjugate may be administered once a week or once every three weeks, and the DNA damaging agent may have a dosing schedule whereby a certain amount of the DNA damaging agent is administered daily over a period of three days followed by a period of non-treatment for 4 days (3 days on/4 days off) over the course of the designated treatment period. In another embodiment, the immunoconjugate may be administered once a week or once every three weeks, and the DNA damaging agent may have a dosing schedule whereby the DNA damaging agent may be administered twice weekly over the course of the designated treatment period. In another embodiment, the immunoconjugate may be administered once a week or once every three weeks, and the DNA damaging agent may have a dosing schedule whereby the DNA damaging agent may be administered once every two weeks over the course of the designated treatment period. In still another embodiment, the immunoconjugate may be administered once a week or once every three weeks, and the DNA damaging agent may have a dosing schedule whereby the DNA damaging agent may be administered on days 1 and 3 of each week over the designated treatment period. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In certain embodiments, treatment with an immunoconjugate as described herein (e.g., an immunoconjugate according to Formula (I-5), optionally comprising the CDRs of Table 5) allows less frequent administration of a DNA-damaging agent than would otherwise be required to achieve a similar reduction in tumor mass or delay in tumor growth. For instance, in some embodiments, adding an immunoconjugate and DNA damaging agent to a therapeutic regimen reduces the frequency of administration of a DNA-damaging agent by at least about 1 day, 2 days, 3 days, 4 days, 5 days, 1 week, 2 weeks, or 3 weeks compared to the frequency of administration of the DNA-damaging agent alone. As a result, the DNA damaging agent may be administered to the patient, e.g., no more often than every 2 days, 3 days, 4 days, 5 days, 1 week, 2 weeks, 3 weeks, 4 weeks, or 5 weeks. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

The DNA damaging agent may be administered before the immunoconjugate (e.g., an immunoconjugate according to Formula (I-5), optionally comprising the CDRs of Table 5), concomitantly, simultaneously with the immunoconjugate, or after the immunoconjugate.

In one embodiment, the combined administration is on a 3 or 4 week dose schedule in which the first dose of the immunoconjugate (e.g., an immunoconjugate according to Formula (I-5), optionally comprising an Ab with the CDRs of Table 5) is administered once a week for 3 or 4 weeks in combination with administration of the DNA damaging agent twice-weekly over the 3 or 4 week period (e.g., the immunoconjugate is administered on day 1 of each week and the DNA damaging agent is administered on days 1 and 2 of each week such that day 1 of the immunoconjugate and day 1 of the DNA damaging agent occur substantially simultaneously or concomitantly within the same day). In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In another embodiment, the combined administration is on a 4 week dose schedule in which the first dose of the immunoconjugate (e.g., an immunoconjugate according to Formula (I-5), optionally comprising an Ab with the CDRs of Table 5) is administered every two weeks over the course of the 4 week schedule in combination with administration of the DNA damaging agent twice-weekly over the 4 week schedule (e.g., the immunoconjugate is administered on days 1 and 14 and the DNA damaging agent is administered on days 1 and 2 of each week over the four week schedule). In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In yet another embodiment, the administration is on a 3 week dose schedule in which the immunoconjugate (e.g., an immunoconjugate according to Formula (I-5), optionally comprising the CDRs of Table 5) is administered once beginning on day 1 followed by 20 days of non-treatment, in combination with a twice weekly administration of the DNA damaging agent over the 3 week schedule e.g., the immunoconjugate is administered on day 1 followed by 20 days of non-treatment and the DNA damaging agent is administered on day 1 and day 2 of each week over the 3 week schedule. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In yet another embodiment, the administration is on a 3 week dose schedule in which the immunoconjugate (e.g., an immunoconjugate according to Formula (I-5), optionally comprising the CDRs of Table 5) is administered once beginning on day 1 followed by 20 days of non-treatment, in combination with a three-times weekly administration of the DNA damaging agent over the 3 week schedule e.g., the immunoconjugate is administered on day 1 followed by 20 days of non-treatment and the DNA damaging agent is administered on days 1, 2 and 3 of each week over the 3 week schedule. In some embodiments, the therapy results in one or more therapeutic effects from List 1 herein.

In yet another embodiment, the administration is on a 3 week dose schedule in which the immunoconjugate (e.g., an immunoconjugate according to Formula (I-5), optionally comprising an Ab with the CDRs of Table 5) is administered once a week, in combination with a DNA damaging agent administered on day 1 and day 2 of each week over the 3 week schedule. In some embodiments, the therapy results in one or more therapeutic effects from List 1 herein.

In yet another embodiment, the administration is on a 3 week dose schedule in which the immunoconjugate (e.g., an immunoconjugate according to Formula (I-5), optionally comprising an Ab with the CDRs of Table 5) is administered once a week, in combination with a DNA damaging agent administered on days 1, 2, and 3 of each week over the 3 week schedule. In some embodiments, the therapy results in one or more therapeutic effects from List 1 herein.

In still another embodiment, the administration is on a 3 week dose schedule whereby the immunoconjugate (e.g., an immunoconjugate according to Formula (I-5), optionally comprising an Ab with the CDRs of Table 5) is administered once a week, in combination with a DNA damaging agent administered on day 1 of the first week over the 3 week schedule. In some embodiments, the therapy results in one or more therapeutic effects from List 1 herein.

In still another embodiment, the administration is on a 3 week dose schedule, whereby the immunoconjugate (e.g., an immunoconjugate according to Formula (I-5), optionally comprising an Ab with the CDRs of Table 5) is administered once a week, in combination with a DNA damaging agent administered on day 1 of each of the first and second weeks over the 3 week schedule. In some embodiments, the therapy results in one or more therapeutic effects from List 1 herein.

In yet another embodiment, the administration is on a 3 week dose schedule, whereby the immunoconjugate (e.g., an immunoconjugate according to Formula (I-5), optionally comprising an Ab with the CDRs of Table 5) is administered once a week, in combination with a DNA damaging agent administered on day 1 and day 3 of each week over the 3 week schedule. In some embodiments, the therapy results in one or more therapeutic effects from List 1 herein.

In some embodiments, the immunoconjugate (e.g., an immunoconjugate according to Formula (I-5), optionally comprising an A with the CDRs of Table 5) and DNA damaging agent each are administered at a dose and schedule typically used for that agent when used as a single agent. In some other embodiments, when the immunoconjugate and DNA damaging agent are administered concomitantly, one or both of the agents can advantageously be administered at a lower dose than typically administered when the agent is used as a single agent, such that the dose falls below the threshold that an adverse side effect is elicited. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

The therapeutically effective amounts or suitable dosages of the immunoconjugate and the DNA damaging agent in combination depends upon a number of factors, including the nature of the severity of the condition to be treated, the particular inhibitor, the route of administration and the age, weight, general health, and response of the individual patient. In certain embodiments, the suitable dose level is one that achieves an effective exposure as measured by increased skin mitotic index, or decreased chromosome alignment and spindle bipolarity in tumor mitotic cells, or other standard measures of effective exposure in patients with cell proliferative disorders. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of immunoconjugates can generally range, in single or divided or multiple doses, from about 10% to about 120% of the maximum tolerated dose as a single agent, when administered in combination with a DNA damaging agent. In certain embodiments, the suitable dosages are from about 20% to about 100% of the maximum tolerated dose as a single agent. In some other embodiments, the suitable dosages are from about 25% to about 90% of the maximum tolerated dose as a single agent. In some other embodiments, the suitable dosages are from about 30% to about 80% of the maximum tolerated dose as a single agent. In some other embodiments, the suitable dosages are from about 40% to about 75% of the maximum tolerated dose as a single agent. In some other embodiments, the suitable dosages are from about 45% to about 60% of the maximum tolerated dose as a single agent. In other embodiments, suitable dosages are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

It will be understood that a suitable dosage of an immunoconjugate may be administered at any time of the day or night. In some embodiments, a suitable dosage of an immunoconjugate is administered in the morning. In some other embodiments, a suitable dosage of an immunoconjugate is administered in the evening. In some other embodiments, a suitable dosage of immunoconjugate is administered both in the morning and the evening.

Suitable daily dosages of DNA damaging agent can generally range, in single or divided or multiple doses, from about 10% to about 120% of the maximum tolerated dose as a single agent when administered in combination with an anti-GCC immunoconjugate of the invention. In certain embodiments, the suitable dosages are from about 20% to about 100% of the maximum tolerated dose as a single agent. In some other embodiments, the suitable dosages are from about 25% to about 90% of the maximum tolerated dose as a single agent. In some other embodiments, the suitable dosages are from about 30% to about 80% of the maximum tolerated dose as a single agent. In some other embodiments, the suitable dosages are from about 40% to about 75% of the maximum tolerated dose as a single agent. In some other embodiments, the suitable dosages are from about 45% to about 60% of the maximum tolerated dose as a single agent. In other embodiments, suitable dosages are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

Accordingly, in some embodiments, the present disclosure provides administering, to a patient in need thereof, an immunoconjugate (e.g., an immunoconjugate according to Formula (I-5), optionally comprising an Ab with the CDRs of Table 5) in combination with a dose of irinotecan (e.g., to treat cancer, such as colon cancer). The dose of the irinotecan may be, e.g., about 10 mg/kg, 15 mg/kg, or 30 mg/kg (e.g., for administration to a mouse) or about 0.81 mg/kg, 1.2 mg/kg, or 2.4 mg/kg (e.g., for administration to a human), or any of these values ±10%, 20%, 30%, 40%, or 50%. In some embodiments, the irinotecan is administered at a dosage range of about 0.4-3.0 mg/kg, 0.4-0.6 mg/kg, 0.6-0.8 mg/kg, 0.8 mg/kg-1.0 mg/kg, 1.0-1.2 mg/kg, 1.2-1.5 mg/kg, 1.5-2.0 mg/kg, 2.0-2.5 mg/kg, 2.5-3.0 mg/kg, or any of these lower and/or upper bounds ±10%, 20%, 30%, 40%, or 50%. The timing of administration can also be considered when calculating a dose per unit time. In some embodiments, the irinotecan is administered (e.g., to a mouse) at a dose of 10 mg/kg on a 2 day on/5 day off schedule, i.e., 2.9 mg/kg/day; a dose of 15 mg/kg on a 2 day on/5 day off schedule, i.e., 4.3 mg/kg/day; or a dose of 30 mg/kg once weekly, i.e., also 4.3 mg/kg/day, or any of these values ±10%, 20%, 30%, 40%, or 50%. In some embodiments, the irinotecan is administered (e.g., to a human) at a dose of 0.81 mg/kg on a 2 day on/5 day off schedule, i.e., 0.24 mg/kg/day; a dose of 1.2 mg/kg on a 2 day on/5 day off schedule, i.e., 0.35 mg/kg/day; or a dose of 2.4 mg/kg once weekly, i.e., 0.35 mg/kg/day, or any of these values ±10%, 20%, 30%, 40%, or 50%. In some embodiments, the irinotecan is administered at a dosage range of about 0.1-0.5 mg/kg/day, 0.1-0.2 mg/kg/day, 0.2-0.3 mg/kg/day, 0.3-0.4 mg/kg/day, 0.2-0.4 mg/kg/day, or 0.4-0.5 mg/kg/day, or any of these lower and/or upper bounds ±10%, 20%, 30%, 40%, or 50%. In some embodiments, the irinotecan is administered at a dose of 80 mg/m² to 200 mg/m², e.g., 80 to 180 mg/m², 80 to 150 mg/m², 90 to 120 mg/m², less than 120 mg/m², or any of these lower and/or upper bounds ±10%, 20%, 30%, 40%, or 50%; optionally, on a schedule of day 1, day 8, day 15, day 22, with an off schedule such that the next cycle begins on day 43, or day 1, day 15 and day 29, with an off schedule such that the next cycle begins on day 43. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In other embodiments, the present disclosure provides administering, to a patient in need thereof, an immunoconjugate (e.g., an immunoconjugate according to Formula (I-5), optionally comprising an Ab with the CDRs of Table 5) in combination with a specified dose of cisplatin (e.g., to treat cancer, such as colon cancer). For instance, in some embodiments, the cisplatin is administered at a dose of about 4.0 mg/kg or 6.0 mg/kg (e.g., to a mouse), or any of these values ±10%, 20%, 30%, 40%, or 50%. The cisplatin may also be administered at a dose of about 0.33 mg/kg or 0.49 mg/kg (e.g., to a human), or any of these values ±10%, 20%, 30%, 40%, or 50%. Accordingly, in some embodiments, the cisplatin may be administered at a dosage range of about 0.1-0.6 mg/kg, 0.1-0.2 mg/kg, 0.2-0.3 mg/kg, 0.3-0.4 mg/kg, 0.4-0.5 mg/kg, or 0.5-0.6 mg/kg, or any of these lower and/or upper bounds ±10%, 20%, 30%, 40%, or 50%. The timing of administration can also be considered when calculating a dose per unit time. For instance, in some embodiments, the cisplatin is administered (e.g., to a mouse) at a dose of 4.0 mg/kg once every week, i.e., 0.57 mg/kg/day; 4.0 mg/kg once two weeks, i.e., 0.29 mg/kg/day; 6.0 mg/kg once every week, i.e., 0.86 mg/kg/day; or 6.0 mg/kg once every two weeks, i.e., 0.43 mg/kg/day, or any of these values ±10%, 20%, 30%, 40%, or 50%. In certain embodiments, the cisplatin is administered (e.g., to a human) at a dose of 0.33 mg/kg once every week, i.e., 0.046 mg/kg/day; 0.33 mg/kg once every two weeks, i.e., 0.023 mg/kg/day; 0.49 mg/kg once every week, i.e., 0.070 mg/kg/day; or 0.49 mg/kg once every two weeks, i.e., 0.035 mg/kg/day, or any of these values ±10%, 20%, 30%, 40%, or 50%. Accordingly, in some embodiments, the cisplatin is administered at a dosage range of about 0.01-0.08 mg/kg/day, 0.01-0.02 mg/kg/day, 0.2-0.4 mg/kg/day, 0.4-0.6 mg/kg/day, 0.2-0.6 mg/kg/day, or 0.06-0.08 mg/kg/day, or any of these lower and/or upper bounds ±10%, 20%, 30%, 40%, or 50%. In some embodiments, the cisplatin is administered at a dose of 30 mg/m² to 120 mg/m², e.g., 30 to 100 mg/m², 40 to 70 mg/m², 50 to 70 mg/m², less than 100 mg/m², or any of these lower and/or upper bounds ±10%, 20%, 30%, 40%, or 50%, optionally, on a schedule of once every three weeks or once a month. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In other embodiments, the present disclosure provides administering, to a patient in need thereof, an immunoconjugate (e.g., an immunoconjugate according to Formula (I-5), optionally comprising an Ab with the CDRs of Table 5) in combination with a specified dose of 5-fluorouracil (5-FU) (e.g., to treat cancer, such as colon cancer). For instance, in some embodiments, the 5-FU is administered at a dose of about 15 mg/kg or 25 mg/kg (e.g., to a mouse), or either of these values ±10%, 20%, 30%, 40%, or 50%. The 5-FU may also be administered at a dose of about 1.2 mg/kg or 2.0 mg/kg (e.g., to a human), or either of these values ±10%, 20%, 30%, 40%, or 50%. Accordingly, in some embodiments, the 5-FU is administered at a dosage range of about 0.8-2.4 mg/kg/day, 0.8-1.0 mg/kg, 1.0-1.2 mg/kg, 1.2-1.4 mg/kg, 1.0-1.4 mg/kg, 1.4-1.6 mg/kg, 1.6-1.8 mg/kg, 1.8-2.0 mg/kg, 2.0-2.2 mg/kg, 1.8-2.2 mg/kg, or 2.2-2.4 mg/kg, or any of these lower and/or upper bounds ±10%, 20%, 30%, 40%, or 50%. The timing of administration can also be considered when calculating a dose per unit time. For instance, in some embodiments, the 5-FU is administered (e.g., to a mouse) at a dose of 15 mg/kg on a 3 day on/4 day off dosing schedule, i.e., 6.4 mg/kg/day, or 25 mg/kg on a 3 day on/4 day off dosing schedule, e.g., 10.7 mg/kg/day, or any of these values ±10%, 20%, 30%, 40%, or 50%. In certain embodiments, the 5-FU is administered (e.g., to a human) at a dose of 1.2 mg/kg on a 3 day on/4 day off dosing schedule, i.e., 0.51 mg/kg/day, or 2.0 mg/kg on a 3 day on/4 day off dosing schedule, e.g., 0.86 mg/kg/day, or any of these values ±10%, 20%, 30%, 40%, or 50%. Accordingly, in some embodiments, the 5-FU is administered at a dosage range of 0.3-0.5 mg/kg/day, 0.5-0.7 mg/kg day, 0.7-0.9 mg/kg/day, 0.9-1.1 mg/kg/day, 0.4-0.6 mg/kg/day, 0.7-1.0 mg/kg/day, or 0.3-1.1 mg/kg/day, or any of these lower and/or upper bounds ±10%, 20%, 30%, 40%, or 50%. In some embodiments, the 5-FU is administered at a dosage range of 3 to 12 mg/kg, e.g., 3 to 9 mg·kg, e.g., 3-6 mg/kg, e.g., less than 6 mg/kg, or any of these lower and/or upper bounds ±10%, 20%, 30%, 40%, or 50%, optionally on a schedule of 4 days on, and then additional administrations given on day 6, day 8, day 10 and day 12. In some embodiments, the 5-FU is administered at a dose of 200 mg/m² to 700 mg/m², e.g., 200 to 600 mg/m², 300 to 600 mg/m², 300 to 500 mg/m², less than 600 or 500 mg/m², or any of these lower and/or upper bounds ±10%, 20%, 30%, 40%, or 50%, optionally, on a schedule of day 1, day 8, day 15 and day 29, with an off schedule such that the next cycle begins on day 43, or on day 1, day 2, day 14, day 15, day 29 and day 30, with an off schedule such that the next cycle begins on day 43. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In other embodiments, the present disclosure provides administering, to a patient in need thereof, an immunoconjugate (e.g., an immunoconjugate according to Formula (I-5), optionally comprising the CDRs of Table 5) in combination with a specified dose of gemcitabine (e.g., to treat a cancer, such as pancreatic cancer). For instance, in some embodiments, the gemcitabine is administered at a dose of about 15 mg/kg or 20 mg/kg, e.g., to a mouse, or either of these values ±10%, 20%, 30%, 40%, or 50%. In some embodiments, the gemcitabine is administered, e.g., to a human, at a dose of about 1.2 mg/kg or 1.6 mg/kg, or either of these values ±10%, 20%, 30%, 40%, or 50%. Accordingly, in some embodiments, the gemcitabine is administered at a dosage range of about 0.8-2.0 mg/kg, 0.8-1.0 mg/kg, 1.0-1.2 mg/kg, 1.2-1.4 mg/kg, 1.0-1.4 mg/kg, 1.4-1.6 mg·g, 1.6-1.8 mg/kg, 1.4-1.8 mg/kg, or 1.8-2.0 mg/kg, or any of these lower and/or upper bounds ±10%, 20%, 30%, 40%, or 50%. The timing of administration can also be considered when calculating a dose per unit time. For instance, in some embodiments, the gemcitabine is administered (e.g., to a mouse) at a dose of 15 mg/kg twice weekly, i.e., 4.3 mg/kg/day, or 20 mg/kg twice weekly, i.e., 5.7 mg/kg/day, or any of these values ±10%, 20%, 30%, 40%, or 50%. The gemcitabine can also be administered, e.g., to a human, at a dose of 1.2 mg/kg twice weekly, i.e., 0.34 mg/kg/day, or 1.6 mg/kg twice weekly, i.e., 0.48 mg/kg/day, or any of these values ±10%, 20%, 30%, 40%, or 50%. Accordingly, in some embodiments, the gemcitabine is administered at a dosage range of 0.1-0.3 mg/kg/day, 0.3-0.5 mg/kg/day, 0.4-0.6 mg/kg/day, 0.5-0.7 mg/kg/day, 0.2-0.6 mg/kg/day, or 0.1-0.7 mg/kg/day, or any of these lower and/or upper bounds ±10%, 20%, 30%, 40%, or 50%. In some embodiments, the gemcitibine can be administered at a dose of 700 to 1300 mg/m², e.g., 800 to 1250 mg/m², e.g., 800 to 1000 mg/m², e.g., less than 1000 mg/m², or any of these lower and/or upper bounds ±10%, 20%, 30%, 40%, or 50%, optionally on a schedule of once a week for 7 weeks, followed by an off schedule of one week, optionally followed by a cycle of once a week for three weeks. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

It will be understood that a suitable dosage of a DNA damaging agent may be administered at any time of the day or night. In some embodiments, a suitable dosage of a DNA damaging agent is administered in the morning. In some other embodiments, a suitable dosage of a DNA damaging agent is administered in the evening. In some other embodiments, a suitable dosage of a DNA damaging agent is administered both in the morning and the evening.

In some embodiments, a first treatment period in which a first amount of the DNA damaging agent is administered can be followed by another treatment period in which a same or different amount of the same or a different DNA damaging agent is administered. A wide variety of therapeutic agents may have a therapeutically relevant added benefit in combination with the combination of an immunoconjugate and DNA damaging agent according to the present invention. Combination therapies that comprise an immunoconjugate described herein and a DNA damaging agents with one or more other therapeutic agents can be used, for example, to: 1) enhance the therapeutic effect(s) of the methods of the present invention and/or the one or more other therapeutic agents; 2) reduce the side effects exhibited by the methods of the present invention and/or the one or more other therapeutic agents; and/or 3) reduce the effective dose of the immunoconjugate and DNA damaging agent and/or the one or more other therapeutic agents. For example, such therapeutic agents may combine with an immunoconjugate described herein and a DNA damaging agent to inhibit undesirable cell growth, such as inappropriate cell growth resulting in undesirable benign conditions or tumor growth. In some embodiments, the therapy results in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

Examples of therapeutic agents that may be used in further combination with the immunoconjugates described herein and DNA damaging agents include, but are not limited to, anti-proliferative agents, anticancer agents, antibiotic agents, hormonal agents, plant-derived agents, and biologic agents.

Antibiotic agents are a group of drugs that produced in a manner similar to antibiotics as a modification of natural products. Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. Combination therapy including an antibiotic agent, in addition to the immunoconjugate and DNA damaging agent, may have therapeutic synergistic effects on cancer and reduce sides affects associated with these chemotherapeutic agents.

Hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, and flutamide), aromatase inhibitors (e.g., aminoglutethimide, anastrozole and tetrazole), ketoconazole, goserelin acetate, leuprolide, megestrol acetate and mifepristone. Combination therapy including a hormonal agent, in addition to the immunoconjugate and DNA damaging agent, may have therapeutic synergistic effects on cancer and reduce sides affects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), and taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including a plant-derived agent, in addition to the immunoconjugate and DNA damaging agent, may have therapeutic synergistic effects on cancer and reduce sides affects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy including a biologic agent, in addition to the immunoconjugate and DNA damaging agent, may have therapeutic synergistic effects on cancer, enhance the patient's immune responses to tumorigenic signals, and reduce potential sides affects associated with this chemotherapeutic.

In another aspect, the invention features the use of an anti-GCC immunoconjugate as described herein (e.g., an immunoconjugate according to Formula (I-5), optionally comprising an Ab with the CDRs of Table 5) in combination with a DNA-damaging agent in the manufacture of a medicament. In an embodiment, the medicament is useful for treating cancer, e.g., a gastrointestinal cancer such as primary or metastatic colorectal, gastric, pancreatic or esophageal cancer. In some embodiments, the medicament comprises an anti-GCC antibody molecule having one or more feature summarized in Tables 1-6. In some embodiments, the medicament comprises a 5F9 antibody molecule. In some embodiments, the medicament has a property of resulting in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

In some aspects, the invention features the use of an immunoconjugate comprising an anti-GCC antibody molecule having one or more feature summarized in Tables 1-6 in the manufacture of a medicament that further contains a DNA damaging agent. Such a medicament is useful for treating cancer, including but not limited to a gastrointestinal cancer such as colorectal, gastric, pancreatic or esophageal cancer. In one embodiment, the immunoconjugate of the medicament is characterized by Formula (I-5), wherein the Ab is an anti-GCC antibody molecule described herein e.g., a 5F9 antibody molecule, and m is about 4, and the DNA damaging agent of the medicament is a topoisomerase I inhibitor, such as irinotecan. In another embodiment, the immunoconjugate of the medicament is characterized by Formula (I-5), wherein the Ab is an anti-GCC antibody molecule described herein, e.g., a 5F9 antibody molecule, and m is about 4, and the DNA damaging agent of the medicament is an anthracycline, such as cisplatin or oxaliplatin. In yet another embodiment, the immunoconjugate of the medicament is characterized by Formula (I-5), wherein the Ab is an anti-GCC antibody molecule described herein, e.g., a 5F9 antibody molecule, and m is about 4, and the DNA damaging agent of the medicament is an antimetabolite such as gemcitabine. In some embodiments, the medicament has a property of resulting in one or more therapeutic effects from List 1 herein, e.g., one or more of (a) synergy, (c) prevention of tumor regrowth after ending administration, effect on a tumor resistant to the immunoconjugate (f) or DNA damaging agent (h), or (i) tumor growth inhibition upon administration of the therapy.

Tailoring Anti-GCC Therapies Based on a Tumor's GCC Expression and/or Anti-GCC Immunoconjugate Sensitivity

The methods described herein can be used on cancers that express GCC. In some embodiments, the methods can include detecting the presence of GCC, e.g., to detect the presence of GCC in a biological sample, or to detect the presence or distribution of GCC in a subject, e.g., using an anti-GCC antibody, e.g., a labeled anti-GCC antibody, or a ligand, e.g., peptide ligand, e.g., labeled peptide ligand, that binds to GCC. The term “detecting” as used herein encompasses quantitative or qualitative detection. Detecting GCC or GCC protein, as used herein, means detecting intact GCC protein or detecting a portion of the GCC protein that comprises the epitope to which a detecting anti-GCC antibody molecule or GCC binding ligand binds. In some embodiments, the method includes detecting a level of GCC on the target tumor, and an anti-tumor therapy is selected based on the results of the detection step. In some embodiments, the method includes acquiring information regarding GCC expression levels in a subject, and selecting the anti-tumor therapy based upon the information. In some embodiments, the method includes treating a subject having a cancer which expresses GCC. Additional details on the use of anti-GCC antibodies for detecting GCC expression on tumors is found in International Application WO2013/163633, which is herein incorporated by reference in its entirety.

In some embodiments, the antibody used for detecting GCC expression on the tumor is an antibody having the VH or VL sequences of Table 22 herein, or an antibody having one or more (e.g., six) CDRs from within the VH or VL sequences of Table 22 herein, e.g., those CDRs set out in Table 24 herein. For instance, the antibody may comprise the anti-GCC rabbit mAb MIL-44-148-2 or a portion thereof.

Accordingly, in another aspect, the methods can include detecting GCC protein, e.g., detecting a GCC expressing cell or tissue, e.g., a tumor cell, or a tumor having cells, that express GCC. The method comprises: contacting a material, e.g., a cell or tissue, e.g., a sample of a tumor which expresses GCC, with an anti-GCC antibody molecule, e.g., an anti-GCC antibody molecule described herein, or a GCC binding ligand, under conditions which allow formation of a complex between the anti-GCC antibody molecule or ligand and the GCC protein; and detecting formation of a complex between antibody molecule or ligand and the GCC protein, to thereby detect the presence of GCC protein, e.g., to detect a GCC expressing cell or tumor.

In certain embodiments, the tissues include normal and/or cancerous tissues that express GCC at higher levels relative to other tissues, for example other tissue such as B cells and/or B cell associated tissues.

In another aspect, the methods described herein can include detecting the presence of GCC protein in vitro (e.g., in a biological sample, such as a tissue biopsy, e.g., from a tumor tissue, from a subject) or in vivo (e.g., by in vivo imaging in a subject). The method comprises: (i) contacting a sample with an anti-GCC antibody molecule or a GCC binding ligand, or administering to a subject, an anti-GCC antibody molecule or GCC binding ligand; and (ii) detecting formation of a complex between the anti-GCC antibody molecule or ligand the and GCC protein. Complex formation is indicative of the presence or level of GCC. The method optionally further comprises treating the subject with an immunoconjugate described herein in combination with a DNA-damaging agent, e.g., as described below.

In embodiments the level of complex detected in the sample or subject is compared with a reference value, e.g., a value for complex formation or level of GCC. In an embodiment a level of GCC which exceeds a reference value is indicative of a GCC-mediated disorder and guides the physician as to a suitable treatment regimen with an immunoconjugate described herein and a DNA damaging agent.

In an embodiment the method comprises contacting a reference sample, e.g., a control sample (e.g., a control biological sample, such as plasma, tissue, biopsy) or a control subject) with an anti-GCC antibody molecule or GCC binding ligand and comparing the level of complex detected therein with the level detected in the sample or subject.

The GCC antigen density in a subject or sample may be classified as (in descending order) high, relatively high, moderate, or low. The GCC antigen density may be measured by any suitable method. For instance, the H-score method may be used; this method is described in the following paragraphs and in Internation Application WO/2013/163633 which is hereby incorporated by reference in its entirety. As another example, the GCC antigen density in one or more of a patient's tumors may be determined by an in vivo detection method, e.g., by administering an anti-GCC antibody conjugated to a detectable label and detecting the conjugate with an imaging system such as MRI. In certain embodiments, the antigen density detected in the sample or subject is described using a semi-quantitative IHC score system, e.g., as described in Examples 3 and 5 herein. For example, in some embodiments, an IHC score of 4+ is classified as high GCC antigen density, an IHC score of 2-3+ is classified as a relatively high GCC antigen density, an IHC score of 2+ is classified as moderate GCC antigen density, and an IHC score of 1+ is classified as low GCC antigen density. It is understood that other methods may also be used to determine the GCC antigen density in a subject or sample.

To calculate an H-score, staining can be performed as follows. The sample is incubated overnight with an anti-GCC antibody. This procedure can be completely automated using the TechMate 500 or TechMate 1000 (Roche Diagnostics). After staining, slides are dehydrated through an alcohol series to absolute ethanol followed by xylene rinses. Slides are permanently coverslipped with glass coverslips and CytoSeal. Slides are examined under a microscope to assess staining. Positive staining is indicated by the presence of a brown (DAB-HRP) reaction product. Hematoxylin counterstain provides a blue nuclear stain to assess cell and tissue morphology.

The H-score method may be performed on the stained cells as follows. The percentage of cells (0-100) within a tumor with staining intensities ranging from 0-3+ are provided. For example, scores with intensities of 0, 0.5, 1, 2 and 3 are provided. Depending on the marker, 0.5 staining can be scored as positive or negative, and reflects light but perceptible staining for the marker. To obtain an H-score, the percentage of tumor cells are multiplied by each intensity and added together:

H score=(% tumor*1)+(% tumor*2)+(% tumor*3). For example, if a tumor is 20% negative (0), 30%+1, 10%+2, 40%+3, this would give an H score of 170.

The maximum H-score is 300 (100%*+3), per sub-cellular localization (i.e., apical or cytoplasmic), if 100% of tumor cells label with 3+ intensity. In some embodiments, e.g., as a control, the total H-score alone is not used to compare samples, but evaluated in addition to a review of the break-down of the percentage of cells at each intensity. For example, a score of 90 could represent 90% of tumor cells staining with 1+ intensity or 30% of cells with 3+ intensity. These samples have the same H-score but very different GCC expression. The percentage of cells to be scored at each intensity can vary, but are normally scored in increments of 10%; however, a small percentage of scoring of a single component can be estimated at 1% and 5% as well in order to demonstrate that some level of staining is present. For GCC, apical staining may be considered for evaluating at low level increments, such as 1 and 5%.

Different sub-cellular localizations can be scored for GCC using the H-score approach. These include cytoplasmic staining and apical associated staining. The cytoplasmic staining pattern is generally observed as diffuse throughout the cytoplasm of tumor cells. However, in some cases there are variations of the cytoplasmic staining, which include intense globular staining or punctate staining, coarse granular staining. Intense globular staining can be scored as 3+ cytoplasmic staining. The punctate staining is associated with apical staining and is not given a separate score for this type of cytoplasmic staining. GCC apical staining is observed when lumen were present. Other GCC staining patterns observed included membrane-like, non-lumen staining (one case) and extra-cellular staining present in tumor lumen. In normal colon tissues, staining is generally apical along with diffuse cytoplasmic staining.

Since H scores can be obtained for both cytoplasmic and apical GCC expression, all data may be captured and in some instances, an aggregate H score can be generated by using the sum of both apical and cytoplasmic GCC expression. In such instances, the maximum H score becomes 600 for the aggregate score (300 apical+300 cytoplasmic).

In an embodiment the level of GCC, in a sample from the subject, or in the subject, is compared with a reference level, e.g., the level of GCC in a control material, e.g., a normal cell of the same tissue origin as the subject's cell or a cell having GCC at levels comparable to such a normal cell. The method can comprise, e.g., responsive to the detected level of GCC, providing a diagnosis, a prognosis, an evaluation of the efficacy of treatment, or the staging of a disorder. A higher level of GCC in the sample or subject, as compared to the control material, indicates the presence of a disorder associated with increased expression of GCC. A higher level of GCC in the sample or subject, as compared to the control material, can also indicate, the relative lack of efficacy of a treatment, a relatively poorer prognosis, or a later stage of disease. The level of GCC can also be used to evaluate or select future treatment, e.g., the need for more or less aggressive treatment, or the need to switch from one treatment regimen to another.

The level of GCC can also be used to select or evaluate patients.

The responsiveness of a tumor to an anti-GCC immunoconjugate (e.g., immunoconjugate (I-5)), e.g., comprising an Ab with the CDRs of Table 5, can guide a treating physician in determining the appropriate antineoplastic therapy, e.g., therapy with immunoconjugate (I-5), e.g., comprising an Ab with the CDRs of Table 5, in combination with a DNA damaging agent. Example 5 herein discloses methods of measuring a tumor's responsiveness to immunoconjugate therapy, and Example 6 herein discloses appropriate modes of treatment for tumors having various degrees of sensitivity to an anti-GCC immunoconjugate.

The sensitivity or resistance of a tumor or cancer cell to a given therapeutic (e.g., an immunoconjugate or a DNA damaging agent) may be categorized as (in descending order) strong, moderate-to-strong, moderate, or resistant. The sensitivity may be determined by any suitable method. For instance, in some embodiments, “strong anti-tumor activity” or “strong sensitivity” refers to at least about the level of anti-tumor activity observed when PHTX-09c cells are treated with 5F9 vcMMAE as shown in Example 5 herein; “moderate to strong” anti-tumor activity or sensitivity refers to about the level of anti-tumor activity observed when PHTX-21c cells are treated with 5F9 vcMMAE; “moderate anti-tumor activity” or “moderate sensitivity” refers to about the level of anti-tumor activity observed when PHTX-17c cells are treated with 5F9 vcMMAE; and “resistant” refers to about the level of or less than the level of anti-tumor activity observed when PHTX-11c cells are treated with 5F9 vcMMAE. As another example, drug sensitivity can be determined using a cell culture assay, wherein the drug of interest is administered to a relevant cancer cell (e.g., a cell biopsied from a tumor of a cancer patient) and cytotoxic activity is determined. As yet another example, the tumor sensitivity to a therapeutic agent may be determined by monitoring a subject's responsiveness to that agent, e.g., by visualizing the tumor size at different time points while the patient is placed on therapy with the agent. It is understood that other methods may also be used to determine the sensitivity of a subject or sample to an immunoconjugate or DNA damaging agent.

In some aspects, the present disclosure provides a method of testing a tumor for sensitivity to an anti-GCC therapy (e.g., an immunoconjugate of (I-5)) to identifying the sensitivity (e.g., strong sensitivity, strong-to-moderate sensitivity, moderate sensitivity, or resistant).

E.g., in embodiments patients whose tumor cells express high amounts of GCC on their surfaces would be considered good candidates for treatment with toxin-conjugated anti-GCC antibody molecules. In embodiments patients whose tumor cells express low amounts of GCC on their surfaces might be candidates for combining the anti-GCC antibody molecule with an additional treatment method, e.g., a DNA damaging agent. In another example, the dose of the anti-GCC antibody molecule could be adjusted to reflect the number of GCC molecules expressed on the surfaces of tumor cells. Patients with high numbers of GCC molecules on their tumor cell surfaces might be treated with lower doses than patients with low numbers of GCC molecules. Detecting the presence of GCC-expressing tumor cells in vivo can allow identification of tissues into the primary GCC-expressing tumor has metastasized. Knowledge of which tissues have metastases can lead to targeted application of tumor therapy.

As discussed above, anti-GCC antibody molecules and GCC binding ligands permit assessment of the presence of a GCC protein in normal versus neoplastic tissues, through which the presence or severity of disease, disease progress and/or the efficacy of therapy can be assessed. For example, therapy (e.g., therapy with an immunoconjugate such as (I-5) in combination with a DNA damaging agent) can be monitored and efficacy assessed. In one example, a GCC protein can be detected and/or measured in a first sample obtained from a subject having a cell proliferative disease (e.g., colon cancer, gastric cancer, and esophageal cancer) and therapy can be initiated. Later, a second sample can be obtained from the subject and GCC protein in the sample can be detected and/or measured. A decrease in the quantity of GCC protein detected or measured in the second sample can be indicative of therapeutic efficacy.

### Anti-GCC Antibody Sequences

Anti-GCC antibodies were generated by several methods, as is discussed in more detail in the Examples. One particular anti-GCC antibody, designated as “5F9” was first generated using transgenic mice that generate fully human IgG2 antibodies, utilizing Abgenix XENOMOUSE transgenic technology, and isolated using hybridoma technology. (Antibody 5F9 was subsequently produced in CHO cells as described in Example 4 herein.) Human mAb Abx-229 was generated using transgenic mice that generate fully human IgG2 antibodies. Single antibodies were isolated using Abgenix SLAM technology. These were used to make fully human IgG1 antibodies. Specificity of the antibodies against GCC was tested by ELISA and flow cytometry (FCM).

Table 1 below summarizes a method used to make an antibody molecule 5F9, the immunogen used to generate the antibody, the animal used, the source, the species, and the isotype isolates.

TABLE 1

| Ab | Immunogen | Animal | Source | Species | Isotype |
|---|---|---|---|---|---|
| 5F9 | TOK107-hIg | XenoMouse | Hybridoma | Human | IgG2, k |

The sequences of the light and heavy chain variable regions were determined. (Table 2). The amino acid and nucleic acid sequences for the variable regions of each of the heavy and light chains for the 5F9 anti-GCC antibody is shown in Tables 3 and 4, respectively. The amino acid and nucleic acid sequences for each of the CDRs of the heavy and light chains for the 5F9 anti-GCC antibody is shown in Tables 5 and 6, respectively.

Sequencing of the CDRs allowed determination of the abundance of residues that might serve as toxin conjugation sites. An unpaired free cysteine in the antigen binding region could be a site for auristatin conjugation and a lysine could be a site for maytansine conjugation. Toxin conjugation to an amino acid of the CDR would raise the concern of altering the binding affinity of the antibody to GCC. Thus, in embodiments the CDRs lack an amino acid which can be conjugated to a therapeutic agent.

TABLE 2
Summary of SEQ ID NOs for the variable
regions of monoclonal antibodies.

| mAb | IgG chain | NA SEQ ID | AA SEQ ID |
|---|---|---|---|
| 5F9 | Heavy chain | 17 | 18 |
| | Light chain | 19 | 20 |

TABLE 3
Amino acid sequence of mAb variable region

| | IgG | SEQ | |
|---|---|---|---|
| mAb | chain | ID NO: | Amino Acid Sequence |
| 5F9 | Heavy | 18 | QVQLQQWGAGLLKPSETLSLTCAVFGGSFSGYYWSWIRQPPGKGLE |
| | chain | | WIGEINHRGNTNDNPSLKSRVTISVDTSKNQFALKLSSVTAADTAVY |
| | | | YCARERGYTYGNFDHWGQGTLVTVSS |
| 5F9 | Light | 20 | EIVMTQSPATLSVSPGERATLSCRASQSVSRNLAWYQQKPGQAPRLL |
| | chain | | IYGASTRATGIPARFSGSGSGTEFTLTIGSLQSEDFAVYYCQQYKTWP |
| | | | RTFGQGTNVEIK |

TABLE 4
Nucleic acid sequence of mAb variable region

| | | | SEQ | |
|---|---|---|---|---|
| | | IgG | ID | |
| | mAb | chain | NO: | Nucleic Acid Sequence |
| 9 | 5F9 | Heavy | 17 | CAGGTGCAGCTACAGCAGTGGGGCGCAGGACTGTTGAAGCCTT |
| | | chain | | CGGAGACCCTGTCCCTCACCTGCGCTGTCTTTGGTGGGTCCTTC |
| | | | | AGTGGTTACTACTGGAGCTGGATCCGCCAGCCCCCAGGGAAGG |
| | | | | GGCTGGAGTGGATTGGGGAAATCAATCATCGTGGAAACACCAA |
| | | | | CGACAACCCGTCCCTCAAGAGTCGAGTCACCATATCAGTAGAC |
| | | | | ACGTCCAAGAACCAGTTCGCCCTGAAGCTGAGTTCTGTGACCG |
| | | | | CCGCGGACACGGCTGTTTATTACTGTGCGAGAGAACGTGGATA |
| | | | | CACCTATGGTAACTTTGACCACTGGGGCCAGGGAACCCTGGTC |
| | | | | ACCGTCTCCTCA |
| 10 | 5F9 | Light | 19 | GAAATAGTGATGACGCAGTCTCCAGCCACCCTGTCTGTGTCTCC |
| | | chain | | AGGGGAAAGAGCCACCCTCTCCTGCAGGGCCAGTCAGAGTGTT |
| | | | | AGCAGAAACTTAGCCTGGTATCAGCAGAAACCTGGCCAGGCTC |
| | | | | CCAGGCTCCTCATCTATGGTGCATCCACCAGGGCCACTGGAATC |
| | | | | CCAGCCAGGTTCAGTGGCAGTGGGTCTGGGACAGAGTTCACTC |
| | | | | TCACCATCGGCAGCCTGCAGTCTGAAGATTTTGCAGTTTATTAC |
| | | | | TGTCAGCAGTATAAAACCTGGCCTCGGACGTTCGGCCAAGGGA |
| | | | | CCAACGTGGAAATCAAA |

TABLE 5
Amino acid sequence of CDRs

| | IgG | SEQ ID | |
|---|---|---|---|
| mAb | chain | NO: | Amino Acid Sequence |
| 5F9 | VH | 25 | GYYWS |
| | CDR1 | | |
| 5F9 | VH | 26 | EINHRGNTNDNPSLKS |
| | CDR2 | | |
| 5F9 | VH | 27 | ERGYTYGNFDH |
| | CDR3 | | |
| 5F9 | VL | 28 | RASQSVSRNLA |
| | CDR1 | | |
| 5F9 | VL | 29 | GASTRAT |
| | CDR2 | | |
| 5F9 | VL | 30 | QQYKTWPRT |
| | CDR3 | | |

TABLE 6
Nucleic acid sequence of CDRs

| | IgG | SEQ ID | |
|---|---|---|---|
| mAb | chain | NO: | Amino Acid Sequence |
| 5F9 | VH | 10 | GGTTACTACTGGAGC |
| | CDR1 | | |
| 5F9 | VH | 11 | GAAATCAATCATCGTGGAAACACCAACGACAACCCG |
| | CDR2 | | TCCCTCAAG |
| 5F9 | VH | 12 | GAACGTGGATACACCTATGGTAACTTTGACCAC |
| | CDR3 | | |
| 5F9 | VL | 13 | AGGGCCAGTCAGAGTGTTAGCAGAAACTTAGCC |
| | CDR1 | | |
| 5F9 | VL | 14 | GGTGCATCCACCAGGGCCACT |
| | CDR2 | | |
| 5F9 | VL | 15 | CAGCAGTATAAAACCTGGCCTCGGACG |
| | CDR3 | | |

Expression vectors were created as described above which contain coding sequence for both the heavy and light chain of mAb 5F9.

The invention is illustrated by the following examples, which should not be construed as further limiting.

### EXAMPLES

### Example 1

### Generation of Anti-GCC Antibodies and Characterization

The generation of GCC protein for immunization and screening was performed as follows. GCC antigen was prepared by subcloning a portion of the GCC gene encoding a sequence comprising the following GCC sequence (signal sequence and extracellular domain) into an expression vector.

(SEQ ID NO: 16)
MKTLLLDLALWSLLFQPGWLSFSSQVSQNCHNGSYEISVLMMGNSAFAEP
LKNLEDAVNEGLEIVRGRLQNAGLNVTVNATFMYSDGLIHNSGDCRSSTC
EGLDLLRKISNAQRMGCVLIGPSCTYSTFQMYLDTELSYPMISAGSFGLS
CDYKETLTRLMSPARKLMYFLVNFWKTNDLPFKTYSWSTSYVYKNGTETE
DCFWYLNALEASVSYFSHELGFKVVLRQDKEFQDILMDHNRKSNVIIMCG
GPEFLYKLKGDRAVAEDIVIILVDLFNDQYFEDNVTAPDYMKNVLVLTLS
PGNSLLNSSFSRNLSPTKRDFALAYLNGILLFGHMLKIFLENGENITTPK
FAHAFRNLTFEGYDGPVTLDDWGDVDSTMVLLYTSVDTKKYKVLLTYDTH
VNKTYPVDMSPTFTWKNSKL

The expression vector (pLKTOK107) provided a C-terminal IgG1 Fc region to fuse with the GCC sequence. This vector comprised an exon with the IgG1 hinge, CH2 and CH3 domains, mutated to eliminate an unpaired cysteine from the CH1 fragment in the exon. This IgG1 Fc region was further mutated at lysine 235 and glycine 237 to alanines. The construct was expressed recombinantly in human embryonic kidney (HEK) 293 cells transfected with the gene for SV40 T-antigen as secreted GCC sequence (amino acid residues 24 to 430 of SEQ ID NO:3) fused to a C-terminal human IgG1 Fc. The protein, named TOK107-hIg (alt. name hGCC-ECD/hIgG1 Fc, SEQ ID NO: 62), was purified by protein A chromatography and size exclusion chromatography.

GCC antigen was also prepared by subcloning the above fusion protein into an expression vector such as pLKTOK111, which allows for fusion of the murine IgG2a transmembrane region onto the C-terminus. When this construct is expressed recombinantly in CHO cells, the GCC extracellular domain is detected on the cell surface. High cell surface expression of the GCC-Ig fusion protein (SEQ ID NO: 61) is achieved when the pLKTOK111 vector is co-transfected with pLKTOK123, which comprises murine CD79a (MB-1) and CD79b (B29). Clone #27 from this transfection (CHO-GCC#27) was used as immunogen. HT-29-GCC#2 cells also were used as immunogen.

For screening of hybridoma supernatants and purified mAbs by ELISA, the nucleic acid encoding a GCC fusion construct was cloned into a pCMV1 expression vector (Sigma). Purification tags: FLAG-tag (in the N-terminus) and His-tag (in the C-terminus) were cloned into the construct as well. The fusion protein construct was transfected into 293 cells, expressed, and recombinant protein was purified over and Anti-FLAG® M2-Agarose Affinity column (Sigma).

Reagents and Cell Lines.

HEK293 cells, CHO, and T84 human colon cancer cells and were obtained from ATCC and maintained according to ATCC protocols.

Mice:

Female C57BL/6 mice, 4-6 weeks old, were purchased from Taconic Farms, Inc. (Germantown, N.Y.) for the generation of the murine hybridomas. Xenomice, bred in-house until 4-6 weeks old, producing human IgG2 antibodies were obtained from Abgenix, Inc. (Fremont, Calif.) for the generation of the human hybridomas. All animals were acquired and maintained according to the guidelines of the Institutional Animal Care and Use Committee of Millennium Pharmaceuticals, Inc.

Cell Lines:

The cell lines used for functional assays were cell pairs of GCC transfected cells and vector control HEK293 or HT29 cells. HT29 cells were transfected with the full length GCC under control of the EF-1α promoter or empty vector (pLKTOK4) and selected in G418. The GCC in these cells was confirmed to have a cGMP response when contacted with the ST peptide (1-18 or 5-18). HEK293 cells were transfected full length GCC under control of the CMV promoter or empty vector (pN8mycSV40) and selected in blasticidin. The GCC in these cells has a myc tag. The clones selected for highest GCC expression were 293-GCC#2, HT29-GCC#2 and HT29-GCC#5. The HT29-GCC#2 also were used as immunogens for generating anti-GCC antibody molecules. Additional GCC-expressing cells are CT26 cells. To develop the GCC-expressing CT26 cell line, pTOK58D vector was used. Full length GCC was cloned into the site normally used for heavy chain cloning and luciferase was cloned into the site normally used for light chain cloning. After transfection into CT26 cells, independent expression of both GCC and luciferase was confirmed. Surface expression of GCC was confirmed by flow cytometry using the 5F9 antibody. Clone #32 was selected for further studies.

The T84 colon cancer cell line endogenously expresses GCC. Taqman analysis of GCC in a broad cell line panel revealed that T84 was the only cell line that express mRNA for GCC. Staining for GCC with a GCC selective mAb on cell pellets of T84 cells showed significant GCC protein expression.

Quantitation of GCC receptor levels with radiolabeled ligand (ST-toxin) suggested that the 293-GCC#2 cell expressed more GCC than T84 cells while the HT29-GCC#2 or #5 expressed the fewest GCC molecules per cell.

| Cell line | Whole cell binding assay (receptor/cell) |
|---|---|
| HT-29-GCC#2/#5 | 100,000 |
| T84 endogenous GCC | 300,000 |
| 293-GCC | 600,000 |

Generation of Human mAbs.

XENOMOUSE genetically engineered mice (Abgenix, Fremont, Calif.) (8 to 10 weeks old) were immunized for production of human monoclonal antibodies. See, Mendez et al. Nature Genetics 15:146-156 (1997), Green and Jakobovits J. Exp. Med. 188:483-495 (1998). Several immunization schemes were employed. In one scheme, one hundred micrograms of human GC-C extracellular domain/human Ig fusion protein (TOK107-hIg) were suspended in Dulbecco's phosphate buffered saline (PBS; GIBCO, Grand Island, N.Y.) and emulsified with an equal volume of complete Freund's adjuvant (Sigma Chemical Co., St. Louis, Mo.). XENOMOUSE™ were immunized by injection of the emulsion at three subcutaneous sites, base of tail and one intraperitoneal (i.p.) site. Fourteen days after the initial immunization, the mice were given a booster immunization with 50 μg TOK107-hIg in incomplete Freund's adjuvant. Sera testing indicated insufficient titer so after a few weeks rest, a second booster of 50 μg human TOK107-hIg was given. Two weeks later, a small amount of blood was collected from the tail vein and the serum activity against TOK107-Ig was titered by ELISA and against HT29-GCC#2 cells by FACS. Mice were selected for fusion when their titer exceeded 1:24, 300 by ELISA or 1:500 by FACS. Nearly three months after that boost, mice were boosted with 10⁷ HT-29 #2 cells and the next day boosted with 50 μg TOK107-hIg, both in incomplete Freund's adjuvant. A mouse immunized with this scheme produced the 5F9 and the 1D2 human anti-GCC antibody molecules. A mouse immunized with this scheme produced the 5F9 and the 1D2 human anti-GCC antibody molecules.

Four days later, the mice were euthanized and spleen cell suspensions were prepared and washed with PBS for the fusion. The fused cells were tested for production of antibodies which specifically bound to GCC by ELISA for binding TOK107-hIg compared to a nonGCC antigen or to the Fc region of IgG and by FACS for binding to T84 cells or HT-29 clone #2 cells compared to vector control and compared to non-GCC-expressing MCF-7 cells. Isotype was determined using ELISA or by FACS using IgG or IgM specific secondary antibodies. A mouse immunized with this scheme produced the 5F9 human anti-GCC antibody molecules.

Hybridomas that Produce Human mAb:

Spleen cells were counted and mixed with SP 2/0 myeloma cells (ATCC No. CRL8-006, Rockville, Md.) that are incapable of secreting either heavy or light chain immunoglobulin chains at a spleen:myeloma ratio of 2:1. Cells were fused with polyethylene glycol 1450 (ATCC) in 12 96-well tissue culture plates in HAT selection medium according to standard procedures. Between 10 and 21 days after fusion, hybridoma colonies became visible and culture supernatants were harvested then screened by ELISA and FACS.

Analysis of mAb by ELISA.

High-protein binding 96-well EIA plates (Costar/Corning, Inc. Corning, N.Y.) were coated with 50 μl/well of a 2 μg/ml solution (0.1 μg/well) of TOK107-hIg and incubated overnight at 4° C. The excess solution was aspirated and the plates were washed with PBS/0.05% Tween-20 (three times), then blocked with 1% bovine serum albumin (BSA, fraction V, Sigma Chemical Co., MO) for 1 hr at room temperature (RT) to inhibit non-specific binding. The BSA solution was removed and 50 μl/well of hybridoma supernatant from each fusion plate well were added. The plates were then incubated for 45 min. at 37° C. and washed three times with PBS/0.05% Tween-20. Horseradish peroxidase (HRP)-conjugated goat anti-mouse or anti-human IgG F(ab)2 (H&L) (Jackson Research Laboratories, Inc., West Grove, Pa.) diluted 1:4000 in 1% BSA/PBS was added to each well and then the plates were incubated for 45 min. at 37° C. After washing, 50 μl/well of ABTS solution (Zymed, South San Francisco, Calif.) was added. The intensity of the green color of positive wells at 405 nm was assessed on a Vmax microtitre plate reader (Molecular Devices Corp., Sunnyvale, Calif.). All hybridoma wells that gave a positive response were then expanded to 24-well cultures, subcloned by limiting dilution and analyzed by ELISA and FACS. The three best producing subclones were expanded further.

Analysis of mAb by Flow Cytometry.

Flow cytometry (FACS) screening was done on all the fusion plate supernatants in parallel to the ELISA screening. HT-29 clone #2 or untransfected HT-29 cells were grown in T225 flasks (Costar/Corning, Inc., Corning, N.Y.) in DMEM (GIBCO) supplemented 10% fetal bovine serum (GIBCO). Cells were detached from the flask surface using Versene (GIBCO), collected and washed twice with DMEM, then once with 1% BSA/PBS solution. The cells were re-suspended in 1% BSA/PBS and 2×10⁶ cells were added to each well of V-bottomed 96-well plates (Costar) and centrifuged for 5 min. at 2500 RPM (wash). The wash solution was discarded and 50 μl/well of supernatant from each fusion plate well wash added. A plate sealer (Linbro/MP Biomedicals, LLC, Solon, Ohio) was applied and the plates were then gently vortexed to resuspend and mix the cells with the supernatants and incubated at 4° C. (on ice) for 30 min. The plates were then washed with cold 1% BSA/PBS (three times) and 50 μl/well FITC-conjugated donkey anti-mouse IgG F(Ab)₂ (H&L) or FITC-conjugated goat anti-human IgG F(Ab)₂ (H&L) (Jackson) diluted 1:50 was added to each well for 30 min. at 4° C. (on ice in dark). The plates were again washed three times in cold 1% BSA/PBS and fixed in cold 1% paraformaldehyde (Sigma)/PBS. The cells were transferred to cluster tubes (Costar) and analyzed on a FACScalibur flow cytometer (Becton Dickenson, San Jose, Calif.). Any hybridoma wells that showed a positive shift were then expanded to 24-well cultures, subcloned by limiting dilution.

Internalization Assay.

Internalization of anti-GCC antibody molecules was tested in both GCC-expressing cells and vector control cells, using immunofluorescence microscopy. Cells were grown on coverslips and placed on ice for 10 minutes prior to incubation with 10 μg/ml antibody in cold culture medium for 20 minutes on ice. For internalization, antibody-containing medium was replaced with fresh culture medium and the cells were shifted to 37° C. for 2-3 hours or maintained on ice. After rinsing in PBS and a brief fixation in 4% paraformaldehyde at room temperature, cells were permeabilized for 15 min in 0/5% TRITON X-100. The localization of the test antibody was determined using a fluorescently labeled anti-IgG antibody by laser scanning confocal microscopy. Antibody molecules localized to the cell surface of GCC-expressing cells when on ice. Upon incubation at 37° C., 5F9 showed punctuate staining within the cell membrane, indicative of internalization. No internalization was detected with vector cells.

Summary of Properties of an Anti-GCC Antibody Molecule.

Table 7 summarizes the in vitro properties for a 5F9 mAb. (T84=human colon tumor cells, MCF7=human breast tumor cells, WB=western blot, IP=immunoprecipitation, IHC=immunohistochemistry; internalization used T84 cells compared to MCF-7 cells).

TABLE 7
Properties of anti-GCC antibody molecules

| | ELISA | | FACS | | | | | | | |
|---|---|---|---|---|---|---|---|---|---|---|
| | TOK107- | TOK82- | HT- | HT- | | MCF | | | | Internali- |
| Ab | hIg | hIg | 29#2 | 29 | T84 | 7 | WB | IP | IHC | zation |
| 5F9 | + | − | + | − | + | − | + | + | + | + |

Additionally, the 5F9 antibody molecule was tested for their ability to inhibit the ST peptide-induced calcium ion flux in GCC-expressing cells. The cGMP assay was performed in HT29-GCC#18 cells in the presence of 50 nM ST in the presence or absence of anti-GCC antibody molecules. There was dose-dependent inhibition of the calcium ion flux by 5F9.

Estimate of Relative Affinity of Anti-GCC Antibody Molecules.

The relative affinity (EC50; antibody concentration for half maximal binding) of the 5F9 anti-GCC antibody molecule was estimated from ELISA measurements against TOK107-hIg and by FACS measurements with GCC-expressing cells. The following table displays the results.

TABLE 8
EC50 of anti-GCC antibody molecules

| Antibody | EC50, TOK107-hIg, M | EC50, Cells, M |
|---|---|---|
| 5F9 | 3.65 × 10⁻⁸ | 1.24 × 10⁻⁹ |

Measurement of Affinity of Anti-GCC Antibody Molecules.

A BIACORE™ T100 system (GE Healthcare, Piscataway, N.J.) was used to measure the affinity of anti-GCC 5F9 antibody at 22° C.

Step 1: MAb 5F9 (Prep A) was diluted to 20 μg/mL in 10 mM sodium acetate, pH 4.0 and Reference 5F9 MAb (Prep B) was diluted to 10 μg/mL in 10 mM sodium acetate, pH 4.0. Each mAb was covalently immobilized to several CM4 BIACORE chips using standard amine coupling. For each CM4 chip prepared, Prep A 5F9 was immobilized over two flow cells at around 75-100 RU while Prep B 5F9 was immobilized to one flow cell at around 70-80 RU. The remaining fourth flow cell of each CM4 chip was used as the reference flow cell.

Step 2: The stock concentration of GCC-ECD-Fc (TOK107-hIg) was determined using the methods detailed by Pace et al. in Protein Science, 4:2411 (1995), and Pace and Grimsley in Current Protocols in Protein Science 3.1.1-3.1.9 (2003).

Step 3: For each prepared CM4 chip described in Step 1, GCC-ECD-Fc was injected for 2 minutes at a concentration range of 202 nM-1.6 nM (2× serial dilution) followed by a 7 minute dissociation. Samples were randomly injected in triplicate with several buffer inject cycles interspersed for double referencing. To obtain more significant off-rate decay data, three additional 101 nM GCC-ECD-Fc injections and three additional buffer injections were performed with a 2 minute injection and a 4 hour dissociation time. A flow rate of 100 μL/min was used for all experiments and all surfaces were regenerated with a 20 second pulse of 10 mM Glycine-HCl (pH 2.0). All samples were prepared in the running buffer which was Hepes-buffered saline, 0.005% polysorbate 20, pH 7.4 (HBS-P) with 100 μg/mL of BSA added.

Step 4: All sensorgram (plot of surface plasmon resonance vs time) data were processed with Scrubber 2.0 software (BioLogic Software, Campbell, Australia) and globally fit to a 1:1 interaction model including a term for the mass transport constant kₘ using CLAMP™ software (Myszka and Morton Trends Biochem. Sci. 23:149-150 (1998)).

The 1:1 model provided a very good fit to the data as long as the mAb immobilization levels were kept low enough so that the Rₘₐₓ resulting from the global analysis of the sensorgram data was at least below 12 RU for each surface. In most cases, one of the two Prep A 5F9 surfaces had an Rₘₐₓ too low (below 2 RU) for reliable kinetic measurements. Data from two flow cells of GCC-ECD-Fc binding to Prep A 5F9 from the same CM4 chip were simultaneously fit whenever possible, however. When mAb surfaces were prepared resulting in a higher Rₘₐₓ (>12 RU), sensorgrams clearly showed complex kinetics and thus a 1:1 model fit the data poorly. This isn't surprising due to the fact that GCC-ECD-Fc is a bivalent construct and a higher surface density of immobilized mAb most likely increases the probability that the GCC-ECD-Fc binds avidly to the surface. Replicates reported for this study include only those data that fit well to the 1:1 interaction model. The resulting K_{D}'s and rate constants of all replicates for Prep A 5F9 and the Prep B reference mAb are listed in Table 9 and Table 10, respectively.

TABLE 9
GCC-Fc binding to immobilized Prep A 5F9 mAb

| | Rₘₐₓ | kₐ | k_{d} | K_{D} |
|---|---|---|---|---|
| Replicate | (RU) | (M⁻¹s⁻¹) | (s⁻¹) | (pM) |
| A | 11 | 1.06 × 10⁵ | 1.19 × 10⁻⁵ | 112 |
| B | 8 | 1.20 × 10⁵ | 1.10 × 10⁻⁵ | 91.7 |
| C | 5 | 1.07 × 10⁵ | 2.15 × 10⁻⁵ | 201 |
| D | 9 | 1.22 × 10⁵ | 1.11 × 10⁻⁵ | 91.0 |
| E | 6, 4 | 9.64 × 10⁴ | 1.77 × 10⁻⁵ | 184 |
| Avg. (95% | | 1.10 (0.13) × 10⁵ | 1.46 (0.59) × 10⁻⁵ | 136 (65) |
| Conf. Int.) | | | | |

TABLE 10
GCC-Fc binding to immobilized Prep B 5F9 mAb

| | Rₘₐₓ | kₐ | k_{d} | K_{D} |
|---|---|---|---|---|
| Replicate | (RU) | (M⁻¹s⁻¹) | (s⁻¹) | (pM) |
| F | 9 | 9.68 × 10⁴ | 7.64 × 10⁻⁶ | 78.9 |
| G | 8 | 1.20 × 10⁵ | 1.24 × 10⁻⁵ | 103 |
| H | 7 | 9.09 × 10⁴ | 9.57 × 10⁻⁶ | 105 |
| I | 12 | 1.21 × 10⁵ | 1.54 × 10⁻⁵ | 127 |
| Avg. (95% | | 1.07 (0.25) × 10⁵ | 1.13 (0.54) × 10⁻⁵ | 103 (31) |
| Conf. Int.) | | | | |

### Conjugation of Toxins to Antibodies

Auristatins.

Conjugation by auristatins can be performed using published procedures (e.g., Doronina et al., Nature Biotech., 21: 778-784 (2003)). In general, auristatins are linked to cysteines of antibody chains. Linkage to cysteines is accomplished first by reduction of disulfide bonds in the antibody molecule. Control of the reduction process seeks to limit the reduction to some, but not necessarily all, interchain disulfide bonds. Consequently, auristatins are able to bind at the free cysteines. Quenching of the conjugation reaction is followed by removal of reaction by-products and buffer exchange to the desired formulation.

In brief, an anti-GCC antibody molecule at 7.6 mg/mL is pre-equilibrated at 37° C., and then a 15% volume of 500 mM sodium borate, pH 8.0 is added to raise the pH to 7.5-8.0. The solution also contains 1 mM DTPA. The antibody is partially reduced by adding 2.6 equivalents of tris(2-carboxyethyl)phosphine (TCEP) per mole of anti-GCC antibody molecule and stirring at 37° C. After 28 minutes, the solution of reduced anti-GCC antibody molecule is placed on ice, then treated immediately with 4.8-4.9 molar equivalents (relative to anti-GCC antibody molecule) of drug linker (e.g., mc-vc-MMAF or mc-vc-MMAE or mc-MMAF) as a 20.5 mM solution in DMSO. Additional DMSO is introduced to bring the mixture to 10% DMSO by volume. The reaction mixture is stirred on ice for ˜90 minutes before treatment with a 5-fold molar excess of N-acetyl cysteine (relative to mc-vc-MMAF). The conjugate is isolated by tangential flow filtration, first being concentrated to ˜10 mg/mL, then diafiltered with ˜10 diavolumes of PBS. The resulting antibody drug conjugates had an average drug loading of about four drug-linker units per antibody. For convenience, in the following Examples and attached Figures, auristatin immunoconjugates are referred to in the following abbreviated format, irrespective of drug loading: “Ab-vc-MMAF” refers to an anti-GCC antibody molecule conjugated with mc-vc-MMAF; “Ab-vc-MMAE” refers to an anti-GCC antibody molecule conjugated with mc-vc-MMAE; and “Ab-mc-MMAF” refers to an anti-GCC antibody molecule conjugated with mc-MMAF. Immunoconjugates comprising specific anti-GCC antibody molecules are referred to in the same format, .e.g., 5F9-vc-MMAF, 5F9-vc-MMAE, and 5F9-mc-MMAF.

To prepare antibody drug conjugates with an average drug loading of about two drug-linker units per antibody, the protocol (above) is modified by reducing the amount of TCEP by 50%. The amount of drug linker is also reduced by 50%. The corresponding antibody drug conjugate is abbreviated as Ab-vc-MMAF(2).

Preparation of 5F9 vcMMAE

Using a method similar to the general method set forth above, the 5F9 mAb was conjugated to an auristatin derivative designated MMAE (Formula (XIII)) using a vc (Val-Cit) linker described herein to create the immunoconjugate designated 5F9 vcMMAE. The conjugation of the vc linker to MMAE (Seattle Genetics, Inc., Bothell, Wash.) was completed as previously described (see, e.g., US 2006/0074008).

Briefly, a 17.8 mg/mL solution of the 5F9 mAb in 100 mM acetate at pH 5.8 was adjusted to pH 8 with 0.3 M sodium phosphate dibasic, yielding a final mAb concentration of 11.3 mg/ml. Then, DTPA was added for a 1 mM final concentration in the reaction mixture. The mAb was then partially reduced by adding 2.28 molar equivalents of TCEP (relative to moles of mAb), and then stirred at 37° C. for 1.5 hours. The partially reduced mAb solution was then cooled to 4° C., and 4.4 molar equivalents of vcMMAE (relative to moles of antibody) were added as a 20.3 mM solution in DMSO. The mixture was stirred for 30 minutes at 22° C., then for 15 additional minutes following the addition of 5 molar equivalents of N-acetylcysteine (relative to moles of vcMMAE). Excess quenched vcMMAE and other reaction components were removed by ultrafiltration/diafiltration of the immunoconjugate with 10 diavolumes of PBS, pH 7.4. The resulting immunoconjugate was designated 5F9 vcMMAE and has the following formula:

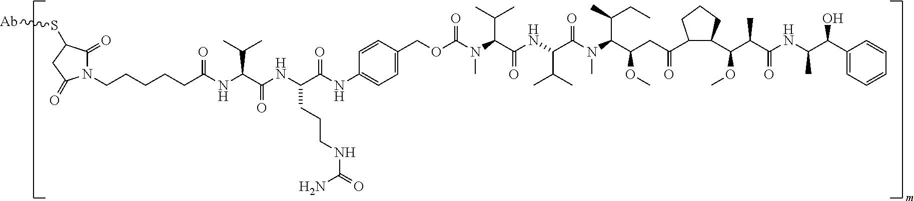

where Ab is the 5F9 mAb, and m is from 1 to 8. The average drug loading (m) was about 3.6. In a composition comprising a plurality of conjugates, the average number of MMAE molecules attached to an antibody is 3.6.

Cytotoxicity Assays.

To measure each antibody's ability to bind, internalize and kill target expressing cells, cytotoxicity assays were performed. In this assay, cells were incubated with various concentrations of the unconjugated primary anti-GCC antibody and a fixed non-toxic concentration of DM1-conjugated anti-human Fc secondary antibody (indirect cytotoxicity) or with various concentrations of toxin conjugated anti-GCC mAb (direct cytotoxicity). Cell viability was measured by WST assay after 4 days incubation. The relative potency of human anti-GCC antibodies on 293-GCC#2 cells is shown in Table 8 and was determined using a DM1 conjugated mouse anti-human IgG mAb (MAH-IgG was purified from clone HP607 (CRL1753, ATCC). 5F9 and 229 are the most potent anti-GCC mAb with LD50's of 26 and 78 pM. Although not shown here, the error is generally within 20% of these averages, as measured by range of replicates or standard deviation of >2 replicates.

TABLE 11
Cytotoxicity assay results for anti-
GCC antibodies on 293-GCC#2 cells

| Anti-GCC Ab | LD50 (pM) |
|---|---|
| 5F9 | 26 |

Cell Surface Binding.

Binding of unconjugated 5F9 or 5F9 conjugated to auristatins, was evaluated by in indirect immunofluorescence assay using flow cytometry. 1×10⁶ cells/well were plated in a V-bottom 96 well plate and incubated on ice for 1 hour with serial antibody dilutions of 1-0.001 μg/ml. Cells were washed twice with 3% FBS in ice cold PBS and incubated with 1:200 mouse anti-human PE IgG (Southern Biotech 2043-09) for 1 hour on ice. Cells were washed again and analyzed by flow cytometry on a BD FACS Canto II flow cytometer. Data was analyzed using FACS Canto II system software and mean fluorescence intensities were determined.

Cell Surface Binding to GCC Truncation Mutants.

Truncation mutants of the GCC ECD were generated (FL mature peptide and 8 truncations (Δ1-32, Δ1-49, Δ1-94, Δ1-128, Δ1-177, Δ1-226, Δ1-279, Δ1-229 and Δ1-379), as FLAG tagged constructs (pFLAG-CMV-3), representing approximately 50 amino acid deletion increments. Constructs were expressed in 293 cells, followed by immunoprecipitation by the anti-GCC antibody molecule and Western blotting for the FLAG epitope in lysates of 293 cells transfected with the GCC ECD mutants. Antibody 5F9 binds cells with the Δ1-32 mutation, but not cells with the Δ1-49 mutation. The binding of 5F9 to GCC was lost when the protein is truncated between amino acids 33-50 suggesting that this region is involved in the recognition of 5F9 to its binding epitope on GCC. However, since the rat and mouse GCC sequences are identical to human GCC in this region, and 5F9 does not bind mouse or rat GCC, 5F9 antibody likely binds a conformational epitope formed by the presence of amino acids 33 to 50 of human GCC.

### Example 2

### Toxin-Linker Selection/ADC Characterization

In an antibody drug conjugate (ADC) strategy, the conjugation of highly potent toxins to antibodies, the cytotoxicity of the toxin can be directed to tumors in a target-specific manner, delivering the toxin to antigen expressing tumor cells without affecting antigen negative cells in normal tissues thus reducing systemic toxicity. Auristatin (analog of dolastatin 10) and maytansine class toxins were evaluated as ADCs with anti-GCC mAb. These toxins are all inhibitors of microtubule polymerization, acting as anti-mitotics. Tests in which cells were contacted with free toxins indicated that the cytotoxicity of free toxins did not distinguish between cells with GCC expression and control cells without GCC. These free toxins were potent against the 293-vector, 293-GCC#2 cells, in HT29-vector vs. HT29-GCC#5 cells as shown in Table 12.

TABLE 12
Cytotoxicity of free toxins

| | MMAE | | MMAF | | DM1 | | DM4 | |
|---|---|---|---|---|---|---|---|---|
| Cell line | LD50 | SD | LD50 | SD | LD50 | SD | LD50 | SD |
| 293 vector | 0.07 | 6 | 3.37 | 2.34 | 2.83 | 2.00 | 0.79 | 0.79 |
| 293 GCC#2 | 4 | 3 | 2.07 | 1.48 | 2.96 | 2.23 | 0.83 | 1.03 |
| HT29 | 4 | 1 | 5.21 | 1.91 | 2.69 | 1.09 | 0.57 | 0.27 |
| HT29 GCC#5 | 7 | 4 | 7.65 | 3.32 | 1.00 | 0.14 | 0.40 | 0.17 |

The chemistry by which the different toxins are conjugated to antibodies differs and affects linker stability. Linker stability affects the therapeutic window by impacting drug release in the blood or nontarget tissues vs. drug release at the tumor. The ideal ADC linker has high stability while in the blood, but efficient release upon target mediated cell entry.

### Auristatins

Three auristatin-linker pairs were evaluated. To first evaluate these conjugates in vitro and then determine which toxin-linker for large scale in vivo studies, 5F9 was conjugated to vcMMAE, vcMMAF and mcMMAF (20 mgs per conjugate).

Auristatins are synthetic toxins related to the natural product dolastatin 10. MMAE and MMAF differ subtly, with the MMAF form having a carboxylic acid group in the R2 position, reducing cell permeability and potency as free toxin. MMAE is a Pgp drug pump substrate, while MMAF is not.

Auristatins are conjugated to interchain cysteines through a process of partial antibody reduction, reaction with a maleimido drug derivative, quenching with excess cysteine, concentration and buffer exchange into PBS. The auristatins can be attached with a cathepsin B sensitive dipeptide linker, which is cleaved upon cellular uptake, or with a noncleavable linker.

In the vcMonoMethylAuristatin linker, a valine citrulline dipeptide linkage is attached to the drug through a p-amino benzyl carbamate (PAB) group and to the antibody through a maleidimido caproyl conjugation group. Upon internalization, the dipeptide linker is cleaved by the lysosomal protease cathepsin B, the PAB group self destructs, and free toxin is released. This linker was designed to maintain serum stability while maximizing intracellular drug release by cathepsin B.

Auristatins can also be linked to antibodies through noncleavable linkers such as MMAF directly attached to the maleimido conjugation group, with no peptidase sensitive linker. MC conjugated ADC's are also effective at target mediated cell kill

The mechanism of drug release for noncleavable auristatin conjugates is thought to be through general antibody degradation in lysosomes. Through LC/MS studies it has been reported that Ab-mcMMAF conjugates release toxin in the form of a single cysteine-adduct.

### Binding of Antibody Drug Conjugates

All of the 5F9 antibody drug conjugates bound to 293-GCC#2 cells equally well. Table 10 shows the mean fluorescence intensity of 5F9 conjugates at increasing concentrations 293-GCC#2 cells. Other studies determined that the 5F9-SPDB-DM4 conjugate bound 293-GCC#2 cells in a concentration-dependent manner, whereas the 209-SPDB-DM4 antibody did not bind.

TABLE 13
Chart of MFIs from binding assay of 5F9 and 5F9
conjugates in 293 GCC #2 cells:

| | μg/ml of 5F9 or 5F9 conjugate | | | | | |
|---|---|---|---|---|---|---|
| | 0.001 | 0.004 | 0.016 | 0.063 | 0.25 | 1 |
| 5F9 | 500 | 937 | 2465 | 6615 | 7816 | 8026 |
| vcMMAE | 445 | 696 | 1787 | 4854 | 7296 | 7416 |
| vcMMAF | 440 | 707 | 1502 | 4830 | 7563 | 7779 |
| mcMMAF | 483 | 776 | 2106 | 5353 | 7398 | 7585 |

The 5F9-auristatin toxin conjugates were tested in direct cytotoxicity assays of a variety of cells which been transfected with GCC nucleic acid and selected for expression of GCC. A survey of the level of surface expression of GCC found that 293 GCC#2 cells express high amounts of GCC; HT 29 #2 and CT 26 #2.5 cells express GCC at intermediate to low levels; CT 26 #32 cells express GCC at high levels; and HT 29 GCC #5 and HT 29 GCC #18 express low amounts of GCC. Table 11 shows a compilation of multiple studies yielding cytotoxicity data for the three auristatin conjugates in cells expressing target, or in wild-type cells or vector control cells. Target enhanced killing is observed in all cases of 5F9-conjugated toxins on 293 GCC #2 cells, with a greatly increased window when using MMAF vs. MMAE. Both the cleavable and noncleavable forms of MMAF were similarly potent. As a negative control, antibody drug conjugates were also made with sc209 antibody, which is a human IgG1 monoclonal antibody raised against an unrelated target, with no reactivity to GCC. Direct cytotoxicity assays with 209 ADCs vs. 5F9 vcMMAF ADC showed target enhanced cell kill in the 293 cell model and the HT29 cell model. A comparison of 5F9-conjugated toxin activity among the cell lines indicates that the level of cytotoxicity has some correlation with the amount of GCC expressed by the cells. These data suggest that at least some cell lines expressing the most GCC were more susceptible to the cytotoxic activity of the conjugate than were cells expressing lower amounts of GCC. See Example 1 for relative GCC numbers per cell. Another factor in differences of cytotoxicity levels may be differences in internalization or intracellular processing of the conjugates, which may vary among wild type cell lines.

TABLE 14
Cytotoxicity of anti-GCC auristatin ADCs.

| | | | | LD50 (nM) |
|---|---|---|---|---|
| | LD 50 (nM) of 5F9 conjugates | | | of 209 |
| Cell line | vcMMAE | vcMMAF | mcMMAF | conjugates |
| 293 Vector | 128 | >10 | >10 | |
| 293 GCC # 2 | 0.37 | 0.001 | 0.002 | |
| 293 Vector | 1.8 | >10 | >10 | |
| 293 GCC # 2 | 0.13 | 0.005 | 0.007 | |
| 293 Vector | | >10 | | >10 |
| 293 GCC # 2 | | 0.0004 | | >10 |
| HT 29 WT | 84 | >10 | >10 | |
| HT 29 GCC #2 | 24.1 | 0.127 | 3.1 | |
| CT 26 WT | >500 | >10 | >10 | |
| CT 26 GCC | >500 | >10 | >10 | |
| #2.5 | | | | |
| CT 26 GCC | 267 | 0.004 | 0.064 | |
| #32 | | | | |
| HT 29 vector | 520.6 | >10,000 | >10,000 | |
| HT 29 GCC #5 | 653.5 | 563.2 | >10,000 | |
| HT 29 GCC | 554.6 | >10,000 | >10,000 | |
| #18 | | | | |
| HT29 | 0.93 | >10 | >10 | |
| HT29 GCC#5 | 0.59 | 0.32 | >10 | |
| HT29 | | >10 | | >10 |
| HT29 GCC#5 | | 0.035 | | [symbol] 10 |

If these potencies translate in vivo, with equal efficacy between mc and vcMMAF, the higher predicted MTD for mcMMAF would suggest a larger therapeutic window for this conjugate.

### Example 3

### In Vivo Evaluation

### Tumor Models:

In vivo cytotoxicity of 5F9 ADCs were evaluated in mouse xenograft models. The initial in vivo work was done with the HT29-GCC#5 and #18 cell lines. The 293-GCC#2 cell line also was tested for in vivo growth and was developed as a serially transplantable trocar model.

To address the question of whether the GCC expression level in the xenograft model was relevant to the level of GCC expression in patients with metastatic colon cancer, GCC expression levels were compared by IHC analyses of xenograft tissue, human primary colon tumors and metastases. A panel of fresh frozen cell lines and tissues were utilized for GCC quantitation by IHC with a mouse mAb to GCC 3G1. For IHC quantitation, scoring was done using a semi-quantitative 0-3 score system. If tumor model GCC levels≦clinical GCC levels, modeling will likely be accurate or overestimate the exposure needed clinically. If tumor model GCC levels>clinical GCC levels, modeling may underestimate the exposure needed clinically.

While there was some variability in the expression of GCC in metastatic samples, expression by the HT29-GCC#5 and #18 cells was in the range of many of the metastatic samples. By IHC, the staining of GCC on the HT29-GCC#5 and #18 cells was equivalent or lower than on the metastatic cells. This data suggests that our tumor models express GCC at levels comparable to the levels found in clinical samples of metCRC.

Table 16 represents the scintillation counts for various tissues harvested at the 192 hour timepoint with averages for three animals represented. 5F9 preferentially accumulated in HT29-GCC#5 tumors vs. HT29-vector tumors, while 209 did not show much differential accumulation. This result provided support that a 5F9 antibody drug conjugates could be expected to accumulate in GCC expressing tumors. In all other tissues evaluated, there was little difference in the levels of 5F9 vs. mAb 209 antibody accumulation.

### In Vivo Distribution of Radiolabeled 5F9 in HT29-GCC#5 & HT29-Vector Tumor-Bearing Mice

A radioimaging study in tumor bearing mice was performed to evaluate tumor targeting and in vivo biodistribution of the anti-GCC antibody 5F9 and a negative control antibody sc209 (human IgG1 monoclonal antibody targeting an unrelated cell surface target). The antibodies were radiolabeled with ¹¹¹In using DTPA as a bifunctional chelator. The in vivo behavior, including tumor targeting and biodistribution in normal tissues over time, was investigated with a murine dual-tumor model with both GCC(−) and GCC(+) tumors. In vivo images (SPECT/CT) were acquired, and tissue radioactivity counting was used to supplement the spatial resolution.

Subcutaneous tumors were grown in nude mice, with HT29-vector tumors on the right and HT29-GCC#5 tumors on the left. Antibodies were dosed at 0.3 mCi=15 μg per animal. There were three animals per group, and groups were harvested at 1 h, 24 h, 48 h, 72 h, 120 h and 192 h.

A survey of tissues from animals in the 192 h group indicated that both 5F9 and 209 accumulated to a similar degree in most normal tissues (e.g., blood, heart, gastric, small intestine, large intestine, muscle and skin) and in HT29-vector control tumors. Ab 209 accumulated to slightly higher levels than 5F9 in liver and 5F9 accumulated to slightly higher levels than 209 in lungs, spleen and kidneys. In the HT29-GCC#5 tumors, 5F9 preferentially accumulated at levels more than two-fold higher than the 209 levels. This result provided support that a 5F9 antibody drug conjugates could be expected to accumulate in GCC expressing tumors.

To understand the kinetics of antibody accumulation in tumors, tumor data for each antibody was obtained for all time points throughout the study. The only tissue that showed accumulation is the 5F9 antibody in the GCC expressing tumor. The radioactivity level in all other tissues remained relatively flat, with little difference between the 5F9 levels and 209 levels. 5F9 preferentially accumulated in HT29-GCC#5 tumors, while 209 did not show any accumulation. This result provided support that a 5F9 antibody drug conjugate could be expected to accumulate in GCC expressing tumors.

TABLE 16
Accumulation of ¹¹¹In-labeled GCC specific mAb
but not control mAb to tumors expressing GCC.

| | | Mean % ID 5F9 | Mean % ID ctr IgG |
|---|---|---|---|
| 1 | hr | 2.816 +/− 0.133 | 2.494 +/− 0.167 |
| 24 | hr | 3.057 +/− 0.107 | 3.010 +/− 0.630 |
| 72 | hr | 4.485 +/− 1.029 | 3.564 +/− 0.152 |
| 120 | hr | 5.162 +/− 1.012 | 3.412 +/− 0.048 |
| 192 | hr | 6.550 +/− 1.015 | 2.782 +/− 0.085 |

Accumulation of radiolabeled 5F9 in GCC expressing tumors over 7 days supported a once weekly dosing schedule.

Pilot Efficacy Studies in HT29-GCC#5 s.c. Tumors

Studies were performed to determine efficacious conjugates and dosage regimens in mice bearing HT29-GCC#5 tumors. Mice were dosed with single or multiple doses. These studies determined that there was toxicity at too frequent dosing at higher levels of toxin conjugate (e.g., 150 μg/kg 5F9vcMMAF on a q3d×5 schedule). Another study determined that q14d×5 schedule was too infrequent in this model to allow some toxin conjugates to show significant efficacy vs. controls. Additionally, a PD study with maytansinoid-antibody conjugates in this model demonstrated dose-dependent phosphohistone accumulation only with the DM4 toxin, not the DM1 toxin. Another study in this model showed some tumor growth inhibition by the non-GCC specific 209—toxin conjugate. These results suggested that other in vivo models needed to be evaluated.

PK/PD Study with 5F9 ADCs in Mice Carrying 293-GCC#2 Tumors.

An alternative tumor model used 293-GCC#2 cells. A PD study for 5F9 ADCs in 293-GCC#2 tumor bearing mice was performed. Mice were dosed with single doses of 5F9vcMMAF at 75 ug/kg or 150 ug/kg and serum was taken at timepoints from 1 hr through 4 days for PD analysis of phosphohistone H3 to test antimitotic effects of the toxin on tumor cells. Phospho-histone H3 was detected by antibody (Upstate Biotechnology, now Millipore, Billerica, Mass.) staining of paraffin embedded sections of tumors. The data in Table 17 shows that each of the ADCs caused a significant increase in the pH3 positive cell population in the tumors indicating that each of them at both 75 and 150 μg/kg toxin dose equivalents was able to reach the tumors and have the desired anti-mitotic effect on the tumor cells.

TABLE 17
The PD response as assessed by arrest of cells in mitosis (% pH
3 positive tumor cells) following a single iv dose of 5F9 ADCs.

| | Average % | |
|---|---|---|
| | Tumor cells | |
| | pH 3 positive | SD |
| Vehicle control | 2.556801 | 2.37707 |
| 5F9-vcMMAE 75 μg/kg 1 hr | 4.525187 | 0.178882 |
| 4 hr | 2.551616 | 1.688255 |
| 8 hr | 4.243988 | 0.352938 |
| 24 hr | 9.8199 | 4.82057 |
| 48 hr | 8.692061 | 4.756786 |
| 96 hr | 8.628345 | 1.065456 |
| 5F9-vcMMAE 150 μg/kg 1 hr | 3.334943 | 1.351667 |
| 4 hr | 2.78543 | 1.690216 |
| 8 hr | 4.575611 | 1.130484 |
| 24 hr | 13.78776 | 3.343155 |
| 48 hr | 14.26067 | 5.448921 |
| 96 hr | 14.67942 | 1.827724 |
| 5F9-vcMMAF 75 μg/kg 1 hr | 4.235245 | 0.617585 |
| 4 hr | 4.18364 | 0.846752 |
| 8 hr | 4.930098 | 0.54746 |
| 24 hr | 20.22484 | 2.453935 |
| 48 hr | 9.920771 | 3.788795 |
| 96 hr | 10.38187 | 1.896461 |
| 5F9-vcMMAF 150 μg/kg 1 hr | 3.465674 | 1.341187 |
| 4 hr | 4.416646 | 0.807636 |
| 8 hr | 8.594385 | 4.005021 |
| 24 hr | 21.53718 | 7.25212 |
| 48 hr | 15.15814 | 4.28407 |
| 96 hr | 11.12288 | 2.150476 |
| 5F9-mcMMAF 75 μg/kg 1 hr | 5.365582 | 1.14198 |
| 4 hr | 4.044478 | 0.992449 |
| 8 hr | 8.228597 | 3.098222 |
| 24 hr | 14.10734 | 1.611093 |
| 48 hr | 19.37223 | 8.146504 |
| 96 hr | 7.749388 | 1.180759 |
| 5F9-mcMMAF 150 μg/kg 1 hr | 3.212482 | 0.509604 |
| 4 hr | 4.722554 | 1.577531 |
| 8 hr | 9.105349 | 5.963128 |
| 24 hr | 27.51416 | 10.96057 |
| 48 hr | 13.34043 | 3.414961 |
| 96 hr | 15.60917 | 3.386154 |

Similar studies measured the phosphohistone levels in 293-GCC#2 tumor-bearing mice treated with 5F9vcMMAF, 5F9-SPDB-DM4 and 5F9-SMCC-DM1. Mice were dosed with single doses at 150 ug/kg and serum was taken at timepoints from 1 hr through 21 days for PK analysis of both total antibody and toxin-conjugated antibody. The percentage of phosphohistone H3 positive cells in 293-GCC#2 tumors increased in response to all three ADCS: 5F9vcMMAF, 5F9-SMCC-DM1 and 5F9-SPDB-DM4. Maximal phosphohistone H3 levels were 3- to 5-fold increased over baseline, with peaks at 24 hours post-injection.

Efficacy Study with 5F9vcMMAF and 5F9-DMx in 293-GCC#2 s.c. Tumors

5F9-SPDB-DM4, 5F9-SMCC-DM1 and 5F9vcMMAF were tested for efficacy in the 293-GCC#2 tumor model at two doses (75 μg/kg and 150 Ξg/kg toxin), on a q14d×5 schedule. Specifically, this study included vehicle-treated control, Sc209-DM1 (150 μg/kg DM1 eq), Sc209-DM4 (150 μg/kg DM4 eq), Sc209-vcMMAF (150 μg/kg MMAF eq), 5F9-DM1 (150 μg/kg DM1 eq), 5F9-DM1 (75 μg/kg DM1 eq), 5F9-DM4 (150 μg/kg DM4 eq), 5F9-DM4 (75 μg/kg DM4 eq), 5F9-vcMMAF (150 μg/kg MMAF eq), and 5F9-vcMMAF (75 μg/kg MMAF eq). Taconic female mice bearing 293-GCC#2 cells (10 mice per group) were used.

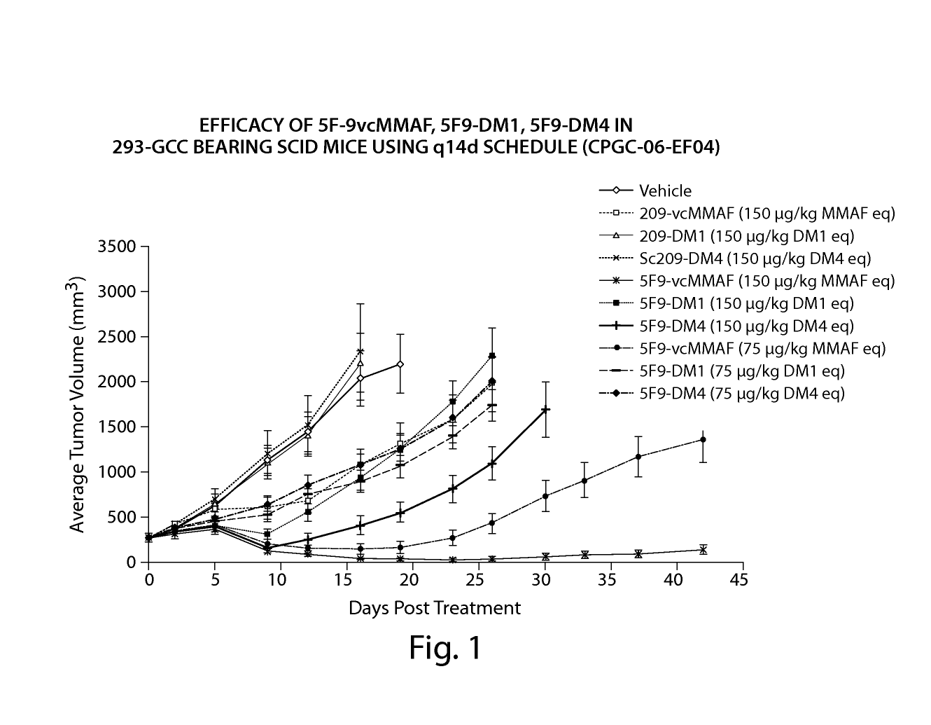

FIG. 1 depicts tumor growth in 293-GCC#2 bearing SCID mice treated with 5F9vc-MMAF, -DM1, and -DM4 on a q14d schedule. Dose-dependent efficacy was observed with 5F9-SPDB-DM4 in the 293-GCC#2 model, while the 209-SPDB-DM4 control had no effect. 5F9-SMCC-DM1 was also efficacious, however less so than 5F9-SPDB-DM4 at 150 ug/kg. 5F9vcMMAF (75 ug/kg and 150 ug/kg) was the most efficacious, however 209vcMMAF also had some activity. Therefore at these doses and schedules, 5F9-SPDB-DM4 had the greatest efficacious differential from its control conjugate.

Efficacy Study with 5F9vcMMAF and 5F9-DMx in 293-GCC#2 s.c. Tumors

5F9-SPDB-DM4 and 5F9-SMCC-DM1 were tested for efficacy in the 293-GCC#2 tumor model at two doses (75 ug/kg and 150 ug/kg toxin) on a q7d×5 schedule. Specifically, this study included vehicle-treated control, 5F9 alone (15 mg/kg), DM1 (300 μg/kg), DM4 (300 μg/kg), Sc209-DM1 (150 μg/kg DM1 eq), Sc209-DM4 (150 μg/kg DM4 eq), Sc209-vcMMAF (150 μg/kg MMAF eq), 5F9-DM1 (150 μg/kg DM1 eq), 5F9-DM1 (75 μg/kg DM1 eq), 5F9-DM4 (150 μg/kg DM4 eq), and 5F9-DM4 (75 μg/kg DM4 eq). Taconic female mice bearing 293-GCC#2 cells (10 mice per group) were used.

Efficacy Study with Auristatin Conjugates in 293 GCC#2 Tumors

293 GCC#2 tumor-bearing SCID mice were treated with 5F9 conjugates with vc MMAE, vcMMAF or mcMMAF at three doses in comparison with 209 conjugates of these toxins or with free toxins or vehicle control. Doses were administered iv on a q7d×4 schedule. Tumors were harvested at days 3, 7, 10, 13 and 17. The tumors in mice treated with control reagents demonstrated a continual increase in volume. Tumors treated with 5F9 auristatin conjugates showed dose- and time-dependent inhibition of this tumor growth. Table 18 provides a summary of the results (TGI=tumor growth inhibition, T/C=treatment/control, TGD=tumor growth delay, CR/PR=complete response/partial response; p value=measure to judge statistical significance, NS=not significant).

TABLE 18
Analysis of Auristatin ADCs in 293 GCC#2 tumor-bearing mice.

| Groups | TGI | T/C | TGD | CR/PR | P value |
|---|---|---|---|---|---|
| 209-vcMMAE 300 μg/kg | 24.5 | 0.76 | 0.9 | 0 | 0.38 > 0.05 |
| | | | | | NS |
| 209-vcMMAF 150 μg/kg | 29.4 | 0.71 | 1.4 | 0 | 0.27 > 0.05 |
| | | | | | NS |
| 209-mcMMAE 150 μg/kg | 36.5 | 0.63 | 1.4 | 0 | 0.15 > 0.05 |
| | | | | | NS |
| Free MMAE 300 μg/kg | 35.6 | 0.64 | 2 | 0 | 0.18 > 0.05 |
| | | | | | NS |
| Free mcMMAF 150 μg/kg | −3.4 | 1.03 | −1.3 | 0 | 0.73 > 0.05 |
| | | | | | NS |
| 5F9-vcMMAE 300 μg/kg | 96.9 | 0.03 | | 9/10 PR | <0.001 |
| 5F9-vcMMAE 150 μg/kg | 83.5 | 0.17 | | 2/10 PR | <0.01 |
| 5F9-vcMMAF 150 μg/kg | 97 | 0.03 | | 9/10 PR | <0.001 |
| 5F9-mcMMAF 150 μg/kg | 97 | 0.03 | | 9/10 PR | <0.001 |
| 5F9-vcMMAE 75 μg/kg | 54.5 | 0.45 | 4.4 | | 0.01 < |
| | | | | | p < 0.05 |
| 5F9-vcMMAF 75 μg/kg | 88.5 | 0.12 | | 6/10 PR | <0.001 |
| 5F9-mcMMAF 75 μg/kg | 87.5 | 0.13 | | 7/10 PR | =0.001 |
| 5F9-vcMMAF 37.5 μg/kg | 65.2 | 0.35 | 7.1 | | =0.01 |
| 5F9-mcMMAF 37.5 μg/kg | 63.6 | 0.36 | 5.8 | | =0.01 |

All three of these ADCs were efficacious in the 293 GCC#2 model on a q7d schedule. 5F9-vcMMAF and 5F9-mcMMAF are more potent than 5F9-vcMMAE, which correlates with in vivo PD (pH is H3) and in vitro cytotoxicity data.

Efficacy Study with 5F9vcMMAF and 5F9-DMx in T84 s.c. Tumors

5F9-SPDB-DM4 and 5F9-SMCC-DM1 were tested for efficacy in the T84 tumor model at two doses (75 ug/kg and 150 ug/kg toxin) on a q7d×5 schedule. Specifically, this study included vehicle-treated control, 5F9 alone (15 mg/kg), DM1 (300 μg/kg), DM4 (300 μg/kg), Sc209-DM1 (150 μg/kg DM1 eq), Sc209-DM4 (150 μg/kg DM4 eq), Sc209-vcMMAF (150 μg/kg MMAF eq), 5F9-DM1 (150 μg/kg DM1 eq), 5F9-DM1 (75 μg/kg DM1 eq), 5F9-DM4 (150 μg/kg DM4 eq), and 5F9-DM4 (75 μg/kg DM4 eq). Taconic females mice bearing T84 cells (10 mice per group) were used. (“Sc209-[toxin]” or “209-[toxin]” refers to a non-GCC targeting ADC used as a control in the studies described herein).

### Antitumor Activity of ADCs in a Primary Tumor Model

Two similar studies were conducted to determine the in vivo antitumor activity of 5F9-vcMMAE and to compare the antitumor activity of 5F9-vcMMAE to free toxin MMAE and to a non-GCC vcMMAE antibody toxin conjugate (209-vcMMAE) in PHTX-9c primary human colon tumor xenograft mice at various doses and dosing schedules and to determine the re-growth kinetics following treatment. Female CB-17 SCID mice (eight weeks old) were inoculated subcutaneously (SC) into the flank with PHTX-9c tumor fragments (2 mm×2 mm). Tumor growth was monitored twice per week using vernier calipers and the mean tumor volume was calculated using the formula (0.5×[length×width²]). When the mean tumor volume reached approximately 150 mm³ (Study A) or 160 mm³ (Study B), animals were randomized into treatment groups (n=10/group for Study A and n=9/group for Study B).

Mice were treated (Study A) on a once weekly (QW) dosing schedule (3 doses) with 0.938, 1.875, 3.75, or 7.5 mg/kg 5F9-vcMMAE intravenously (IV) for 20 days or controls, which included vehicle (0.9% saline), 0.075 or 0.15 mg/kg MMAE IV on a QW (once weekly) schedule, or 1.875 or 3.75 mg/kg 209-vcMMAE IV on QW dosing schedules for 20 Days. In the second study (Study B), mice were treated with 0.938, 1.875, 3.75, 7.5, or 10.0 mg/kg 5F9-vcMMAE IV on a QW schedule (3 doses) or 3.75 mg/kg IV on a twice weekly (BIW) schedule (6 doses), or controls including vehicle, 7.5 or 10 mg/kg 209-vcMMAE, or 0.135 or 0.18 mg/kg MMAE administered IV on a QW schedule for 20 days. Doses were administered on Days 1, 8, and 15 for the QW schedule and Days 1, 4, 8, 11, 15, and 18 for the BIW schedule. The dose of free MMAE was calculated to match the amount of MMAE in the immunoconjugate doses by the following rationale: The equivalent dose of MMAE is 1.8% of the MLN0264 dose. The equivalent dose of linker+MMAE is 4% of the 5F9-vcMMAE dose. These calculations are based on a mean 3.9 MMAE molecules per antibody and a free antibody molecular weight of 150 kD. Actual antibody molecular weight will vary slightly due to degree of glycosylation.

Tumor volume and body weight were measured twice weekly and were continued beyond the treatment period to measure regrowth kinetics, as evidenced by tumor growth delay (TGD). Tumor volume measurements were continued until tumor volume reached 10% of the body weight in a single mouse within a treatment group, at which time the group was terminated. The percentage of tumor growth inhibition (TGI) ([mean tumor volume of the control group—mean tumor volume of a treated group]/mean tumor volume of the control group; a T/C ratio) was determined on Day 20. The T/C ratios across a treatment group were compared to the T/C ratios of the control group using a two-tailed Welch's t-test. Because the entire group was terminated if one tumor reached the size limit (approx. 1000 mm³), TGD could not be calculated for groups where the average regrowth was slow.

The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value (<0.05) suggests that the trends over time for the two treatment groups were different. Results are summarized in Tables 19 and 20, below.

Antitumor activity was observed in all 5F9-vcMMAE-treated groups in both studies and the effect was shown to be dose-dependent. The results of the two studies were comparable. In mice treated with 5F9-vcMMAE at 0.938 mg/kg, IV on a QW schedule, TGI was 20.7-21.4%, p value was <0.05 compared with vehicle group. In the 1.875 mg/kg treated group administered IV, on a QW schedule TGI was 41.3-44.7%, p value was <0.001. In 3.75 mg/kg treated group administered IV, on a QW schedule TGI was 65.3-65.7% (p<0.001) compared with vehicle group. 5F9-vcMMAE administered at 7.5 mg/kg IV QW yielded a TGI of 84.1-84.3% (p<0.001) and 10 mg/kg IV QW (Study B only) yielded a TGI of 91.2% (p<0.001). When 3.75 mg/kg was administered IV on a BIW schedule (S), significant inhibition was observed with a TGI of 84.9% (p<0.001).

Moderate antitumor activity was observed at the higher doses of 209-vcMMAE of 7.5 and 10.0 mg/kg with TGI of 35.7 and 45.4%, respectively (p<0.001) but low doses of 209-vcMMAE (1.875 and 3.75 mg/kg) exhibited no inhibition (p>0.05). The antitumor activity observed by the high 209-vcMMAE is likely due to nonspecific activity by the MMAE portion of the immunoconjugate.

Administration of free toxin MMAE yielded mixed results: 0.075, 0.135, and 0.15 mg/kg administered IV QW yielded no tumor growth inhibition (p>0.05) but administration of 0.18 mg/kg IV QW resulted in TGI of 50.4% (p<0.001).

The greatest maximum body weight loss observed during treatment period was 2.3% on day 7 in the free toxin 0.18 mg/kg MMAE group of Study B and the 0.938 mg/kg 5F9-vcMMAE group of the same study. This indicates the drug was well-tolerated.

Tumor volume measurements were continued beyond the treatment period until tumor volume reached 10% of the body weight in a single mouse within a treatment group and then the treatment group was terminated. In these studies, tumor re-growth appeared to be dose-dependent.

TABLE 19
Study A results of treatment of primary human colon tumor Xenograft in SCID mice.

| | | | | BW change | TGD (days)/ |
|---|---|---|---|---|---|
| | | Method of | | (mean | (or days until |
| | Dose | Administration/ | | maximum | first tumor >1000 |
| Treatment | (mg/kg) | frequency | TGI | percent) | mm³) |
| Vehicle | 0 | IV QW × 3 doses | N/A | −0.2 | 0 |
| 209-vcMMAE | 1.875 | IV QW × 3 doses | −2.0 (p > 0.05) | 8.3 | 0.6 |
| 209-vcMMAE | 3.75 | IV QW × 3 doses | 4.5 (p > 0.05) | 6.9 | 0.4 |
| MMAE | 0.075 | IV QW × 3 doses | 5.0 (p > 0.05) | 8.3 | 1.0 |
| MMAE | 0.15 | IV QW × 3 doses | 11.1 (p > 0.05) | 10.8 | 2.0 |
| 5F9-vcMMAE | 0.938 | IV QW × 3 doses | 21.4 (p > 0.05) | 9.0 | 3.3 |
| 5F9-vcMMAE | 1.875 | IV QW × 3 doses | 44.7 (p > 0.001) | 10.6 | 8.2 |
| 5F9-vcMMAE | 3.75 | IV QW × 3 doses | 65.3 (p > 0.001) | 9.0 | 17.8 |
| 5F9-vcMMAE | 7.5 | IV QW × 3 doses | 84.1 (p > 0.001) | 7.5 | (>58 days |
| | | | | | first tumor) |

TABLE 20
Study B results of treatment of primary human colon tumor Xenograft in SCID mice.

| | | | | BW change | TGD (days)/ |
|---|---|---|---|---|---|
| | | Method of | | (mean | (or days until |
| | Dose | Administration/ | | maximum | first tumor >1000 |
| Treatment | (mg/kg) | frequency | TGI | percent) | mm³) |
| Vehicle | 0 | IV QW × 3 doses | N/A | −1.8 | 0 |
| 209-vcMMAE | 7.5 | IV QW × 3 doses | 35.7 (p > 0.001) | 4.2 | 4.2 |
| 209-vcMMAE | 10.0 | IV QW × 3 doses | 45.4 (p > 0.001) | 2.6 | 11.3 |
| MMAE | 0.135 | IV QW × 3 doses | 2.2 (p > 0.05) | 9.3 | 0.2 |
| MMAE | 0.18 | IV QW × 3 doses | 50.4 (p > 0.001) | −2.3 | 8.6 |
| 5F9-vcMMAE | 0.938 | IV QW × 3 doses | 20.7 (p > 0.001) | −2.3 | 3.8 |
| 5F9-vcMMAE | 1.875 | IV QW × 3 doses | 41.3 (p > 0.001) | 5.6 | 6.6 |
| 5F9-vcMMAE | 3.75 | IV QW × 3 doses | 65.7 (p > 0.001) | −2.2 | 16 |
| 5F9-vcMMAE | 3.75 | IV BIW × 6 doses | 84.9 (p > 0.001) | −1.8 | 31.9 |
| 5F9-vcMMAE | 7.5 | IV QW × 3 doses | 84.3 (p > 0.001) | 6.5 | (>47 days |
| | | | | | first tumor) |
| 5F9-vcMMAE | 10.0 | IV QW × 3 doses | 91.2 (p > 0.001) | 7.2 | (>56 days |
| | | | | | first tumor) |

Pilot Efficacy Study with Naked 5F9 Anti hGCC Antibody in CT26 hGCC/luc #32 Disseminated Model (balb/c Mice)

This model tests the ability of naked antibodies to bind to GCC-expressing tumor cells in the circulation and prevent establishment of new tumors. Female balb/c mice were inoculated by i.v. with CT26 hGCC/luc #32 cells at 1×10⁵/mouse and 5×10⁵/mouse. Vehicle 0.9% NaCl and non-specific antibody (naked 209) were administered to control groups for comparison to administration of naked 5F9. Both antibodies were engineered (in the pLKTOK58 vector) to have the IgG1 isotype, so their Fc regions could elicit an antibody-dependent cell-mediated cytotoxic response after binding to cell surface antigens (i.e., GCC for 5F9 and an unrelated target for 209). Dosing was started one day before inoculation by i.v, with a dosing schedule of once/week i.v ×4 (q7d×4). Tumor growth was monitored by Xenogen imaging system twice a week. Body weight and survival were monitored twice a week as well. Lung weight and images including MRI images were taken at the end of this study.

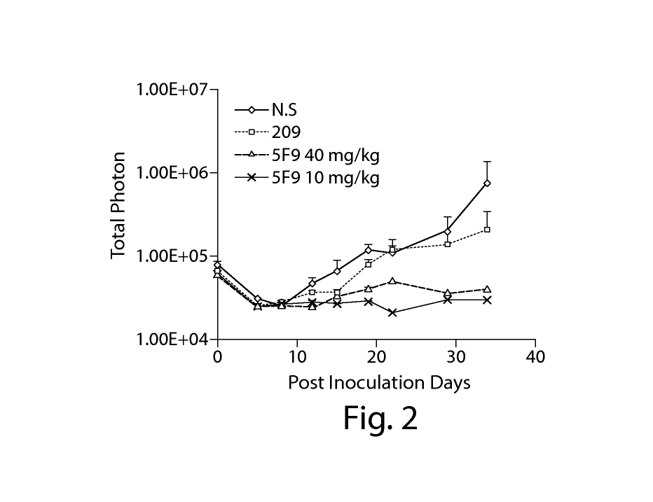

As shown in FIG. 2, both 5F9 groups (40 mg/kg and 10 mg/kg; 1×10⁵/mouse) show efficacy (on day 34p.i: T/C (treatment/control) is 0.04 to 0.05). T/C for the 5F9 group is 0.18 to 0.14 on day 34p.i compared to the 209 group. T/C for the 209 40 mg/kg group is 0.64 compared to 0.9% NaCl (Normal Saline) group. No benefit was seen with 5F9 in 5×10⁵ groups.

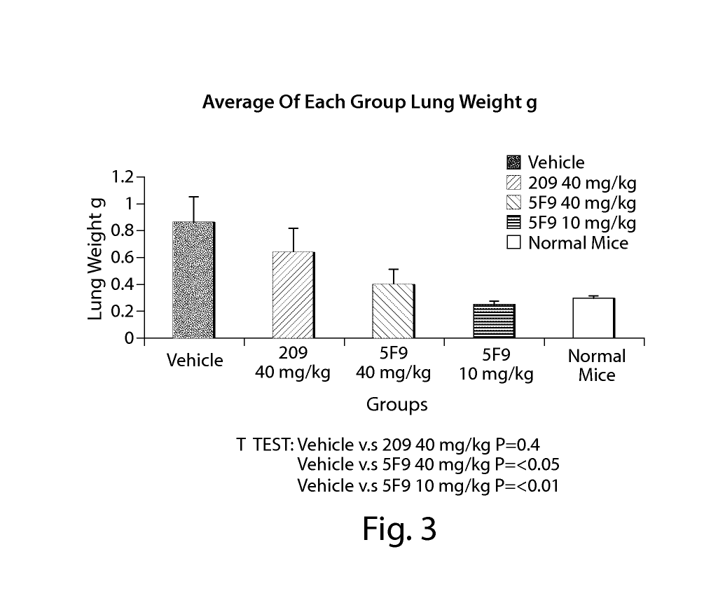

Lung weight of each group at the end of this study was shown in FIG. 3. T test: vehicle vs. 209 40 mg/kg P=0.4; vehicle vs. 5F9 40 mg/kg P<0.05; vehicle vs. 5F9 10 mg/kg P<0.01. Visual inspection of lungs confirmed fewer tumor nodules in the 5F9-treated groups than in the vehicle or 209-treated groups. In vivo MRI of the mice showed massive lung tumor exfiltration to surrounding tissues and heart displacement in vehicle-treated mice. In a 5F9 40 mg/kg-treated mouse, normal lung presentation was seen without evidence of tumor.

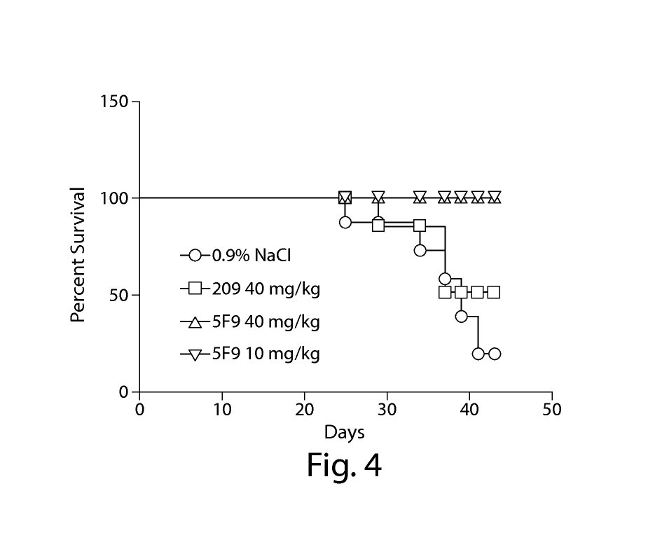

A survival curve is shown in FIG. 4. Significant increase in survival with 5F9 treated groups (1×10⁵) was observed and there was no difference between 5F9 10 and 40 mg/kg groups.

### Example 4

### Generating an Antibody Production Cell Line

To generate a stable CHO cell line clone expressing 5F9 with a productivity of >600 mg/L, expression vectors for 5F9 were generated by subcloning light chain variable region (SEQ ID NO:19) and heavy chain variable region (SEQ ID NO:17) into the pLKTOK58 expression vector, containing WT human IgG1 Fc and the neomycin resistance gene. Expression of the 5F9 variable region-IgG1 fusion product is under control of the EF-1a promoter.

### Cloning and Sequencing of the Anti-GCC Human Monoclonal Antibody 5F9 Variable Regions

Total RNA was isolated (Qiagen's RNeasy kit) from human hybridoma 46.5F9 subclone 8.2. This hybridoma carries the “standard” published Kappa constant region of the light chain (GenBank accession #AW383625, or BM918539) and the “standard” published IgG2 constant region of the heavy chain (GenBank accession #BX640623, or AJ294731). 5′ race-ready, poly-G tailed cDNA was synthesized by traditional methods (Nature Methods 2, 629-630 (2005)). The light chain variable region was PCR amplified from cDNA by 5′ race using a poly-C anchor oligo in combination with a reverse primer specific for the Kappa constant region. The heavy chain variable region was amplified with a reverse primer specific for the IgG2 constant region in multiple combinations with forward primers specific to the known heavy chain leader sequences. PCR products were TOPO® cloned (Invitrogen™, Life Technologies, Inc.) and sequenced with M13F and M13R primers.

### Construction of Mammalian Expression Vectors Carrying Anti-GCC Human Monoclonal Antibody 5F9

Mammalian expression vectors carrying the 5F9 light and heavy variable regions were constructed to generate production CHO cell lines. For the native construct, the variable regions of the 5F9 light and heavy chains were sub-cloned into pLKTOK58D (US Patent Application #20040033561). This vector carries two mammalian selection markers: neomycin resistance and DHFR/methotrexate (for amplification). The vector allows co-expression of both light and heavy chains from tandem EF1alpha promoters, each located upstream of the vector's leader-Kappa constant and leader-IgG1 (wild type Fc) constant regions. For sub-cloning, the variable regions of the light and heavy chains were PCR amplified from sequence-confirmed TOPO clones with gene-specific primers containing unique restriction sites for directional cloning into the junctions of the respective leader-Kappa and leader-IgG1 regions of the vector. The sequences of the primers are as follows (5F9 variable region-specific sequences in bold font):

### Native 5F9 Light Chain Leader-Variable Primers:

forward NotI
(SEQ ID NO: 21)
5′ataagaatGCGGCCGCCTCACCATGGGATGGAGCTGTATCATCCTCTT
CTTGGTAGCAACAGCTACAGGTGTCCACTCCGAAATAGTGATGACGCAGT
CTCCAGCCACCCTG-3′
reverse BsiWI
(SEQ ID NO: 22)
5′-GCCACCGTACGTTTGATTTCCACGTTGGTCCCTTGGCCGAACG
TC-3′

### Native 5F9 Heavy Chain Leader-Variable Primers

forward EcoRI
(SEQ ID NO: 23)
5′ccgGAATTCCTCACCATGGGATGGAGCTGTATCATCCTCTTCTTGGTA
GCAACAGCTACAGGTGTCCACTCCCAGGTGCAGCTACAGCAGTGGGGCGC
AGGAC-3′
reverse Blpl
(SEQ ID NO: 24)
5′-GGAGGCTGAGCTGACGGTGACCAGGGTTCCCTGGCCCCAGTGG
TC-3′

Clones were confirmed by double stranded DNA sequencing of both the light and heavy chains.

Two transfection methods were used to introduce the constructs into CHO cells: the traditional MPI process and the Crucell process. CHO cell transfections were initiated with the native 5F9 construct using the traditional MPI process. Linearized and nonlinearized DNAs were used, with either electroporation or Lipopfectamine 2000 CD transfection. Approximately 30 stable pools were generated through selection in G418, non nucleoside medium and 5 nM methotrexate. Based on FMAT analysis of antibody production levels, three stable pools were chosen for cloning. The pool with the highest production secreted antibody at 12.2 ug/ml. These three pools have been frozen down.

Crucell STAR elements can be evaluated to make 5F9 expression vectors containing a STAR element.

5F9/hIgG1 heavy chain nucleotide sequence is:

(SEQ ID NO: 31)
GAATTCCTCACCATGGGATGGAGCTGTATCATCCTCTTCTTGGTAGCAAC
AGCTACAGGTGTCCACTCCCAGGTGCAGCTACAGCAGTGGGGCGCAGGAC
TGTTGAAGCCTTCGGAGACCCTGTCCCTCACCTGCGCTGTCTTTGGTGGG
TCTTTCAGTGGTTACTACTGGAGCTGGATCCGCCAGCCCCCAGGGAAGGG
GCTGGAGTGGATTGGGGAAATCAATCATCGTGGAAACACCAACGACAACC
CGTCCCTCAAGAGTCGAGTCACCATATCAGTAGACACGTCCAAGAACCAG
TTCGCCCTGAAGCTGAGTTCTGTGACCGCCGCGGACACGGCTGTTTATTA
CTGTGCGAGAGAACGTGGATACACCTATGGTAACTTTGACCACTGGGGCC
AGGGAACCCTGGTCACCGTCAGCTCAGCCTCCACCAAGGGCCCATCGGTC
TTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAG
TCCTCAGGACTCTACTCCCTCAGCAGCGTGGTGACCGTGCCCTCCAGCAG
CTTGGGCACCCAGACCTACATCTGCAACGTGAATCACAAGCCCAGCAACA
CCAAGGTGGACAAGAAAGTTGAGCCCAAATCTTGTGACAAAACTCACACA
TGCCCACCGTGCCCAGCACCTGAACTCCTGGGGGGACCGTCAGTCTTCCT
CTTCCCCCCAAAACCCAAGGACACCCTCATGATCTCCCGGACCCCTGAGG
TCACATGCGTGGTGGTGGACGTGAGCCACGAAGACCCTGAGGTCAAGTTC
AACTGGTACGTGGACGGCGTGGAGGTGCATAATGCCAAGACAAAGCCGCG
GGAGGAGCAGTACAACAGCACGTACCGTGTGGTCAGCGTCCTCACCGTCC
TGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGA
CCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAA
GACCACGCCTCCCGTGCTGGACTCCGACGGCTCCTTCTTCCTCTACAGCA
AGCTCACCGTGGACAAGAGCAGGTGGCAGCAGGGGAACGTCTTCTCATGC
TCCGTGATGCATGAGGCTCTGCACAACCACTACACGCAGAAGAGCCTCTC
CCTGTCTCCGGGTAAATAATAGGGATAACAGGGTAATACTAGAG

5F9/hIgG1 heavy chain protein sequence is:

(SEQ ID NO: 32)
MGWSCIILFLVATATGVHSQVQLQQWGAGLLKPSETLSLTCAVFGGSFSG
YYWSWIRQPPGKGLEWIGEINHRGNTNDNPSLKSRVTISVDTSKNQFALK
LSSVTAADTAVYYCARERGYTYGNFDHWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGK

5F9/hKappa light chain nucleotide sequence is:

(SEQ ID NO: 33)
GCGGCCGCCTCACCATGGGATGGAGCTGTATCATCCTCTTCTTGGTAGCA
ACAGCTACAGGTGTCCACTCCGAAATAGTGATGACGCAGTCTCCAGCCAC
CCTGTCTGTGTCTCCAGGGGAAAGAGCCACCCTCTCCTGCAGGGCCAGTC
AGAGTGTTAGCAGAAACTTAGCCTGGTATCAGCAGAAACCTGGCCAGGCT
CCCAGGCTCCTCATCTATGGTGCATCCACCAGGGCCACTGGAATCCCAGC
CAGGTTCAGTGGCAGTGGGTCTGGGACAGAGTTCACTCTCACCATCGGCA
GCCTGCAGTCTGAAGATTTTGCAGTTTATTACTGTCAGCAGTATAAAACC
TGGCCTCGGACGTTCGGCCAAGGGACCAACGTGGAAATCAAACGTACGGT
GGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAG
GCCAAAGTACAGTGGAAGGTGGATAACGCCCTCCAATCGGGTAACTCCCA
GGAGAGTGTCACAGAGCAGGACAGCAAGGACAGCACCTACAGCCTCAGCA
GCACCCTGACCCTGAGCAAAGCAGACTACGAGAAACACAAAGTCTACGCC
TGCGAAGTCACCCATCAGGGCCTGAGCTCGCCCGTCACAAAGAGCTTCAA
CAGGGGAGAGTGTTAGTCTAGA

5F9/hKappa light chain protein sequence is:

(SEQ ID NO: 34)
MGWSCIILFLVATATGVHSEIVMTQSPATLSVSPGERATLSCRASQSVSR
NLAWYQQKPGQAPRLLIYGASTRATGIPARFSGSGSGTEFTLTIGSLQSE
DFAVYYCQQYKTWPRTFGQGTNVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC

The heavy and light chain nucleic acid sequences for 5F9 listed below were inserted into pTOK58D vector:

(SEQ ID NO: 35)
atgggatggagctgtatcatcctcttcttggtagcaacagctacaggtgt
ccactccgaaatagtgatgacgcagtctccagccaccctgtctgtgtctc
caggggaaagagccaccctctcctgcagggccagtcagagtgttagcaga
aacttagcctggtatcagcagaaacctggccaggctcccaggctcctcat
ctatggtgcatccaccagggccactggaatcccagccaggttcagtggca
gtgggtctgggacagagttcactctcaccatcggcagcctgcagtctgaa
gattttgcagtttattactgtcagcagtataaaacctggcctcggacgtt
cggccaagggaccaacgtggaaatcaaacgtacggtggctgcaccatctg
tcttcatcttcccgccatctgatgagcagttgaaatctggaactgcctct
gttgtgtgcctgctgaataacttctatcccagagaggccaaagtacagtg
gaaggtggataacgccctccaatcgggtaactcccaggagagtgtcacag
agcaggacagcaaggacagcacctacagcctcagcagcaccctgaccctg
agcaaagcagactacgagaaacacaaagtctacgcctgcgaagtcaccca
tcagggcctgagctcgcccgtcacaaagagcttcaacaggggagagtgtt
ag
(SEQ ID NO: 36)
atgggatggagctgtatcatcctcttcttggtagcaacagctacaggtgt
ccactcccaggtgcagctacagcagtggggcgcaggactgttgaagcctt
cggagaccctgtccctcacctgcgctgtctttggtgggtctttcagtggt
tactactggagctggatccgccagcccccagggaaggggctggagtggat
tggggaaatcaatcatcgtggaaacaccaacgacaacccgtccctcaaga
gtcgagtcaccatatcagtagacacgtccaagaaccagttcgccctgaag
ctgagttctgtgaccgccgcggacacggctgtttattactgtgcgagaga
acgtggatacacctatggtaactttgaccactggggccagggaaccctgg
tcaccgtcagctcagcctccaccaagggcccatcggtcttccccctggca
ccctcctccaagagcacctctgggggcacagcggccctgggctgcctggt
caaggactacttccccgaaccggtgacggtgtcgtggaactcaggcgccc
tgaccagcggcgtgcacaccttcccggctgtcctacagtcctcaggactc
tactccctcagcagcgtggtgaccgtgccctccagcagcttgggcaccca
gacctacatctgcaacgtgaatcacaagcccagcaacaccaaggtggaca
agaaagttgagcccaaatcttgtgacaaaactcacacatgcccaccgtgc
ccagcacctgaactcctggggggaccgtcagtcttcctcttccccccaaa
acccaaggacaccctcatgatctcccggacccctgaggtcacatgcgtgg
tggtggacgtgagccacgaagaccctgaggtcaagttcaactggtacgtg
gacggcgtggaggtgcataatgccaagacaaagccgcgggaggagcagta
caacagcacgtaccgtgtggtcagcgtcctcaccgtcctgcaccaggact
ggctgaatggcaaggagtacaagtgcaaggtctccaacaaagccctccca
gcccccatcgagaaaaccatctccaaagccaaagggcagccccgagaacc
acaggtgtacaccctgcccccatcccgggatgagctgaccaagaaccagg
tcagcctgacctgcctggtcaaaggcttctatcccagcgacatcgccgtg
gagtgggagagcaatgggcagccggagaacaactacaagaccacgcctcc
cgtgctggactccgacggctccttcttcctctacagcaagctcaccgtgg
acaagagcaggtggcagcaggggaacgtcttctcatgctccgtgatgcat
gaggctctgcacaaccactacacgcagaagagcctctccctgtctccggg
taaataa

The sequences encoding the Abx-229 heavy and light chains sequence below were inserted into pTOK58D vector:

(SEQ ID NO: 37)
atggagtttgggctgagctggctttttcttgtggctattttaaaaggtgt
ccagtgtgaggtgcagctgttggagtctgggggaggcttggtacagcctg
gggggtccctgagactctcctgtgcagcctctggattcacctttagccgc
tatgccatgaactgggtccgccaggctccagggaaggggctggagtgggt
ctcaggtattagtgggagtggtggtaggacatactacgcagactccgtga
agggccggttcaccatctccagagacaattccaagaacacactatatctg
caaatgaacagcctgagagccgaggacacggccgtatattactgtgcgaa
agatcgcgatttttggagtggtccatttgactactggggccagggaaccc
tggtcaccgtcagctcagcctccaccaagggcccatcggtcttccccctg
gcaccctcctccaagagcacctctgggggcacagcggccctgggctgcct
ggtcaaggactacttccccgaaccggtgacggtgtcgtggaactcaggcg
ccctgaccagcggcgtgcacaccttcccggctgtcctacagtcctcagga
ctctactccctcagcagcgtggtgaccgtgccctccagcagcttgggcac
ccagacctacatctgcaacgtgaatcacaagcccagcaacaccaaggtgg
acaagaaagttgagcccaaatcttgtgacaaaactcacacatgcccaccg
tgcccagcacctgaactcctggggggaccgtcagtcttcctcttcccccc
aaaacccaaggacaccctcatgatctcccggacccctgaggtcacatgcg
tggtggtggacgtgagccacgaagaccctgaggtcaagttcaactggtac
gtggacggcgtggaggtgcataatgccaagacaaagccgcgggaggagca
gtacaacagcacgtaccgtgtggtcagcgtcctcaccgtcctgcaccagg
actggctgaatggcaaggagtacaagtgcaaggtctccaacaaagccctc
ccagcccccatcgagaaaaccatctccaaagccaaagggcagccccgaga
accacaggtgtacaccctgcccccatcccgggatgagctgaccaagaacc
aggtcagcctgacctgcctggtcaaaggcttctatcccagcgacatcgcc
gtggagtgggagagcaatgggcagccggagaacaactacaagaccacgcc
tcccgtgctggactccgacggctccttcttcctctacagcaagctcaccg
tggacaagagcaggtggcagcaggggaacgtcttctcatgctccgtgatg
catgaggctctgcacaaccactacacgcagaagagcctctccctgtctcc
gggtaaataa
(SEQ ID NO: 38)
atgaggctccctgctcagcttctcttcctcctgctactctggctcccaga
taccactggagaaatagtgatgacgccgtcttcagccaccctgtctgtgt
ctccaggggagagagccaccctctcctgcagggccagtcagagtgttagt
agaaacttagcctggtaccagcagaaacctggccaggctcccaggctcct
catctatggtgcatccaccagggccactggtatcccagccaggttcagtg
gcagtgggtctgggacagaattcactctcaccatcagcagcctgcagtct
gaagattttgcagtttattactgtcaccagtatagtaactggatgtgcag
ttttggccaggggaccaagctggagatcaaacgtacggtggctgcaccat
ctgtcttcatcttcccgccatctgatgagcagttgaaatctggaactgcc
tctgttgtgtgcctgctgaataacttctatcccagagaggccaaagtaca
gtggaaggtggataacgccctccaatcgggtaactcccaggagagtgtca
cagagcaggacagcaaggacagcacctacagcctcagcagcaccctgacc
ctgagcaaagcagactacgagaaacacaaagtctacgcctgcgaagtcac
ccatcagggcctgagctcgcccgtcacaaagagcttcaacaggggagagt
gttag

### Example 5

### GCC Expression in Primary Human Metastatic Colorectal Tumors

A rabbit monoclonal anti-GCC antibody (referred to herein as MIL-44-148-2) was developed to quantitatively evaluate GCC expression in four different Primary Human Tumor Xenografts (“PHTX”) derived from mCRC patient samples in female SCID mice (referred to herein as PHTX-09c, PHTX-21 cm PHTX-17c and PHTX-11c). A tumor xenograft model derived from a GCC transfected cell line previously confirmed to have high levels of GCC expression (HEK293-GCC #2) was used as a control.

Anti-GCC rabbit monoclonal antibodies were generated against a recombinant protein that combined the extracellular region of human GCC fused to a mouse IgG2a Fc region in which the two mutated Fc receptor binding regions (FcRs) were mutated to prevent Fc receptor binding (mIgG2a FcRmutII) using the RabMAb® service by Epitomics (Burlingame, Calif.). True rabbit-rabbit hybridomas were generated at Epitomics by fusing isolated B-cells from an immunized rabbit with Epitomics' proprietary fusion partner cell line (see U.S. Pat. Nos. 7,402,409; 7,429,487; 7,462,697; 7,575,896; 7,732,168; and 8,062,867). Specificity of the antibodies against GCC was tested and confirmed by ELISA and flow cytometry (FCM). Specificity of the anti-GCC antibodies were tested and confirmed by ELISA and flow cytometry (FCM). Several clones were screened for use in immunohistochemistry, and the MIL-44-148-2 clone was selected as having optimal activity.

The sequences of the light and heavy chain variable regions were determined. Table 21 below is a summary of the SEQ ID NOs for the variable regions of the MIL-44-148-2 anti-GCC antibody. The amino acid and nucleic acid sequences for the variable regions of each of the heavy and light chains are shown in Tables 22 and 23, respectively.

The amino acid and nucleic acid sequences for each of the CDRs of the heavy and light chains for the MIL-44-148-2 antibody is shown in Tables 24 and 25, respectively.

TABLE 21
Summary of SEQ ID NOs for heavy and
light chains of anti-GCC rabbit mAb

| | IgG | Nucleic Acid | Amino Acid |
|---|---|---|---|
| mAb | Chain | SEQ ID NO | SEQ ID NO |
| MIL-44-148-2 H2 | Heavy | 39 | 40 |
| MIL-44-148-2 L5 | Light | 41 | 42 |

MIL-44-148-2 H2 Nucleic Acid
(SEQ ID NO: 39)
ATGGAGACTGGGCTGCGCTGGCTTCTCCTGGTCGCTGTGCTCAAAGGTGT
CCAGTGTCAGTCAGTGAAGGAGTCCGGGGGAGGCCTCTTCAAGCCAACGG
ATACCCTGACACTCACCTGCACCGTCTCTGGATTCTCCCTCAGTAGTCAT
AGAATGAACTGGGTCCGCCAGACTCCAGGGAAGGGGCTGGAATGGATCGC
AATCATTACTCATAATAGTATCACATACTACGCGAGCTGGGCGAAAAGCC
GATCCACCATCACCAGAAACACCAGCGAGAACACGGTGACTCTGAAAATG
ACCAGTCTGACAGCCGCGGACACGGCCACTTATTTCTGTGCCAGAGAGGA
TAGTATGGGGTATTATTTTGACTTGTGGGGCCCAGGCACCCTGGTCACCA
TCTCCTCAGGGCAACCTAAGGCTCCATCAGTCTTCCCACTGGCCCCCTGC
TGCGGGGACACACCCAGCTCCACGGTGACCCTGGGCTGCCTGGTCAAAGG
GTACCTCCCGGAGCCAGTGACCGTGACCTGGAACTCGGGCACCCTCACCA
ATGGGGTACGCACCTTCCCGTCCGTCCGGCAGTCCTCAGGCCTCTACTCG
CTGAGCAGCGTGGTGAGCGTGACCTCAAGCAGCCAGCCCGTCACCTGCAA
CGTGGCCCACCCAGCCACCAACACCAAAGTGGACAAGACCGTTGCGCCCT
CGACATGCAGCAAGCCCACGTGCCCACCCCCTGAACTCCTGGGGGGACCG
TCTGTCTTCATCTTCCCCCCAAAACCCAAGGACACCCTCATGATCTCACG
CACCCCCGAGGTCACATGCGTGGTGGTGGACGTGAGCCAGGATGACCCCG
AGGTGCAGTTCACATGGTACATAAACAACGAGCAGGTGCGCACCGCCCGG
CCGCCGCTACGGGAGCAGCAGTTCAACAGCACGATCCGCGTGGTCAGCAC
CCTCCCCATCGCGCACCAGGACTGGCTGAGGGGCAAGGAGTTCAAGTGCA
AAGTCCACAACAAGGCACTCCCGGCCCCCATCGAGAAAACCATCTCCAAA
GCCAGAGGGCAGCCCCTGGAGCCGAAGGTCTACACCATGGGCCCTCCCCG
GGAGGAGCTGAGCAGCAGGTCGGTCAGCCTGACCTGCATGATCAACGGCT
TCTACCCTTCCGACATCTCGGTGGAGTGGGAGAAGAACGGGAAGGCAGAG
GACAACTACAAGACCACGCCGGCCGTGCTGGACAGCGACGGCTCCTACTT
CCTCTACAGCAAGCTCTCAGTGCCCACGAGTGAGTGGCAGCGGGGCGACG
TCTTCACCTGCTCCGTGATGCACGAGGCCTTGCACAACCACTACACGCAG
AAGTCCATCTCCCGCTCTCCGGGTAAATGA
MIL-44-148-2 H2 Amino Acid
(SEQ ID NO: 40)
METGLRWLLLVAVLKGVQCQSVKESGGGLFKPTDTLTLTCTVSGFSLSSH
RMNWVRQTPGKGLEWIAIITHNSITYYASWAKSRSTITRNTSENTVTLKM
TSLTAADTATYFCAREDSMGYYFDLWGPGTLVTISSGQPKAPSVFPLAPC
CGDTPSSTVTLGCLVKGYLPEPVTVTWNSGTLTNGVRTFPSVRQSSGLYS
LSSVVSVTSSSQPVTCNVAHPATNTKVDKTVAPSTCSKPTCPPPELLGGP
SVFIFPPKPKDTLMISRTPEVTCVVVDVSQDDPEVQFTWYINNEQVRTAR
PPLREQQFNSTIRVVSTLPIAHQDWLRGKEFKCKVHNKALPAPIEKTISK
ARGQPLEPKVYTMGPPREELSSRSVSLTCMINGFYPSDISVEWEKNGKAE
DNYKTTPAVLDSDGSYFLYSKLSVPTSEWQRGDVFTCSVMHEALHNHYTQ
KSISRSPGK
MIL-44-148-2 L5 Nucleic Acid
(SEQ ID NO: 41)
ATGGACACGAGGGCCCCCACTCAGCTGCTGGGGCTCCTGCTGCTCTGGCT
CCCAGGTGCCAGATGTGCCTATGATATGACCCAGACTCCAGCCTCTGTGG
AGGTAGCTGTGGGAGGCACAGTCACCATCAAGTGCCAGGCCAGTCAGAGC
ATTAGTAACTGGTTAGCCTGGTATCAGCAGAAACCAGGGCAGTCTCCCAA
GCCCCTGATCTACAGGGCATCCACTCTGGCATCTGGGGTCTCATCGCGGT
TCAGAGGCAGTGGATCTGGGACACAGTTCACTCTCACCATCAGTGGCGTG
GAGTGTGCCGATGCTGCCACTTACTACTGTCAGCAGACTTATACTAATAA
TCATCTTGATAATGGTTTCGGCGGAGGGACCGAGGTGGTGGTCAAAGGTG
ATCCAGTTGCACCTACTGTCCTCATCTTCCCACCAGCTGCTGATCAGGTG
GCAACTGGAACAGTCACCATCGTGTGTGTGGCGAATAAATACTTTCCCGA
TGTCACCGTCACCTGGGAGGTGGATGGCACCACCCAAACAACTGGCATCG
AGAACAGTAAAACACCGCAGAATTCTGCAGATTGTACCTACAACCTCAGC
AGCACTCTGACACTGACCAGCACACAGTACAACAGCCACAAAGAGTACAC
CTGCAGGGTGACCCAGGGCACGACCTCAGTCGTCCAGAGCTTCAATAGGG
GTGACTGTTAG
MIL-44-148-2 L5 Amino Acid
(SEQ ID NO: 42)
MDTRAPTQLLGLLLLWLPGARCAYDMTQTPASVEVAVGGTVTIKCQASQS
ISNWLAWYQQKPGQSPKPLIYRASTLASGVSSRFRGSGSGTQFTLTISGV
ECADAATYYCQQTYTNNHLDNGFGGGTEVVVKGDPVAPTVLIFPPAADQV
ATGTVTIVCVANKYFPDVTVTWEVDGTTQTTGIENSKTPQNSADCTYNLS
STLTLTSTQYNSHKEYTCRVTQGTTSVVQSFNRGDC

TABLE 22
Amino Acid Sequences of mAb variable regions
of an anti-GCC rabbit mAb

| | | SEQ | |
|---|---|---|---|
| | IgG | ID | |
| mAb | Chain | NO: | Amino Acid Sequence |
| MIL- | Heavy | 43 | QSVKESGGGLFKPTDTLTLTCTVSG |
| 44- | | | FSLSSHRMNWVRQTPGKGLEWIAII |
| 148-2 | | | THNSITYYASWAKSRSTITRNTSEN |
| | | | TVTLKMTSLTAADTATYFCAREDSM |
| | | | GYYFDLWGPGTLVTISS |
| MIL- | Light | 44 | AYDMTQTPASVEVAVGGTVTIKCQA |
| 44- | | | SQSISNWLAWYQQKPGQSPKPLIYR |
| 148-2 | | | ASTLASGVSSRFRGSGSGTQFTLTI |
| | | | SGVECADAATYYCQQTYTNNHLDNG |
| | | | FGGGTEVVVK |

TABLE 23
Nucleic Acid Sequences of mAb variable regions
of anti-GCC rabbit mAb

| | | SEQ | |
|---|---|---|---|
| | IgG | ID | |
| mAb | Chain | NO: | Nucleic Acid Sequence |
| MIL-44- | Heavy | 45 | CAGTCAGTGAAGGAGTCCGGGGGAGG |
| 148-2 | | | CCTCTTCAAGCCAACGGATACCCTGA |
| | | | CACTCACCTGCACCGTCTCTGGATTC |
| | | | TCCCTCAGTAGTCATAGAATGAACTG |
| | | | GGTCCGCCAGACTCCAGGGAAGGGGC |
| | | | TGGAATGGATCGCAATCATTACTCAT |
| | | | AATAGTATCACATACTACGCGAGCTG |
| | | | GGCGAAAAGCCGATCCACCATCACCA |
| | | | GAAACACCAGCGAGAACACGGTGACT |
| | | | CTGAAAATGACCAGTCTGACAGCCGC |
| | | | GGACACGGCCACTTATTTCTGTGCCA |
| | | | GAGAGGATAGTATGGGGTATTATTTT |
| | | | GACTTGTGGGGCCCAGGCACCCTGGT |
| | | | CACCATCTCCTCA |
| MIL-44- | Light | 47 | GCCTATGATATGACCCAGACTCCAGC |
| 148-2 | | | CTCTGTGGAGGTAGCTGTGGGAGGCA |
| | | | CAGTCACCATCAAGTGCCAGGCCAGT |
| | | | CAGAGCATTAGTAACTGGTTAGCCTG |
| | | | GTATCAGCAGAAACCAGGGCAGTCTC |
| | | | CCAAGCCCCTGATCTACAGGGCATCC |
| | | | ACTCTGGCATCTGGGGTCTCATCGCG |
| | | | GTTCAGAGGCAGTGGATCTGGGACAC |
| | | | AGTTCACTCTCACCATCAGTGGCGTG |
| | | | GAGTGTGCCGATGCTGCCACTTACTA |
| | | | CTGTCAGCAGACTTATACTAATAATC |
| | | | ATCTTGATAATGGTTTCGGCGGAGGG |
| | | | ACCGAGGTGGTGGTCAAA |

TABLE 24
Amino Acid Sequences of CDRs of an
anti-GCC rabbit mAb

| | | SEQ ID | Amino Acid |
|---|---|---|---|
| mAb | IgG | NO: | Sequence |
| MIL-44-148-2-H2 | VH CDR1 | 49 | SHRMN |
| MIL-44-148-2-H2 | VH CDR2 | 50 | IITHNSITYYASWAKS |
| MIL-44-148-2-H2 | VH CDR3 | 51 | EDSMGYYFDL |
| MIL-44-148-2-L5 | VK CDR1 | 52 | QASQSISNWLA |
| MIL-44-148-2-L5 | VK CDR2 | 53 | RASTLAS |
| MIL-44-148-2-L5 | VK CDR3 | 54 | QQTYTNNHLDNG |

TABLE 25
Nucleic Acid Sequences of CDRs of an
anti-GCC rabbit mAb

| | | SEQ ID | Nucleic Acid |
|---|---|---|---|
| mAb | IgG | NO: | Sequence |
| MIL-44-148-2- | VH | 55 | AGTCATAGAATGAAC |
| H2 | CDR1 | | |
| MIL-44-148-2- | VH | 56 | ATCATTACTCATAATAGTATC |
| H2 | CDR2 | | ACATACTACGCGAGCTGGGCG |
| | | | AAAAGC |
| MIL-44-148-2- | VH | 57 | GAGGATAGTATGGGGTATTAT |
| H2 | CDR3 | | TTTGACTTG |
| MIL-44-148-2- | VK | 58 | CAGGCCAGTCAGAGCATTAGT |
| L5 | CDR1 | | AACTGGTTAGCC |
| MIL-44-148-2- | VK | 59 | AGGGCATCCACTCTGGCATCT |
| L5 | CDR2 | | |
| MIL-44-148-2- | VK | 60 | CAGCAGACTTATACTAATAAT |
| L5 | CDR3 | | CATCTTGATAATGGT |

Immunohistochemistry with MIL-44-148-2

GCC protein levels in Formalin-Fixed, Paraffin-Embedded (FFPE) tissues of the primary human tumor xenografts were assessed on 5 μm thick sections and incubated with the MIL-44-148-2 antibody (3.5 μg/mL) for 1 hour on the Ventana Medical Systems (Tucson, Ariz.) Discovery XT® automated stainer. Antibodies were biotinylated with a rabbit anti-goat secondary antibody (Vector Laboratories) and developed with the 3,3′-diaminobexidine (DAB) substrate map system (Ventana Medical Systems). Slides were counterstained with hematoxylin and imaged using the Aperio whole slide scanning system.

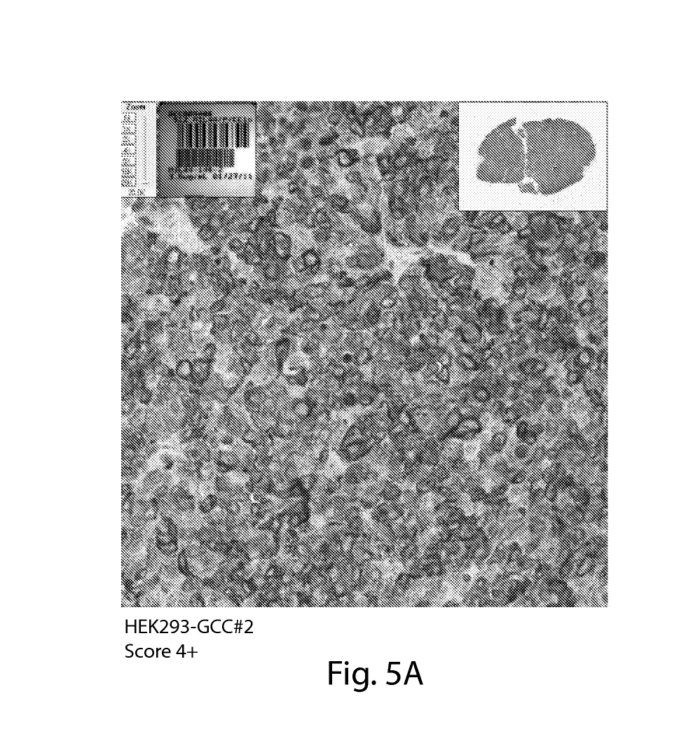

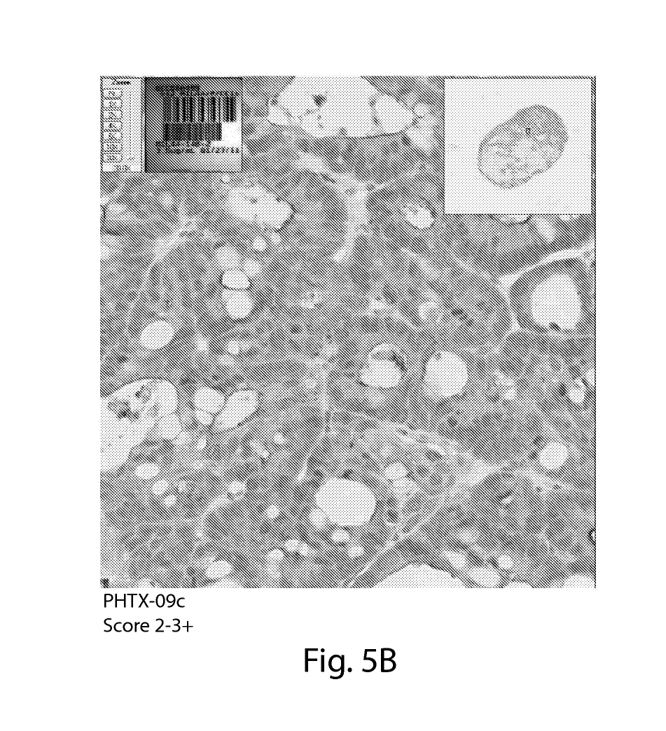

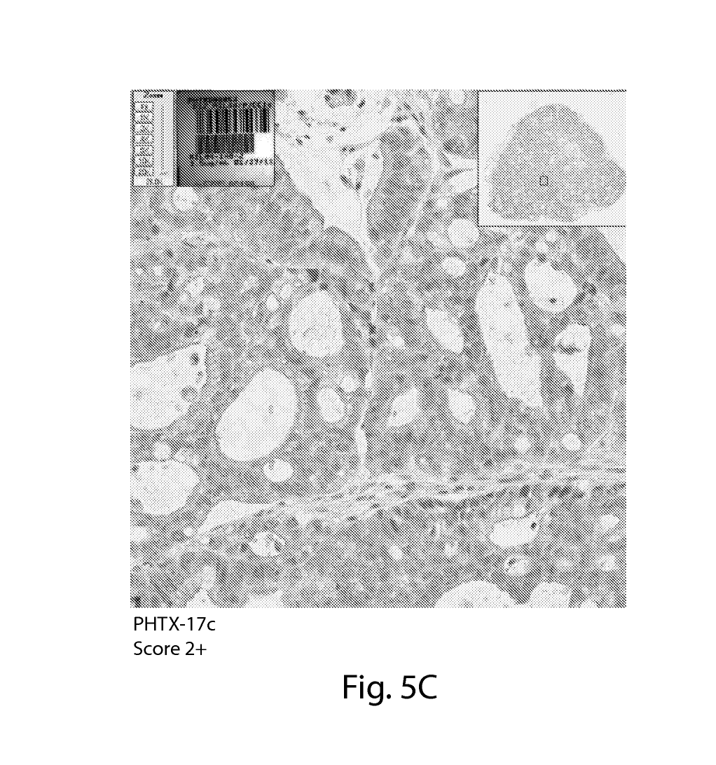

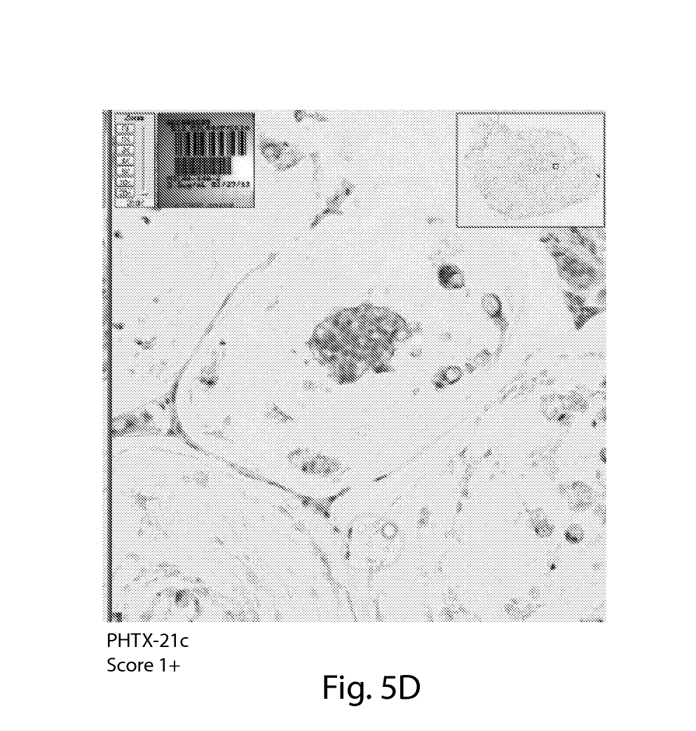

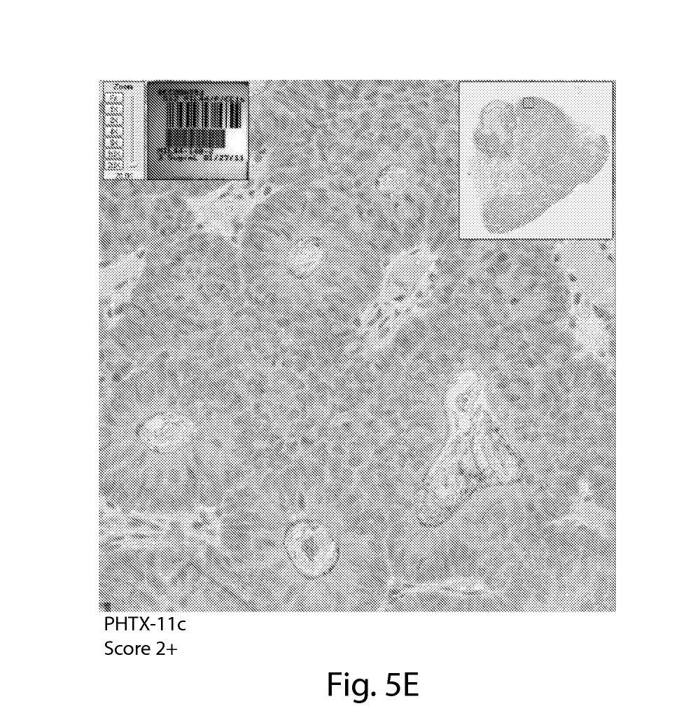

FIGS. 5A-5E depicts the results of the IHC assay in the various tumor xenograft models. As shown throughout FIGS. 5A-5E, GCC levels differed among the primary human tumor xenografts with scores ranging from 4+ in the cell derived HEK293-GCC tumor xenografts, and from 1+, 2+, and 2-3+, in the various PHTX tumor xenografts derived from mCRC patients.

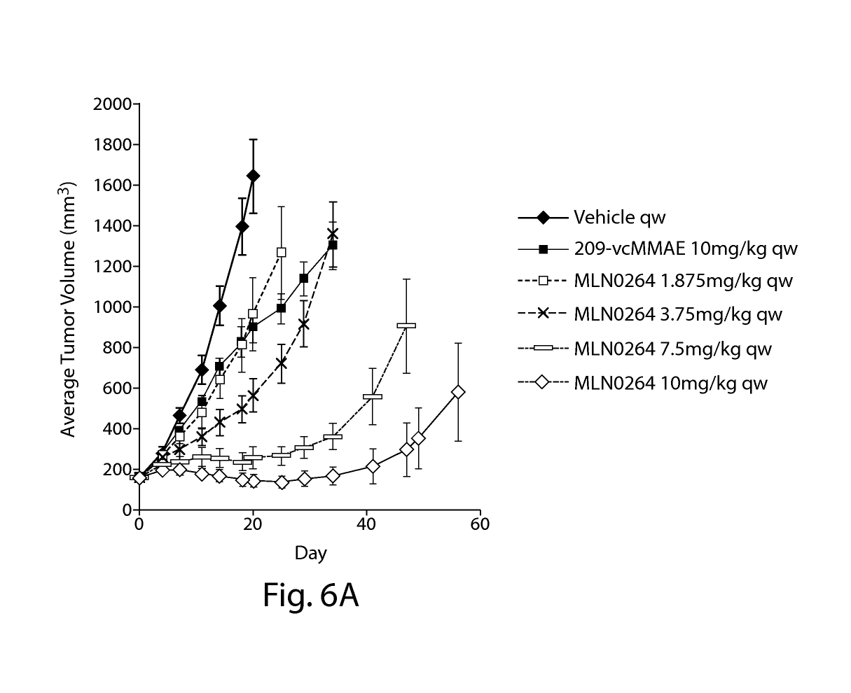

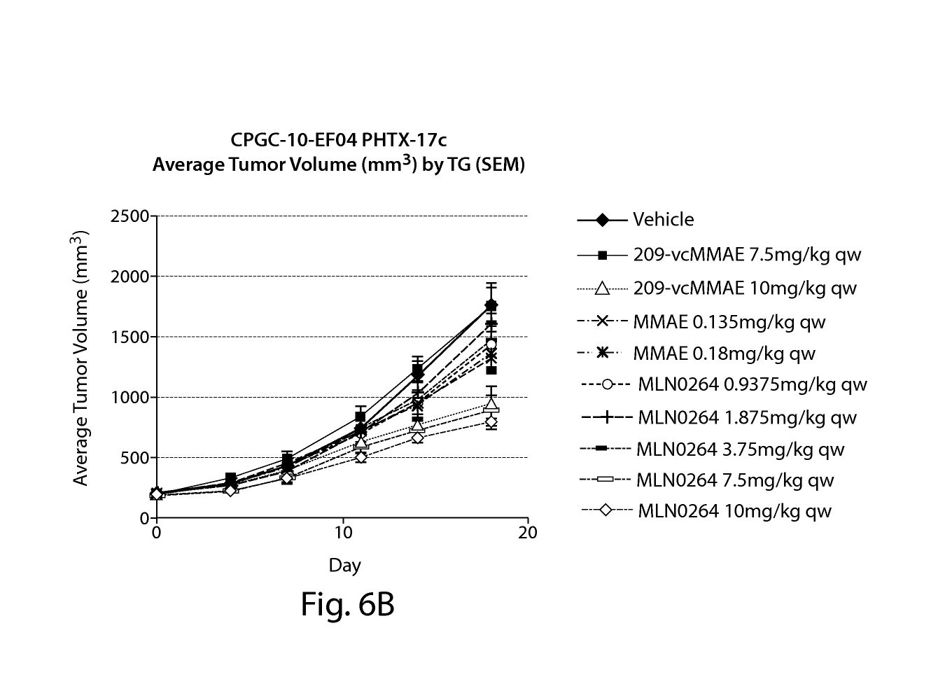

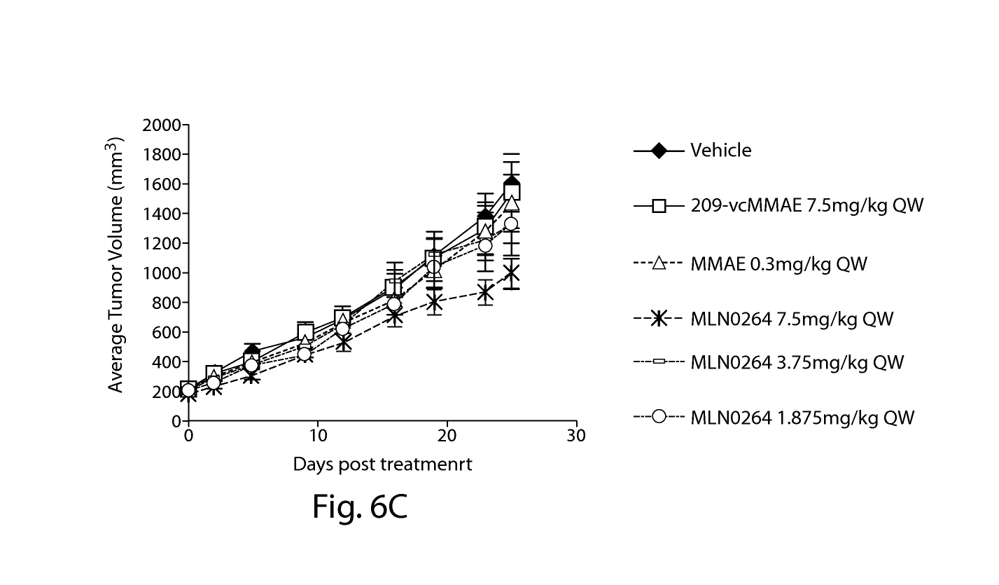

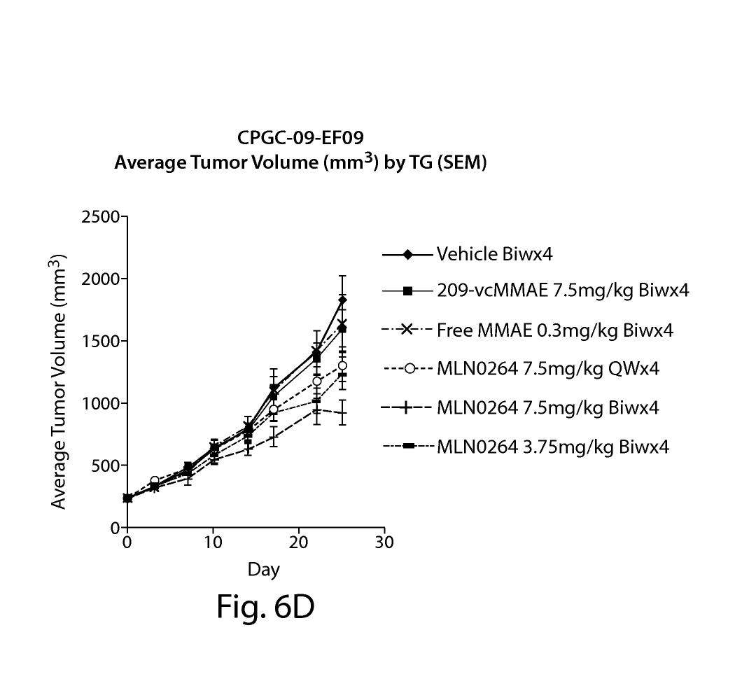

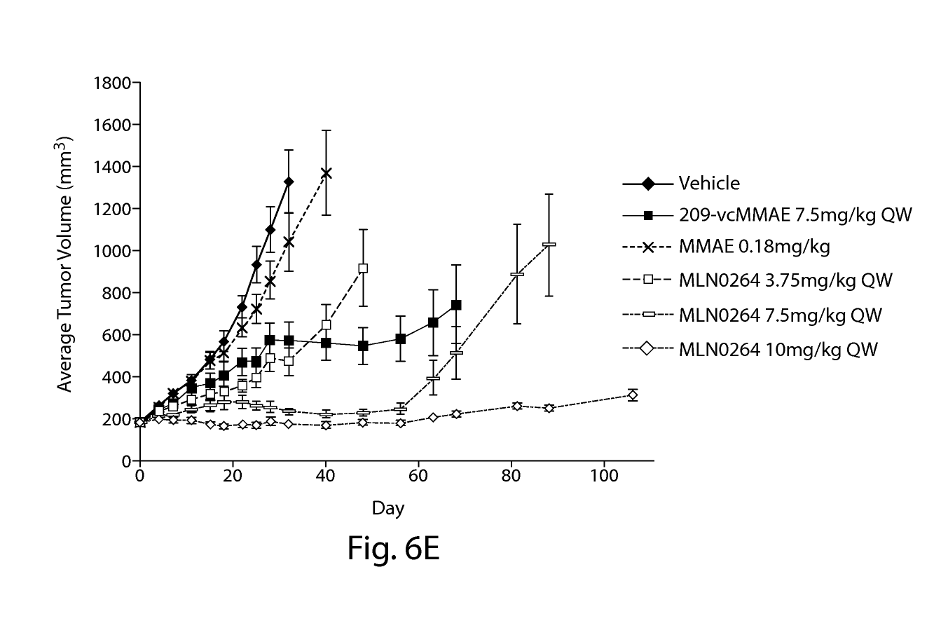

The 5F9 vcMMAE immunoconjugate was tested for single agent anti-tumor activity in the four primary human tumor xengraft models at varying concentrations in order to explore whether GCC expression levels played a role in tumor sensitivity. An MMAE-based antibody drug conjugate made with a human IgG1 monoclonal antibody raised against an unrelated target with no cross-reactivity to GCC (referred to as 209-vcMMAE in FIGS. 6A-6E) was used as a negative control. Free-MMAE was also used as a negative control. A summary of the 5F9 vcMMAE dose ranges, dosing schedules, and the corresponding efficacy in the various xenograft models is shown in Table 26 below (T/C=volume of tumor/control).

TABLE 26
5F9 vcMMAE in vivo efficacy studies in
primary human tumor xenograft models

| | # of | | | | |
|---|---|---|---|---|---|
| models | expt | dose | schedule | T/C | P |
| 293 GCC#2 | 2 | 1.875-7.5 mg/kg | QW | 0.03 | <0.001 |
| PHTX-09c | 3 | 0.938-10 mg/kg | QW | 0.09 | <0.001 |
| PHTX-21c | 2 | 3.75-10 mg/kg | QW | 0.23 | <0.001 |
| PHTX-17c | 2 | 0.938-10 mg/kg | QW | 0.46 | <0.05 |
| PHTX-11c | 2 | 1.875-7.5 mg/kg | QW | 0.5 on | <0.05 |
| | | | &BIW | BIW 0.62 | |
| | | | | on QW | |

The results of the single agent in vivo activity in the various xenograft models are depicted in FIGS. 6A-6E. As shown in FIG. 6A, strong 5F9 vcMMAE induced anti-tumor activity at doses ranging from 1.875-10 mg/kg (administered I.V. QW×3 weeks on days 1, 8 and 15) was seen in the PHTX-09c model which has relatively high GCC antigen density as demonstrated in the IHC assay described above (Score 2-3+). As further shown in FIG. 6A, anti-tumor activity of the immunoconjugate was maintained for an additional 3 weeks post-dosing (last dose on day 15) at the 7.5 mg/kg and 10 mg/kg levels. Tumors did not start to re-grow until approximately day 40.

Moderate sensitivity to the 5F9 vcMMAE immunoconjugate was seen in the PHTX-17c model at doses ranging from 0.9375 mg/kg-10 mg/kg (administered I.V. QW×3 weeks on days 1, 8 and 15) (FIG. 6B), which has moderate GCC antigen density (IHC score 2+).

Moderate to strong in vivo anti-tumor activity was also seen in the PHTX-21c model at doses ranging from 3.75-10 mg/kg (administered I.V. QW×4 on days 1, 8, 15 and 22) (FIG. 6E), which was somewhat surprising given its low level of antigen expression (IHC Score 1+). As shown in FIG. 6E, 3.75 mg/kg and 10 mg/kg doses effectively prevented tumor growth during the dosing schedule. Surprisingly, anti-tumor activity was maintained for an additional 7-8 weeks post-dosing (last dose on day 22) at the 7.5 mg/kg level, and for an unexpectedly even longer period extending beyond 15 weeks post-dosing at the 10 mg/kg level.

In contrast, the PHTX-11c model was relatively resistant to the immunoconjugate at doses ranging from 1.875-7.5 mg/kg (administered I.V. QW×3 weeks on days 1, 8 and 15) despite having a moderate level of GCC expression (IHC Score 2+). As shown in FIG. 6C, no anti-tumor activity was observed during the QW×3 dosing schedule or post dosing. Biweekly administration at the higher concentrations did not show any improvement in anti-tumor efficacy (FIG. 6D).

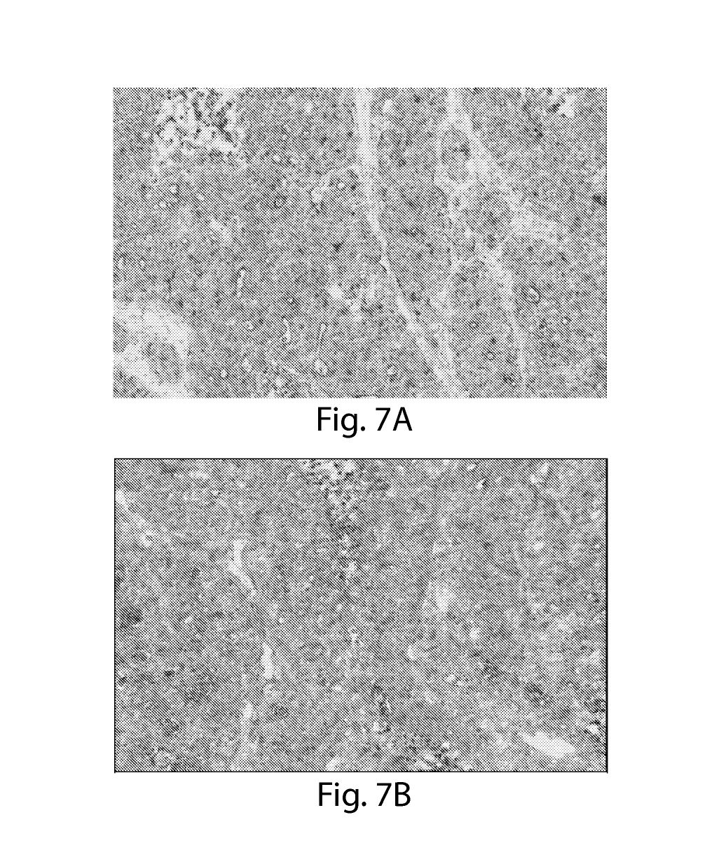

FIG. 7A shows GCC staining and FIG. 7B shows MLN0264 staining (both IHC) in the PHTX-11c model, and it can be seen that the staining patterns overlap. Thus, the 5F9 vcMMAE immunoconjugate was confirmed to bind to GCC in the refractory PHTX-11c model. Free-MMAE levels in serum vs. tumor were also assessed to ensure the 5F9 vcMMAE immunoconjugate was being processed by the tumors (i.e., that the immunoconjugate was being internalized and that MMAE was being cleaved). A comparison of free-MMAE serum and tumor levels in the MMAE-refractory PHTX-11c model and the sensitive PHTX-09c and HEK293 GCC #2 models is shown Table 27 below. The presence of drug was confirmed within the tumors during dosing and post-dosing. Additionally, the free-MMAE levels in this tumor model were the same as the levels seen in tumor xenograft models that were sensitive to the immunoconjugate. These results demonstrate that preclinical models refractory to 5F9 vcMMAE, such as the PHTX-11c model, bind and process anti-GCC ADCs efficiently.

TABLE 27

| MMAE | | 293 GCC #2 | PHTX-11C | PHTX-9C |
|---|---|---|---|---|
| AUC_{inf} | Serum | 56.3 | 44.1 | 68.3 |
| (hr*ng/mL) | | | | |
| AUC_{inf} | Tumor | 24953 | 13400 | 8670 |
| (hr*ng/mL) | | | | |
| | Tumor/Serum | 443 | 304 | 127 |

Oncogenic mutational status was also taken into consideration as a potential factor in tumor sensitivity to the 5F9 vcMMAE immunoconjugate. The genotype for the various xenograft models tested is shown in Table 28 below. Table 29 summarizes the antigen density, mutational status, dosing regimen and in vivo efficacy for the various tumor xenograft models. While sensitivity to the 5F9 vcMMAE immunoconjugate appears to be partly driven by antigen density as demonstrated in FIGS. 6A-6D, it appears to be independent of oncogenic mutational status. It is noted that the refractory PHTX-11c model exhibits a different KRAS mutation than the sensitive PHTX-09c and PHTX17c models. Without intending to be bound by any theory, there is no known biological distinction between codon 12 and codon 61 mutations in KRAS, and the present inventors have demonstrated that cells that have these mutations are sensitive to the 5F9 vcMMAE immunoconjugate in an in vitro cytotoxicity assay.

TABLE 28
mutational status of tumor xenograft models

| | PHTX-09c | PHTX-11c | PHTX-17c | PHTX-21c |
|---|---|---|---|---|
| p110 | WT | WT | WT | 545E > K/E |
| BRAF | WT | WT | WT | 600V > E/V |
| EGFR | WT | WT | WT | WT |
| AKT | WT | WT | WT | WT |
| KRAS | 12G > D/G | 61Q > H | 12G > G/C | WT |
| NRAS | WT | WT | WT | WT |
| HRAS | WT | WT | WT | WT |
| P53 | 120K > E | 273R > C | WT | WT |

In summary, 5F9 vcMMAE induced anti-tumor activity appears to be partly dependent on antigen density in tumors. Xenograft tumor models with high GCC antigen density (293HEK GCC#2 and PHTX-09c) were shown to be highly sensitive to the immunoconjugate, whereas xenograft models with lower antigen density levels were moderately sensitive in comparison, with the exception of the PHTX-11c model, which was resistant to the immunoconjugate despite confirmation of binding to GCC within the tumor and efficient tumor processing of the immunoconjugate The mechanism for the resistance in the PHTX-11c model is being further explored.

TABLE 29
Summary of GCC antigen density, mutational status,
dosing schedule and in vivo efficacy

| | GCC | | | Volume of | P |
|---|---|---|---|---|---|
| Xenograft | IHC | | Dosing | Tumor/ | Value |
| Models | Score | Genotype | Schedule | Control | (T/C) |
| 293 GCC#2 | 4+ | NA | QW | 0.03 | <0.001 |
| PHTX-09c* | 2-3+ | kRas G12D, | QW | 0.09 | <0.001 |
| | | p53mu | | | |
| PHTX-21c* | 1+ | bRaf V600E, | QW | 0.23 | <0.001 |
| | | p110a E545K | | | |
| PHTX-17c* | 2+ | kRas G12C | QW | 0.46 | <0.05 |
| PHTX-11c* | 2+ | kRas Q51H | QW | 0.5 on | <0.05 |
| | | | &BIW | BIW to | |
| | | | | 0.52 on | |
| | | | | QW | |

### Example 6

### In Vivo Evaluation of Combined Administration of an Anti-GCC Immunoconjugate and CPT-11 (Irinotecan)

The purpose of this study was to evaluate in vivo anti-tumor activity induced by combined administration of the 5F9 vcMMAE immunoconjugate (also referred to herein as “MLN0264”) and CPT-11 (a topoisomerase I inhibitor, also known as irinotecan or Camptosar®), in the four primary human tumor xenograft models described in Example 5 (PHTX-09c, PHTX-21c, PHTX-17c and PHTX-11c). The studies were conducted as follows.

Anti-Tumor Activity of 5F9 vcMMAE and CPT-11 Administered Intravenously to Female CB-17 SCID Mice Bearing PHTX-09C s.c Xenografts (Study No. CPGC-11-EF07)

As demonstrated in Example 5, the PHTX-09c model was highly sensitive to single agent activity of the 5F9 vcMMAE immunoconjugate at various concentrations. The objective of this study was to evaluate the 5F9 vcMMAE immunoconjugate in combination with CPT-11. More specifically, the goal of this study was to evaluate the anti-tumor activity of 5F9 vcMMAE at 1.875 and 3.75 mg/kg on a once weekly dosing schedule in combination with CPT-11 at 10 mg/kg on a two day on/5 day off dosing schedule administered intravenously in PHTX-09c s.c. xenograft in CB17 SCID mice. Dose formulations were prepared in accordance with Table 30. The study design is shown in Table 31.

TABLE 30
Preparation of the dose formulations

| | | | Dosing Solution | | |
|---|---|---|---|---|---|
| | | amt (ml | concentration | Vehi- | |
| Agent | Dose | liquid) | (mg/mL) | cle | Drug:Ab |
| 5F9 vcMMAE | 1.875 | | 0.375 | 0.9% | 4 |
| (MLN0264) | mg/kg | | | saline | |
| 5F9 vcMMAE | 3.75 | 0.316 | 0.75 | 0.9% | 4 |
| (MLN0264) | mg/kg | | | saline | |
| CPT-11 | 10.0 | 0.35 | 2 | 0.9% | N/A |
| | mg/kg | | | saline | |

TABLE 31
CPGC-11-EF07 Study Design

| | mouse#/ | | | | | |
|---|---|---|---|---|---|---|
| group | group | ADC first | schedule | CPT-11 | schedule | duration |
| 1 | 10 | vehicle i.v | D1 QW | | D1, 2 ×3 weeks | W1, W2, W3, |
| 2 | 10 | MLN0264 1.875 mg/kg i.v | D1 QW | | | W1, W2, W3, |
| 3 | 10 | MLN0264 3.75 mg/kg i.v | D1 QW | | | W1, W2, W3 |
| 4 | 10 | | | 10 mg/kg | D1, 2 ×3 weeks | W1, W2, W3 |
| 5 | 10 | MLN0264 1.875 mg/kg i.v | D1 QW | 10 mg/kg | D1, 2 ×3 weeks | W1, W2, W3 |
| 6 | 10 | MLN0264 3.75 mg/kg i.v | D1 QW | 10 mg/kg | D1, 2 ×3 weeks | W1, W2, W3 |

The endpoints for each treatment group was either a) tumor volume reaches 10% body weight; or b) body weight loss of >20%. The projected tumor volume at the study initiation date was 200 mm³.

Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 25-30 gauge, ½-¾ inch in length. Animals were dosed based on an average body weight of 20 grams.

Tumor volume measurements were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD). TGI was assessed on an arbitrary day (when the maximum tumor volume (MTV of the control treatment group reaches the maximum allowable tumor volume), using the following formula:

TGI=100−[MTV treated/MTV control]×100.

TGD was assessed by calculating T−C, where T=mean time (in days) for treatment group tumors to reach a predetermined size, and where C=mean time (in days) for the control group tumors to reach a predetermined size (e.g., 1,000 mm³).

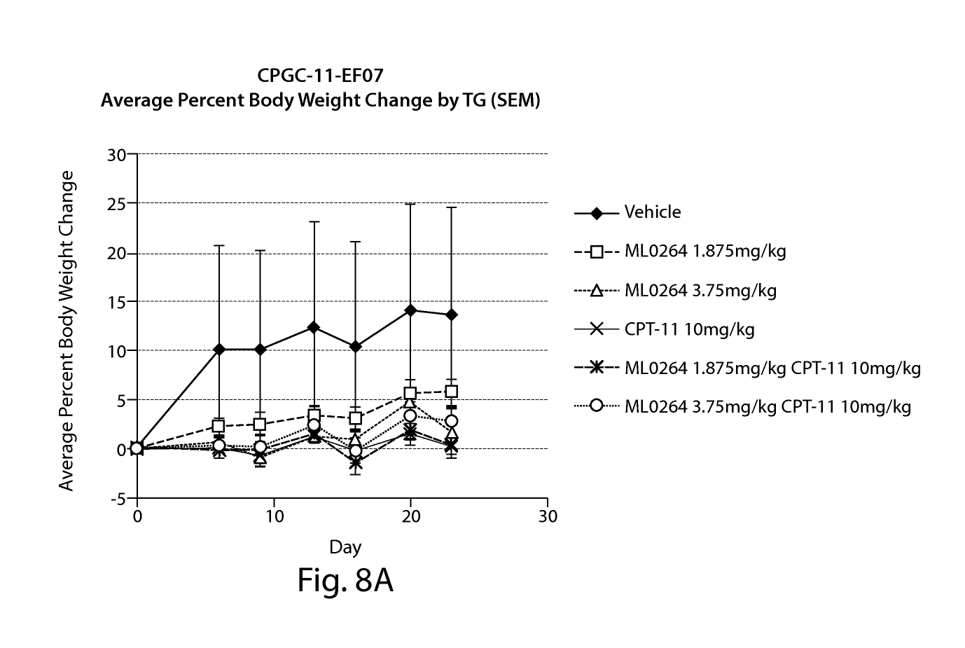

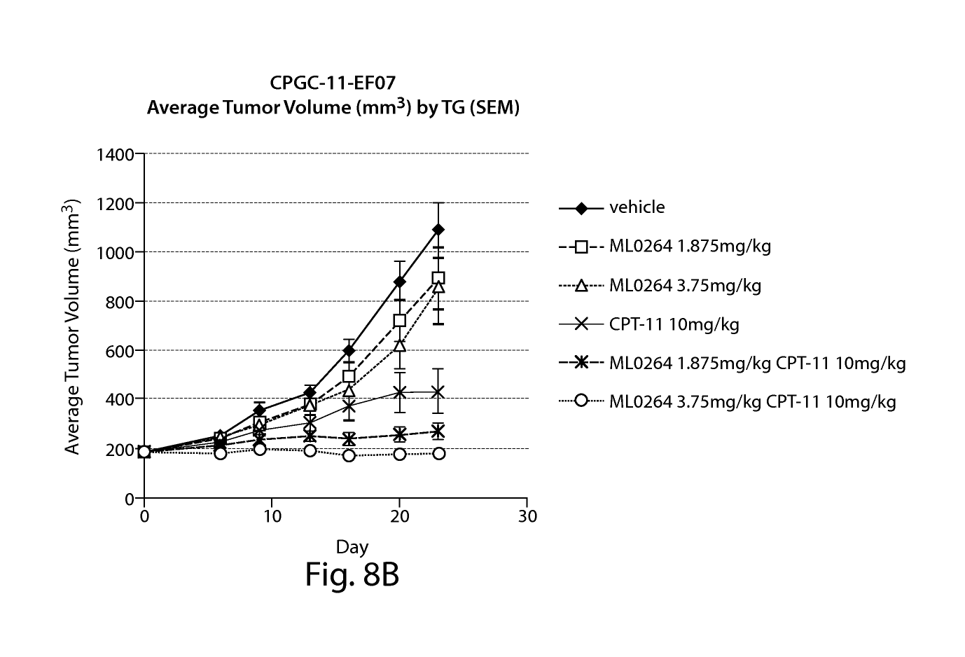

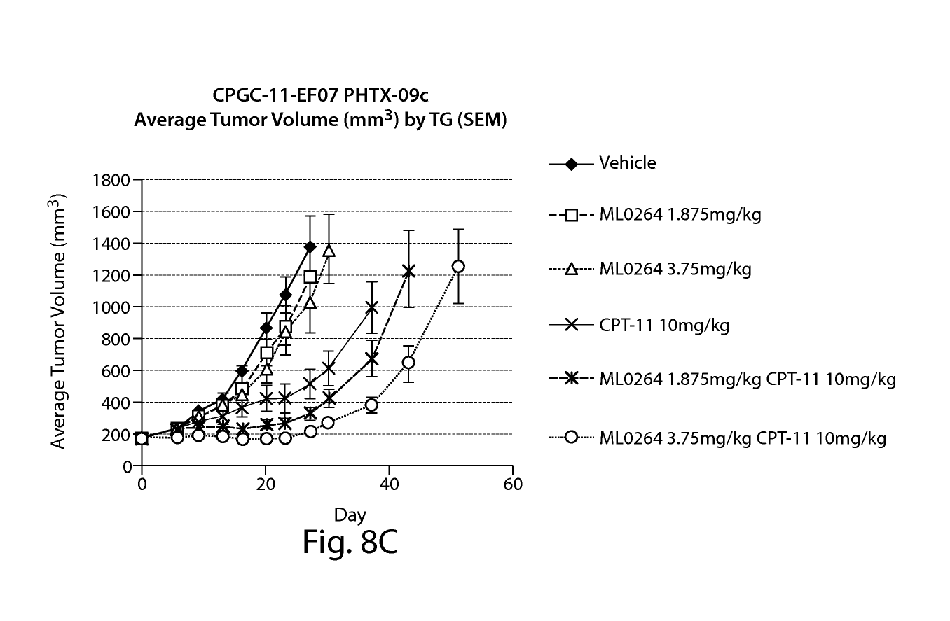

As shown in FIG. 8A, the doses were well tolerated in all treatment groups. The average tumor volume curves for the various treatment groups is shown in FIG. 8B. Tumor re-growth kinetics after dosing was stopped is shown in FIG. 8C. The results of the study are summarized in Table 32.

TABLE 32
Results of CPGC-11-EF07 Study

| | Mean tumor | | | | |
|---|---|---|---|---|---|
| | volume ± S.E.M | | Days to | | |
| Group | (mm3) | TGI | 1000 mm\hat{ }3 | TGD | T/C |
| Vehicle | 1085.1 ± 114.1 | | 21.8 | 0 | |
| ML0264 1.875 | 888.5 ± 126.2 | 18.1 | >23 | | 0.82 |
| mg/kg | | | | | |
| ML0264 3.75 | 861.1 ± 155.9 | 20.6 | >23 | | 0.79 |
| mg/kg | | | | | |
| CPT-11 10 | 430.6 ± 88.1 | 60.3 | >23 | | 0.4 |
| mg/kg | | | | | |
| ML0264 1.875 | 269.4 ± 32.2 | 75.2 | >23 | | 0.25 |
| mg/kg CPT-11 | | | | | |
| 10 mg/kg | | | | | |
| ML0264 3.75 | 178.1 ± 11.9 | 83.6 | >23 | | 0.16 |
| mg/kg CPT-11 | | | | | |
| 10 mg/kg | | | | | |

A longitudinal analysis of the CPGC-11-EF07 study using mixed effects linear regression models was performed to determine, in part, whether the combined anti-tumor efficacy of the 5F9 vcMMAE immunonjugate and CPT-11 was additive or synergistic. All tumor values (tumor volumes or photon flux) had a value of 1 added to them before log₁₀ transformation. These values were compared across treatment groups to assess whether the differences in the trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖ+treatᵢ+dayⱼ+dayⱼ²+(treat*day)ᵢⱼ+(treat*day²)ᵢⱼ+eᵢⱼₖ (1)

where Yᵢⱼₖ is the log₁₀ tumor value at the j^{th} time point of the k^{th} animal in the i^{th} treatment, Yᵢ₀ₖ is the day 0 (baseline) log₁₀ tumor value in the k^{th} animal in the i^{th} treatment, dayⱼ was the median-centered time point and (along with day²ⱼ) was treated as a continuous variable, and eᵢⱼₖ is the residual error. A spatial power law covariance matrix was used to account for the repeated measurements on the same animal over time. Interaction terms as well as day²ⱼ terms were removed if they were not statistically significant.

A likelihood ratio test was used to assess whether a given pair of treatment groups exhibited differences which were statistically significant. The −2 log likelihood of the full model was compared to one without any treatment terms (reduced model) and the difference in the values was tested using a Chi-squared test. The degrees of freedom of the test were calculated as the difference between the degrees of freedom of the full model and that of the reduced model. The predicted differences in the log tumor values (Yᵢⱼₖ−Yᵢ₀ₖ, which can be interpreted as log₁₀ (fold change from day 0)) were taken from the above models to calculate mean AUC values for each treatment group. A dAUC value was then calculated as:

$$dAUC = \frac{mean ( {AUC}_{ctl} ) - mean ( {AUC}_{trt} )}{mean ( {AUC}_{ctl} )} * 100 \qquad ( 2 )$$

This assumed AUC_{ctl} was positive. In instances where AUC_{ctl} was negative, the above formula was multiplied by −1.

For synergy analyses, the observed differences in the log tumor values were used to calculate AUC values for each animal. In instances when an animal in a treatment group was removed from the study, the last observed tumor value was carried forward through all subsequent time points. The AUC for the control, or vehicle, group was calculated using the predicted values from the pairwise models described above. To address the question of whether the effects of the combination treatments were synergistic, additive, or sub-additive relative to the individual treatments, the following statistics were calculated:

$$\begin{matrix} {Frac}_{{A}_{k}} = \frac{{AUC}_{ctl} - {AUC}_{{A}_{k}}}{{AUC}_{ctl}} & ( 3 ) \\ {Frac}_{{B}_{k}} = \frac{{AUC}_{ctl} - {AUC}_{{B}_{k}}}{{AUC}_{ctl}} & ( 4 ) \\ {Frac}_{{AB}_{k}} = \frac{{AUC}_{ctl} - {AUC}_{{AB}_{k}}}{{AUC}_{ctl}} & ( 5 ) \\ synergy score = ( mean ( {Frac}_{A} ) + mean ( {Frac}_{B} ) - mean ( {Frac}_{AB} ) ) * 100 & ( 6 )\end{matrix}$$

where Aₖ and Bₖ are the k^{th} animal in the individual treatment groups and ABₖ is the k^{th} animal in combination treatment group. AUC_{ctl} is the model-predicted AUC for the control group and was treated as a constant with no variability. The effect of the combination treatment was considered synergistic if the synergy score was less than 0, additive if the synergy score equaled 0, and sub-additive if the synergy score was greater than 0. The standard error of the synergy score was calculated as the square root of the sum of squared standard errors across groups A, B, and AB. The degrees of freedom were estimated using the Welch-Satterthwaite equation. P values were calculated by dividing the synergy score by its standard error and tested against a t-distribution (two-tailed) with the above-calculated degrees of freedom.

Given the exploratory nature of this study, there were no adjustments pre-specified for the multiple comparisons and endpoints examined. All P values <0.05 were called statistically significant in this analysis.

Table 33 is the annotation table for group symbols. Table 34 lists the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Table 35 lists the synergy analysis. Statistically significant negative synergy scores indicate a synergistic combination (“Syn.”). Statistically significant positive synergy scores indicate a sub-additive or antagonistic combination (“Antag.”). Scores that are not statistically significant should be considered additive (“Add.”).

TABLE 33
Annotation Table

| | Group | Notation |
|---|---|---|
| 1 | vehicle/0.9percentsaline | V |
| 2 | ML02641.875mg/kg/264 | A |
| 3 | ML02643.75mg/kg/264 | B |
| 4 | CPT-1110mg/kg/CPT-11 | C |
| 5 | ML02641.875mg/kgCPT-1110mg/kg/CPT-11+264 | D |
| 6 | ML02643.75mg/kgCPT-1110mg/kg/CPT-11+264 | E |

TABLE 34
Results of Pairwise Comparisons

| | Reference | Treated | dAUC | P. Value |
|---|---|---|---|---|
| 1 | V | A | 16.1 | 0.077 |
| 2 | V | B | 25.4 | <0.001 |
| 3 | V | C | 49.5 | <0.001 |
| 4 | V | D | 77.6 | <0.001 |
| 5 | V | E | 104.7 | <0.001 |

TABLE 35
Results of Combination Analysis

| | Score | SEM | P. Value | Assess |
|---|---|---|---|---|
| D | −10.5 | 14.5 | 0.474 | Add. |
| E | −32.5 | 12.4 | 0.017 | Syn. |

As shown in Table 35, synergistic activity was achieved using a dosing regimen of 3.75 mg/kg dose of the immunoconjugate administered I.V. QW×3 weeks, and 10 mg/kg dose of CPT-11 administered I.V. on day 1 and day 2 (2 day on, 5 day off schedule). The lower 1.875 mg/kg dose of the immunoconjugate administered I.V. QW in combination with 10 mg/kg of CPT-11 administered I.V. on a 2 day on, 5 day off schedule for 3 weeks resulted in an additive effect.

As shown in FIGS. 8B and 8C, both the 5F9 vcMMAE immunoconjugate and CPT-11 had strong and moderate single agent activity, respectively, in the PHTX-09c model, a model demonstrated to have relatively high GCC antigen density (IHC score 2-3+). Suprisingly the individual efficacy of each agent could be further improved by administering the two agents in combination at dosing regimens utilizing sub-optimal concentration of the immunoconjugate. The combination of the immunoconjugate and CPT-11 worked to prevent tumors from growing during the combined dosing regimens and further prevented tumor re-growth after dosing was stopped (last dose day 15) for an additional 3-4 weeks.

Anti-Tumor Activity of MLN0264, CPT-11 Administered Intravenously to Female CB-17 SCID Mice Bearing PHTX-21C s.c Xenografts (Study No. CPGC-11-04)

As demonstrated in Example 5, the PHTX-21c model was moderately sensitive to single agent activity of the 5F9 vcMMAE immunoconjugate at various concentrations despite having a low GCC antigen density level (IHC score 1+). The objective of this study was to evaluate the anti-tumor activity of 5F9 vcMMAE and CPT-11 in the PHTX-21c model. As demonstrated in the CPGC-11-07 study described above, 3.75 mg/kg dose of the immunoconjugate administered I.V. QW, in combination with a 10 mg/kg dose of CPT-11 administered I.V. on a 2 day on, 5 day off schedule yielded synergistic activity. Applying what was learned from the CPGC-11-07 study, and exploring additional dosing regimens of CPT-11, the administration of 5F9 vcMMAE at 3.75 mg/kg I.V. on a QW dosing schedule was evaluated in the PHTX-21c model in combination with CPT-11 at 10 mg/kg or 15 mg/kg on a 2 day on/5 day off dosing schedule and a 30 mg/kg once weekly schedule. Dose formulations were prepared in accordance with Table 36. The study design is shown in Table 37.

TABLE 36
Preparation of the dose formulations

| | | | Dosing Solution | | |
|---|---|---|---|---|---|
| | | amt (ml | concentration | Vehi- | |
| Agent | Dose | liquid) | (mg/mL) | cle | Drug:Ab |
| 5F9 vcMMAE | 3.75 | 0.465 | 0.6975 | 0.9% | 3.9 |
| (MLN0264) | mg/kg | | | saline | |
| CPT-11 | 10.0 | | 1.86 | 0.9% | |
| | mg/kg | | | saline | |
| CPT-11 | 15.0 | | 2.79 | 0.9% | |
| | mg/kg | | | saline | |
| CPT-11 | 30.0 | 1.0602 | 5.58 | 0.9% | |
| | mg/kg | | | saline | |

The 15 mg/kg dosing solution was prepared by a 1:1 dilution of the 30 mg/kg dosing solution. The 10 mg/kg dosing solution was prepared by a 2:1 dilution of the 15 mg/kg dosing solution.

TABLE 37
CPGC-11-EF04 Study Design

| | mouse#/ | | | CPT-11 | | CPT-11 | |
|---|---|---|---|---|---|---|---|
| group | group | ADC | schedule | i.v | schedule | total weekly | duration |
| 1 | 9 | vehicle i.v | D1 qw | | D1, D2 | 0 | W1, W2,, |
| 2 | 9 | MLN0264 | D1 qw | | | 0 | W1, W2,, |
| | | 3.75 mg/kg i.v | | | | | |
| 3 | 9 | | | 10 mg/kg | D1, D2 | 20 | Wl, W2,, |
| | | | | | QD | | |
| 4 | 9 | | | 15 mg/kg | D1, D2 | 30 | W1, W2,, |
| | | | | | QD | | |
| 5 | 9 | | | 30 mg/kg | D1 QW | 30 | W1, W2,, |
| 6 | 9 | MLN0264 | D1 qw | 10 mg/kg | D1, D2 | 20 | W1, W2,, |
| | | 3.75 mg/kg i.v | | | QD | | |
| 7 | 9 | MLN0264 | D1 qw | 15 mg/kg | D1, D2 | 30 | W1, W2,, |
| | | 3.75 mg/kg i.v | | | QD | | |
| 8 | 9 | MLN0264 | D1 qw | 30 mg/kg | D1 QW | 30 | W1,W2,, |
| | | 3.75 mg/kg i.v | | | | | |

The endpoints for each treatment group was either a) tumor volume reaches 10% body weight; or b) body weight loss of >20%. The projected tumor volume at the study initiation date was 200 mm³.

Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 27-30 gauge, ½-¾ inch in length. Animals were dosed based on an average body weight of 18.6 grams.

Tumor volume measurements were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD). TGI was assessed on an arbitrary day (when the maximum tumor volume (MTV of the control treatment group reaches the maximum allowable tumor volume), using the following formula:

TGI=100−[MTV treated/MTV control]×100.

TGD was assessed by calculating T−C, where T=mean time (in days) for treatment group tumors to reach a predetermined size (e.g., 1,000 mm³), and where C=mean time (in days) for the control group tumors to reach a predetermined size.

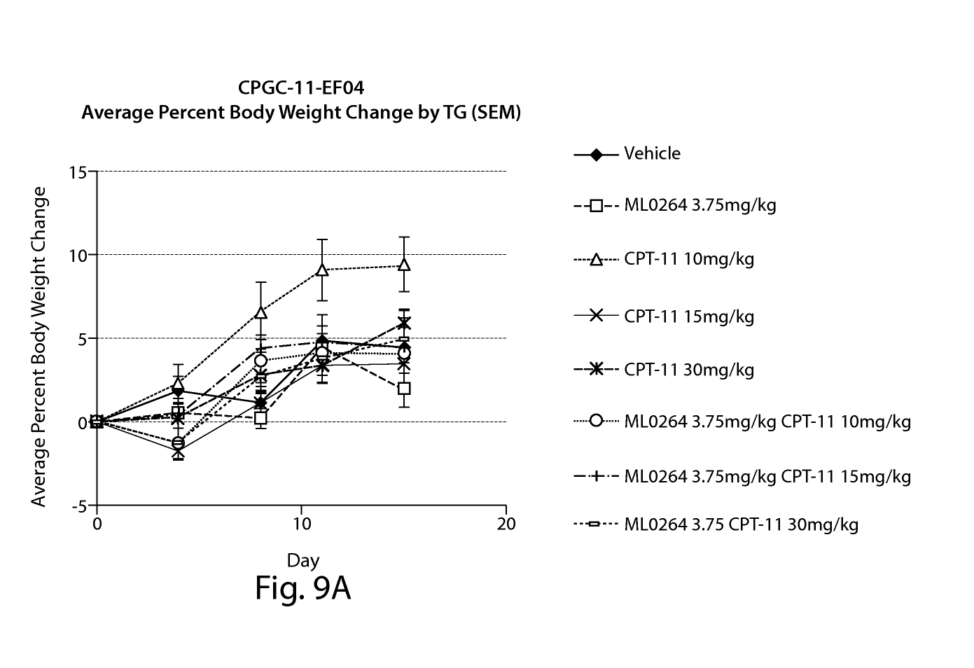

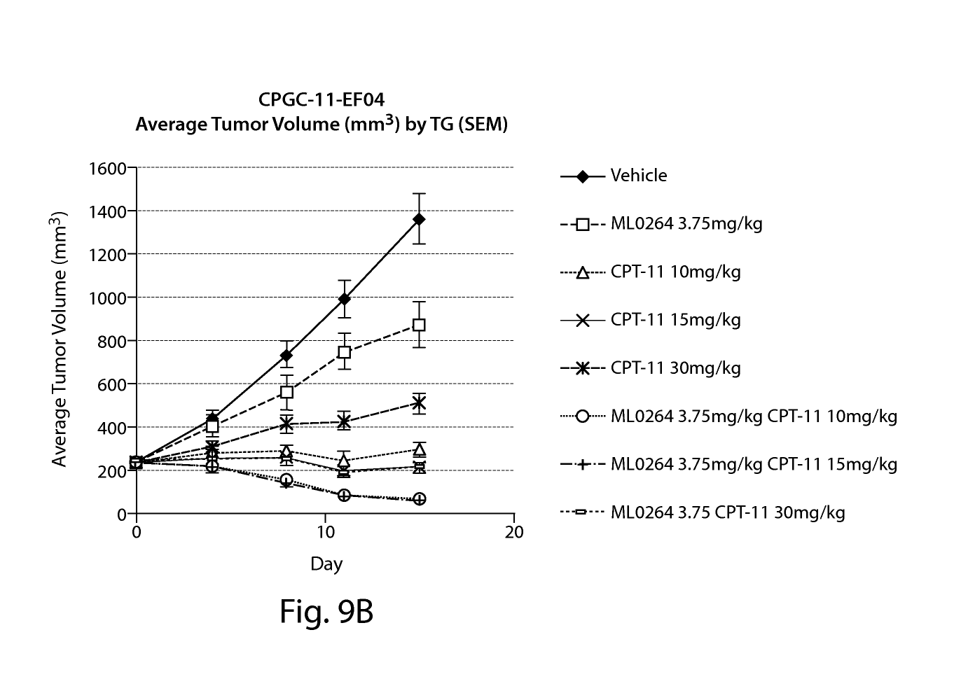

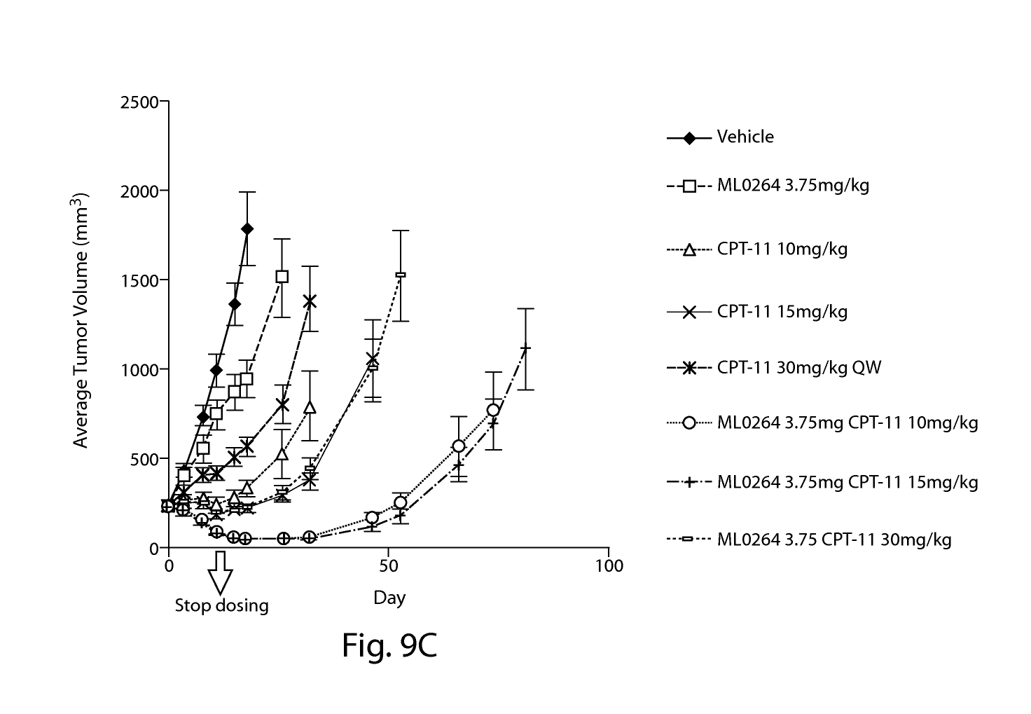

As shown in FIG. 9A, the doses were well tolerated in all treatment groups. Average tumor volume curves for the various treatment groups are shown in FIG. 9B. As shown in FIG. 8B, the immunoconjugate had relatively low anti-tumor activity as a single agent at the 3.75 mg/kg dose, whereas the CPT-11 had moderate to high single agent activity at the two lower doses administered. The tumor re-growth kinetics curve is shown in FIG. 9C. As shown in FIGS. 9B and 9C, there was no difference between the combination treatment groups with 10 mg/kg and 15 mg/kg CPT-11 groups in terms of tumor volume during dosing and the number of days by which the tumor re-growth post-dosing reached a pre-determined volume of 1000 mm³. Both dosing regimens not only effectively prevented tumor growth during dosing, but surprisingly induced reduction in tumor volume post-dosing (see FIG. 9B). Both dosing regiments were also effective in preventing tumor regrowth for an additional 5 weeks post-dosing. Tumors did not start to re-grow until around day 50. The results of the study are summarized in Table 38.

TABLE 38
Results of CPGC-11-EF04 Study

| | Mean tumor | | | | |
|---|---|---|---|---|---|
| | volume ± S.E.M | | Days to | | |
| Group | (mm3) | TGI | 1000 mm\hat{ }3 | TGD | T/C |
| Vehicle | 1365.1 ± 115.8 | | 11.1 | 0 | |
| ML0264 3.75 | 869.1 ± 105.2 | 36.3 | >15 | | 0.64 |
| mg/kg | | | | | |
| CPT-11 10 | 294.2 ± 29.6 | 78.4 | >15 | | 0.22 |
| mg/kg | | | | | |
| CPT-11 15 | 212.9 ± 18.9 | 84.4 | >15 | | 0.16 |
| mg/kg | | | | | |
| CPT-11 30 | 508.3 ± 49.4 | 62.8 | >15 | | 0.37 |
| mg/kg | | | | | |
| ML0264 3.75 | 61.9 ± 7.2 | 95.5 | >15 | | 0.05 |
| mg/kg CPT11 | | | | | |
| 10 mg/kg | | | | | |
| ML0264 3.75 | 58.6 ± 7 | 95.7 | >15 | | 0.04 |
| mg/kg CPT-11 | | | | | |
| 15 mg/kg | | | | | |
| ML0264 3.75 | 207.9 ± 20 | 84.8 | >15 | | 0.15 |
| CPT-11 30 mg/kg | | | | | |

A longitudinal analysis of the CPGC-11-EF04 study using mixed effects linear regression models was performed to determine, in part, whether the combined anti-tumor efficacy of the 5F9 vcMMAE immunonjugate and CPT-11 was additive or synergistic. All tumor values (tumor volumes or photon flux) had a value of 1 added to them before log₁₀ transformation. These values were compared across treatment groups to assess whether the differences in the trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖ+treatᵢ+dayⱼ+dayⱼ²+(treat*day)ᵢⱼ+(treat*day²)ᵢⱼ+eᵢⱼₖ (1)

where Yᵢⱼₖ is the log₁₀ tumor value at the j^{th} time point of the k^{th} animal in the i^{th} treatment, Yᵢₒₖ is the day 0 (baseline) log₁₀ tumor value in the k^{th} animal in the i^{th} treatment, dayⱼ was the median-centered time point and (along with day²ⱼ) was treated as a continuous variable, and eᵢⱼₖ is the residual error. A spatial power law covariance matrix was used to account for the repeated measurements on the same animal over time. Interaction terms as well as day²ⱼ terms were removed if they were not statistically significant.

A likelihood ratio test was used to assess whether a given pair of treatment groups exhibited differences which were statistically significant. The −2 log likelihood of the full model was compared to one without any treatment terms (reduced model) and the difference in the values was tested using a Chi-squared test. The degrees of freedom of the test were calculated as the difference between the degrees of freedom of the full model and that of the reduced model.

The predicted differences in the log tumor values (Yᵢⱼₖ−Yᵢ₀ₖ, which can be interpreted as log₁₀ (fold change from day 0)) were taken from the above models to calculate mean AUC values for each treatment group. A dAUC value was then calculated as:

$$dAUC = \frac{mean ( {AUC}_{ctl} ) - mean ( {AUC}_{trt} )}{mean ( {AUC}_{ctl} )} * 100 \qquad ( 2 )$$

This assumed AUC_{ctl} was positive. In instances where AUC_{ctl} was negative, the above formula was multiplied by −1.

For synergy analyses, the observed differences in the log tumor values were used to calculate AUC values for each animal. In instances when an animal in a treatment group was removed from the study, the last observed tumor value was carried forward through all subsequent time points. The AUC for the control, or vehicle, group was calculated using the predicted values from the pairwise models described above. To address the question of whether the effects of the combination treatments were synergistic, additive, or sub-additive relative to the individual treatments, the following statistics were calculated:

$$\begin{matrix} {Frac}_{{A}_{k}} = \frac{{AUC}_{ctl} - {AUC}_{{A}_{k}}}{{AUC}_{ctl}} & ( 3 ) \\ {Frac}_{{B}_{k}} = \frac{{AUC}_{ctl} - {AUC}_{{B}_{k}}}{{AUC}_{ctl}} & ( 4 ) \\ {Frac}_{{AB}_{k}} = \frac{{AUC}_{ctl} - {AUC}_{{AB}_{k}}}{{AUC}_{ctl}} & ( 5 ) \\ synergy score = ( mean ( {Frac}_{A} ) + mean ( {Frac}_{B} ) - mean ( {Frac}_{AB} ) ) * 100 & ( 6 )\end{matrix}$$

where Aₖ and Bₖ are the k^{th} animal in the individual treatment groups and ABₖ is the k^{th} animal in combination treatment group. AUC_{ctl} is the model-predicted AUC for the control group and was treated as a constant with no variability. The standard error of the synergy score was calculated as the square root of the sum of squared standard errors across groups A, B and AB. The degrees of freedom were estimated using the Welch-Satterthwaite equation. A hypothesis test was performed to determine if the synergy score differed from O. P values were calculated by dividing the synergy score by its standard error and tested against a t-distribution (two-tailed with the above-calculated degrees of freedom.

The effect was classified into four different categories. It was considered synergistic if the synergy score was less than 0 and additive if the synergy score wasn't statistically different from 0. If the synergy score was greater than zero, but the mean AUC for the combination was lower than the lowest mean AUC among the two single agent treatments, then the combination was sub-additive. If the synergy score was greater than zero, and the mean AUC for the combination was greater than the mean AUC for at least one of the single agent treatments, then the combination was antagonistic.

Interval analysis, if requested, involved a specified treatment group and time interval compared with another treatment group and time interval. For a given group, time interval, and animal, the tumor growth rate per day was estimated by

Rate=100*(10^{ΔY/Δt}−1) (7)

where ΔY is the difference in the log₁₀ tumor volume over the interval of interest, and Δt is the length of the time interval. If one or both of the time points were missing, then the animal was ignored. The mean rates across the animals were then compared using a two-sided unpaired t-test with unequal variances.

Given the exploratory nature of this study, there were no adjustments pre-specified for the multiple comparisons and endpoints examined. All P values <0.05 were called statistically significant in this analysis.

Table 39 is the annotation table for group symbols. Table 40 lists the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Table 41 lists the synergy analysis. Statistically significant negative synergy scores indicate a synergistic combination (“Syn.”). Statistically significant positive synergys cores indicate a sub-additive combination (“Sub-add”) when the combination performs better (i.e., has a lower AUC) than the best performing single agent. Statistically significant positive synergy scores indicate an antagonistic combination (“Antag.”) when the combination performs worse than the best performing single agent. Scores that are not statistically significant should be considered additive (“Add.”).

TABLE 39
Annotation Table

| | Group | Notation |
|---|---|---|
| 1 | vehicle/0.9percentsaline | V |
| 2 | ML02643.75mg/kg/264 | A |
| 3 | CPT-1110mg/kg/CPT-11 | B |
| 4 | CPT-1115mg/kg/CPT-11 | C |
| 5 | CPT-1130mg/kg/CPT-11 | D |
| 6 | ML02643.75mg/kgCPT1110mg/kg/CPT-11+264 | E |
| 7 | ML02643.75mg/kgCPT1115mg/kg/CPT-11+264 | F |
| 8 | ML02643.75mg/kgCPT1130mg/kg/CPT-11+264 | G |

TABLE 40
Results of Pairwise Comparisons

| | Reference | Treated | dAUC | P. Value |
|---|---|---|---|---|
| 1 | V | A | 25.1 | <0.001 |
| 2 | V | B | 90.9 | <0.001 |
| 3 | V | C | 103.1 | <0.001 |
| 4 | V | D | 56.2 | <0.001 |
| 5 | V | E | 169.7 | <0.001 |
| 6 | V | F | 173.7 | <0.001 |
| 7 | V | G | 106.1 | <0.001 |

TABLE 41
Results of Combination Analysis

| Treatment | | | | | |
|---|---|---|---|---|---|
| Group | | Score | SEM | P. Value | Assess |
| E | ML0264 3.75 mg/kg QW | −62.7 | 17.3 | 0.003 | Syn. |
| | CPT-11 10 mg/kg | | | | |
| | D 1, D 2/week | | | | |
| F | ML0264 3.75 mg/kg QW | −70.0 | 17.3 | 0.001 | Syn. |
| | CPT-11 | | | | |
| | 15 mg/kg D 1, D 2/week | | | | |
| G | ML0264 3.75 QW | −12.7 | 9.1 | 0.178 | Add. |
| | CPT-11 30 mg/kg | | | | |
| | D 1/QW | | | | |

As shown in Table 41 synergistic activity was achieved across the MLN0264 3.75 mg/kg QW CPT-11 10 mg/kg D1,D2/week (Group E) and ML0264 3.75 mg/kg QW CPT-11 15 mg/kg D1,D2/week (Group F) treatment groups. An additive effect was seen for the third treatment Group G, which used an alternative schedule for CPT-11 (once a week as opposed to day 1 and day 2 per week). Without intending to be bound by any theory, this alternative dosing schedule may have had an influence on the additive versus synergistic effect of the combination. The dose of CPT-11 was also higher in this treatment group.

In summary, the combination of 5F9 vcMMAE immunoconjugate and CPT-11 worked surprisingly well in the low antigen expressing PHTX-21c model at different dosing levels on a day 1 and day 2 per week dosing schedule. The combination of agents not only prevented tumor growth during dosing, but unpredictably reduced tumor size post-dosing. The combination also prevented tumor re-growth for an unexpectedly prolonged period of time after dosing was stopped. As demonstrated herein, the 5F9 vcMMAE immunoconjugate worked to sensitize the tumor to the DNA damaging activity such that the combination of the two agents worked synergistically at sub-optimal doses.

Anti-Tumor Activity of MLN0264, CPT-11 Administered Intravenously to Female CB-17 SCID Mice Bearing PHTX-17C s.c Xenografts (Study No. CPGC-1′-EF06)

As described above, combined administration of 5F9 vcMMAE immunoconjugate and CPT-11 was shown above to have synergistic activity in two primary human tumor xenograft models having moderate to high sensitivity to single agent activity of the immunoconjugate but very different levels of GCC antigen (PHTX-09c having an IHC Score 2-3+; PHTX-21c having an IHC score 1+). The objective of this study was to evaluate the combined activity in a model having moderate level of GCC antigen density and moderate sensitivity to the immunoconjugate alone. As shown in Example 5, the PHTX-17c model exhibits moderate antigen expression (IHC Score 2+) and moderate sensitivity to single agent activity of the 5F9 vcMMAE immunoconjugate. Thus, the PHTX-17c model was used to evaluate 5F9 vcMMAE at 3.75 mg/kg I.V. on a QWdosing schedule in combination with CPT-11 at 10 mg/kg or 15 mg/kg on a two day on/5 day off dosing schedule administered I.V. Dose formulations were prepared in accordance with Table 42. The study design is shown in Table 43.

TABLE 42
Preparation of the dose formulations

| | | | Dosing Solution | | |
|---|---|---|---|---|---|
| | | amt (ml | concentration | Vehi- | |
| Agent | Dose | liquid) | (mg/mL) | cle | Drug:Ab |
| 5F9 vcMMAE | 7.5 | 1.13 | 1.538 | 0.9% | 3.9 |
| (MLN0264) | mg/kg | | | saline | |
| CPT-11 | 10.0 | | 2.05 | 0.9% | |
| | mg/kg | | | saline | |
| CPT-11 | 15.0 | | 3.075 | 0.9% | |
| | mg/kg | | | saline | |
| CPT-11 | 30.0 | 1.35 | 6.15 | 0.9% | |
| | mg/kg | | | saline | |

The 15 mg/kg dosing solution was prepared by a 1:1 dilution of the 30 mg/kg dosing solution. The 10 mg/kg dosing solution was prepared by a 2:1 dilution of the 15 mg/kg dosing solution.

TABLE 43
CPGC-11-EF06 Study Design

| | mouse#/ | | | CPT-11 | | CPT-11 | |
|---|---|---|---|---|---|---|---|
| group | group | ADC first | schedule | i.v | schedule | total weekly | duration |
| 1 | 10 | vehicle i.v | D1 qw | | D1, D2 | 0 | W1, W2, W3, |
| 2 | 10 | MLN0264 | D1 qw | | | 0 | W1, W2, W3, |
| | | 7.5 mg/kg i.v | | | | | |
| 3 | 10 | | | 10 mg/kg | D1, D2 | 20 | W1, W2, W3 |
| | | | | | twice/w | | |
| 4 | 10 | | | 15 mg/kg | D1, D2 | 30 | W1, W2, W3 |
| | | | | | twice/w | | |
| 5 | 10 | | | 30 mg/kg | D1 QW | 30 | W1, W2, W3 |
| 6 | 10 | MLN0264 | D1 qw | 10 mg/kg | D1, D2 | 20 | W1, W2, W3 |
| | | 7.5 mg/kg i.v | | | twice/w | | |
| 7 | 10 | MLN0264 | D1 qw | 15 mg/kg | D1, D2 | 30 | W1, W2, W3 |
| | | 7.5 mg/kg i.v | | | twice/w | | |
| 8 | 10 | MLN0264 | D1 qw | 30 mg/kg | D1 QW | 30 | W1, W2, W3 |
| | | 7.5 mg/kg i.v | | | | | |

The endpoints for each treatment group was either a) tumor volume reaches 10% body weight; or b) body weight loss of >20%. The projected tumor volume at the study initiation date was 200 mm³.

Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 25-30 gauge, ½-¾ inch in length. Animals were dosed based on an average body weight of 20.5 grams.

Tumor volume measurements (0.01 mm) were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD). TGI was assessed on an arbitrary day (when the maximum tumor volume (MTV of the control treatment group reaches the maximum allowable tumor volume), using the following formula:

TGI=100−[MTV treated/MTV control]×100.

TGD was assessed by calculating T−C, where T=mean time (in days) for treatment group tumors to reach a predetermined size (e.g., 1,000 mm³), and where C=mean time (in days) for the control group tumors to reach a predetermined size.

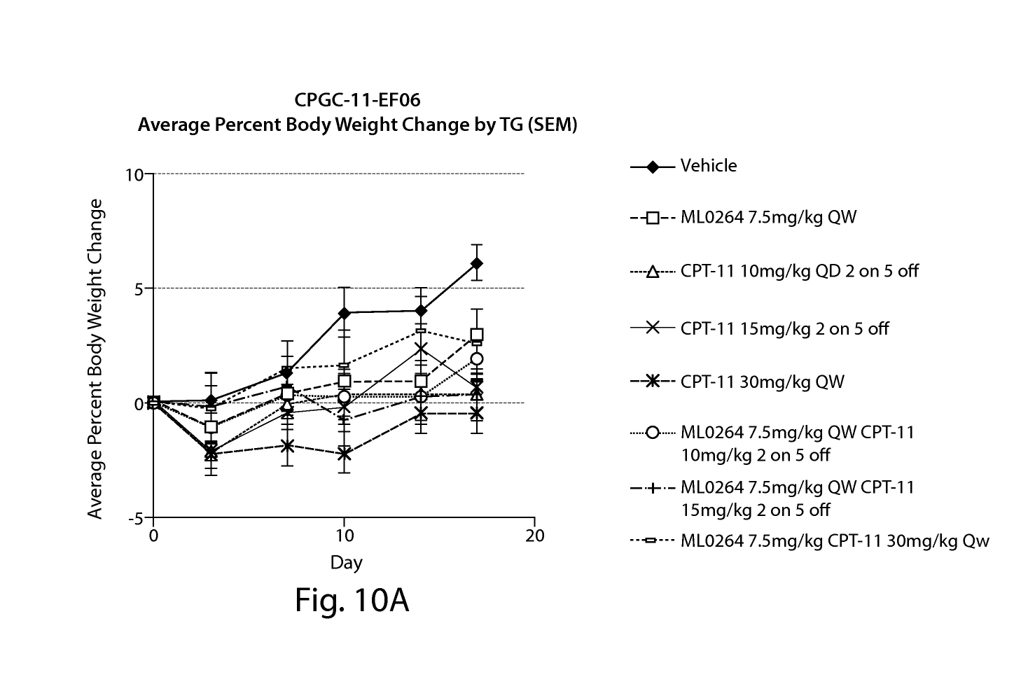

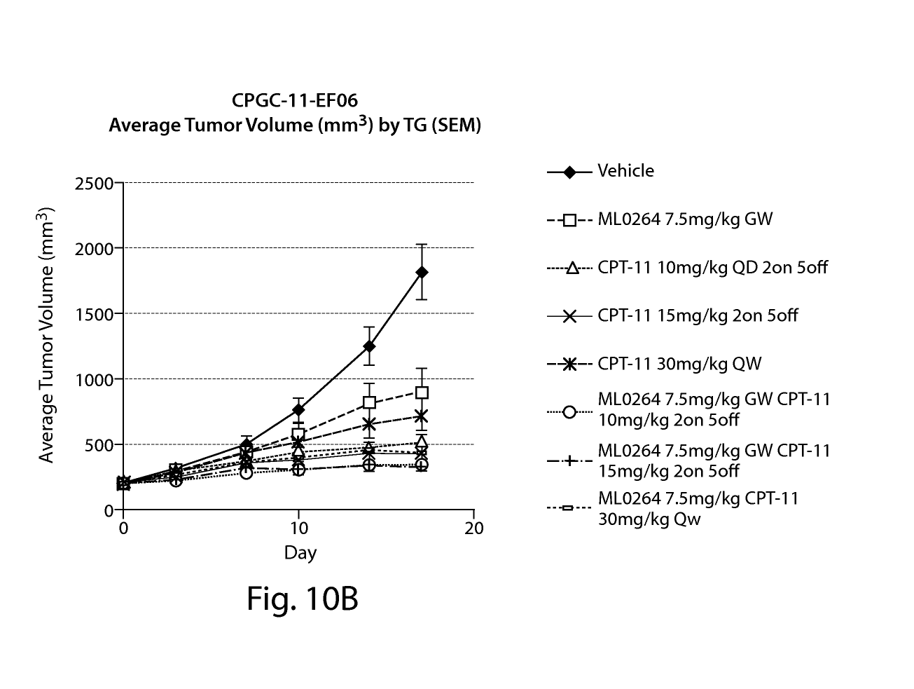

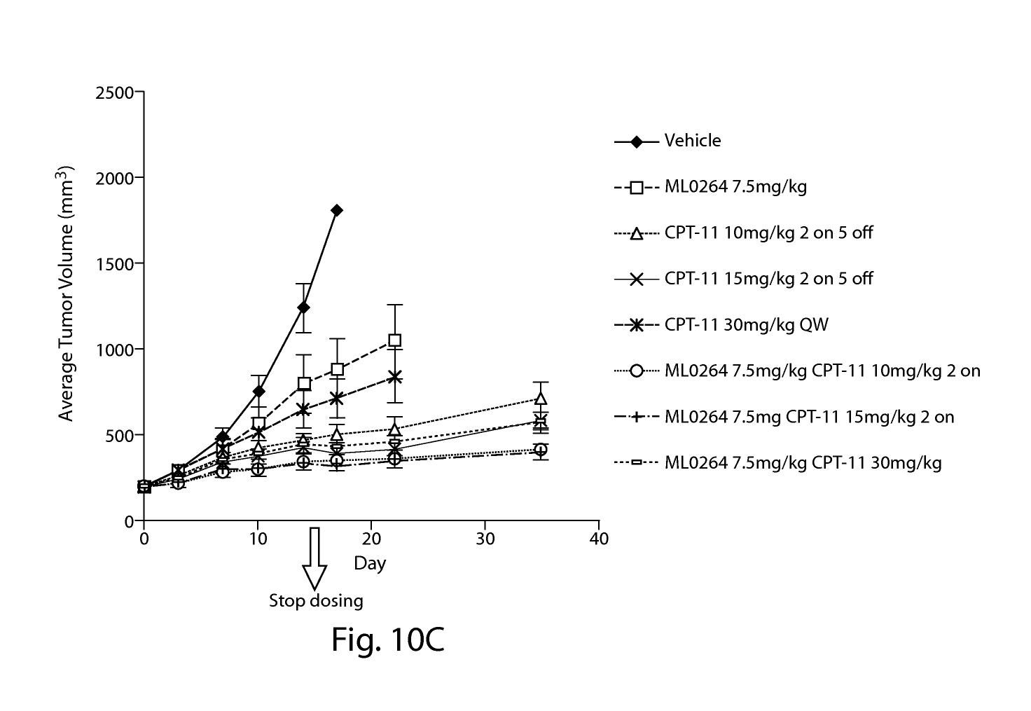

As shown in FIG. 10A, the doses were well tolerated in all treatment groups. Average tumor volume curves for the various treatment groups are shown in FIG. 10B. Tumor re-growth after dosing was stopped kinetics are shown in FIG. 10C. As shown in FIG. 10C, there was no difference between the CPT-11 10 mg/kg and 15 mg/kg combination treatment groups; both prevented tumor re-growth for an additional 3 weeks+post-dosing. The results of the study are summarized in Table 44.

TABLE 44
Results of CPGC-11-EF06 Study

| | Mean tumor | | | | |
|---|---|---|---|---|---|
| | volume ± | | Days to | | |
| Group | S.E.M (mm3) | TGI | 1000 mm\hat{ }3 | TGD | T/C |
| vehicle | 1810.1 ± 214.8 | | 12 | 0 | |
| ML0264 7.5 | 886.1 ± 187.9 | 51 | >17 | | 0.49 |
| mg/kg QW | | | | | |
| CPT-11 10 | 513.1 ± 52.2 | 71.7 | >17 | | 0.28 |
| mg/kg QD 2on 5off | | | | | |
| CPT-11 15 | 402.8 ± 36.6 | 77.7 | >17 | | 0.22 |
| mg/kg 2 on 5off | | | | | |
| CPT-11 30 | 715.1 ± 116.4 | 60.5 | >17 | | 0.4 |
| mg/kg QW | | | | | |
| ML0264 7.5 | 342.1 ± 45.6 | 81.1 | >17 | | 0.19 |
| mg/kg QW CPT-11 | | | | | |
| 10 mg/kg 2on 5off | | | | | |
| ML0264 7.5 | 327.2 ± 35.4 | 81.9 | >17 | | 0.18 |
| mg/kg QW CPT-11 | | | | | |
| 15 mg/kg 2 on 5 off | | | | | |
| ML0264 7.5 | 426.1 ± 40.6 | 76.5 | >17 | | 0.24 |
| mg/kg CPT-11 | | | | | |
| 30 mg/kg Qw | | | | | |

A longitudinal analysis of the CPGC-11-EF06 study using mixed effects linear regression models was performed as previously described with respect to the CPGC-11-EF07 study above to determine, in part, whether the combined anti-tumor efficacy of the 5F9 vcMMAE immunonjugate and CPT-11 was additive or synergistic.

Table 45 is the annotation table for group symbols. Table 46 lists the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Table 47 lists the synergy analysis. Statistically significant negative synergy scores indicate a synergistic combination (“Syn.”). Statistically significant positive synergy scores indicate a sub-additive or antagonistic combination (“Antag.”). Scores that are not statistically significant should be considered additive (“Add.”).

TABLE 45
Annotation Table

| | Group | Notation |
|---|---|---|
| 1 | vehicle/0.9percentsaline | V |
| 2 | ML02647.5mg/kgQW/264 | A |
| 3 | CPT-1110mg/kgQD2on5off/CPT-11 | B |
| 4 | CPT-1115mg/kg2on5off/CPT-11 | C |
| 5 | CPT-1130mg/kgQW/CPT-11 | D |
| 6 | ML02647.5mg/kgQWCPT-1110mg/kg2on5off/CPT-11+264 | E |
| 7 | ML02647.5mg/kgQWCPT-1115mg/kg2on5off/CPT-11+264 | F |
| 8 | ML02647.5mg/kgQWCPT-1130mg/kgQW/CPT-11+264 | G |

TABLE 46
Results of Pairwise Comparisons

| | Reference | Treated | dAUC | P. Value |
|---|---|---|---|---|
| 1 | V | A | 28.9 | <0.001 |
| 2 | V | B | 48.0 | <0.001 |
| 3 | V | C | 54.6 | <0.001 |
| 4 | V | D | 33.7 | <0.001 |
| 5 | V | E | 68.8 | <0.001 |
| 6 | V | F | 70.7 | <0.001 |
| 7 | V | G | 50.9 | <0.001 |

TABLE 47
Results of Combination Analysis

| | Score | SEM | P. Value | Assess |
|---|---|---|---|---|
| E | 7.3 | 12.7 | 0.573 | Add. |
| F | 22.3 | 11.6 | 0.305 | Add. |
| G | 11.2 | 13.5 | 0.415 | Add. |

As shown in Table 47 additive activity was achieved across all three combination treatment groups.

In summary, the PHTX-17c model having a moderate GCC antigen density level (IHC score 2+) and moderate sensitivity to the 5F9 vcMMAE immunoconjugate when administered as a single agent also displayed moderate sensitivity to combined administration of the immunoconjugate with CPT-11 in that the combination treatment was additive, rather than synergistic as seen with the PHTX-09c and PHTX-21c models, both having moderate to high sensitivity to the immunoconjugate, but high and low expression levels of the GCC antigen, respectively.

Anti-Tumor Activity of MLN0264, CPT-11 Administered Intravenously to Female CB-17 SCID Mice Bearing PHTX-11c s.c Xenografts (Study No. CPGC-12-EF01)

As described above, combined administration of 5F9 vcMMAE immunoconjugate and CPT-11 was shown above to have synergistic activity in two primary human tumor xenograft models having moderate to high sensitivity to single agent activity of the immunoconjugate but very different levels of GCC antigen (PHTX-09c having an IHC Score 2-3+; PHTX-21c having an IHC score 1+), but additive activity in a model having moderate sensitivity to the immunoconjugate and a moderate level of GCC antigen density (PHTX-17c having an IHC Score of 2+). The objective of this study was to explore the combination activity in a model having moderate level of GCC antigen density yet resistance to the immunoconjugate alone. As shown in Example 5, the PHTX-11c model exhibits moderate antigen expression (IHC Score 2+) but is resistant to single agent activity of the 5F9 vcMMAE immunoconjugate (FIGS. 6C and 6D)). Thus, the PHTX-11c model was used to evaluate combination therapy starting at a dose of 7.5 mg/kg of the 5F9 vcMMAE administered I.V. QW in combination with CPT-11 at 10 mg/kg, 15 mg/kg on a two day on/5 day off dosing schedule, and at 30 mg/kg QW. Dose formulations were prepared in accordance with Table 48. The study design is shown in Table 49.

TABLE 48
Preparation of the dose formulations

| | | | Dosing Solution | | |
|---|---|---|---|---|---|
| | | amt (ul | concentration | Vehi- | |
| Agent | Dose | liquidr) | (mg/mL) | cle | Ab/drug |
| 5F9 vcMMAE | 7.5 | 714.1 ui | 1.5075 | 0.9% | 4:1 |
| (MLN0264) | mg/kg | | | Saline | |
| CPT-11 | 10.0 | | 2.01 | 0.9% | |
| | mg/kg | | | Saline | |
| CPT-11 | 15.0 | | 3.02 | 0.9% | |
| | mg/kg | | | Saline | |
| CPT-11 | 30.0 | 1206 | 6.03 | 0.9% | |
| | mg/kg | | | Saline | |

The 15 mg/kg dosing solution was prepared by a 1:1 dilution of the 30 mg/kg dosing solution. The 10 mg/kg dosing solution was prepared by a 2:1 dilution of the 15 mg/kg dosing solution.

TABLE 49
CPGC-12-EF01 Study Design

| | mouse#/ | | | CPT-11 | | CPT-11 | |
|---|---|---|---|---|---|---|---|
| group | group | ADC first | schedule | i.v | schedule | total weekly | duration |
| 1 | 10 | vehicle i.v | D1 qw | | D1, D2 | 0 | W1, W2, W3 |
| 2 | 10 | MLN0264 | D1 qw | | | 0 | W1, W2, W3 |
| | | 7.5 mg/kg i.v | | | | | |
| 3 | 10 | | | 10 mg/kg | D1, D2 | 20 | W1, W2, W3 |
| | | | | | QD | | |
| 4 | 10 | | | 15 mg/kg | D1, D2 | 30 | W1, W2, W3 |
| | | | | | QD | | |
| 5 | 10 | | | 30 mg/kg | D1 QW | 30 | W1,W2, W3 |
| 6 | 10 | MLN0264 | D1 qw | 10 mg/kg | D1, D2 | 20 | W1, W2, W3 |
| | | 7.5 mg/kg i.v | | | QD | | |
| 7 | 10 | MLN0264 | D1 qw | 15 mg/kg | D1, D2 | 30 | W1, W2, W3 |
| | | 7.5 mg/kg i.v | | | QD | | |
| 8 | 10 | MLN0264 | D1 qw | 30 mg/kg | D1 QW | 30 | W1, W2, W3 |
| | | 7.5 mg/kg i.v | | | | | |

The endpoints for each treatment group was either a) tumor volume reaches 10% body weight; or b) body weight loss of >20%. The projected tumor volume at the study initiation date was 200 mm³.

Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 25-30 gauge, ½-¾ inch in length. Animals were dosed based on an average body weight of 20.1 grams.

Tumor volume measurements (0.01 mm) were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD). TGI was assessed on an arbitrary day (when the maximum tumor volume (MTV of the control treatment group reaches the maximum allowable tumor volume), using the following formula:

TGI=100−[MTV treated/MTV control]×100.

TGD was assessed by calculating T−C, where T=mean time (in days) for treatment group tumors to reach a predetermined size, and where C=mean time (in days) for the control group tumors to reach a predetermined size (e.g., 1,000 mm³).

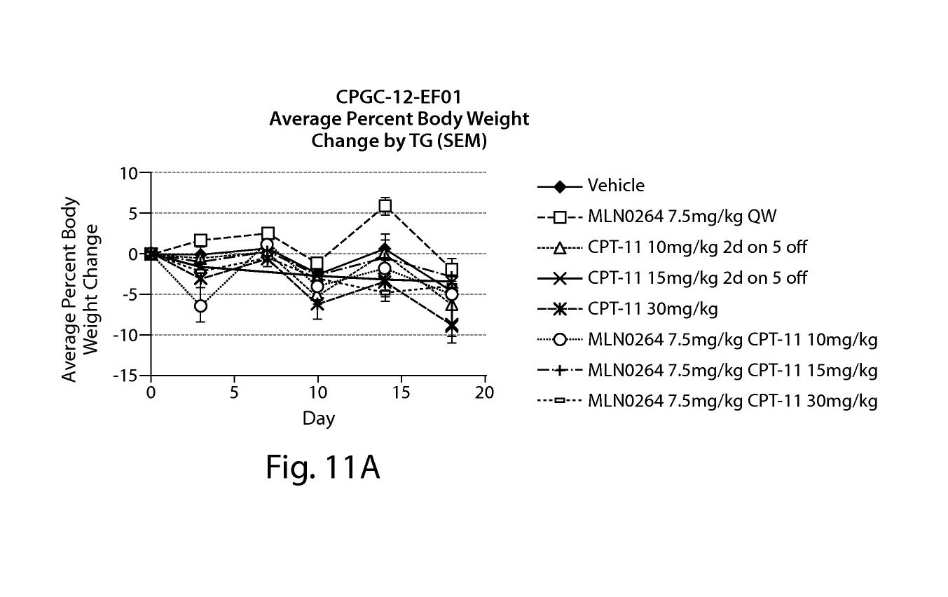

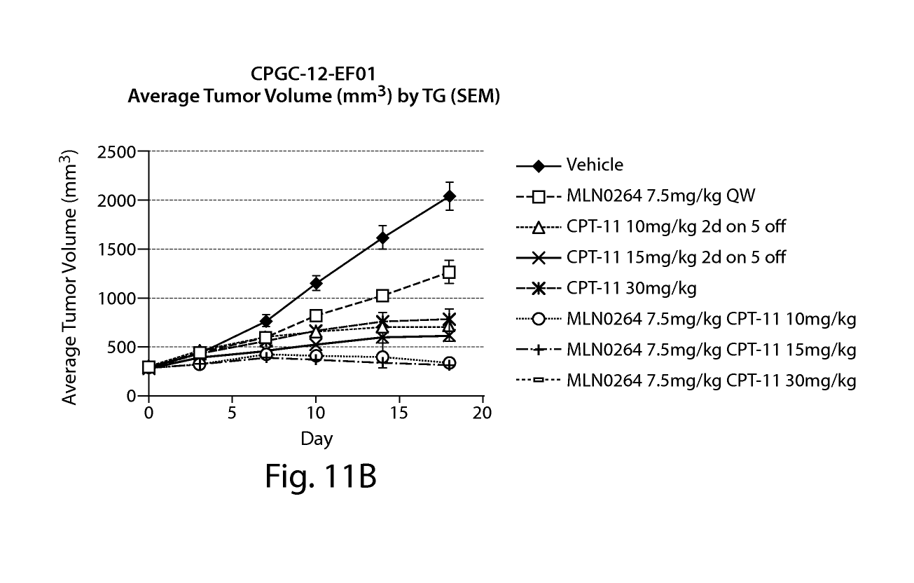

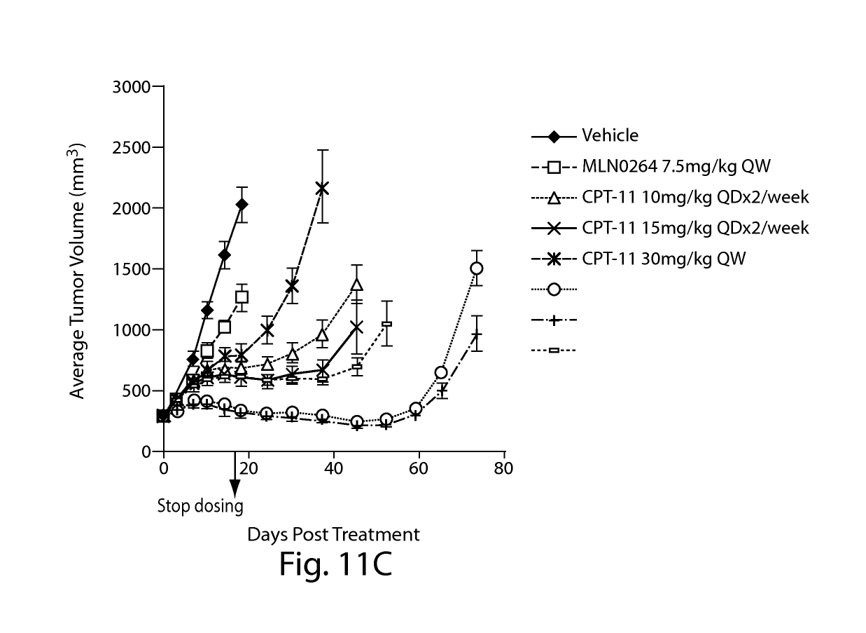

As shown in FIG. 11A, the doses were well tolerated in all treatment groups. The average tumor volume curves for the various treatment groups are shown in FIG. 11B. Tumor re-growth kinetics is shown in FIG. 11C.

As shown in FIG. 11B, the immunoconjugate alone had very low activity at the high 7.5 mg/kg dose, and there was very little difference in the activity seen with CPT-11 alone at the three different doses administered. However, the combined treatment with both 10 mg/kg and 15 mg/kg of CPT-11 completely inhibited tumor growth during dosing, and further prevented tumor re-growth after dosing was stopped for an an additional 6-7 weeks. Tumors did not start to regrow until around day 60. The results of the study are summarized in Table 50.

TABLE 50
Results of CPGC-12-EF01 Study

| | Mean tumor | | | | |
|---|---|---|---|---|---|
| | volume ± S.E.M | | Days to | | |
| Group | (mm3) | TGI | 1000 mm\hat{ }3 | TGD | T/C |
| Vehicle | 2028.9 ± 144.2 | | 8.8 | 0 | |
| MLN0264 7.5 | 1263.4 ± 115.8 | 37.7 | 13.5 | 4.7 | 0.62 |
| mg/kg QW | | | | | |
| CPT-11 10 | 691.4 ± 53.1 | 65.9 | >18 | | 0.34 |
| mg/kg 2 d on | | | | | |
| 5 off | | | | | |
| CPT-11 15 | 625.5 ± 73.8 | 69.2 | >18 | | 0.31 |
| mg/kg 2 d on | | | | | |
| 5 off | | | | | |
| CPT-11 30 mg/kg | 789.6 ± 94.2 | 61.1 | >18 | | 0.39 |
| MLN0264 7.5 | 346 ± 33.1 | 82.9 | >18 | | 0.17 |
| mg/kg CPT-11 | | | | | |
| 10 mg/kg | | | | | |
| MLN0264 7.5 | 311.5 ± 36.9 | 84.6 | >18 | | 0.15 |
| mg/kg CPT-11 | | | | | |
| 15 mg/kg | | | | | |
| MLN0264 7.5 | 629.3 ± 43.8 | 69 | >18 | | 0.31 |
| mg/kg CPT-11 | | | | | |
| 30 mg/kg | | | | | |

A longitudinal analysis of the CPGC-12-EF01 study using mixed effects linear regression models was performed to determine, in part, whether the combined anti-tumor efficacy of the 5F9 vcMMAE immunonjugate and CPT-11 was additive or synergistic. All tumor values (tumor volumes or photon flux) had a value of 1 added to them before log₁₀ transformation. These values were compared across treatment groups to assess whether the differences in the trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖ+treatᵢ+dayⱼ+dayⱼ²+(treat*day)ᵢⱼ+(treat*day²)ᵢⱼ+eᵢⱼₖ (1)

where Yᵢⱼₖ is the log₁₀ tumor value at the j^{th} time point of the k^{th} animal in the i^{th} treatment, Yᵢ₀ₖ is the day 0 (baseline) log₁₀ tumor value in the k^{th} animal in the i^{th} treatment, dayⱼ was the median-centered time point and (along with day²ⱼ) was treated as a continuous variable, and eᵢⱼₖ is the residual error. A spatial power law covariance matrix was used to account for the repeated measurements on the same animal over time. Interaction terms as well as day²ⱼ terms were removed if they were not statistically significant.

A likelihood ratio test was used to assess whether a given pair of treatment groups exhibited differences which were statistically significant. The −2 log likelihood of the full model was compared to one without any treatment terms (reduced model) and the difference in the values was tested using a Chi-squared test. The degrees of freedom of the test were calculated as the difference between the degrees of freedom of the full model and that of the reduced model.

The predicted differences in the log tumor values (Yᵢⱼₖ−Yᵢ₀ₖ, which can be interpreted as log₁₀ (fold change from day 0)) were taken from the above models to calculate mean AUC values for each treatment group. A dAUC value was then calculated as:

$$dAUC = \frac{mean ( {AUC}_{ctl} ) - mean ( {AUC}_{trt} )}{mean ( {AUC}_{ctl} )} * 100 \qquad ( 2 )$$

This assumed AUC_{ctl} was positive. In instances where AUC_{ctl} was negative, the above formula was multiplied by −1.

For synergy analyses, the observed differences in the log tumor values were used to calculate AUC values for each animal. In instances when an animal in a treatment group was removed from the study, the last observed tumor value was carried forward through all subsequent time points. The AUC for the control, or vehicle, group was calculated using the predicted values from the pairwise models described above. To address the question of whether the effects of the combination treatments were synergistic, additive, or sub-additive relative to the individual treatments, the following statistics were calculated:

$$\begin{matrix} {Frac}_{{A}_{k}} = \frac{{AUC}_{ctl} - {AUC}_{{A}_{k}}}{{AUC}_{ctl}} & ( 3 ) \\ {Frac}_{{B}_{k}} = \frac{{AUC}_{ctl} - {AUC}_{{B}_{k}}}{{AUC}_{ctl}} & ( 4 ) \\ {Frac}_{{AB}_{k}} = \frac{{AUC}_{ctl} - {AUC}_{{AB}_{k}}}{{AUC}_{ctl}} & ( 5 ) \\ synergy score = ( mean ( {Frac}_{A} ) + mean ( {Frac}_{B} ) - mean ( {Frac}_{AB} ) ) * 100 & ( 6 )\end{matrix}$$

where Aₖ and Bₖ are the k^{th} animal in the individual treatment groups and ABₖ is the k^{th} animal in combination treatment group. AUC_{ctl} is the model-predicted AUC for the control group and was treated as a constant with no variability. The standard error of the synergy score was calculated as the square root of the sum of squared standard errors across groups A, B, and AB. The degrees of freedom were estimated using the Welch-Satterthwaite equation. P values were calculated by dividing the synergy score by its standard error and tested against a t-distribution (two-tailed) with the above-calculated degrees of freedom.

The effect was classified into four different categories. It was considered synergistic if the synergy score was less than 0 and additive if the synergy score wasn't statistically different from 0. If the synergy score was greater than zero, but the mean AUC for the combination was lower than the lowest mean AUC among the two single agent treatments, then the combination was sub-additive. If the synergy score was greater than zero, and the mean AUC for the combination was greater than the mean AUC for at least one of the single agent treatments, then the combination was antagonistic. Interval analysis, if requested, involved a specified treatment group and time interval compared with another treatment group and time interval. For a given group, time interval, and animal, the tumor growth rate per day was estimated by

Rate=100*(10^{ΔY/Δt}−1) (7)

where ΔY is the difference in the log₁₀ tumor volume over the interval of interest, and Δt is the length of the time interval. If one or both of the time points were missing, then the animal was ignored. The mean rates across the animals were then compared using a two-sided unpaired t-test with unequal variances.

Given the exploratory nature of this study, there were no adjustments pre-specified for the multiple comparisons and endpoints examined. All P values <0.05 were called statistically significant in this analysis.

Table 51 is the annotation table for group symbols. Table 52 lists the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Table 53 lists the synergy analysis. Statistically significant negative synergy scores indicate a synergistic combination (“Syn.”). Statistically significant positive synergy scores indicate a sub-additive (“Sub-add”) combination when the combination performs better (i.e., has a lower AUC) than the best performing single agent. Statistically significant positive synergy scores indicate an antagonistic combination (“Antag”) when the combination performs worse than the best performing single agent. Scores that are not statistically significant should be considered additive (“Add.”).

TABLE 51
Annotation Table

| | Group | Notation |
|---|---|---|
| 1 | vehicle/0.9percentsaline | V |
| 2 | MLN02647.5mg/kgQW/264 | A |
| 3 | CPT-1110mg/kg2don5off/CPT-11 | B |
| 4 | CPT-1115mg/kg2don5off/CPT-11 | C |
| 5 | CPT-1130mg/kg/CPT-11 | D |
| 6 | MLN02647.5mg/kgCPT-1110mg/kg/CPT-11+264 | E |
| 7 | MLN02647.5mg/kgCPT-1115mg/kg/CPT-11+264 | F |
| 8 | MLN02647.5mg/kgCPT-1130mg/kg/CPT-11+264 | G |

TABLE 52
Results of Pairwise Comparisons

| | Reference | Treated | dAUC | P. Value |
|---|---|---|---|---|
| 1 | V | A | 22.6 | <0.001 |
| 2 | V | B | 42.6 | <0.001 |
| 3 | V | C | 51.3 | <0.001 |
| 4 | V | D | 41.6 | <0.001 |
| 5 | V | E | 80.2 | <0.001 |
| 6 | V | F | 86.8 | <0.001 |
| 7 | V | G | 49.1 | <0.001 |

TABLE 53
Results of Combination Analysis

| Treatment | | | | | |
|---|---|---|---|---|---|
| Group | | Score | SEM | P. Value | Assess |
| E | MLN0264 7.5 mg/kg | −26.8 | 7.8 | 0.003 | Syn. |
| | QW CPT-11 10 mg/kg | | | | |
| | D 1, d 2/week | | | | |
| F | MLN0264 7.5 mg/kg | −20.8 | 10 | 0.056 | Add. |
| | QW CPT-11 15 mg/kg | | | | |
| | D 1, d 2/week | | | | |
| G | MLN0264 7.5 mg/kg | −10.6 | 9.2 | 0.262 | Add. |
| | QW CPT-11 30 mg/kg | | | | |
| | D 1/week | | | | |

As shown in Table 53 the combination activity was synergistic in the MLN0264 7.5 mg/kg QW CPT-11 10 mg/kg D1,d2/week (Treatment Group E), and additive in the other two combination treatment groups (Groups F and G). It should be noted that the synergy analysis only takes into account the tumor volume/tumor growth inhibition. It does not take into account tumor growth delay. As shown in FIG. 11C, significant tumor re-growth delay (˜80 days) was seen with CPT-11 at 10 and 15/mg kg in combination with 7.5 mg/kg of the 5F9 vcMMAE immunoconjugate activity. Thus, the tumor re-growth kinetics shown in FIG. 11C suggests synergistic activity for both Treatment Groups E and F. Given the surprising length of tumor growth delay observed for both Treatment Groups E and F, and the similarity of the respective curves shown in FIG. 11C, the synergy analysis for Treatment Group F will be reassessed using a different statistical method than the one described above. Treatment Group G (MLN0264 7.5 mg/kg QW CPT-11 30 mg/kg D1/week) used a once/week dosing schedule versus the day 1 and day 2 per week schedule of Treatment Groups E and F. Without intending to be bound by any theory, this alternative dosing schedule may have an effect on the additive versus synergistic effect for this treatment group.

In summary, the PHTX-11c model having a moderate GCC antigen density (IHC score 2+) but resistance to the 5F9 vcMMAE immunoconjugate when administered as a single agent was surprisingly sensitivity to the combination therapy at multiple dosing regimens, particularly with respect to delayed tumor re-growth. Despite its resistance to single agent activity of the immunoconjugate, the immunoconjugate effectively sensitized the tumor to the DNA damaging agent activity. As mentioned above, synergy analysis is being repeated to confirm synergistic activity seen with respect to prolonged delay in tumor re-growth.

Taken together, the in vivo combination studies described herein demonstrate that the combination of an anti-GCC immunoconjugate and CPT-11 work synergistically to inhibit tumor growth, regardless of the tumor sensitivity to the immunoconjugate alone, and regardless of GCC antigen density. The anti-tumor activity is maintained even after dosing is stopped for surprisingly prolonged periods of time. In each of the pre-clinical models evaluated, synergistic or enhanced activity was achieved at sub-optimal doses of each agent demonstrating that the combination of an anti-GCC immunoconjugate and CPT-11 provides a therapeutic benefit over the administration of either agent alone. The synergistic or enhanced effects seen in these preclinical models are expected to translate into the clinic. The combination provides a promising treatment alternative for patients whose cancer is resistant to the immunoconjugate alone.

### Example 7

### In Vivo Evaluation of Combined Administration of an anti-GCC Immunoconjugate and Cisplatin

The purpose of this study was to evaluate in vivo anti-tumor activity induced by combined administration of the 5F9 vcMMAE immunoconjugate (sometimes referred to herein as “MLN0264”) and cisplatin in the PHTX-09c primary human tumor xenografts (“PHTX”) derived from mCRC described in Example 5. The study was conducted as follows.

Anti-Tumor Activity of 5F9 vcMMAE and Cisplatin Administered Intravenously to Female CB-17 SCID Mice Bearing PHTX-09c s.c. Xenografts (Study No. CPGC-11-EF05)

As demonstrated in Example 5, the PHTX-09c model was highly sensitive to single agent activity of the 5F9 vcMMAE immunoconjugate at various concentrations. The objective of this study was to evaluate the 5F9 vcMMAE immunoconjugate in combination with cisplatin. More specifically, the goal of this study was to evaluate the anti-tumor activity of 5F9 vcMMAE at 1.875 and 3.75 mg/kg on a once weekly dosing schedule in combination with cisplatin at 4 mg/kg and 6 mg/kg administered intravenously (IV) or intraperitoneal (IP) on a once weekly or every two week dosing schedule in PHTX-09c s.c. xenograft in CB17 SCID mice. Dose formulations were prepared in accordance with Table 54. The study design is shown in Table 55.

TABLE 54
Preparation of the dose formulations

| | | | Dosing | | |
|---|---|---|---|---|---|
| | | | Solution | | |
| | | amt (ul | concentration | Vehi- | |
| Agent | Dose | liquid) | (mg/mL) | cle | Drug:Ab |
| MLN0264- | 1.875 | | 0.392 | 0.9% | 3.9 |
| | mg/kg | | | saline | |
| MLN0264- | 3.75 | 653.1 | 0.784 | 0.9% | 3.9 |
| | mg/kg | | | saline | |
| Cisplatin | 4.0 | 2170 | 0.836 | 0.9% | |
| | mg/kg | | | saline | |
| Cisplatin | 6.0 | 130/mouse | 1.254 | 0.9% | |
| | mg/kg | | | saline | |

TABLE 55
CPGC-11-EF05 Study Design

| | | | | | Dose | | | |
|---|---|---|---|---|---|---|---|---|
| | | | | | Route/Dose | Dose | | |
| | | | # of | Dose | regimen/ | Solution | *Dose | |
| Treatment | Test | | animals/ | level | Dose | Conc. | Volume | |
| Group | Article | Vehicle | Group | (mg/kg) | Duration | (mg/ml) | (ml) | Endpoints |
| 1 | 0.9% | N/A | 9 | N/A | IV/QW ×3 | 0 | 0.1 | Tumor |
| | saline | | | | week | | | volume |
| | | | | | | | | reach |
| | | | | | | | | 10% BW |
| | | | | | | | | or BW |
| | | | | | | | | loss > 20% |
| 2 | MLN0264 | 0.9% | 9 | 1.875 | IV/QW ×3 | 0.392 | 0.1 | Tumor |
| | | saline | | | week | | | volume |
| | | | | | | | | reach |
| | | | | | | | | 10% BW |
| | | | | | | | | or BW |
| | | | | | | | | loss > 20% |
| 3 | MLN0264 | 0.9% | 9 | 3.75 | IV/QW ×3 | 0.784 | 0.1 | Tumor |
| | | saline | | | week | | | volume |
| | | | | | | | | reach |
| | | | | | | | | 10% BW |
| | | | | | | | | or BW |
| | | | | | | | | loss > 20% |
| 4 | Cisplatin | 0.9% | 9 | 4.0 | IV/QW ×2 | 0.836 | 0.1 | Tumor |
| | | saline | | | week | | | volume |
| | | | | | | | | reach |
| | | | | | | | | 10% BW |
| | | | | | | | | or BW |
| | | | | | | | | loss > 20% |
| 5 | Cisplatin | 0.9% | 9 | 6.0 | IV/QW ×1 | 1.254 | 0.13 | Tumor |
| | | saline | | | week | | | volume |
| | | | | | | | | reach |
| | | | | | | | | 10% BW |
| | | | | | | | | or BW |
| | | | | | | | | loss > 20% |
| 6 | Cisplatin, | 0.9% | 9 | 6.0, | IV/QW ×3 | 1.254 | 0.13 | Tumor |
| | MLN0264 | saline, | | 1.875 | week and | 0.392 | | volume |
| | | 0.9% | | | QW ×1 | | | reach |
| | | saline | | | | | | 10% BW |
| | | | | | | | | or BW |
| | | | | | | | | loss > 20% |
| 7 | Cisplatin, | 0.9% | 9 | 6.0, | IV/QW ×3 | 1.254 | 0.13 | Tumor |
| | MLN0264 | saline, | | 3.75 | week and | 0.784 | | volume |
| | | 0.9% | | | QW ×1 | | | reach |
| | | saline | | | | | | 10% BW |
| | | | | | | | | or BW |
| | | | | | | | | loss > 20% |
| 8 | Cisplatin, | 0.9% | 9 | 4.0, | IV/QW ×3 | 0.836 | 0.1 | Tumor |
| | MLN0264 | saline, | | 3.75 | week and | 0.784 | | volume |
| | | 0.9% | | | QW ×2 | | | reach |
| | | saline | | | | | | 10% BW |
| | | | | | | | | or BW |
| | | | | | | | | loss > 20% |

The endpoints for each treatment group was either a) tumor volume reaches 10% body weight; or b) body weight loss of >20%. The projected tumor volume at the study initiation date was 200 mm³.

Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 27-30 gauge, ½-¾ inch in length. Animals were dosed based on an average body weight of 20.9 grams.

Tumor volume measurements were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD); the difference in days for treated vs. control tumors to reach final study volume).

TGI was assessed on an arbitrary day (when the maximum tumor volume (“MTV” of the control treatment group reaches the maximum allowable tumor volume), using the following formula:

TGI=100−[MTV treated/MTV control]×100.

TGD was assessed by calculating T−C, where T=mean time (in days) for treatment group tumors to reach a predetermined size, and where C=mean time (in days) for the control group tumors to reach a predetermined size (e.g., 1,000 mm³).

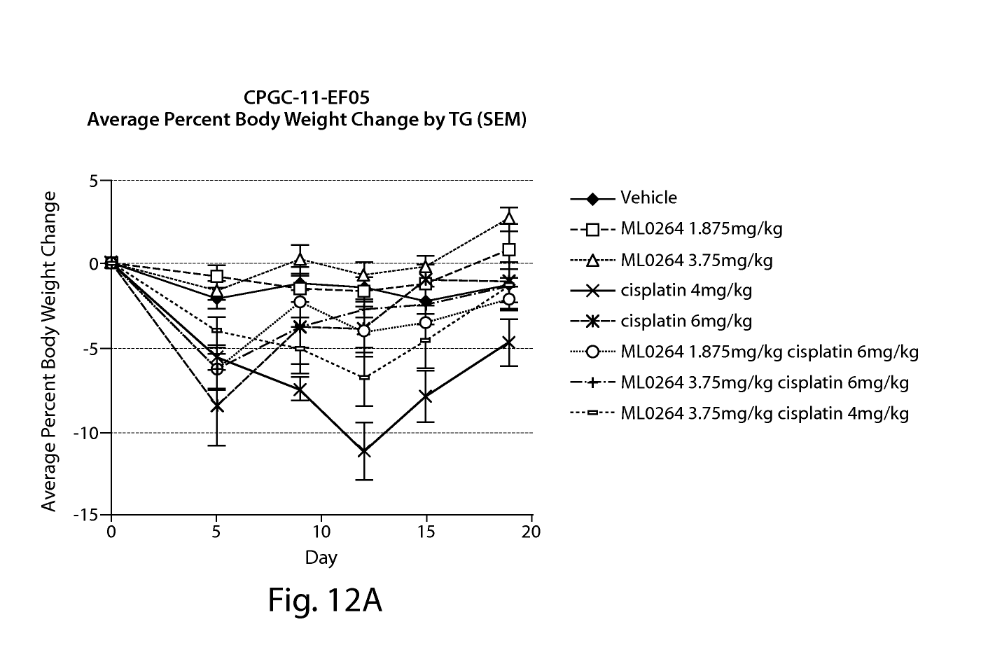

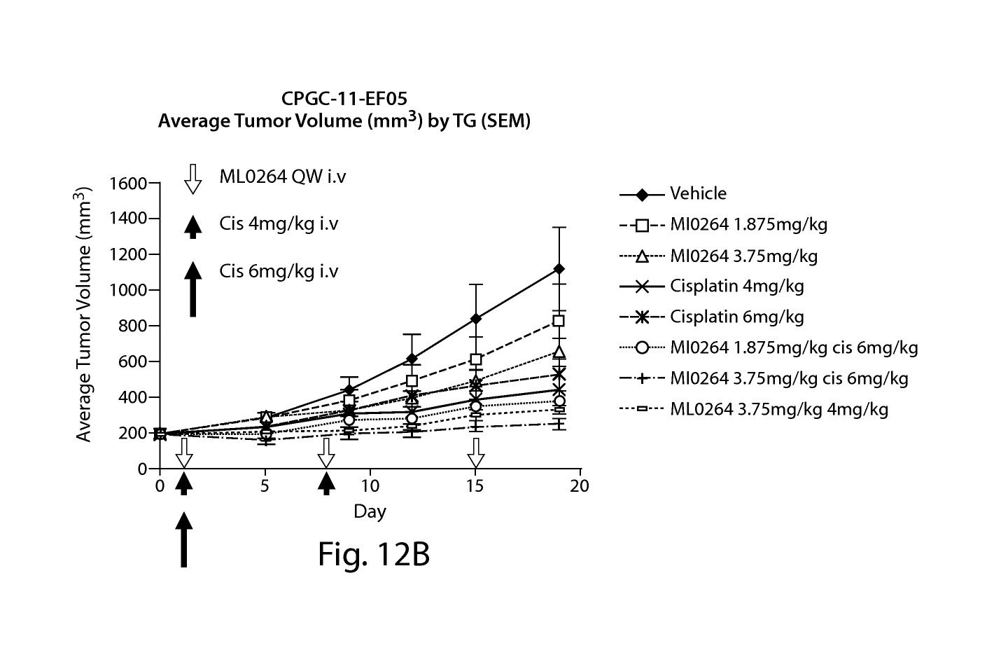

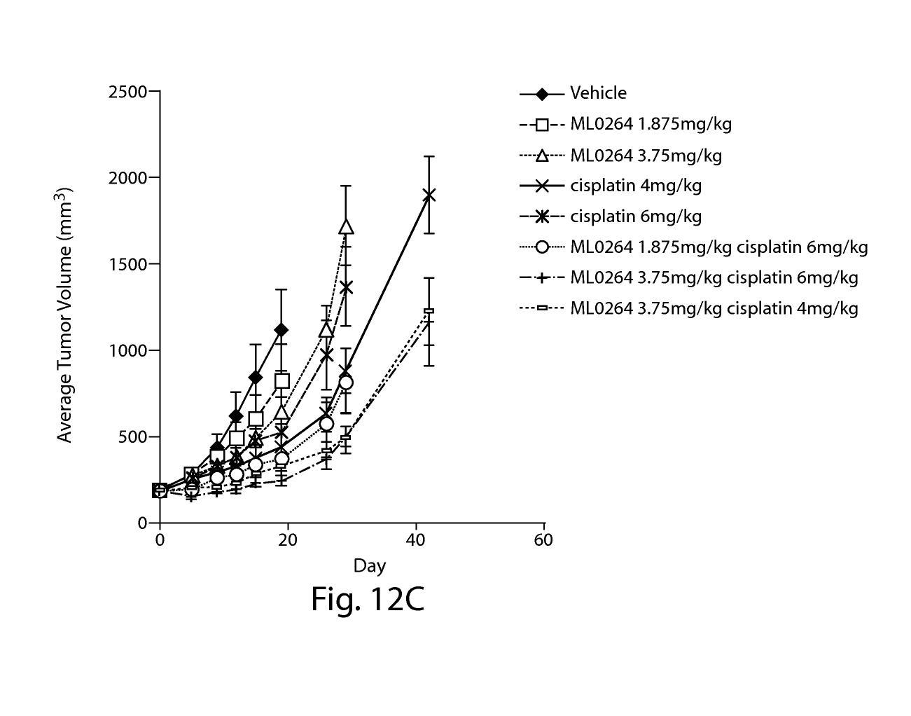

In general, i.v. dosing route was determined to be better tolerated than the i.p. dosing. As shown in FIG. 12A, the doses were well tolerated in all combination treatment groups, and the combination treatment was better tolerated than cisplatin alone where body weight loss >15% was seen. Cisplatin administered once weekly at 4 mg/kg was tolerated for 2 doses, administration at 6 mg/kg once weekly was tolerated for only 1 dose. The average tumor volume curves for the various treatment groups is shown in FIG. 12B.

A longitudinal analysis of the CPGC-11-EF05 study using mixed effects linear regression models was performed to determine, in part, whether the combined anti-tumor efficacy of the 5F9 vcMMAE immunonjugate and cisplatin was additive or synergistic. All tumor values (tumor volumes or photon flux) had a value of 1 added to them before log₁₀ transformation. These values were compared across treatment groups to assess whether the differences in the trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖ+treatᵢ+dayⱼ+dayⱼ²+(treat*day)ᵢⱼ+(treat*day²)ᵢⱼ+eᵢⱼₖ (1)

where Yᵢⱼₖ is the log₁₀ tumor value at the j^{th} time point of the k^{th} animal in the i^{th} treatment, Yᵢ₀ₖ is the day 0 (baseline) log₁₀ tumor value in the k^{th} animal in the i^{th} treatment, dayⱼ was the median-centered time point and (along with day²ⱼ) was treated as a continuous variable, and eᵢⱼₖ is the residual error. A spatial power law covariance matrix was used to account for the repeated measurements on the same animal over time. Interaction terms as well as day²ⱼ terms were removed if they were not statistically significant.

A likelihood ratio test was used to assess whether a given pair of treatment groups exhibited differences which were statistically significant. The −2 log likelihood of the full model was compared to one without any treatment terms (reduced model) and the difference in the values was tested using a Chi-squared test. The degrees of freedom of the test were calculated as the difference between the degrees of freedom of the full model and that of the reduced model. The predicted differences in the log tumor values (Yᵢⱼₖ−Yᵢ₀ₖ, which can be interpreted as log₁₀ (fold change from day 0)) were taken from the above models to calculate mean AUC values for each treatment group. A dAUC value was then calculated as:

$$dAUC = \frac{mean ( {AUC}_{ctl} ) - mean ( {AUC}_{trt} )}{mean ( {AUC}_{ctl} )} * 100 \qquad ( 2 )$$

This assumed AUC_{ctl} was positive. In instances where AUC_{ctl} was negative, the above formula was multiplied by −1.

For synergy analyses, the observed differences in the log tumor values were used to calculate AUC values for each animal. In instances when an animal in a treatment group was removed from the study, the last observed tumor value was carried forward through all subsequent time points. The AUC for the control, or vehicle, group was calculated using the predicted values from the pairwise models described above. A measure of synergy was defined as follows:

$$\begin{matrix} {Frac}_{{A}_{k}} = \frac{{AUC}_{ctl} - {AUC}_{{A}_{k}}}{{AUC}_{ctl}} & ( 3 ) \\ {Frac}_{{B}_{k}} = \frac{{AUC}_{ctl} - {AUC}_{{B}_{k}}}{{AUC}_{ctl}} & ( 4 ) \\ {Frac}_{{AB}_{k}} = \frac{{AUC}_{ctl} - {AUC}_{{AB}_{k}}}{{AUC}_{ctl}} & ( 5 ) \\ synergy score = ( mean ( {Frac}_{A} ) + mean ( {Frac}_{B} ) - mean ( {Frac}_{AB} ) ) * 100 & ( 6 )\end{matrix}$$

where Aₖ and Bₖ are the k^{th} animal in the individual treatment groups and ABₖ is the k^{th} animal in combination treatment group. AUC_{ctl} is the model-predicted AUC for the control group and was treated as a constant with no variability. The standard error of the synergy score was calculated as the square root of the sum of squared standard errors across groups A, B, and AB. The degrees of freedom were estimated using the Welch-Satterthwaite equation. A hypothesis test was performed to determine if the synergy score differed from 0. P values were calculated by dividing the synergy score by its standard error and tested against a t-distribution (two-tailed) with the above-calculated degrees of freedom.

The effect was classified into four different categories. It was considered synergistic if the synergy score was less than 0, and additive if the synergy score wasn't statistically different from 0. If the synergy score was greater than zero, but the mean UAC for the combination was lower than the lowest mean AUC among the two single agent treatments, then the combination was sub-additive. If the synergy score was greater than zero, and the mean AUC for the combination was greater than the mean AUC for at least one of the single agent treatments, the combination was antagonistic.

Interval analysis, if requested, involved a specified treatment group and time interval compared with another treatment group and time interval. For a given group, timer interval, and animal, the tumor growth rate per day was estimated by

Rate=100*(10^{ΔY/Δt}−1) (7)

where ΔY is the difference in the log₁₀ tumor volume over the interval of interest, and Δt is the length of the time interval. If one or both of the time points were missing, then the animal was ignored. The mean rates across the animals were then compared using a two-sided unpaired t-test with unequal variances.

Given the exploratory nature of this study, there were no adjustments pre-specified for the multiple comparisons and endpoints examined. All P values <0.05 were called statistically significant in this analysis.

Table 56 is the annotation table for group symbols. Table 57 lists the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Table 58 lists the synergy analysis. Statistically significant negative synergy scores indicate a synergistic combination (“Syn.”). Statistically significant positive synergy scores indicate a sub-additive combination (“Sub-add.”) when the combination performs better (i.e., has a lower AUC) than the best performing single agent. Statistically significant positive synergy scores indicate an antagonistic combination (“Antag.”) when the combination performs worse than the best performing single agent. Scores that are not statistically significant should be considered additive (“Add.”).

TABLE 56
Annotation Table

| | Group | Notation |
|---|---|---|
| 1 | vehicle/0.9percentSaline | V |
| 2 | ML02641.875mg/kg/264 | A |
| 3 | ML02643.75mg/kg/264 | B |
| 4 | cisplatin4mg/kg/Cisplatin | C |
| 5 | cisplatin6mg/kg/Cisplatin | D |
| 6 | ML02641.875mg/kgcisplatin6mg/kg/Cisplatin+264 | E |
| 7 | ML02643.75mg/kgcisplatin6mg/kg/Cisplatin+264 | F |
| 8 | ML02643.75mg/kgcisplatin4mg/kg/Cisplatin+264 | G |

TABLE 57
Results of Pairwise Comparisons

| | Reference | Treated | dAUC | P. Value |
|---|---|---|---|---|
| 1 | V | A | 17.8 | 0.051 |
| 2 | V | B | 28.4 | 0.004 |
| 3 | V | C | 50.6 | <0.001 |
| 4 | V | B | 44.8 | 0.001 |
| 5 | V | E | 67.5 | <0.001 |
| 6 | V | F | 98.8 | <0.001 |
| 7 | V | G | 74.0 | <0.001 |

TABLE 58
Results of Combination Analysis

| Treatment | | | | | |
|---|---|---|---|---|---|
| Group | | Score | SEM | P. Value | Assess |
| E | ML0264 1.875 mg/kg | −11.3 | 19.1 | 0.56 | Add. |
| | cisplatin 6 mg/kg D 1 | | | | |
| | week 1 | | | | |
| F | ML0264 3.75 mg/kg | −55 | 18.5 | 0.007 | Syn. |
| | cisplatin 6 mg/kg D 1 | | | | |
| | week 1 | | | | |
| G | ML0264 3.75 mg/kg | −31.7 | 13.4 | 0.029 | Syn. |
| | cisplatin 4 mg/kg D 1 | | | | |
| | week 1 and week 2 | | | | |

As shown in Table 58, synergistic activity was achieved using a dosing regimen of ML0264 3.75 mg/kg cisplatin 6 mg/kg D1 week 1 (Treatment Group F) as well as ML0264 3.75 mg/kg cisplatin 4 mg/kg D1 week 1 and week 2 (Treatment Group G). Additive activity was seen using a lower dose of the immunoconjugate (1.875 mg/kg) in combination with cisplatin 6 mg/kg D1 week 1 (Treatment Group E).

As shown in FIG. 12B, improved antitumor activity was seen in all combination groups, with synergistic activity seen in Treatment Groups F and G. As shown in FIG. 12C, significant tumor re-growth delay (˜50 days) was seen with cisplatin treatment at 4 and 6/mg kg in combination with 3.75 mg/kg of the 5F9 vcMMAE immunoconjugate activity.

In summary, PHTX-09c model which has relatively high GCC antigen density as demonstrated in the IHC assay described above (Score 2-3+) and high sensitivity to various concentrations of the 5F9 vcMMAE immunoconjugate when administered as a single agent displayed synergistic sensitivity to combined administration of the immunoconjugate and cisplatin at the higher dose of the immunoconjugate tested (3.75 mg/kg) in combination with different doses and dosing schedules of cisplatin.

### Example 8

### In Vivo Evaluation of Combined Administration of an anti-GCC Immunoconjugate and 5-Fluorouracil

The purpose of this study was to evaluate in vivo anti-tumor activity induced by combined administration of the 5F9 vcMMAE immunoconjugate (sometimes referred to herein as “MLN0264”) and 5-Fluorouracil (5-FU) in the PHTX-21c primary human tumor xenografts (“PHTX”) derived from mCRC described in Example 5. The study was conducted as follows.

Anti-Tumor Activity of MLN0264 and 5-Fluorouracil Administered Intravenously to Female CB-17 SCID Mice Bearing PHTX-21C s.c Xenografts (Study No. CPGC-11-EF01)

As demonstrated in Example 5, the PHTX-21c model was moderately sensitive to single agent activity of the 5F9 vcMMAE immunoconjugate at various concentrations despite having a low GCC antigen density level (IHC score 1+). The objective of this study was to evaluate the anti-tumor activity of 5F9 vcMMAE and 5-Fluorouracil (5-FU) in the PHTX-21c model. The administration of 5F9 vcMMAE at 3.75 mg/kg and 7.5 mg/kg on once weekly dosing schedules was evaluated in combination with 5-FU at 15 mg/kg and 25 mg/kg on a 3 day on/4 day off dosing schedule administered IV for 4 weeks. A vc-MMAE immunoconjugate having an antibody non-specific for GCC (referred to as 209-vcMMAE) was used as a control. Dose formulations were prepared in accordance with Table 59. The study design is shown in Table 60.

TABLE 59
Preparation of the dose formulations

| | | | Dosing Solution | | |
|---|---|---|---|---|---|
| Treatment | | amt (ul | concentration | Vehi- | |
| Group | Dose | liquid) | (mg/mL) | cle | Drug/Ab |
| 209-vcMMAE | 3.75 | | 0.7313 | 0.9% | 4.1 |
| (non-GCC | mg/kg | | | saline | |
| control | | | | | |
| immuno- | | | | | |
| conjugate) | | | | | |
| 209-vcMMAE | 7.5 | 274.8 | 1.4625 | 0.9% | 4.1 |
| (non-GCC | mg/kg | | | saline | |
| control | | | | | |
| immuno- | | | | | |
| conjugate) | | | | | |
| 5F9-vcMMAE | 3.75 | | 0.7313 | 0.9% | 3.9 |
| | mg/kg | | | saline | |
| 5F9-vcMMAE | 7.5 | 731.3 | 1.4625 | 0.9% | 3.9 |
| | mg/kg | | | saline | |
| 5-Fu | 15.0 | | 2.4375 | 0.9% | pH 8.6 |
| | mg/kg | | | saline | |
| 5-Fu | 25.0 | 390 | 4.875 | 0.9% | pH 8.6 |
| | mg/kg | | | saline | |

3.75 mg/kg dosing solution was prepared by diluting 7.5 mg/kg dosing solution 1:1.
15 mg/kg dosing solution was prepared by diluting 25 mg/kg dosing solution 3:2.

TABLE 60
CPGC-11-EF01 Study Design

| | | | | | | Dose | | | |
|---|---|---|---|---|---|---|---|---|---|
| | | | | | | Route/ | | | |
| | | | | | | Dose | Dose | | |
| | | | | # of | Dose | regimen/ | Solution | *Dose | |
| Treatment | Test | Sequenc- | | animals/ | level | Dose | Conc. | Volume | |
| Group | Article | ing | Vehicle | Group | (mg/kg) | Duration | (mg/ml) | (ml) | Endpoints |
| 1 | 0.9% | | N/A | 10 | N/A | IV/QD | 0 | 0.1 | Tumor weight |
| | saline | | | | | 3 days | | | reach 10% BW |
| | | | | | | on, 4 | | | or BW loss > |
| | | | | | | days | | | 20% |
| | | | | | | off ×4 | | | |
| | | | | | | week | | | |
| 2 | ML00732042 | | 0.9% | 10 | 3.75 | IV/QW ×4 | 0.7313 | 0.1 | Tumor weight |
| | | | saline | | | week | | | reach 10% BW |
| | | | | | | | | | or BW loss > |
| | | | | | | | | | 20% |
| 3 | ML00732042 | | 0.9% | 10 | 7.5 | IV/QW ×4 | 1.4625 | 0.1 | Tumor weight |
| | | | saline | | | week | | | reach 10% BW |
| | | | | | | | | | or BW loss > |
| | | | | | | | | | 20% |
| 4 | MLN0264 | | 0.9% | 10 | 3.75 | IV/QW ×4 | 0.7313 | 0.1 | Tumor weight |
| | | | saline | | | week | | | reach 10% BW |
| | | | | | | | | | or BW loss > |
| | | | | | | | | | 20% |
| 5 | MLN0264 | | 0.9% | 10 | 7.5 | IV/QW ×4 | 1.4625 | 0.1 | Tumor weight |
| | | | saline | | | week | | | reach 10% BW |
| | | | | | | | | | or BW loss > |
| | | | | | | | | | 20% |
| 6 | 5-Fu | | 0.9% | 10 | 15.0 | IV/QD | 2.4375 | 0.1 | Tumor weight |
| | | | saline | | | 3 days | | | reach 10% BW |
| | | | | | | on, 4 | | | or BW loss > |
| | | | | | | days | | | 20% |
| | | | | | | off 4 | | | |
| | | | | | | weeks | | | |
| 7 | 5-Fu | | 0.9% | 10 | 25.0 | IV/QD | 4.875 | 0.1 | Tumor weight |
| | | | saline | | | 3 days | | | reach 10% BW |
| | | | | | | on, 4 | | | or BW loss > |
| | | | | | | days | | | 20% |
| | | | | | | off ×4 | | | |
| | | | | | | weeks | | | |
| 8 | 5-Fu, | 5- | 0.9% | 10 | 15.0, | IV/QD | 2.4375 | 0.1 | Tumor weight |
| | MLN0264 | Fu// | saline, | | 3.75 | 3 days | 0.7313 | 0.1 | reach 10% BW |
| | | MLN0264 | 0.9% | | | on, 4 | | | or BW loss > |
| | | | saline | | | days | | | 20% |
| | | | | | | off & | | | |
| | | | | | | QW ×4 | | | |
| | | | | | | weeks | | | |
| 9 | 5-Fu, | 5- | 0.9% | 10 | 25.0, | IV/QD | 4.875 | 0.1 | Tumor weight |
| | MLN0264 | Fu// | saline, | | 3.75 | 3 days | 0.7313 | 0.1 | reach 10% BW |
| | | MLN0264 | 0.9% | | | on, 4 | | | or BW loss > |
| | | | saline | | | days | | | 20% |
| | | | | | | off & | | | |
| | | | | | | QW ×4 | | | |
| | | | | | | weeks | | | |

The endpoints for each treatment group was either a) tumor volume reaches 10% body weight; or b) body weight loss of >20%. The projected tumor volume at the study initiation date was 200 mm³.

Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 27-30 gauge, ½-¾ inch in length. Animals were dosed based on an average body weight of 19.5 grams.

Tumor volume measurements were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD). TGI was assessed on an arbitrary day (when the maximum tumor volume (MTV of the control treatment group reaches the maximum allowable tumor volume), using the following formula:

TGI=100−[MTV treated/MTV control]×100.

TGD was assessed by calculating T−C, where T=mean time (in days) for treatment group tumors to reach a predetermined size (e.g., 1,000 mm³), and where C=mean time (in days) for the control group tumors to reach a predetermined size.

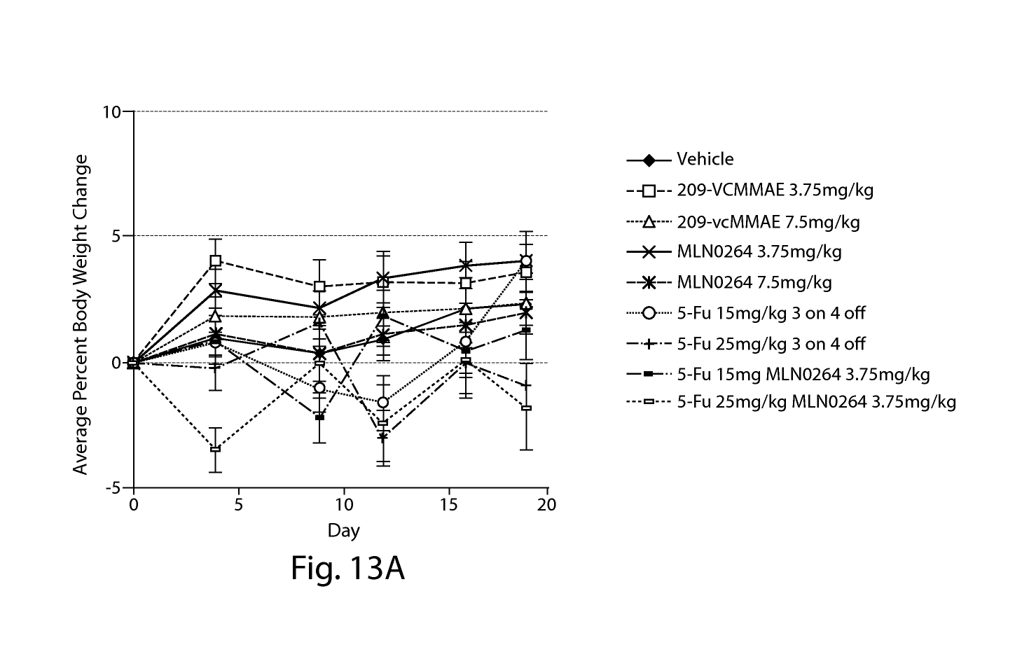

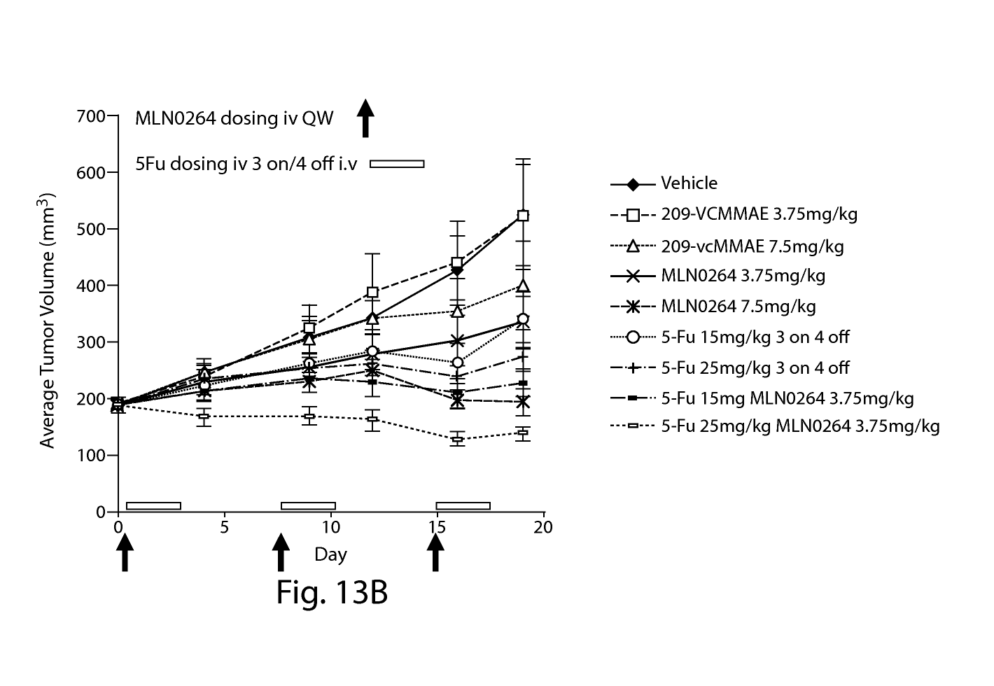

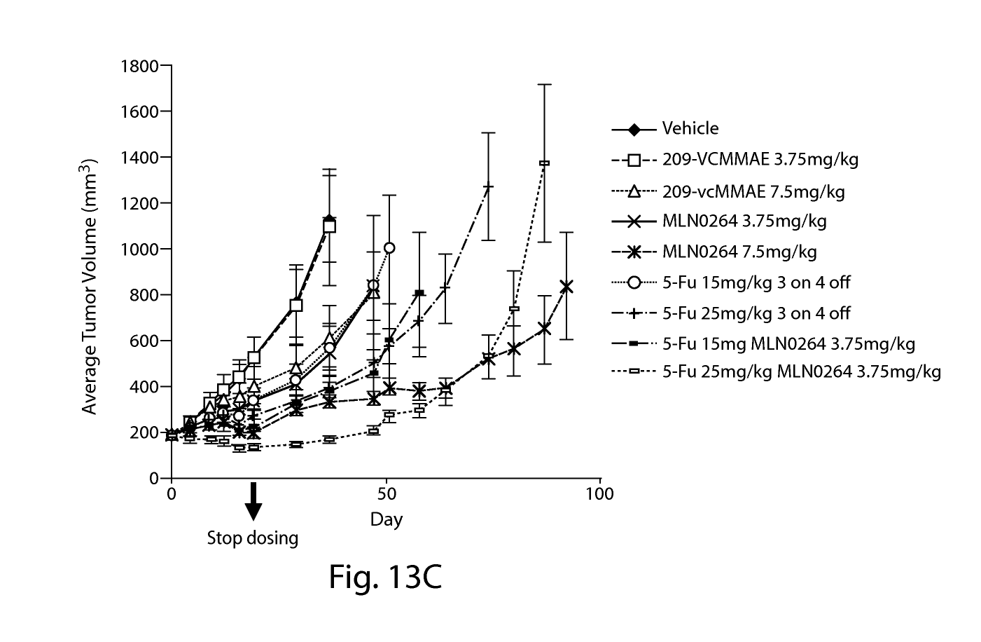

As shown in FIG. 13A, the doses were well tolerated in all treatment groups. Average tumor volume curves for the various treatment groups are shown in FIG. 13B. As shown in FIG. 8B, the 5F9-vcMMAE immunoconjugate had relatively low anti-tumor activity as a single agent at the 3.75 mg/kg dose, the single agent anti-tumor activity of 5-FU at both 15 mg/kg and 25 mg/kg were similar, each having relatively low anti-tumor activity as well. Surprisingly, 3.75 mg/kg of the immunoconjugate with 25 mg/kg of 5-FU of completely eradicated tumor volume during the treatment course.

The tumor re-growth kinetics curve is shown in FIG. 13C. As shown in FIG. 13C, the combined treatment regimen with 3.75 mg/kg of the immunoconjugate and 25 mg/kg 5-FU as described herein not only effectively prevented tumor growth during dosing, but prevented tumor regrowth for an additional 45 weeks post-dosing. Tumors did not start to re-grow until around day 50.

A longitudinal analysis of the CPGC-11-EF01 study using mixed effects linear regression models was performed to determine, in part, whether the combined anti-tumor efficacy of the 5F9 vcMMAE immunonjugate and 5-FU was additive or synergistic, using the methods as described in Example 7 above.

Table 61 is the annotation table for group symbols. Table 62 lists the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Table 63 lists the synergy analysis. Statistically significant negative synergy scores indicate a synergistic combination (“Syn.”). Statistically significant positive synergys cores indicate a sub-additive combination (“Sub-add”) when the combination performs better (i.e., has a lower AUC) than the best performing single agent. Statistically significant positive synergy scores indicate an antagonistic combination (“Antag.”) when the combination performs worse than the best performing single agent. Scores that are not statistically significant should be considered additive (“Add.”).

TABLE 61
Annotation Table
FIG. 1: Results by Group

| | Group | Notation |
|---|---|---|
| 1 | vehicle/0.9percentSaline | V |
| 2 | 209-vcMMAE3.75mg/kgQW/042 | A |
| 3 | 209-vcMMAE7.5mg/kgQW/042 | B |
| 4 | MLN02643.75mg/kgQW/264 | C |
| 5 | MLN02647.5mg/kgQW/264 | D |
| 6 | 5-Fu15mg/kg3on/5-Fu | E |
| 7 | 5-Fu25mg/kg3on/5-Fu | E |
| 8 | MLN02643.75mg/kg5-Fu15mg/kg/264+5-Fu | G |
| 9 | MLN02643.75mg/kg5-Fu25mg/kg/5-Fu+264 | H |

TABLE 62
Results of Pairwise Comparisons

| | Reference | Treated | dAUC | P. Value |
|---|---|---|---|---|
| 1 | V | A | −0.3 | 0.831 |
| 2 | V | B | 23.5 | 0.107 |
| 3 | V | C | 38.5 | 0.022 |
| 4 | V | D | 74.0 | <0.001 |
| 5 | V | E | 44.1 | 0.010 |
| 6 | V | F | 52.6 | <0.001 |
| 7 | V | G | 77.6 | <0.001 |
| 8 | V | B | 143.6 | <0.001 |

TABLE 63
Results of Combination Analysis

| | Score | SEM | P. Value | Assess |
|---|---|---|---|---|
| G | 7.0 | 20.0 | 0.728 | Add. |
| H | −56.2 | 19.7 | 0.009 | Syn. |

As shown in Table 63 synergistic activity was achieved in the MLN0264 3.75 mg/kg and 5-FU 25 mg/kg treatment group.

### Example 9

### GCC Expression in Human Pancreatic Tumor Microarrays and Clinical Samples

An automated protocol based on the IHC assay described in Example 5 above was used to evaluate GCC expression in primary human tumor xenografts (PHTX) derived from pancreatic cancer patient samples in female SCID mice, and in pancreatic tumor microarrays (TMAs) purchased from commercial sources (e.g., US BioMax and Pantomics). These tumors covered a range of tumor grades. GCC expression was also assessed via IHC in human pancreatic tumor samples obtained from the tissue database of a specialty CRO (QualTek).

Four-micron sections were prepared from the various tissue samples. Tissue sections were dewaxed through 4, 5-minute changes of xylene followed by a graded alcohol series to distilled water. Steam heat induced epitope recovery (SHIER) was used with SHIER2 solution for 20 minutes in the capillary gap in the upper chamber of a Black and Decker Steamer.

TABLE 69A
IHC Procedure

| TechMate | |
|---|---|
| Steps | Ultra Vision Detection (UV) |
| 1. | Ultra Vision Block- 15 minutes |
| 2. | Primary Antibody Incubation - Overnight |
| 3. | Primary Antibody Enhancer- 25 minutes |
| 4. | Hydrogen peroxide block- 3 × 2.5 minutes each |
| 5. | Polymer Detection- 25 minutes |
| 6. | DAB Chromogen- 3 × 5.0 minutes each |
| 7. | Hematoxylin Counter Stain - 1 minute |

TABLE 69B
Antibody Reactivity Spec Sheet

| Anitbody | GCC |
|---|---|
| Supplier | MLNM Takeda in-house antibody |
| Catalog No. | N/A |
| Source/Isotype | RbIgG |
| Supplier Lot # | Not determined |
| QualTek Lot # | R3512 |
| Clone | 148-2 |
| Concentration | 0.475 μg/ml |
| Suggested Dilution | 1.0 μg/ml |
| Incubation Time | Overnight |
| Pretreatments | SHIER2, no enzyme |
| TechMate Protocol | MIP |
| Detection system | Ultra Vision Detection System |
| Sub-Cellular | Cytoplasmic and/or apical |
| Localization | |

One skilled in the art would recognize that the primary antibody enhancer can be an anti-rabbit secondary antibody raised in a species other than rabbit (e.g., human, rat, goat, mouse, etc.) having the same isotype as the MIL-44-148-2 or MIL-44-67 rabbit mAbs (rabbit IgG) or a similar reagent that is suitable to amplify the MIL-44 signal.

The above protocol used an overnight antibody incubation of MIL-44-148-2 at 1.0 μg/ml with a non-biotin based peroxidase detection (Ultravision kit from Thermo/Lab Vision) and DAB as chromogen. This procedure was completely automated using the TechMate 500 or TechMate 1000 (Roche Diagnostics). After staining, slides were dehydrated through an alcohol series to absolute ethanol followed by xylene rinses. Slides were permanently coverslipped with glass coverslips and CytoSeal. Slides were examined under a microscope to assess staining. Positive staining is indicated by the presence of a brown (DAB-HRP) reaction product. Hematoxylin counterstain provides a blue nuclear stain to assess cell and tissue morphology.

An H-score approach was used for quantifying GCC expression. The H-score approach provides optimal data resolution for determining variation in intensity and tumor percentage of staining within and among tumor types. It also provides a good tool for determining thresholds for positive staining. In this method, the percentage of cells (0-100) within a tumor with staining intensities ranging from 0-3+ are provided. With the instant method, scores with intensities of 0, 0.5, 1, 2 and 3 were provided. Depending on the marker, 0.5 staining can be scored as positive or negative, and reflects light but perceptible staining for the marker. To obtain an H-score, the percentage of tumor cells are multiplied by each intensity and added together:

H score=(% tumor*1)+(% tumor*2)+(% tumor*3). For example, if a tumor is 20% negative (O), 30%+1, 10%+2, 40%+3, this would give an H score of 170.

The maximum H-score is 300 (100%*+3), per sub-cellular localization (i.e., apical or cytoplasmic), if 100% of tumor cells label with 3+ intensity. Initially, as a control, the total H-score alone was not be used to compare samples, but evaluated in addition to a review of the break-down of the percentage of cells at each intensity. For example, a score of 90 could represent 90% of tumor cells staining with 1+ intensity or 30% of cells with 3+ intensity. These samples have the same H-score but very different GCC expression. The percentage of cells to be scored at each intensity can vary, but are normally scored in increments of 10%; however, a small percentage of scoring of a single component can be estimated at 1% and 5% as well in order to demonstrate that some level of staining is present. For GCC, apical staining may be considered for evaluating at low level increments, such as 1 and 5%.

Two different sub-cellular localizations were scored for GCC using the H-score approach. These include cytoplasmic staining and apical associated staining. The cytoplasmic staining pattern was generally observed as diffuse throughout the cytoplasm of tumor cells. However, in some cases there were variations of the cytoplasmic staining, which included intense globular staining or punctate, coarse granular staining. Intense globular staining was scored as 3+ cytoplasmic staining. The punctate staining was associated with apical staining and was not given a separate score for this type of cytoplasmic staining (n=4 samples for punctate staining). GCC apical staining was observed when lumen were present. Other GCC staining patterns observed included membrane-like, non-lumen staining (one case) and extra-cellular staining present in tumor lumen. In normal colon tissues, staining was generally apical along with diffuse cytoplasmic staining.

Since H scores were obtained for both cytoplasmic and apical GCC expression, and since it is not known whether one type of localization is more critical over another for efficacy of a GCC-targeted therapy, all data was captured and an some instances, an aggregate H score was generated by using the sum of both apical and cytoplasmic GCC expression. In such instances, the maximum H score became 600 for the aggregate score (300 apical+300 cytoplasmic).

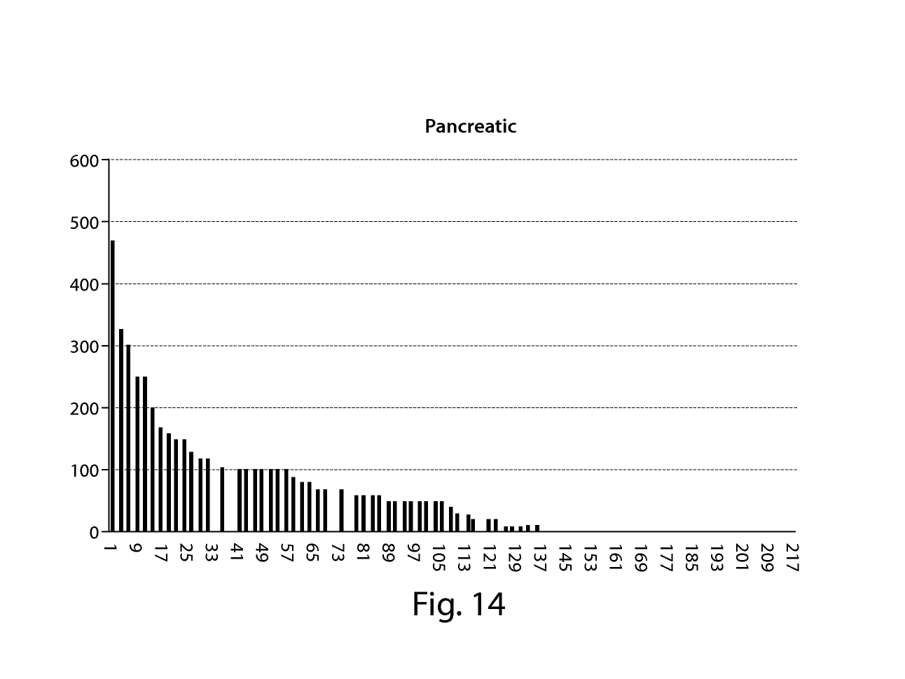

A total of 218 primary and metastatic pancreatic tumors were screened. 137 expressed GCC with 58 samples having a combined cytoplasmic and apical H-score of ≧100. A graphical summary of the H-scores for the 137 GCC positive samples is shown in FIG. 14.

### Example 10

### In Vivo Evaluation of Combined Administration of an Anti-GCC Immunoconjugate and Gemcitabine

The purpose of this study was to evaluate in vivo anti-tumor activity induced by combined administration of the 5F9 vcMMAE immunoconjugate (sometimes referred to herein as “MLN0264”) and gemcitabine in mouse xenograft primary human tumor explants (PHTX models of pancreatic cancer. These models included tumor tissue from patients with wild-type and mutant KRAS. Five pancreatic PHTX were generated by Champions Oncology (Hackensack, N.J.) using their CTG platform. 2 PHTX models were developed in-house. GCC expression was evaluated in each of the 7 PHTX models using the automated IHC assay described above. The GCC IHC H-scores for the five Champions models (CTG models) and the two in-house models (PHTX models) is shown in Table 70.

TABLE 70
GCC expression in Pancreatic PHTX models from Champions

| PHTX | GGG IHC H score | KRAS |
|---|---|---|
| Model | A/C | mutant |
| CTG-0300 | 150/250 | G12V |
| CTG-0301 | 210/250 | WT |
| CTG-0302 | 300/220 | G12R |
| CTG-0303 | 300/250 | WT |
| CTG-0492 | 130/250 | G12V |
| PHTX-215Pa | 230/260 | WT |
| PHTX-249Pa | 0/220 | G12D |

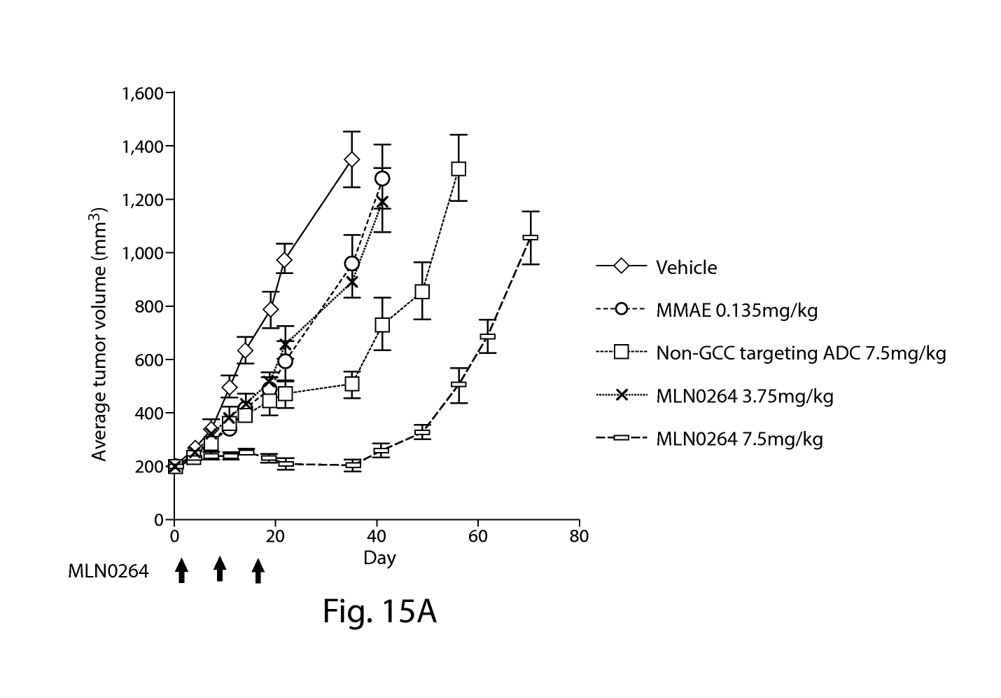

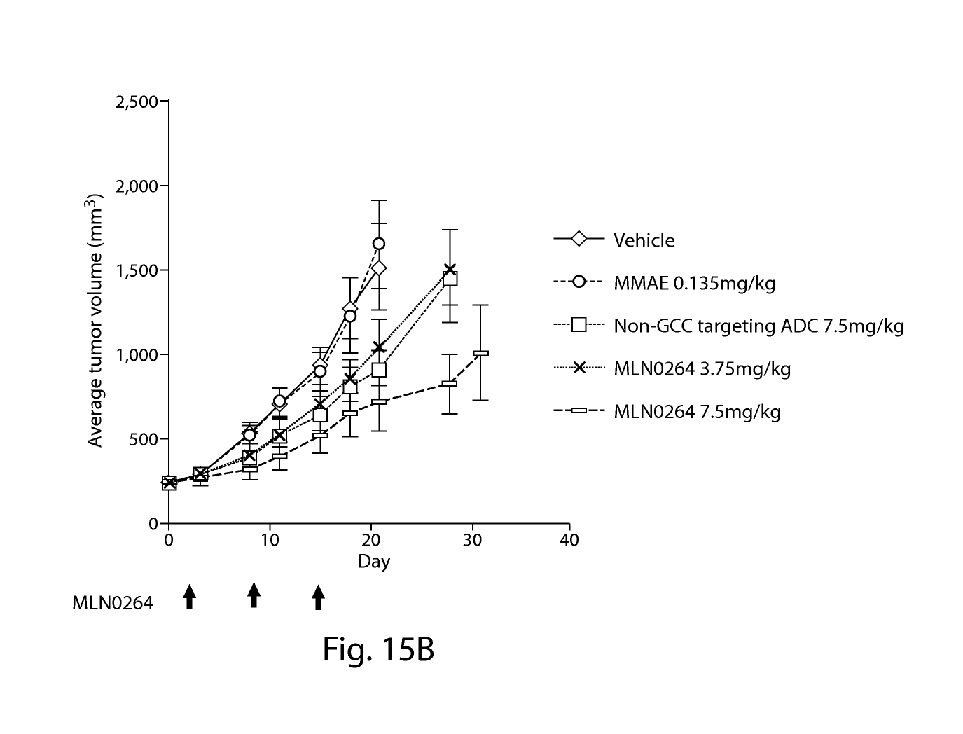

In vivo anti-tumor studies of single-agent 5F9 vcMMAE immunoconjugate was conducted in all seven of the pancreatic subcutanreous xenograft mouse models. Animals were administered vehicle, free MMAE at 0.135 mg/kg once weekly (QW), a non-GCC targeting ADC at 7.5 mg/kg QW, or 5F9 vcMMAE at 3.75 or 7.5 mg/kg QW. The results for single agent activity of the immunoconjugate in PHTX-215 Pa and PHTX-249 Pa models is shown in FIGS. 15A and 15B, respectively.

As shown in FIG. 15A, the 5F9 vcMMAE immunoconjugate at 7.5 mg/kg resulted in significantly great tumor growth inhibition (TGI) versus free MMAE of 5F9 vcMMAE immunoconjugate at 3.75 mg/kg by day 22 in the PHTX-215 Pa model (KRAS wt). This included tumor regressions in 3 out of 8 animals. The 7.5 mg/kg dose of 5F9 vcMMAE also resulted in delayed tumor regrowth. Similarly, as shown in FIG. 15B, in the PHTX-249 Pa model, single agent 5F9 vcMMAE immunoconjugate showed significant TGI versus vehicle or free MMAE by day 21. 5F9 vcMMAE at 7.5 mg/kg was significantly better than the dose at 3.75 mg/kg by day 20-22 in the PHTX-249 Pa model (KRAS G12D). Table 71 summarizes the TGI data with single-agent 5F9 vcMMAE activity across all seven pancreatic tumor xenograft models.

TABLE 71
Single Agent Anti-Tumor Activity in Pancreatic PHTX Models

| | GCC IHC | | | |
|---|---|---|---|---|
| Model | total H-score | KRAS status | TGI, %* | dAUC P-value^{†} |
| PHTX-215 | 490 | Wild-type | 79 | <0.001 |
| CTG-0300 | 400 | Mut G12V | 64 | <0.001 |
| CTG-0492 | 380 | Mut G12V | 62 | 0.001 |
| PHTX-249 | 220 | Mut G12D | 47 | <0.001 |
| CTG-0303 | 550 | Wild-type | 45 | 0.028 |
| CTG-0302 | 520 | Mut G12R | 36 | 0.006 |
| CTG-0301 | 460 | Wild-type | 24 | 0.17 |

*TGI determined on day 21 for all models, using QWx3 dosing, except for CTG-0300 and CTG-0303, which used QWx5 dosing and had TGI determined on day 28 and day 38, respectively
^{†}P-values are based on dAUC

Antitumor Activity of 5F9 vcMMAE Immunoconjugate and Gemcitabine Administered Intravenously in Female CB-17 SCID Mice Bearing PHTX-249 Pa s.c. Xenografts (Study Nos. CPGC-13-EF05 and CPGC-13-EF08)

The objective of this study was to determine synergy or additive antitumor activity of 5F9 vcMMAE immunoconjugate at 7.5 mg/kg on once weekly (QW) schedule combined with Gemcitabine at 15 mg/kg and 20 mg/kg twice weekly (BIW) or Gemcitabine at 15 mg/kg on Day 1 and Day 3 each week, administrated intravenously in PHTX-249 Pa s.c xenografts in CB17 SCID F mice. A vc-MMAE immunoconjugate having an antibody non-specific for GCC (referred to as 209-vcMMAE) was used as a control. Dose formulations were prepared in accordance with Table 71A and Table 71B. The study designs are shown in Table 72A and 72B.

TABLE 71A
Preparation of Dose Formulations-Study CPGC-13-EF05

| | | | Dosing Solution | | |
|---|---|---|---|---|---|
| | | amt (mg | concentration | Vehi- | |
| Agent | Dose | powder) | (mg/mL) | cle | Ab:drug |
| 5F9 vcMMAE | 7.5 | 377.8 ul | 1.4625 | 0.9% | 1:4.3 |
| immuno- | mg/kg | | | Saline | |
| conjugate | | | | | |
| Gemcitabine | 15.0 | 115.4 ul | 2.925 | 0.9% | |
| | mg/kg | | | Saline | |
| Gemcitabine | 20.0 | 307.9 ul | 3.9 | 0.9% | |
| | mg/kg | | | Saline | |

Gemcitabine stock solution concentration is 38 mg/ml

TABLE 71B
Preparation of Dose Formulations-Study CPGC-13-EF08

| | | | Dosing Solution | |
|---|---|---|---|---|
| | | amt (mg | concentration | |
| Agent | Dose | powder) | (mg/mL) | Vehicle |
| 5F9 vcMMAE | 7.5 | 726.9 ul | 1.478 | 0.9% Saline |
| immuno- | mg/kg | | | |
| conjugate | | | | |
| Gemcitabine | 15.0 | 264.4 ul | 2.955 | 0.9% Saline |
| | mg/kg | | | |

MLN0264 stock solution concentration is 8.13 mg/ml for 40 mice based on BW 19.7 g total need MLN0264 5.19 mg and 726.9 ul stock solution into 3273.1 ul NS.
Gemcitabine: stock concentration is 38 mg/ml, for 34 mice, total need 10.047 mg based on BW 19.7 g. 264.4 ul stock Gemcitabine into 3135.6 ul NS.

TABLE 72A
Study Design for CPGC-13-EF05

| | | | | | | Dose | | | |
|---|---|---|---|---|---|---|---|---|---|
| | | | | | | Route/ | | | |
| | | | | | | Dose | Dose | | |
| | | | | # of | Dose | regimen/ | Solution | *Dose | |
| Treatment | Test | Sequenc- | | animals/ | level | Dose | Conc. | Volume | |
| Group | Article | ing | Vehicle | Group | (mg/kg) | Duration | (mg/ml) | (ml) | Endpoints |
| 1 | 0.9% | | N/A | 6 | N/A | IV/QW ×21 | 0 | 0.1 | TV reach 10% |
| | Saline | | | | | days | | | BW or BW |
| | | | | | | | | | loss > 20% |
| 2 | 5F9 | | 0.9% | 6 | 7.5 | IV/QW ×21 | 1.4625 | 0.1 | TV reach 10% |
| | vcMMAE | | Saline | | | days | | | BW or BW |
| | immuno- | | | | | | | | loss > 20% |
| | conjugate | | | | | | | | |
| 3 | Gemcitabine | | 0.9% | 6 | 15.0 | IV/BIW ×21 | 2.925 | 0.1 | TV reach 10% |
| | | | Saline | | | days | | | BW or BW |
| | | | | | | | | | loss > 20% |
| 4 | Gemcitabine | | 0.9% | 6 | 20.0 | IV/BIW ×21 | 3.9 | 0.1 | TV reach 10% |
| | | | Saline | | | days | | | BW or BW |
| | | | | | | | | | loss > 20% |
| 5 | 5F9 | Simul- | 0.9% | 6 | 7.5, | IV/QW, | 1.4625, | 0.1 | TV reach 10% |
| | vcMMAE | taneous | Saline, | | 15.0 | IV/BIW ×21 | 2.925 | | BW or BW |
| | immuno- | | 0.9% | | | days | | | loss > 20% |
| | conjugate, | | Saline | | | | | | |
| | Gemcitabine | | | | | | | | |
| 6 | 5F9 | Simul- | 0.9% | 6 | 7.5, | IV/QW, | 1.4625, | 0.1 | TV reach 10% |
| | vcMMAE | taneous | Saline, | | 20.0 | IV/BIW ×21 | 3.9 | | BW or BW |
| | immuno- | | 0.9% | | | days | | | loss > 20% |
| | conjugate, | | Saline | | | | | | |
| | Gemcitabine | | | | | | | | |

TABLE 72B
Study Design for CPGC-13-EF08

| | | | | | | Dose | | | |
|---|---|---|---|---|---|---|---|---|---|
| | | | | | | Route/ | | | |
| | | | | | | Dose | Dose | | |
| | | | | # of | Dose | regimen/ | Solution | *Dose | |
| Treatment | Test | Sequenc- | | animals/ | level | Dose | Conc. | Volume | |
| Group | Article | ing | Vehicle | Group | (mg/kg) | Duration | (mg/ml) | (ml) | Endpoints |
| 1 | 0.9% | Simul- | N/A | 7 | N/A | IV/QW ×21 | 0 | 0.1 | TV reach 10% |
| | Saline, | taneous | | | | days | | | BW and |
| | 0.5% | | | | | | | | BWL .20% |
| | HPMC + | | | | | | | | |
| | 0.2% | | | | | | | | |
| | TWEEN | | | | | | | | |
| | 80 | | | | | | | | |
| 2 | MLN0264 | | 0.9% | 7 | 7.5 | IV/QW ×21 | 1.478 | 0.1 | TV reach 10% |
| | | | Saline | | | days | | | BW and |
| | | | | | | | | | BWL .20% |
| 3 | Gemcitabine | | 0.9% | 7 | 15.0 | IV/BIW ×21 | 2.955 | 0.1 | TV reach 10% |
| | | | Saline | | | days | | | BW and |
| | | | | | | | | | BWL .20% |
| 4 | Gemcitabine | | 0.9% | 7 | 15.0 | IV/Q2 | 2.955 | 0.1 | TV reach 10% |
| | | | Saline | | | D ×2/ | | | BW and |
| | | | | | | week ×21 | | | BWL .20% |
| | | | | | | days | | | |
| 5 | MLN0264, | Simul- | 0.9% | 7 | 7.5, | IV/QW, | 2.955, | 0.2 | TV reach 10% |
| | Gemcitabine | taneous | Saline, | | 15.0 | IV/BIW ×21 | 1.478 | | BW and |
| | | | 0.9% | | | days | | | BWL .20% |
| | | | Saline | | | | | | |
| 6 | MLN0264, | Simul- | 0.9% | 7 | 7.5, | IV/QW, | 2.955, | 0.2 | TV reach 10% |
| | Gemcitabine | taneous | Saline, | | 15.0 | IV/Q2 | 1.478 | | BW and |
| | | | 0.9% | | | D ×2/ | | | BWL .20% |
| | | | Saline | | | week ×21 | | | |
| | | | | | | days | | | |

The endpoints for each treatment group was either a) tumor volume reaches 10% body weight; or b) body weight loss of >20%. The projected tumor volume at the study initiation date was 200 mm³.

Day D0 was the first day of treatment. Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 27-30 gauge, ½-¾ inch in length. Animals were dosed based on an average body weight of 19.7 grams.

Tumor volume measurements were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD). TGI was assessed on an arbitrary day (when the maximum tumor volume (MTV of the control treatment group reaches the maximum allowable tumor volume), using the following formula:

TGI=100−[MTV treated/MTV control]×100.

Animals were treated when the tumor volume reached ˜230 mm³.

TGD was assessed by calculating T−C, where T=mean time (in days) for treatment group tumors to reach a predetermined size (e.g., 1,000 mm³), and where C=mean time (in days) for the control group tumors to reach a predetermined size.

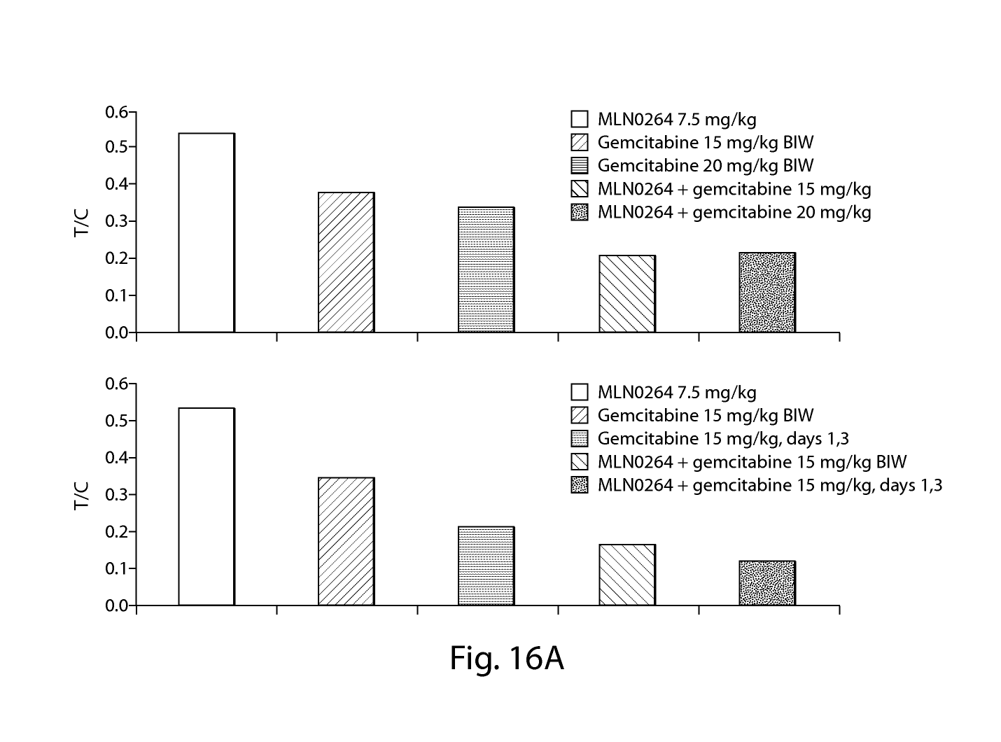

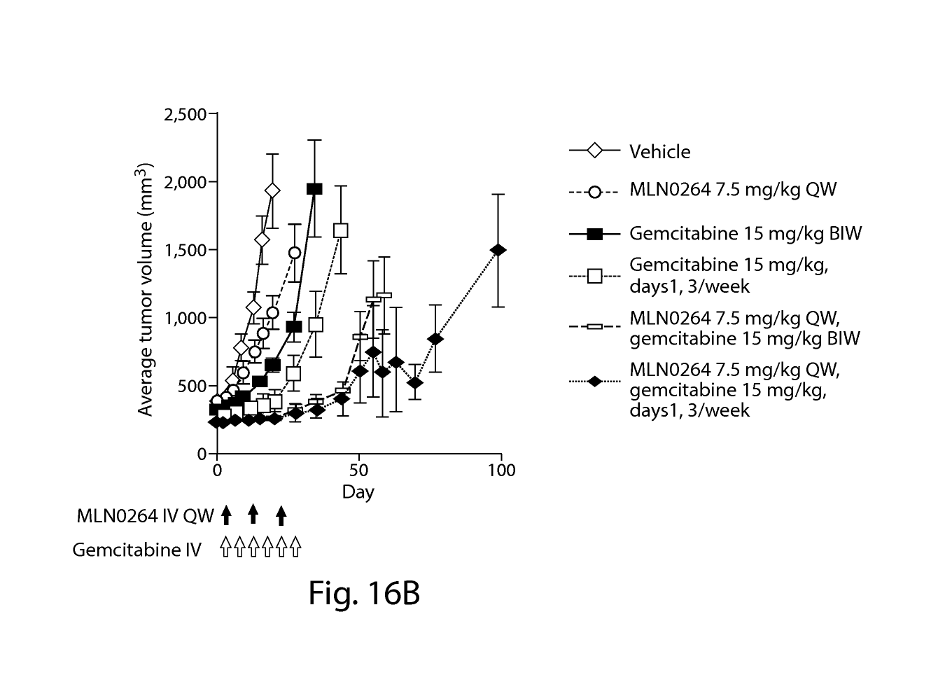

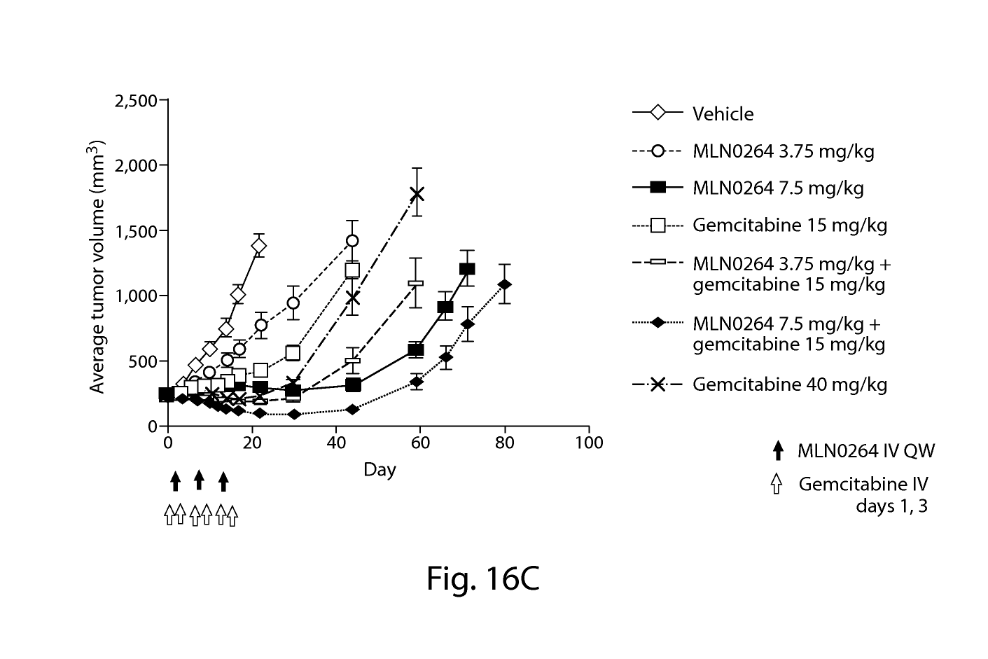

A comparison of the results from the various dosing schedules and concentrations tested in the PHTX-249 Pa model is shown in FIG. 16A. Average tumor volume curves for the various treatment groups in the PHTX-249 Pa model are shown in FIG. 16B. Notably, 3 of the 7 animals had tumor regressions with the combination of 5F9 vcMMAE immunoconjugate plus gemcitabine 15 mg/kg on days 1 and 3, whereas no tumor regressions were seen with single-agent gemcitabine or single agent immunoconjugate at 7.5 mg/kg. Respective tumor growth delay was 70.5 and 17 days. All doses were well tolerated.

Longitudinal analyses of the CPGC-13-EF05 and CPGC-13-EF08 studies using mixed effects linear regression models were performed using the methods as described in Example 7 to determine, in part, whether the combined anti-tumor efficacy of the 5F9 vcMMAE immunonjugate and gemcitabine was additive or synergistic. Tables 72A and 72B are the annotation table for group symbols. Tables 73A and 73B list the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Tables 74A and 74B list the synergy analysis. Statistically significant negative synergy scores indicate a synergistic combination (“Syn.”). Statistically significant positive synergys cores indicate a sub-additive combination (“Sub-add”) when the combination performs better (i.e., has a lower AUC) than the best performing single agent. Statistically significant positive synergy scores indicate an antagonistic combination (“Antag.”) when the combination performs worse than the best performing single agent. Scores that are not statistically significant should be considered additive (“Add.”).

TABLE 72A
Annotation Table for CPGC-13-EF05 Study

| | Group | Notation |
|---|---|---|
| 1 | vehicle/0.9percentSaline | V |
| 2 | MLN02647.5mg/kgQw/264 | A |
| 3 | Gemcitabine15mg/kgBiwi.p./Gemcitabine | B |
| 4 | Gemcitabine20mg/kgBiwi.p./Gemcitabine | C |
| 5 | MLN02647.5mg/kgQwGemcitabine15mg/kgBiw/Gemcitabine + 264 | D |
| 6 | MLN02647.5mg/kgGemcitabine20mg/kgBIW/Gemcitabine + 264 | E |

TABLE 72B
Annotation Table for the CPGC-13-EF08 Study

| | Group | Notation |
|---|---|---|
| 1 | vehicle/0.5percentHPMC + 0.2percentTWEEN80 + 0.9percentSaline | V |
| 2 | MLN02647.5mg/kgQW/264 | A |
| 5 | Gemcitabine15mg/kgBIW/Gemcitabine | D |
| 6 | Gemcitabine15mg/kgD1,D3/week/Gemcitabine | E |
| 7 | MLN02647.5mg/kgQWGemctibine15mg/kgBiw/Gemcitabine + 264 | F |
| 8 | MLN02647.5mg/kgQWGemcitabine15mg/kgD1,D3/week/Gemcitabine + 264 | G |

TABLE 73A
Results of Pairwise Comparisons for the CPGC-13-EF05 Study

| | Reference | Treated | dAUC | P. Value |
|---|---|---|---|---|
| 1 | V | A | 26.4 | 0.013 |
| 2 | V | B | 46.0 | 0.001 |
| 3 | V | C | 46.7 | <0.001 |
| 4 | V | D | 70.1 | <0.001 |
| 5 | V | E | 66.9 | <0.001 |

TABLE 73B
Results of Pairwise Comparisons for the CPGC-13-EF08 Study

| | Reference | Treated | dAUC | P. Value |
|---|---|---|---|---|
| 1 | V | A | 28.9 | <0.001 |
| 4 | V | D | 52.4 | <0.001 |
| 5 | V | E | 76.9 | <0.001 |
| 6 | V | F | 82.1 | <0.001 |
| 7 | V | G | 97.0 | <0.001 |

TABLE 74A
Results of Combination Analysis the CPGC-13-EF05 Study

| | Score | SEM | P. Value | Assess |
|---|---|---|---|---|
| D | 2.9 | 15.9 | 0.858 | Add. |
| E | 4.0 | 13.0 | 0.764 | Add. |

TABLE 74B
Results of Combination Analysis the CPGC-13-EF08 Study

| | Score | SEM | P. Value | Assess |
|---|---|---|---|---|
| F | −0.4 | 0.2 | 0.968 | Add. |
| G | 8.1 | 10.7 | 0.457 | Add. |

As shown in Tables 74A and 74B additive activity was seen across all combination treatment groups.

Antitumor Activity of 5F9 vcMMAE Immunoconjugate and Gemcitabine Administered Intravenously in Female CB-17 SCID Mice Bearing PHTX-215 Pa s.c. Xenografts (Study Nos. CPGC-13-EF10)

The objective of this study was to determine synergy and additive anti-tumor activity of MLN0264 at 3.75 and 7.5 mg/kg on QW schedule combination with Gemcitabine at 15 mg/kg on day 1, 3 schedule in PHTX-215 Pa s.c xenograft in CB17 SCID F mice. Dose formulations were prepared in accordance with Table 75. The study designs are shown in Table 76.

TABLE 75
Preparation of Dose Formulations

| | | amt (mg | Dosing Solution | | |
|---|---|---|---|---|---|
| | | powder) | concentration | Vehi- | |
| Agent | Dose | ul liquid | (mg/mL) | cle | Ab:drug |
| 5F9 vcMMAE | 3.75 | | 0.7275 | 0.9% | 1:4 |
| immuno- | mg/kg | | | Saline | |
| conjugate | | | | | |
| 5F9 vcMMAE | 7.5 | 501.1 ul | 1.455 | 0.9% | 1:4 |
| immuno- | mg/kg | | | Saline | |
| conjugate | | | | | |
| Gemcitabine | 15.0 | 183.8 ul | 2.91 | 0.9% | |
| | mg/kg | | | Saline | |
| Gemcitabine | 20.0 | | 3.88 | 0.9% | |
| | mg/kg | | | Saline | |
| ML00687217- | 40.0 | 408.4 ul | 7.76 | 0.9% | |
| 730-C | mg/kg | | | Saline | |
| (Gemcitabine) | | | | | |

TABLE 76
CPGC-13-EF10 Study Design

| | | | | | | Dose | | | |
|---|---|---|---|---|---|---|---|---|---|
| | | | | | | Route/ | | | |
| | | | | | | Dose | Dose | | |
| | | | | # of | Dose | regimen/ | Solution | *Dose | |
| Treatment | Test | Sequenc- | | animals/ | level | Dose | Conc. | Volume | |
| Group | Article | ing | Vehicle | Group | (mg/kg) | Duration | (mg/ml) | (ml) | Endpoints |
| 1 | 20% | Simul- | N/A | 8 | N/A | SC/BIW, | 0 | 0.1, | TV reach |
| | HPbCD, | taneous | | | | IV/QW ×21 | | 0.1 | 10% BW or |
| | 0.9% | | | | | days | | | BWL > 20% |
| | Saline | | | | | | | | |
| 2 | MLN0264 | | 0.9% | 8 | 3.75 | IV/QW ×21 | 0.7275 | 0.1 | TV reach |
| | | | Saline | | | days | | | 10% BW or |
| | | | | | | | | | BWL > 20% |
| 3 | MLN0264 | | 0.9% | 8 | 7.5 | IV/QW ×21 | 1.455 | 0.1 | TV reach |
| | | | Saline | | | days | | | 10% BW or |
| | | | | | | | | | BWL > 20% |
| 4 | Gemcitabine | | 0.9% | 8 | 15.0 | IV/Q1 | 2.91 | 0.1 | TV reach |
| | | | Saline | | | D ×2/ | | | 10% BW or |
| | | | | | | week ×21 | | | BWL > 20% |
| | | | | | | days | | | |
| 5 | MLN0264, | Simul- | 0.9% | 8 | 3.75, | IV/QW, | 0.7275, | 0.1, | TV reach |
| | Gemcitabine | taneous | Saline, | | 15.0 | IV/Q1 | 2.91 | 0.1 | 10% BW or |
| | | | 0.9% | | | D ×2/ | | | BWL > 20% |
| | | | Saline | | | week ×21 | | | |
| | | | | | | days | | | |
| 6 | MLN0264, | Simul- | 0.9% | 8 | 7.5, | IV/QW, | 1.455, | 0.1, | TV reach |
| | Gemcitabine | taneous | Saline, | | 15.0 | IV/Q1 | 2.91 | 0.1 | 10% BW or |
| | | | 0.9% | | | D ×2/ | | | BWL > 20% |
| | | | Saline | | | week ×21 | | | |
| | | | | | | days | | | |
| 7 | Gemcitabine | | 0.9% | 8 | 20.0 | IV/BIW ×21 | | | TV reach |
| | | | Saline | | | days | 3.88 | 0.1 | 10% BW or |
| | | | | | | | | | BWL > 20% |

The endpoints for each treatment group was either a) tumor volume reaches 10% body weight; or b) body weight loss of >20%. The projected tumor volume at the study initiation date was 200 mm³.

Day D0 was the first day of treatment. Not greater than one day prior to dosing, all animals were weighed to the nearest gram and grouped. Dosing solutions were vortexed prior to each administration to ensure correct delivery of compound. Animals received approximately 0.1 ml doses administered I.V. using a 1 cc syringe, 27-30 gauge, ½-¾ inch in length. Animals were dosed based on an average body weight of 19.4 grams.

Tumor volume measurements were obtained twice per week using Vernier calipers (0.01 mm). Tumor volume was calculated using the formula:

V=W²×L/2

(V=tumor volume, W=width measured along the short axis of the tumor, L=length measured along the short axis of the tumor). Body weight measurements were also taken twice per week using a Mettler scale (0.1 gm).

Efficacy was evaluated in terms of % tumor growth inhibition (TGI) and tumor growth delay (TGD). TGI was assessed on an arbitrary day (when the maximum tumor volume (MTV of the control treatment group reaches the maximum allowable tumor volume), using the following formula:

TGI=100−[MTV treated/MTV control]×100.

Animals were treated when the tumor volume reached ˜230 mm³.

TGD was assessed by calculating T−C, where T=mean time (in days) for treatment group tumors to reach a predetermined size (e.g., 1,000 mm³), and where C=mean time (in days) for the control group tumors to reach a predetermined size.

Average tumor volume curves for the various treatment groups in the PHTX-215 Pa model are shown in FIG. 16C. As shown in FIG. 16C, the 5F9 vcMMAE immunoconjugate in combination with gemcitabine also resulted in enhanced TGI and tumor regressions in the PHTX-215 model. Notably, all 8 animals had tumor regressions with the combination of the immunoconjugate plus gemcitabine 15 mg/kg on days 1 and 3 (TGI 93%), whereas no tumor regressions were seen with single agent gemcitabine (TGI 69%). Respective tumor growth delay was 60.4 and 22.7 days. All doses were well tolerated.

Longitudinal analyses of the CPGC-13-EF10 study using mixed effects linear regression models were performed using the methods as described in Example 7 to determine, in part, whether the combined anti-tumor efficacy of the 5F9 vcMMAE immunonjugate and gemcitabine was additive or synergistic. Table 77A the annotation table for group symbols. Table 77B lists the results of the pairwise comparisons. The dAUC is the percent decrease observed in the treatment AUC relative to that of the reference group. A negative dAUC is interpreted as an increase in AUC relative to the reference. It is important to evaluate not only the P value, but also the dAUC. Comparisons with significant P values, but small dAUC values, may not be of interest.

Table 77C lists the synergy analysis. Statistically significant negative synergy scores indicate a synergistic combination (“Syn.”). Statistically significant positive synergys cores indicate a sub-additive combination (“Sub-add”) when the combination performs better (i.e., has a lower AUC) than the best performing single agent. Statistically significant positive synergy scores indicate an antagonistic combination (“Antag.”) when the combination performs worse than the best performing single agent. Scores that are not statistically significant should be considered additive (“Add.”).

TABLE 77A
Annotation Table

| | Group | Notation |
|---|---|---|
| 1 | vehicle/0.9percentSaline + 20percentHPbCD | V |
| 2 | MLN02643.75mg/kgQW/264 | A |
| 3 | MLN02647.5mg/kgQW/264 | B |
| 4 | Gemcitabine15mg/kgD1,D3/weekx3/Gemcitabine | C |
| 5 | MLN02643.75mg/kgQWGem15mg/kgD1,D3/wx3/Gemcitabine + 264 | D |
| 6 | MLN02647.5mg/kgQWGem15mg/kgD1,D3/wx3/Gemcitabine + 264 | E |
| 7 | Gemcitabine20mg/kgBIW/Gemcitabine | F |

TABLE 77B
Pairwise Comparisons

| | Reference | Treated | dAUC | P. Value |
|---|---|---|---|---|
| 1 | V | A | 36.3 | <0.001 |
| 2 | V | B | 75.1 | <0.001 |
| 3 | V | C | 61.0 | <0.001 |
| 4 | V | D | 112.1 | <0.001 |
| 5 | V | E | 144.0 | <0.001 |
| 6 | V | F | 65.2 | <0.001 |
| 7 | V | G | 30.5 | <0.001 |

TABLE 77C
Synergy Analysis

| | Score | SEM | P. Value | Assess |
|---|---|---|---|---|
| D | −14.0 | 11.4 | 0.233 | Add. |
| E | −7.9 | 12.4 | 0.533 | Add. |

As shown in Table 77, additive activity was seen across the combination treatment groups.

In summary, these data indicate that the 5F9 vcMMAE immunoconjugate demonstrates antitumor activity in GCC-expressing pancreatic cancer xenograft models. The immunoconjugate demonstrates enhanced antitumor activity in combination with gemcitabine in GCC-expressing pancreatic cancer xenograft models compared with either single agent alone. These data suggest that the immunoconjugate, as a single agent, and in combination with gemcitabine, may be suitable for clinical investigation as a potential therapy for patients with pancreatic cancer.

While this invention has been shown and described with references to provided embodiments thereof, it will be understood by those skilled in the art that various changes in form and details may be made therein without departing from the scope of the invention encompassed by the appended claims.

1. A method of treating a gastrointestinal cancer, said method comprising administering to a patient in need of such treatment an immunoconjugate of Formula (I-5): 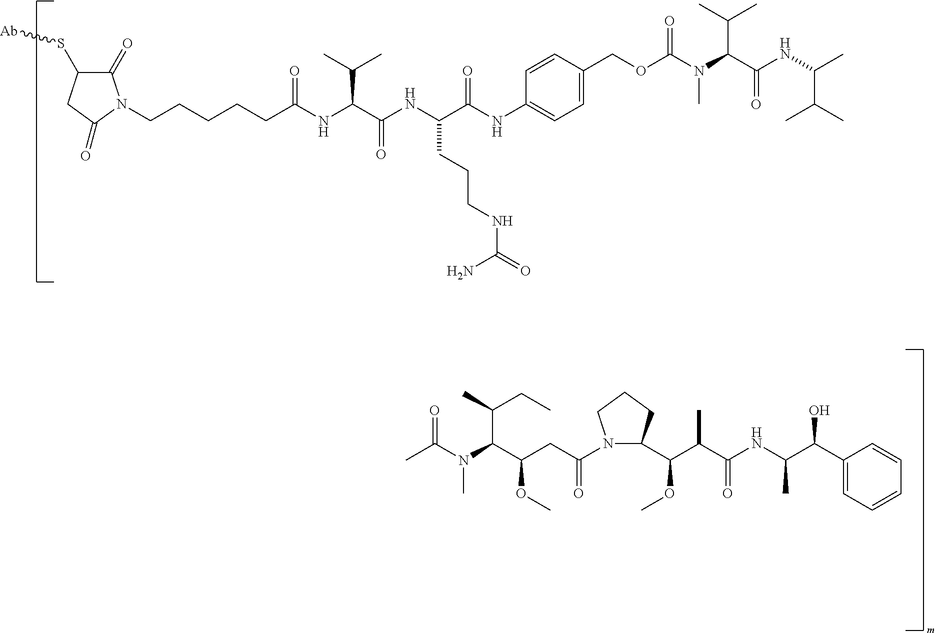
or a pharmaceutically acceptable salt thereof, wherein Ab is an anti-GCC antibody molecule, or antigen binding fragment thereof, and wherein m is an integer from 1-8; in combination with a DNA damaging agent selected from the group consisting of: a topoisomerase I inhibitor, a topoisomerase II inhibitor, an alkylating agent, an alkylating-like agent, an anthracycline, a DNA intercalator, a DNA minor groove-binding agent, and an antimetabolite, wherein the amounts of the immunoconjugate and the DNA damaging agent are therapeutically effective when used in combination.

2. The method of claim 1, wherein the anti-GCC antibody molecule comprises:
a) three heavy chain complementarity determining regions (CDRs) comprising the following amino acid sequences:
VH CDR1
(SEQ ID NO: 25)
GYYWS;
VH CDR2
(SEQ ID NO: 26)
EINHRGNTNDNPSLKS;
and
VH CDR3
(SEQ ID NO: 27)
ERGYTYGNFDH;
and
b) three light chain CDRs comprising the following amino acid sequences:
VL CDR1
(SEQ ID NO: 28)
RASQSVSRNLA;
VL CDR2
(SEQ ID NO: 29)
GASTRAT;
and
VL CDR3
(SEQ ID NO: 30)
QQYKTWPRT.

3. The method of claim 1, wherein m is from 3 to 5.

4. The method of claim 3 wherein m is about 4.

5. The method of claim 1, wherein the gastrointestinal cancer is a GCC-expressing cancer.

6. The method of claim 1, wherein the gastrointestinal cancer is resistant to the activity of the immunoconjugate when administered as a single agent.

7. The method of claim 1, wherein the gastrointestinal cancer is selected from the group consisting of: colorectal cancer, gastric cancer, pancreatic cancer and esophageal cancer, or metastases thereof.

8-9. (canceled)

10. The method of claim 1, wherein the DNA damaging agent is:
a topoisomerase I inhibitor selected from the group consisting of irinotecan, topotecan, and camptothecin,
b) an alkylating-like agent selected from the group consisting of cisplatin, oxaliplatin, carboplatin, nedaplatin, satraplatin and triplatin, or
c) an antimetabolite selected from the group consisting of: fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, and pemetrexed.

11-14. (canceled)

15. The method of claim 1, wherein the gastrointestinal cancer is primary or metastatic colorectal cancer and the DNA damaging agent is irinotecan, cisplatin, or 5-FU.

16. (canceled)

17. The method of claim 1, wherein the DNA damaging agent is gemcitabine.

18-20. (canceled)

21. The method of claim 1, wherein the immunoconjugate and the DNA damaging agent are concomitantly administered.

22. The method of claim 1, wherein the immunoconjugate and the DNA damaging agent are administered sequentially.

23. The method of claim 1, wherein the immunoconjugate and the DNA damaging agent are comprised in separate formulations.

24. The method of claim 1, wherein the anti-GCC antibody molecule is a monoclonal antibody or antigen binding fragment thereof.

25. The method of claim 1, wherein the anti-GCC antibody molecule is an IgG1 antibody.

26. The method of claim 1, wherein the anti-GCC antibody molecule further comprises human or human-derived light and heavy variable region frameworks.

27-81. (canceled)

82. A kit comprising an immunoconjugate of Formula (I-5): 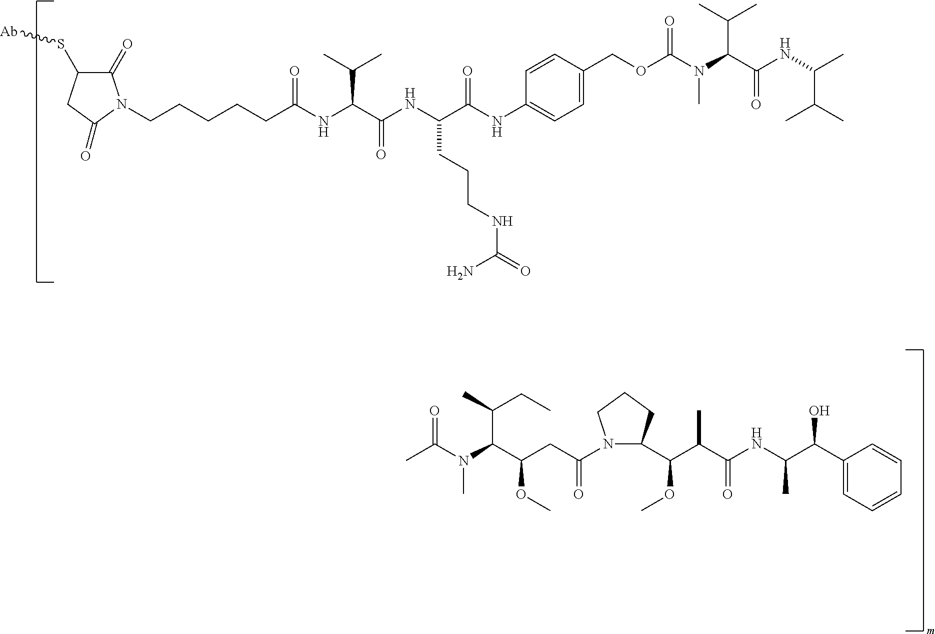
or a pharmaceutically acceptable salt thereof, wherein Ab is an anti-GCC antibody comprising:
a) three heavy chain complementarity determining regions (CDRs) comprising the following amino acid sequences:
VH CDR1
(SEQ ID NO: 25)
GYYWS;
VH CDR2
(SEQ ID NO: 26)
EINHRGNTNDNPSLKS;
and
VH CDR3
(SEQ ID NO: 27)
ERGYTYGNFDH;
and
b) three light chain CDRs comprising the following amino acid sequences:
VL CDR1
(SEQ ID NO: 28)
RASQSVSRNLA;
VL CDR2
(SEQ ID NO: 29)
GASTRAT;
and
VL CDR3
(SEQ ID NO: 30)
QQYKTWPRT.
or antigen binding fragment thereof, and wherein m is about 4; and instructions for administering the immunoconjugate in combination with a DNA damaging agent for the treatment of a gastrointestinal cancer.

83-84. (canceled)

85. The kit of claim 82, wherein the gastrointestinal cancer is selected from the group consisting of: colorectal cancer, gastric cancer, pancreatic cancer and esophageal cancer, or metastases thereof.

86. The kit of claim 82, further comprising a DNA damaging agent selected from the group consisting of: a topoisomerase I inhibitor, a topoisomerase II inhibitor, an alkylating agent, an alkylating-like agent, an anthracycline, a DNA intercalator, a DNA minor groove alkylating agent, and an antimetabolite.

87. (canceled)

88. The kit of claim 86, wherein the DNA damaging agent is
a) topoisomerase I inhibitor selected from the group consisting of irinotecan, topotecan, and camptothecin,
b) an alkylating-like agent selected from the group consisting of cisplatin, oxaliplatin, carboplatin, nedaplatin, satraplatin and triplatin, or
c) an antimetabolite selected from the group consisting of: fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, and pemetrexed.

89-92. (canceled)

93. The kit of claim 88, wherein the DNA damaging agent is irinotecan, cisplatin, or 5-FU and the gastrointestinal cancer is primary or metastatic colorectal cancer.

94-95. (canceled)

96. The kit of claim 88, wherein the DNA damaging agent is gemcitabine and the gastrointestinal cancer is primary or metastatic pancreatic cancer.

97-98. (canceled)

99. The method of claim 1, wherein administration of the immunoconjugate and the DNA damaging agent results in synergistic efficacy.

100-103. (canceled)

104. The method of claim 1, wherein the cancer has relatively high or moderate GCC antigen density.

105-108. (canceled)

109. The method of claim 1, wherein the cancer has low GCC antigen density.

110-113. (canceled)

114. The method of claim 1, further comprising a step of determining the GCC antigen density of the cancer.

115-118. (canceled)

119. The method of claim 1, further comprising a step of determining the cancer's sensitivity to the immunoconjugate alone.

120-123. (canceled)
